SEQUENCE LISTING (155 sequences)

SEQ ID NO: 1 - DNA, 7788 bp, Homo sapiens
atggcttccctgtttcatcagcttcagatcctggtctggaaaaattggctaggtgtaaaaaggcagccgctttggacacttgtcttgatcttatggccagtcattattttcataattttggctattactcggaccaaatttcctccaactgcaaaaccaacttgttacctcgcacctcgaaaccttcctagtactggattctttccattcctgcagaccctactctgtgacacagactctaaatgcaaagacacaccctatggcccacaagatctgcttcgtaggaaaggaattgatgatgcactatttaaagacagtgagattctgagaaagtcatccaacctggataaggacagcagtttatcattccagagcacccaagttccagaaagaaggcatgcatcactagccacagtatttcccagtccaagttctgatttggaaatccccggaacatatactttcaatggcagtcaagtgctcgcacgaattcttggcttggaaaagctgttaaagcaaaattcaacttcagaagatatacgaagagaactatgtgacagctattcaggatacattgtggatgatgccttctcttggacctttctaggaagaaatgtttttaacaaattttgcctttctaacatgacccttttagagtcttctctccaagaactaaacaaacagttctcccagctatccagtgaccccaacaatcagaagatagtgtttcaggaaatagtcagaatgctgtctttcttctcacaagtgcaagagcagaaagctgtgtggcagcttctgtctagttttccaaatgtgtttcagaatgacacatcactaagcaatctatttgatgttcttcgaaaggcaaacagtgtgctgctggttgtgcagaaggtttatccacgttttgcaactaacgaaggtttcagaaccctccagaagtctgttaaacatctgctgtacactctggactccccagctcaaggtgactccgataatataacgcatgtgtggaatgaggatgatggacagaccttatctccaagcagtctggctgcacagctcctaattctggaaaactttgaagatgccctcttaaatatatcagcaaatagtccttatattccttacttggcatgtgtgagaaatgtgactgacagtttggccagaggttcaccagaaaatctaagactcctgcagtccacaatacgatttaaaaaatcttttcttcgcaatggttcctatgaagattactttcctccagttcctgaagtcctaaaatcaaaactgtctcaacttcgaaacttgaccgaacttctttgtgaatctgaaactttcagtttgatagagaagtcatgccagctctctgatatgagctttgggagcctgtgtgaagaaagtgagtttgatctgcaactcctcgaagcggcagagctgggcaccgaaatagcagccagcttactgtaccatgacaatgtcatatctaaaaaagtgagagatttgctgactggagatccaagcaaaattaatttaaatatggatcagtttctagaacaggcactgcaaatgaattacttggaaaatatcactcagttaataccgatcatagaagccatgctgcatgtcaataacagtgcagatgcttctgaaaagccaggtcagttactagaaatgtttaaaaatgttgaagagctgaaagaagatttaaggagaacaacaggaatgtccaacaggactattgacaagttgctggccattcccatccctgataatagagctgagattatttctcaggtgttctggctgcattcctgtgatactaatatcaccactcccaaactagaagatgcaatgaaagaattctgcaacctgtctctttcagagagatcccggcagtcttacctcatcggactcacccttctgcactacttaaacatttacaacttcacatacaaggtgtttttcccgaggaaagatcaaaagccagtagaaaagatgatggagctcttcataagactaaaagagattctcaatcagatggcttctggcacacatccgctgctagacaaaatgagatccctgaagcaaatgcatctgcccagaagtgttccattaacacaggcaatgtacagaagcaaccgaatgaacacaccacaaggatcatttagcaccatctcccaagcattatgttctcaaggaattaccactgaatatttaactgccatgctgccctcttcccagaggccaaaaggcaaccacaccaaggattttttgacttataaattaactaaagagcaaattgcttcaaaatatggaattcccataaattccacaccattttgcttctccctttataaagacatcattaacatgcccgctggacctgtgatttgggctttcttgaaacctatgttgttgggaagaattttgtatgcaccatataacccagtcacaaaggcaataatggaaaagtccaatgtaactctgagacagctggcggaattaagagaaaaatctcaagagtggatggataagtcgccacttttcatgaattccttccatctgttaaaccaggcaattccaatgctccagaatactctaaggaacccttttgtgcaagtttttgtaaagttctccgtgggactcgatgctgttgaactattgaaacagatagatgaactcgatattctaagactgaaattagagaacaacattgacatcatcgatcagcttaacacactatcttccctgacagtaaatatttcctcttgtgtattatatgaccgtattcaggcagcaaaaaccatagatgaaatggagagagaggctaaaaggctctacaaaagcaacgaactctttggaagtgttatttttaagcttccttctaacagaagctggcacagaggctatgactctggaaatgtctttcttcctcctgtcataaaatataccatccggatgagtctcaagaccgcacagaccacaagaagcctaagaaccaagatttgggctccagggccacacaattctccatcacacaaccagatctatggcagggcttttatttatttacaggatagtattgaaagagcaatcattgaattgcaaactggaaggaactcccaggaaatagcagtccaggttcaagcaattccttatccctgcttcatgaaagacaacttcctaaccagtgtctcttattctcttccaattgtgcttatggttgcctgggttgtatttatagctgcctttgtaaaaaagcttgtctatgagaaagacctccggcttcatgagtacatgaagatgatgggtgtgaactcctgcagccatttctttgcctggcttatagagagtgttggatttttactggttaccatcgtgatcctcatcattatactcaagtttggcaatattcttcctaaaacaaatgggttcattttgttcctgtatttttcggactacagcttctcggttattgccatgagctatcttatcagtgtcttcttcaacaacaccaacattgcagctctgatcggaagcctcatctacatcattgccttctttccatttattgttctggttacagtggagaatgagttgagctatgtattgaaagtgttcatgagcctgctgtccccaacagcattcagctatgcaagccaatacattgcacgatacgaagaacagggcattggtcttcagtgggaaaatatgtacacctccccggttcaggatgacaccacctcatttggctggctgtgctgtctaatcctagctgactctttcatttatttccttattgcttggtatgtcaggaatgtcttcccagggacatacggtatggcagctccctggtattttccaattcttccttcctattggaaggagcgatttgggtgtgcagaggtgaagcctgagaagagcaatggcctcatgtttactaacatcatgatgcagaacaccaacccatctgccagtcctgaatacatgttttcctctaacatcgagcctgaacctaaagatctcacagtcggggttgccctgcatggggtcacaaagatctatggctcaaaagttgctgttgataacctcaatctgaacttttatgaagggcatattacttcattgctggggcccaatggagctgggaaaactactaccatttccatgttaactgggctgtttggggcctcagcaggcaccatttttgtatatggaaaagatatcaaaacagacctacacacggtacggaagaacatgggagtctgtatgcagcacgacgtcttgttcagttacctcactactaaggagcaccttctcctatatggttccatcaaagttcctcactggactaaaaagcagctccacgaggaagtaaaaaggactttaaaagatactggactatatagccatcgtcataagagagttggaacactgtcaggaggcatgaagaggaagttatctatatccatagctctcattggtggatcaagggtagtaattttggatgaaccatctactggagttgacccatgttctcgccgaagtatatgggatgttatatccaagaacaaaactgccagaacaatcattctgtcaacgcaccacttggacgaggctgaagtgctgagtgaccgcatcgccttcctggagcagggtgggcttaggtgctgtgggtccccattttacctcaaggaagcctttggcgatgggtatcacctcacgcttaccaagaagaagagtccaaatttaaatgcaaatgcagtatgtgacaccatggccgtgacagcaatgatccaatcacatctccccgaagcctacctcaaggaggatattgggggagagcttgtttatgtacttcctccattcagcaccaaagtctcaggggcctacctgtcactcctacgggcactcgacaatggcatgggtgacctcaacatcgggtgctacggcatttcagataccaccgtggaggaggtctttctgaacttgaccaaagagtcacaaaaaaatagtgctatgagtcttgagcacttaacacaaaagaaaattgggaattccaatgccaatggcatctcaactcctgacgatttatctgtgagcagcagcaatttcacagacagagatgacaaaatcctgacaagaggagagaggctggatggctttggactgttgctgaagaagatcatggctatactcatcaagaggttccaccacacccgcaggaactggaaaggtctcattgctcaggttatcctccccatcgtctttgttaccactgccatgggccttggcacactgagaaattccagcaacagttatccagagattcagatctccccctctctttatggtacctccgaacagacagccttctatgctaattatcacccgagcacggaagcacttgtctcagcaatgtgggacttccctggaattgacaacatgtgtctgaacaccagtgatctacagtgtttaaacaaagacagtctggaaaaatggaacaccagtggagaacccatcactaattttggtgtttgctcctgctcagaaaatgtccaggaatgtcctaaatttaactattccccaccgcacagaagaacttactcatcccaggtaatttataacctcactgggcaacgagtggaaaattatcttatatcaactgcaaatgagtttgtccaaaaaagatatggaggttggagttttgggctgcctttgacaaaagaccttcgttttgatataacaggagtccctgccaatagaacacttgccaaggtatggtatgatccagaaggctatcactcccttccagcttacctcaacagcctgaataatttccttctgcgagttaacatgtcaaaatacgatgctgcccgacatggcatcatcatgtatagccatccttatccaggagtgcaagaccaagaacaagccacaatcagcagtttaatcgatattttagtggcactgtctatcttgatgggctactctgtcaccaccgccagctttgtcacctatgttgtaagggaacatcaaaccaaagccaaacagttgcagcacatttcaggcattggcgtgacatgctactgggtaacaaacttcatttatgacatggttttctacttggtgcctgtagcgttttcaattggtatcattgcgattttcaaattacctgcattctacagtgaaaacaacctaggcgctgtatctctcctacttctcctgtttgggtatgcaacattttcctggatgtacttgctggctgggctcttccatgaaacaggaatggccttcatcacttacgtctgtgtcaacttgttttttggcattaattccattgtttccctgtcagtggtatactttctttccaaggaaaagcctaatgatccgactttagaacttatttctgaaaccctcaagcgcattttcctgattttcccacaattctgttttggctacggtttgattgaactttctcaacaacagtcggtcctagacttcttaaaagcatatggagtggaatacccaaatgaaacctttgagatgaataaactaggtgcaatgtttgtggctttggtttctcagggcaccatgtttttttccttgcgactcttaatcaacgaatccctgataaagaaactcaggcttttcttcagaaaatttaattcttcacatgtaagggagacaatagatgaggatgaagatgtgcgggctgagagattaagagttgagagtggtgcagctgaatttgacttggtccaactttattgtctcacaaagacctaccaacttatccacaaaaagattatagctgtaaacaacatcagcattgggatacctgctggagagtgttttgggcttcttggagtgaatggagcaggaaagaccactatattcaagatgctgacaggagacatcattccttcaagtggaaacattctgatcagaaataagaccggatctctgggtcacgttgattctcacagctcattagttggctactgtcctcaggaagatgccttagatgacctggtaactgtggaagaacatttgtatttctatgccagggtacatggaattccagaaaaggatattaaagaaactgttcataaactccttaggagacttcacctgatgcccttcaaggacagagctacctctatgtgcagttatggcacaaaaagaaaattatccactgcactggccttgatagggaaaccttccattctactgctggatgagccgagctctggcatggatccgaagtcgaaacggcacctctggaagatcatttcagaagaagtacagaacaaatgttccgtcatcctcacatctcacagcatggaagaatgtgaagctctctgtaccaggttggccattatggtgaatggaaagtttcaatgtattggatctttgcagcacataaagagcaggtttggacgaggatttactgtcaaagttcacttgaagaataacaaagtgaccatggagaccctcacaaagttcatgcagctgcactttccaaaaacatacttaaaagatcagcacctcagcatgctagagtatcatgtaccagtcacagcaggaggagtcgcaaacatttttgatctgctggaaaccaacaagactgctttaaatattacaaatttcttagtgagtcagaccactctggaagaggttttcatcaactttgccaaagaccagaagtcctatgaaactgctgataccagcagccaaggttccactataagtgttgactcacaagatgaccagatggagtcttaa

SEQ ID NO: 2 - DNA, 7788 bp, Artificial Sequence
  Synthetic
atggccagcctgtttcaccagctgcagatcctcgtgtggaagaactggctgggcgtgaagagacagcctctgtggaccctggtgctgatcctgtggcctgtgatcatcttcatcatcctggccatcaccaggaccaagtttccacctaccgccaagcctacctgctatctggcccctagaaacctgccttctaccggattcttcccattcctgcagaccctgctgtgcgacaccgacagcaagtgcaaggacacaccatacggccctcaggacctgctgagaagaaagggcattgacgacgccctgttcaaggacagcgagatcctgagaaagagcagcaacctggacaaggactccagcctgagcttccagagcacacaggtgccagaaagaaggcacgcctctctggccaccgtgtttccatctccaagctccgacctggaaatccccggcacctacaccttcaatggctctcaggtgctggccagaatcctgggcctcgagaaactgctgaagcagaacagcaccagcgaggacattcggagagagctgtgcgatagctacagcggctacatcgtggacgacgccttcagctggaccttcctgggcagaaacgtgttcaacaagttctgcctgagcaacatgaccctgctcgagagcagcctgcaagagctgaacaagcagttcagccagctgagcagcgaccccaacaaccagaaaatcgtgttccaagagatcgtgcggatgctgagcttcttcagccaagtccaagagcagaaagccgtgtggcagctgctgtccagctttcccaacgtgttccagaacgacaccagcctgtccaacctgttcgacgtgctgagaaaggccaacagcgtgctgctggtggtgcagaaggtgtaccctagattcgccaccaacgagggcttcagaaccctgcagaaaagcgtgaagcacctcctgtacaccctggactctccagctcagggcgacagcgacaatatcacccacgtgtggaacgaggacgacggccagacactgagcccatcttctctggctgcccagctgctgattctggaaaacttcgaggacgccctgctgaacatcagcgctaacagcccttacatcccctacctggcctgcgtgcggaacgtgacagatagcctggctagaggcagccccgagaacctgagactgctgcagagcaccatccggttcaagaagtccttcctgcggaacggcagctacgaggactacttcccacctgtgcctgaggtgctgaagtccaagctgtcccagctgagaaacctgaccgagctgctgtgtgaaagcgagacattcagcctgatcgagaagtcctgccagctgtccgacatgagctttggcagcctgtgcgaggaaagcgagttcgacctgcaactgctggaagccgccgagctgggaacagaaattgccgccagcctgctgtaccacgacaacgtgatcagcaagaaagtgcgggatctgctgaccggcgatcccagcaagatcaacctgaatatggaccagttcctggaacaggccctgcagatgaactacctggaaaacatcacccagctgatccccatcatcgaggccatgctgcacgtgaacaatagcgccgatgccagcgagaagcctggacagctgctcgaaatgttcaagaacgtcgaggaactgaaagaggacctgcggaggaccaccggcatgagcaacagaaccatcgacaagctgctggctatccccattccagacaaccgggccgagatcatcagccaggtgttctggctgcactcctgcgacaccaacatcaccacacctaagctggaagatgccatgaaggagttctgcaacctgagcctgagcgagcggagcagacagagctatctgatcggcctgactctgctgcactacctgaacatctacaacttcacctacaaggtgttcttcccgcggaaggaccagaaacctgtggaaaagatgatggaactgttcatccggctgaaagaaatcctgaaccagatggccagcggcacacaccctctgctggacaagatgagatccctgaagcaaatgcatctgcccagaagcgtgcccctgacacaggccatgtaccggtccaacagaatgaacacccctcagggcagcttctccaccatctctcaggctctgtgtagccagggcatcaccaccgagtacctgaccgctatgctgcctagctctcagaggcccaagggcaaccacaccaaggactttctgacctacaagctgaccaaagagcagatcgcctctaagtacggcatcccgatcaacagcaccccattctgctttagcctgtacaaggatatcatcaacatgcctgctggccccgtgatctgggccttcctgaaacctatgctgctggggagaatcctgtacgccccttacaaccctgtgaccaaggccatcatggaaaagtccaacgtgaccctgaggcagctggccgagctgagagaaaagtcccaagagtggatggacaagagcccactgttcatgaatagcttccatctgctgaaccaggccattccaatgctgcagaataccctgaggaaccccttcgtgcaggttttcgtgaagttcagcgtcggcctggacgccgttgagctgctcaagcagatcgacgagctggacatcctgcggctgaagctcgagaacaacatcgacatcatcgaccagctgaacaccctgtctagcctgaccgtgaacatctccagctgcgtgctgtacgacagaatccaggccgccaagaccatcgacgaaatggaaagagaggccaagcggctgtacaagagcaacgagctgttcggcagcgtgatcttcaagctgccaagcaaccggtcctggcacaggggctatgatagcggcaatgtgttcctgcctccagtgatcaagtacaccatccgcatgagcctgaaaacagcccagaccaccagaagcctgcggaccaaaatttgggcccctggacctcacaacagccccagccacaatcagatctacggcagggccttcatctacctgcaagacagcatcgagcgggccatcattgagctgcagaccggcagaaacagccaagagattgccgtgcaggtccaggcaatcccctatccttgctttatgaaggacaacttcctgaccagcgtgtcctacagcctgcctatcgtgctgatggtcgcctgggtcgtgtttatcgccgccttcgtgaagaaactggtgtacgagaaggacctgaggctgcacgagtacatgaagatgatgggcgtgaacagctgcagccacttcttcgcttggctgatcgagtccgtgggcttcctgctggtcaccatcgtgatcctgatcatcattctgaagttcggcaacatcctgcctaagaccaacggcttcatcctgtttctgtacttcagcgactactccttctccgtgatcgccatgagctacctgatctccgtgttcttcaacaacaccaatatcgccgcactgatcggctccctgatctatattatcgctttcttccccttcatcgtgctcgtgaccgtggaaaacgagctgagctacgtgctgaaggtgttcatgtccctgctgagccccaccgcctttagctacgccagccagtatatcgccagatacgaggaacaaggcatcggcctgcagtgggagaacatgtacacaagccccgtgcaggacgataccaccagctttggctggctgtgttgcctgatcctggccgacagctttatctacttcctgatcgcttggtacgtccggaatgtgtttcctggcacctacggcatggctgccccttggtactttcccatcctgccaagctactggaaagaaagattcggctgcgccgaagtgaagcctgagaagtccaatggcctgatgtttaccaatatcatgatgcagaacacgaaccccagcgctagccccgagtatatgttctccagcaacatcgagcccgagcctaaggatctgacagtgggagttgctctgcacggcgtgaccaagatctatggcagcaaggtggccgtggacaatctgaacctgaatttctacgagggccacatcactagcctgctgggccctaatggcgccggaaagaccaccaccattagcatgctgactggcctgtttggcgccagcgccggcacaatctttgtgtacggcaaggacatcaagaccgacctgcacaccgtgcggaagaacatgggcgtgtgtatgcagcacgatgtgctgttttcttacctgaccacgaaagagcatctgctgctctacggctccatcaaggtgccccactggaccaagaagcagctccacgaggaagtgaagagaaccctgaaggacaccggcctgtacagccacagacacaagagagtgggcacactgagcggcggcatgaagagaaagctgagcatctctatcgccctgatcggcggctctagagtggtcatcctggatgagcctagcaccggcgtggacccttgctctagaagatccatctgggacgtgatctccaagaacaagacagcccggacaatcatcctgagcacccaccatctggatgaggccgaggtgctgtctgacaggatcgcctttctcgaacaaggcggcctgagatgttgcggcagccccttctatctgaaagaagccttcggcgacggctaccacctgacactgacaaagaagaagtccccaaacctgaacgccaacgccgtgtgcgataccatggccgtgacagccatgatccagagccatctgccagaggcctacctcaaagaggatatcggcggcgagctggtgtatgtgctgcctccattcagcaccaaagtgtccggcgcctacctgtctctgctgagagccctggataacggcatgggcgatctgaacatcggctgctacggcatcagcgacacaaccgtggaagaggtgttcctgaatctgacaaaagagagccagaaaaacagcgccatgagtctcgagcacctgactcagaagaagatcggcaactccaacgccaatggcatcagcacccctgacgatctgtccgtgtcctccagcaatttcaccgaccgggacgacaagatcctgacaagaggcgagagactggacggctttggcctgctgctgaagaaaatcatggccatcctgattaagcggttccaccacaccagacggaactggaagggactgatcgcccaagtgattctgcccatcgtgtttgtgaccaccgccatgggactgggcaccctgagaaacagctccaacagctaccccgagatccagatcagcccaagcctgtacggcacatctgagcagaccgccttctacgccaactatcaccctagcacagaggccctggtgtccgccatgtgggatttccccggcatcgacaacatgtgcctgaacaccagcgacctgcagtgtctgaacaaggattccctggaaaagtggaataccagcggcgagcccatcaccaactttggcgtgtgttcttgcagcgagaacgtgcaagagtgccccaagttcaactacagccctcctcacagacggacctacagcagccaagtgatctacaatctgaccggccagcgcgtggaaaattacctgatcagcacagccaacgagttcgtgcagaagagatacggcggctggtctttcggcctgccactgaccaaggacctcagattcgacatcacaggcgtgcccgccaatcggaccctggccaaagtttggtacgatcccgagggctaccactctctgcccgcctatctgaacagcctgaacaatttcctgctgcgggtcaacatgagcaaatacgacgccgccagacacggcatcatcatgtattctcacccctatccaggcgtgcaggatcaagagcaggccaccatttcttccctgatcgacatcctggtggccctgagcatcctgatgggctactctgtgacaaccgcctcctttgtgacctacgtcgtgcgggaacaccagacaaaagccaaacaactgcagcacatcagcggcatcggagtgacctgttactgggtcaccaatttcatctacgacatggtgttctacctggtgccagtggccttcagcatcggcatcattgccatttttaagctgccagccttctactccgagaacaatctgggcgccgtgtcactgcttctgctgctgtttggctacgccacctttagctggatgtacctgctggctggactgttccacgagacaggcatggcctttatcacatacgtgtgcgtgaacctgttcttcggcatcaactccatcgtgtccctgagcgtggtgtatttcctgtccaaagagaagcccaacgatcccacactggaactgatcagcgagactctgaagcggatctttctgatcttcccgcagttctgctttggatacggcctgattgagctgagccagcaacagagcgtgctggattttctgaaggcctacggcgtggaatatcccaacgaaaccttcgagatgaacaagctgggagccatgttcgtggctctggtgtctcagggaaccatgtttttttctctgcggctgctgatcaacgagtccctgatcaagaagctgcggctgttcttcagaaagttcaacagctcccatgtccgcgagacaatcgacgaggatgaggatgtgcgggccgagcgactgagagttgaatctggcgccgctgagttcgatctggtgcagctgtactgcctgaccaagacataccagctcatccacaagaagatcattgccgtcaacaatatctccatcggaatccctgccggcgagtgctttggactgcttggagttaatggcgctggcaaaacaaccatcttcaagatgctgacaggggacatcatccccagctccgggaatatcctgatccggaacaagaccggctctctgggccatgtggattcccactctagcctcgtgggctactgccctcaagaggacgctctggatgatctggtcaccgttgaggaacatctgtacttttacgccagagtgcacggaatccccgagaaagacatcaaagaaaccgtgcacaaactgctgcggcggctgcacctgatgcctttcaaagatagagccaccagcatgtgcagctacggcaccaagcggaagctgtctacagctctggccctgattggcaagcccagcatcctcctgctggacgagccatctagtggcatggaccccaagagcaagcggcatctgtggaagatcatctccgaagaggtgcagaacaagtgctccgtgattctgaccagccacagcatggaagagtgcgaggccctgtgtaccagactggccattatggtcaacggcaagtttcagtgcatcggaagcctgcagcatatcaagtccagattcggcagaggcttcaccgtgaaagtgcacctcaagaacaacaaagtgaccatggaaaccctcaccaagttcatgcaactgcacttccctaagacctaccttaaggaccagcatctgtccatgctggaatatcacgtgccagtgacagccggcggagtggccaatatctttgacctgctggaaacaaacaagacggccctgaatattaccaactttctggtgtcccagacgacccttgaggaagtgttcatcaacttcgccaaggaccaaaagagctacgagacagccgacacaagcagccagggaagcaccatctccgtggactctcaggacgaccagatggaaagctga

SEQ ID NO: 3 - DNA, 6834 bp, Homo sapiens
atgtttacctatataaaaattattacttcaggtgactccgataatataacgcatgtgtggaatgaggatgatggacagaccttatctccaagcagtctggctgcacagctcctaattctggaaaactttgaagatgccctcttaaatatatcagcaaatagtccttatattccttacttggcatgtgtgagaaatgtgactgacagtttggccagaggttcaccagaaaatctaagactcctgcagtccacaatacgatttaaaaaatcttttcttcgcaatggttcctatgaagattactttcctccagttcctgaagtcctaaaatcaaaactgtctcaacttcgaaacttgaccgaacttctttgtgaatctgaaactttcagtttgatagagaagtcatgccagctctctgatatgagctttgggagcctgtgtgaagaaagtgagtttgatctgcaactcctcgaagcggcagagctgggcaccgaaatagcagccagcttactgtaccatgacaatgtcatatctaaaaaagtgagagatttgctgactggagatccaagcaaaattaatttaaatatggatcagtttctagaacaggcactgcaaatgaattacttggaaaatatcactcagttaataccgatcatagaagccatgctgcatgtcaataacagtgcagatgcttctgaaaagccaggtcagttactagaaatgtttaaaaatgttgaagagctgaaagaagatttaaggagaacaacaggaatgtccaacaggactattgacaagttgctggccattcccatccctgataatagagctgagattatttctcaggtgttctggctgcattcctgtgatactaatatcaccactcccaaactagaagatgcaatgaaagaattctgcaacctgtctctttcagagagatcccggcagtcttacctcatcggactcacccttctgcactacttaaacatttacaacttcacatacaaggtgtttttcccgaggaaagatcaaaagccagtagaaaagatgatggagctcttcataagactaaaagagattctcaatcagatggcttctggcacacatccgctgctagacaaaatgagatccctgaagcaaatgcatctgcccagaagtgttccattaacacaggcaatgtacagaagcaaccgaatgaacacaccacaaggatcatttagcaccatctcccaagcattatgttctcaaggaattaccactgaatatttaactgccatgctgccctcttcccagaggccaaaaggcaaccacaccaaggattttttgacttataaattaactaaagagcaaattgcttcaaaatatggaattcccataaattccacaccattttgcttctccctttataaagacatcattaacatgcccgctggacctgtgatttgggctttcttgaaacctatgttgttgggaagaattttgtatgcaccatataacccagtcacaaaggcaataatggaaaagtccaatgtaactctgagacagctggcggaattaagagaaaaatctcaagagtggatggataagtcgccacttttcatgaattccttccatctgttaaaccaggcaattccaatgctccagaatactctaaggaacccttttgtgcaagtttttgtaaagttctccgtgggactcgatgctgttgaactattgaaacagatagatgaactcgatattctaagactgaaattagagaacaacattgacatcatcgatcagcttaacacactatcttccctgacagtaaatatttcctcttgtgtattatatgaccgtattcaggcagcaaaaaccatagatgaaatggagagagaggctaaaaggctctacaaaagcaacgaactctttggaagtgttatttttaagcttccttctaacagaagctggcacagaggctatgactctggaaatgtctttcttcctcctgtcataaaatataccatccggatgagtctcaagaccgcacagaccacaagaagcctaagaaccaagatttgggctccagggccacacaattctccatcacacaaccagatctatggcagggcttttatttatttacaggatagtattgaaagagcaatcattgaattgcaaactggaaggaactcccaggaaatagcagtccaggttcaagcaattccttatccctgcttcatgaaagacaacttcctaaccagtgtctcttattctcttccaattgtgcttatggttgcctgggttgtatttatagctgcctttgtaaaaaagcttgtctatgagaaagacctccggcttcatgagtacatgaagatgatgggtgtgaactcctgcagccatttctttgcctggcttatagagagtgttggatttttactggttaccatcgtgatcctcatcattatactcaagtttggcaatattcttcctaaaacaaatgggttcattttgttcctgtatttttcggactacagcttctcggttattgccatgagctatcttatcagtgtcttcttcaacaacaccaacattgcagctctgatcggaagcctcatctacatcattgccttctttccatttattgttctggttacagtggagaatgagttgagctatgtattgaaagtgttcatgagcctgctgtccccaacagcattcagctatgcaagccaatacattgcacgatacgaagaacagggcattggtcttcagtgggaaaatatgtacacctccccggttcaggatgacaccacctcatttggctggctgtgctgtctaatcctagctgactctttcatttatttccttattgcttggtatgtcaggaatgtcttcccagggacatacggtatggcagctccctggtattttccaattcttccttcctattggaaggagcgatttgggtgtgcagaggtgaagcctgagaagagcaatggcctcatgtttactaacatcatgatgcagaacaccaacccatctgccagtcctgaatacatgttttcctctaacatcgagcctgaacctaaagatctcacagtcggggttgccctgcatggggtcacaaagatctatggctcaaaagttgctgttgataacctcaatctgaacttttatgaagggcatattacttcattgctggggcccaatggagctgggaaaactactaccatttccatgttaactgggctgtttggggcctcagcaggcaccatttttgtatatggaaaagatatcaaaacagacctacacacggtacggaagaacatgggagtctgtatgcagcacgacgtcttgttcagttacctcactactaaggagcaccttctcctatatggttccatcaaagttcctcactggactaaaaagcagctccacgaggaagtaaaaaggactttaaaagatactggactatatagccatcgtcataagagagttggaacactgtcaggaggcatgaagaggaagttatctatatccatagctctcattggtggatcaagggtagtaattttggatgaaccatctactggagttgacccatgttctcgccgaagtatatgggatgttatatccaagaacaaaactgccagaacaatcattctgtcaacgcaccacttggacgaggctgaagtgctgagtgaccgcatcgccttcctggagcagggtgggcttaggtgctgtgggtccccattttacctcaaggaagcctttggcgatgggtatcacctcacgcttaccaagaagaagagtccaaatttaaatgcaaatgcagtatgtgacaccatggccgtgacagcaatgatccaatcacatctccccgaagcctacctcaaggaggatattgggggagagcttgtttatgtacttcctccattcagcaccaaagtctcaggggcctacctgtcactcctacgggcactcgacaatggcatgggtgacctcaacatcgggtgctacggcatttcagataccaccgtggaggaggtctttctgaacttgaccaaagagtcacaaaaaaatagtgctatgagtcttgagcacttaacacaaaagaaaattgggaattccaatgccaatggcatctcaactcctgacgatttatctgtgagcagcagcaatttcacagacagagatgacaaaatcctgacaagaggagagaggctggatggctttggactgttgctgaagaagatcatggctatactcatcaagaggttccaccacacccgcaggaactggaaaggtctcattgctcaggttatcctccccatcgtctttgttaccactgccatgggccttggcacactgagaaattccagcaacagttatccagagattcagatctccccctctctttatggtacctccgaacagacagccttctatgctaattatcacccgagcacggaagcacttgtctcagcaatgtgggacttccctggaattgacaacatgtgtctgaacaccagtgatctacagtgtttaaacaaagacagtctggaaaaatggaacaccagtggagaacccatcactaattttggtgtttgctcctgctcagaaaatgtccaggaatgtcctaaatttaactattccccaccgcacagaagaacttactcatcccaggtaatttataacctcactgggcaacgagtggaaaattatcttatatcaactgcaaatgagtttgtccaaaaaagatatggaggttggagttttgggctgcctttgacaaaagaccttcgttttgatataacaggagtccctgccaatagaacacttgccaaggtatggtatgatccagaaggctatcactcccttccagcttacctcaacagcctgaataatttccttctgcgagttaacatgtcaaaatacgatgctgcccgacatggcatcatcatgtatagccatccttatccaggagtgcaagaccaagaacaagccacaatcagcagtttaatcgatattttagtggcactgtctatcttgatgggctactctgtcaccaccgccagctttgtcacctatgttgtaagggaacatcaaaccaaagccaaacagttgcagcacatttcaggcattggcgtgacatgctactgggtaacaaacttcatttatgacatggttttctacttggtgcctgtagcgttttcaattggtatcattgcgattttcaaattacctgcattctacagtgaaaacaacctaggcgctgtatctctcctacttctcctgtttgggtatgcaacattttcctggatgtacttgctggctgggctcttccatgaaacaggaatggccttcatcacttacgtctgtgtcaacttgttttttggcattaattccattgtttccctgtcagtggtatactttctttccaaggaaaagcctaatgatccgactttagaacttatttctgaaaccctcaagcgcattttcctgattttcccacaattctgttttggctacggtttgattgaactttctcaacaacagtcggtcctagacttcttaaaagcatatggagtggaatacccaaatgaaacctttgagatgaataaactaggtgcaatgtttgtggctttggtttctcagggcaccatgtttttttccttgcgactcttaatcaacgaatccctgataaagaaactcaggcttttcttcagaaaatttaattcttcacatgtaagggagacaatagatgaggatgaagatgtgcgggctgagagattaagagttgagagtggtgcagctgaatttgacttggtccaactttattgtctcacaaagacctaccaacttatccacaaaaagattatagctgtaaacaacatcagcattgggatacctgctggagagtgttttgggcttcttggagtgaatggagcaggaaagaccactatattcaagatgctgacaggagacatcattccttcaagtggaaacattctgatcagaaataagaccggatctctgggtcacgttgattctcacagctcattagttggctactgtcctcaggaagatgccttagatgacctggtaactgtggaagaacatttgtatttctatgccagggtacatggaattccagaaaaggatattaaagaaactgttcataaactccttaggagacttcacctgatgcccttcaaggacagagctacctctatgtgcagttatggcacaaaaagaaaattatccactgcactggccttgatagggaaaccttccattctactgctggatgagccgagctctggcatggatccgaagtcgaaacggcacctctggaagatcatttcagaagaagtacagaacaaatgttccgtcatcctcacatctcacagcatggaagaatgtgaagctctctgtaccaggttggccattatggtgaatggaaagtttcaatgtattggatctttgcagcacataaagagcaggtttggacgaggatttactgtcaaagttcacttgaagaataacaaagtgaccatggagaccctcacaaagttcatgcagctgcactttccaaaaacatacttaaaagatcagcacctcagcatgctagagtatcatgtaccagtcacagcaggaggagtcgcaaacatttttgatctgctggaaaccaacaagactgctttaaatattacaaatttcttagtgagtcagaccactctggaagaggttttcatcaactttgccaaagaccagaagtcctatgaaactgctgataccagcagccaaggttccactataagtgttgactcacaagatgaccagatggagtcttaa

SEQ ID NO: 4 - DNA, 6834 bp, Artificial Sequence
  Synthetic
atgttcacctacatcaagatcatcaccagcggcgacagcgacaacatcacccacgtgtggaacgaggacgacggccagacactgagcccatcttctctggctgcccagctgctgatcctggaaaacttcgaggacgccctgctgaacatcagcgctaacagcccttacatcccctacctggcctgcgtgcggaacgtgacagatagcctggctagaggcagccccgagaacctgagactgctgcagagcaccatccggttcaagaagtccttcctgcggaacggcagctacgaggactacttcccacctgtgcctgaggtgctgaagtccaagctgagccagctgagaaacctgaccgagctgctgtgcgagagcgagacattcagcctgatcgagaagtcctgccagctgtccgacatgagctttggcagcctgtgcgaggaaagcgagttcgacctgcagctgctggaagccgctgagctgggaacagaaattgccgccagcctgctgtaccacgacaacgtgatcagcaagaaagtgcgggacctgctgaccggcgatcccagcaagatcaacctgaatatggaccagttcctggaacaggccctgcagatgaactacctcgagaatatcacccagctgatccccatcatcgaggccatgctgcacgtgaacaatagcgccgatgccagcgagaagcctggacagctgctcgagatgttcaagaacgtggaagaactgaaagaggacctgcggcggaccaccggcatgagcaatagaaccatcgacaagctgctggctatccccattcctgacaaccgggccgagatcatctcccaggtgttctggctgcactcctgcgacaccaacatcacaacccctaagctggaagatgccatgaaggaattctgcaacctgagcctgagcgagcggagcagacagagctatctgatcggcctgactctgctgcactacctgaacatctacaactttacctacaaggtgttcttcccgcggaaggaccagaaacctgtggaaaagatgatggaactgttcatccggctgaaagaaatcctgaaccagatggccagcggcacacaccctctgctggacaagatgagatccctgaagcagatgcacttgcccagaagcgtgccactgacacaggccatgtaccggtccaacagaatgaacacccctcagggcagcttctccaccatctctcaggctctgtgtagccagggcatcaccaccgagtacctgaccgctatgctgcctagctctcagaggcccaagggcaaccacaccaaggactttctgacctacaagctgaccaaagagcagatcgcctctaagtacggcatcccgatcaacagcaccccattctgctttagcctgtacaaggacatcatcaacatgcctgctggccccgtgatctgggccttcctgaaacctatgctgctgggcagaatcctgtacgccccttacaaccctgtgaccaaggccatcatggaaaagtccaacgtgaccctgaggcagctggccgagctgagggaaaagtctcaagagtggatggacaagagcccactcttcatgaatagcttccatctgctgaaccaggcaatcccaatgctgcagaataccctgaggaaccccttcgtgcaggttttcgtgaagttcagcgtcggcctggacgccgtggaactgctgaaacagatcgacgagctggacatcctgcggctgaagctggaaaacaacatcgacatcatcgaccagctgaacaccctgagcagcctgaccgtgaatatcagctcctgcgtgctgtacgacagaatccaggccgccaagaccatcgatgagatggaaagagaggccaagcggctgtacaagagcaacgagctgttcggcagcgtgatcttcaagctgccaagcaacagaagctggcaccggggctacgatagcggcaatgtgtttctgcctcctgtgatcaagtacaccatccgcatgagcctgaaaacagcccagaccaccagaagcctgcggaccaaaatttgggcccctggacctcacaacagccccagccacaatcagatctacggcagggccttcatctacctgcaagacagcatcgagcgggccatcattgagctgcagaccggcagaaactcccaagagatcgccgttcaggtgcaggccattccttatccttgctttatgaaggacaacttcctgaccagcgtgtcctacagcctgcctatcgtgctgatggtggcctgggtcgtgtttatcgccgccttcgtgaagaaactggtgtacgagaaggacctgaggctgcacgagtacatgaagatgatgggcgtgaacagctgcagccacttcttcgcctggctgatcgagtccgtgggctttctgctggtcaccatcgtgatcctgatcatcatcctgaagttcggcaacatcctgcctaagaccaacggcttcatcctgtttctgtacttcagcgactactccttctccgtgatcgccatgagctacctgatctccgtgttcttcaacaacaccaatatcgccgcactgatcggctccctgatctatattatcgccttctttcccttcatcgtgctcgtgaccgtggaaaacgagctgagctacgtgctgaaggtgttcatgtccctgctgagccctaccgcctttagctacgccagccagtatatcgccagatacgaggaacaaggcatcggcctgcagtgggagaacatgtacacaagccccgtgcaggacgacaccaccagctttggatggctgtgttgcctgattctggccgacagctttatctacttcctgatcgcttggtacgtccggaacgtgttccctggcacatacggaatggctgccccttggtacttccccatcctgccaagctactggaaagaaagattcggctgcgccgaagtgaagcctgagaagtccaatggcctgatgtttaccaatatcatgatgcagaacacgaaccccagcgctagccccgagtatatgttcagctccaacatcgagcccgagcctaaggatctgacagtgggagttgctctgcacggcgtgaccaagatctatggctctaaggtggccgtggacaatctgaacctgaatttctacgagggccacatcactagcctgctgggacctaatggcgccggaaagaccaccaccattagcatgctgactggcctgtttggcgccagcgccggcacaatctttgtgtacggcaaggatatcaagaccgacctgcacaccgtgcggaagaacatgggcgtgtgtatgcagcacgatgtgctgttctcttacctgaccacaaaagagcatctgctgctctacggctccatcaaggtgccccactggaccaagaagcagctccacgaggaagtgaagagaaccctgaaggacaccggcctgtacagccacagacacaagagagtgggcacactgagcggcggcatgaagagaaagctgagcatctctatcgccctgatcggcggctccagagtggtcattctggatgagcctagcaccggcgtggacccttgctctagaagatccatctgggacgtgatctccaagaacaagaccgccagaaccatcattctgagcacccaccacctggacgaggccgaggttctgtctgatagaatcgccttcctcgaacaaggcggcctgagatgttgcggcagccccttctatctgaaagaagccttcggcgacggctaccacctgacactgacaaagaagaagtccccaaacctgaacgccaacgccgtgtgcgataccatggccgtgacagccatgatccagagccatctgcctgaggcctacctcaaagaggatatcggcggcgagctggtgtatgtgctgcctccattcagcaccaaagtgtccggcgcctacctgtctctgctgagagccctggataacggcatgggcgatctgaacatcggctgctacggcatcagcgacacaaccgtggaagaggtgttcctgaatctgacaaaagagagccagaaaaacagcgccatgtctctggaacatctgacccagaagaagatcggcaactccaacgccaatggcatcagcacccctgacgatctgtccgtgtcctccagcaacttcaccgaccgggacgacaagatcctgacaagaggcgagagactggacggctttggcctgctgctgaagaaaatcatggccatcctgattaagcggttccaccacaccagacggaactggaagggactgatcgcccaagtgattctgccaatcgtgtttgtgaccaccgccatgggcctgggcaccctgagaaatagcagcaacagctaccccgagatccagatcagcccaagcctgtacggcacatctgagcagaccgccttctacgccaactatcaccctagcacagaggccctggtgtccgccatgtgggatttccccggcatcgacaacatgtgcctgaacaccagcgatctgcagtgtctgaacaaggacagcctggaaaagtggaatacctccggcgagcccatcaccaactttggcgtgtgttcctgcagcgagaacgtgcaagagtgccccaagttcaactacagccctcctcacagacggacctacagcagccaagtgatctacaatctgaccggccagcgcgtggaaaattacctgatcagcaccgccaacgagttcgtgcagaagagatacggcggctggtctttcggactgcccctgaccaaggacctcagattcgacatcacaggcgtgcccgccaacagaaccctggccaaagtttggtacgaccccgagggctaccatagcctgccagcctacctgaatagcctgaacaatttcctgctgcgggtcaacatgagcaaatacgacgccgccagacacggcatcatcatgtattctcacccctatcctggcgtgcaggatcaagagcaggccacaatctcttccctgatcgacatcctggtggccctgtctatcctgatgggctacagcgtgaccacagccagcttcgtgacctacgttgtgcgggaacaccagacaaaggccaaacagctccagcacatcagcggcatcggagtgacctgttactgggtcaccaatttcatctacgacatggtgttctacctggtgcctgtggccttcagcatcggcatcattgccatttttaagctgcctgccttctactccgagaacaacctgggcgccgtttctctgctgctgctcctgtttggatacgccacctttagctggatgtacctgctcgccggcctgtttcacgaaaccggcatggcctttatcacatacgtgtgcgtgaacctgttcttcggcatcaactccatcgtgtccctgagcgtggtgtatttcctgagcaaagagaagcccaacgatcccacactggaactgatcagcgagactctgaagcggatctttctgatcttccctcagttctgcttcggctacggcctgattgagctgtcacagcagcagtccgtgctggacttcctgaaggcctacggcgtggaatatcccaacgaaaccttcgagatgaacaagctgggagccatgttcgtggctctggtgtctcagggaaccatgtttttcagtctgcgcctgctgatcaacgagtccctgatcaagaagctgcggctgttcttcagaaagttcaacagctcccacgtgcgcgagacaatcgacgaggatgaagatgtgcgggccgagcggctgagagttgaatctggcgccgctgagttcgatctggtgcagctgtactgtctgaccaagacataccagctcatccacaagaagatcattgccgtcaacaacatctccatcggaatcccagccggcgagtgttttggactgctgggagtgaatggcgcaggcaaaacaaccatcttcaagatgctgacgggcgacatcattcccagcagcggcaatatcctgatccggaacaagacaggcagcctgggccatgtggatagccactcttctctcgtgggctactgccctcaagaggacgctctggatgatctggtcaccgtcgaggaacatctctacttttacgccagagtgcacggaatccccgagaaagacatcaaagaaaccgtgcacaaactgctgcggcggctgcatctgatgcccttcaaagatagagccaccagcatgtgcagctacggcaccaagcggaagctgtctacagctctggccctgattggcaagcccagcatcctcctgctggacgagccatcttctggcatggaccccaagagcaagcggcatctgtggaagattatcagcgaagaggtgcagaacaagtgctccgtgattctgaccagccacagcatggaagagtgcgaggccctgtgtacccggctggccattatggtcaacggcaagtttcagtgcatcggcagcctgcagcacattaagtccagattcggcagaggcttcaccgtgaaggtccacctcaagaacaacaaagtgaccatggaaaccctcaccaagttcatgcaactgcacttccccaagacctatctcaaggaccagcacctgtccatgctggaataccacgtgccagtgactgctggcggagtggccaacatcttcgatctgctggaaacaaacaagacggccctgaatattaccaactttctggtgtcccagacgacccttgaggaagtgttcatcaacttcgccaaggaccaaaagagctacgagacagccgacacctccagccagggaagcacaatctccgtggacagccaggacgaccagatggaatcctga

SEQ ID NO: 5 - DNA, 1050 bp, Homo sapiens
atggcggcggaggaggaggaggtggactctgccgacaccggagagaggtcaggatggctaactggttggctccccacatggtgccctacgtctatatcacaccttaaagaagctgaagagaagatgttaaaatgtgtgccttgcacatacaaaaaagaacctgttcgtatatctaatggaaataaaatatggacactgaagttctctcataatatttcaaataagactccacttgtccttctccatggttttggaggaggtcttgggctctgggcactgaattttggagatctttgcaccaacagacctgtctatgcttttgacctattgggttttggacgaagtagtagacccaggtttgacagtgatgcagaagaagtggagaatcagtttgtggaatccattgaagagtggagatgtgccctaggattggacaaaatgatcttgcttgggcacaacctaggtggattcttggctgctgcttactcgctgaagtacccatcaagggttaatcatctcattttagtggagccttggggtttccctgaacgaccagaccttgctgatcaagacagaccaattccagtttggatcagagccttgggagcagcattgactccctttaaccctttagctggcctaaggattgcaggaccctttggtttaagtctagtgcagcgtttaaggcctgatttcaaacgaaagtattcttcaatgttcgaagacgatactgtgacagaatacatctaccactgtaatgtgcagactccaagtggtgagacagctttcaagaatatgactattccttatggatgggcaaaaaggccaatgctccagcgaattggtaaaatgcaccctgacattccagtttcagtgatctttggcgcccgatcctgcatagatggcaattctggcaccagcatccagtccttacgaccacattcatatgtgaagacaatagctattcttggggcaggacattatgtatatgcagatcaaccagaagaattcaaccagaaagtaaaggagatctgcgacactgtggactga

SEQ ID NO: 6 - DNA, 1050 bp, Artificial Sequence
  Synthetic
atggccgccgaagaggaagaagtggatagcgccgataccggcgagagatctggatggctgacaggctggctgcctacctggtgtcctaccagcatcagccacctgaaagaggccgaagagaaaatgctgaagtgcgtgccctgcacctacaagaaagaacccgtgcggatcagcaacggcaacaagatctggaccctgaagttcagccacaacatcagcaacaagacccctctggtgctgctgcacggatttggcggaggacttggactgtgggccctgaactttggcgacctgtgcaccaacagacccgtgtacgcctttgacctgctcggcttcggcagaagcagcagacccagatttgacagcgacgccgaggaagtggaaaaccagttcgtggaatccatcgaagagtggcgctgtgccctgggcctcgacaagatgattctgctgggccacaacctcggcggctttctggctgctgcctatagcctgaagtaccccagcagagtgaaccacctgatcctggtggaaccctggggcttccccgaaagacctgatctggctgaccaggacagacccattcctgtgtggatcagagccctgggagctgccctgacacctttcaatcctctggccggcctgagaatcgccggaccttttggactgagcctggtgcagagactgcggcccgacttcaagagaaagtacagcagcatgttcgaggacgacaccgtgaccgagtacatctaccactgcaacgtgcagacccctagcggcgagacagccttcaagaacatgacaatcccttacggctgggccaagcggcccatgctgcagagaattggcaagatgcaccctgacatccccgtgtccgtgatctttggcgccagaagctgcatcgacggcaatagcggcaccagcatccagtctctgaggccccacagctacgtgaaaacaatcgccatcctcggagccggccactatgtgtatgccgaccagcctgaagagttcaaccagaaagtgaaagaaatctgcgacaccgtcgactga

SEQ ID NO: 7 - DNA, 1527 bp, Homo sapiens
atggagctcgaagtccggcgggtccgacaggcgttcctgtccggccggtcgcgacctctgcggtttcggctgcagcagctggaggccctgcggaggatggtgcaggagcgcgagaaggatatcctgacggccatcgccgccgacctgtgcaagagtgaattcaatgtgtacagtcaggaagtcattactgtccttggggaaattgattttatgcttgagaatcttcctgaatgggttactgctaaaccagttaagaagaacgtgctcaccatgctggatgaggcctatattcagccacagcctctgggagtggtgctgataatcggagcttggaattaccccttcgttctcaccattcagccactgataggagccatcgctgcaggaaatgctgtgattataaagccttctgaactgagtgaaaatacagccaagatcttggcaaagcttctccctcagtatttagaccaggatctctatattgttattaatggtggtgttgaggaaaccacggagctcctgaagcagcgatttgaccacattttctatacgggaaacactgcggttggcaaaattgtcatggaagctgctgccaagcatctgacccctgtgactcttgaactgggagggaaaagtccatgttatattgataaagattgtgacctggacattgtttgcagacgcataacctggggaaaatacatgaattgtggccaaacctgcattgcacccgactatattctctgtgaagcatccctccaaaatcaaattgtatggaagattaaggaaacagtgaaggaattttatggagaaaatataaaagagtctcctgattatgaaaggatcatcaatcttcgtcattttaagaggatactaagtttgcttgaaggacaaaagatagcttttggtggggagactgatgaggccacacgctacatagccccaacagtacttaccgatgttgatcctaaaaccaaggtgatgcaagaagaaatttttggaccaattcttccaatagtgcctgtgaaaaatgtagatgaggccataaatttcataaatgaacgtgaaaagcctctggctctttatgtattttcgcataaccataagctcatcaaacggatgattgatgagacatccagtggaggtgtcacaggcaatgacgtcattatgcacttcacgctcaactctttcccatttggaggagtgggttccagtgggatgggagcttatcacggaaaacatagttttgatactttttctcatcagcgtccctgtttattaaaaagtttaaagagagaaggtgctaacaaactcagatatcctcccaacagccagtcaaaggtggattggggaaaattttttctcttgaaacggttcaacaaagaaaaactcggtctcctgttgctcactttcctgggtattgtagccgctgtgcttgtcaagaaataccaagctgtgctgaggagaaaggccctgttgatttttctggtagttcacagactgcgttggtccagtaagcagagatga

SEQ ID NO: 8 - DNA, 1527 bp, Artificial Sequence
  Synthetic
atggaactggaagtgcggagagtgcggcaggcctttctgagcggaagaagcagacccctgcggttcagactgcagcagctggaagccctgcggagaatggtgcaagagagagagaaggacatcctgaccgccattgccgccgatctgtgcaagagcgagttcaacgtgtacagccaagaagtgatcaccgtcctgggcgagatcgacttcatgctggaaaacctgcctgagtgggtcaccgccaagcctgtgaagaaaaacgtgctgaccatgctggacgaggcctacatccagcctcagcctctgggagtcgtgctgatcatcggcgcctggaactaccccttcgtgctgacaatccagccactgatcggagccattgctgccggcaatgccgtgatcatcaagccctctgagctgagcgagaataccgccaagatcctggccaagctgctgccccagtacctggaccaggatctgtacatcgtgatcaacggcggcgtggaagagacaaccgagctgctgaagcagagattcgaccacatcttctacaccggcaacaccgccgtgggcaagatcgtgatggaagccgccgctaagcacctgacacctgtgacactggaactcggcggaaagagcccctgctacatcgacaaggactgcgacctggacatcgtgtgcagaagaatcacctggggcaagtacatgaactgcggccagacctgtatcgccccagactacatcctgtgcgaagccagcctgcagaaccagattgtgtggaagatcaaagaaaccgtgaaagagttctacggcgagaacatcaaagagagccccgactacgagcggatcatcaacctgcggcacttcaagagaatcctgagcctgctggaaggccagaagatcgcctttggcggcgagacagatgaggccaccagatatatcgcccctacagtgctgaccgacgtggaccccaagaccaaagtgatgcaagaggaaatcttcggccccatcctgcctatcgtgcccgtgaagaatgtggacgaagccatcaacttcatcaacgagcgcgagaagcccctggctctgtacgtgttcagccacaaccacaagctgatcaagcggatgatcgacgagacaagctctggcggcgtgaccggcaacgatgtgatcatgcactttaccctgaacagcttcccattcggcggagtgggcagctctggaatgggagcctatcacggcaagcacagcttcgacaccttcagccaccagaggccttgcctgctgaagtccctgaaaagagagggcgccaacaagctgagataccctcctaacagccagagcaaggtggactggggaaagttctttctgctgaagaggttcaacaaagagaagctgggcctgctgctgctgaccttcctgggaattgtggccgccgtgctggtcaagaaataccaggccgtgctgagaagaaaggccctgctgatctttctggtggtgcaccggctgagatggtccagcaaacagagatga

SEQ ID NO: 9 - DNA, 2106 bp, Homo sapiens
atggccacctacaaagtcagggtggccacaggcaccgacctcttgtcgggaacacgggactccatctcactgaccattgtggggacacaaggagagagccataagcagctgctgaaccactttgggagagactttgcaactggggcggtgggccagtacaccgtgcagtgccctcaggacctgggtgagctcatcatcatccgcctgcacaaagagcggtacgccttcttccccaaggacccttggtactgcaactatgtgcagatctgtgcccccaacggccgtatctaccacttccccgcctaccagtggatggatggctacgagaccctggcactccgggaggccacaggaaagacaacagcagatgactcgctccccgtcctcctggagcacagaaaagaggagatcagagccaagcaggacttctaccactggcgagtctttcttcctggcctgcccagctatgtgcacattcccagttaccgccctccggtgcggaggcatcgcaaccccaaccggcctgagtggaatggctatattccgggattcccaattctcatcaactttaaggccaccaagttcctgaacttaaatctccgctactccttcctcaagacggcctccttcttcgtccgcctggggcccatggcactggctttcaaagtccgcggcctgttggactgcaaacattcgtggaagaggctgaaggacattaggaaaattttccctggcaagaaatctgtcgtctccgagtacgtggccgagcactgggcagaggacaccttctttgggtaccagtacctcaacggcgtcaaccccggcctgatccgccgctgcacgcggatcccagacaagttccccgtcacagacgacatggtggctccgttcctgggcgagggaacgtgcttgcaagcggagctggagaaggggaacatttacctggccgactaccgcatcatggagggcatccccaccgtggagctcagcggccggaagcagcaccactgcgcccccctctgcctgctgcactttggacccgagggcaagatgatgcccatcgccatccagctcagccagacccctgggccagattgccccatcttcctgcccagtgattctgagtgggactggctgctagccaagacgtgggtacgctatgcggagttctacagccacgaggccatcgcccacctgctggagacacacctcattgctgaggccttctgcctggccttgctgaggaacctgcccatgtgccaccccctctacaagctcctcatcccccatacccgatacaccgtccagatcaacagcattggccgggccgttctcctcaatgagggggggctctctgccaagggcatgtccctgggcgtggaaggctttgctggggtgatggtacgggctctgtcggagctcacctatgacagcctctacctccccaatgactttgtggagcgtggggtccaggacctgcctggatattactaccgcgatgacagcttggcggtgtggaatgcactggagaagtatgtgacggagatcatcacctattattacccgagtgacgcagccgtggagggtgatccggaattgcagtcttgggtgcaggaaatatttaaagagtgcctcctggggcgggagagctcaggcttccctaggtgcttgcgaaccgtgcctgagctgatccgatatgtcactatagtcatctacacctgctctgccaagcacgctgctgtcaacacaggccagatggagttcaccgcctggatgcccaacttcccagcgtccatgcggaatccaccgattcagactaaggggctgaccactctggagaccttcatggacacgttgccggatgtgaagaccacgtgcatcacgctgctggtgctctggaccctcagccgagagcctgacgacaggcggcccctgggacacttcccggacattcacttcgtggaggaggccccgcggaggagcatagaggcgttccgccagcgcctgaaccagatctcacacgacatccgccagcgcaacaagtgccttcccatcccctactactacctggacccggtgctgattgagaacagcatttctatttag

SEQ ID NO: 10 - DNA, 2106 bp, Artificial Sequence
  Synthetic
atggccacctacaaagtgcgagtggccaccggcacagatctgctgtctggcaccagagacagcatcagcctgaccatcgttggcacacagggcgagtctcacaaacagctgctgaaccacttcggccgggattttgctacaggcgccgtgggccagtacaccgttcagtgtcctcaggatctgggcgagctgatcatcatccggctgcacaaagagcgctacgcattcttcccaaaggacccctggtactgcaactacgtgcagatttgcgcccctaacggccggatctaccactttcctgcctaccagtggatggacggctacgagacactggccctgagagaggccacaggcaagaccacagccgatgattctctgcccgtgctgctggaacaccggaaagaggaaatccgggccaagcaggacttctaccattggagagtgtttctgcccggcctgcctagctacgtgcacatccctagctacagacctccagtgcggagacacagaaaccccaacagacccgagtggaacggctacatccctggcttccccatcctgatcaacttcaaggccaccaagtttctgaacctgaacctgcggtacagcttcctgaaaaccgccagcttctttgtgcggctgggccctatggctctggcctttaaagttcggggcctgctggactgcaagcacagctggaagagactgaaggacatccgcaagatcttccccggcaagaaaagcgtggtgtccgagtatgtggccgagcactgggccgaagataccttcttcggctaccagtacctgaacggcgtgaaccccggcctgatcagacggtgcacaagaatccccgacaagttccccgtgaccgacgatatggtggccccatttctcggcgagggcacatgtctgcaggccgagctggaaaagggcaacatctacctggccgactaccggatcatggaaggcatccctaccgtggaactgagcggcagaaagcagcaccattgtgcccctctgtgcctgctgcactttggacctgagggcaagatgatgcctatcgccatccagctgagccagacacctggacctgactgccctatcttcctgccaagcgacagcgagtgggattggctgctggccaagacctgggtccgatacgccgagttttacagccacgaggctatcgcccatctgctggaaacacacctgatcgccgaggccttttgtctggccctgctgagaaacctgcctatgtgtcaccctctgtacaagctgctgatccctcacaccagatacaccgtgcagatcaacagcatcggcagagctgtgctgctgaatgaaggcggcctgtctgccaagggcatgtctctgggagttgagggatttgccggcgtgatggttcgagccctgagcgagctgacatacgacagcctgtacctgcctaacgacttcgtggaacggggcgtgcaagatctgcctggctactactacagggacgactctctggccgtgtggaacgccctcgagaaatacgtgaccgagatcatcacctactactatcccagcgacgccgccgtggaaggcgatcctgaactgcagtcttgggtgcaagagatcttcaaagagtgtctgctgggcagagagagcagcggcttccctagatgcctgagaacagtgcccgagctgattcgctacgtgacaatcgtgatctacacctgtagcgccaagcacgccgctgtgaataccggccagatggaattcaccgcctggatgcctaacttccccgccagcatgagaaaccctccaatccagaccaagggcctgaccacactggaaaccttcatggacaccctgcctgacgtgaaaaccacctgtatcaccctgctggtgctgtggaccctgagcagagagcctgacgatagaaggcccctgggccactttccagacatccactttgtggaagaggcccctaggcggagcatcgaggctttcagacagaggctgaaccagatcagccacgacatccggcagcggaacaagtgcctgcctattccttactattatctggaccccgtgctgatcgagaacagcatctccatctga

SEQ ID NO: 11 - DNA, 2136 bp, Homo sapiens
atggcagtgtaccgcctgtgtgtgaccactggtccctacctgagggccggcacactggacaacatctctgtcacactggtgggcacgtgtggtgaaagccccaagcagcggctagatcgaatgggcagggacttcgcccctggatcggtacagaagtacaaggtgcgttgcacagcggagctgggtgagctcttgctgctgcgtgtacacaaggagcgctacgctttcttccgcaaggactcttggtactgtagccgcatctgtgtcaccgaaccggatggtagtgtatcccacttcccctgctatcagtggattgaaggctactgcaccgtggagctgaggccaggaacagcaagaactatttgtcaggactctcttcccctcctcctggatcacaggacacgggagctccgggcccgacaagaatgctaccgctggaagatctatgcccctggcttcccctgcatggtagacgtcaacagctttcaggagatggagtcagacaagaaatttgccttgacaaagacgacaacttgtgtagaccagggtgacagcagtgggaatcggtacctgcccggcttccccatgaaaattgacatcccatccctgatgtacatggagcccaatgttcgatactcagccaccaagacgatctcgctgctcttcaatgccatccctgcgtccttgggaatgaagcttcgagggctgttggatcgcaagggctcctggaagaagctggatgacatgcagaacatcttctggtgccataagaccttcacgacaaagtatgtcacagagcactggtgtgaagatcacttctttgggtaccagtacctgaatggtgtcaatcccgtcatgctccactgcatctctagcttgcccagcaagctgcctgtcaccaatgacatggtggcccccttgctgggacaggacacatgcctgcagacagagctagagagggggaacatcttcctagcggactactggatcctggcggaggcccccacccactgcctaaacggccgccagcagtacgtggccgccccactgtgcctgctgtggctcagcccccagggggcgctggtgcccttggccatccagctcagccagacccccgggcctgacagccccatcttcctgcccactgactccgaatgggactggctgctggccaagacgtgggtgcgcaactctgagttcctggtgcacgaaaacaacacgcactttctgtgcacgcatttgctgtgcgaggccttcgccatggccacgctgcgccagctgccgctctgccaccccatctacaagctcctactcccccacactcgatacacgctgcaggtgaacaccatcgcgagggccacgctgctcaaccccgagggcctcgtggaccaggtcacgtccatcgggaggcaaggcctcatctacctcatgagcacgggcctggcccacttcacctacaccaatttctgccttccggacagcctgcgggcccgcggcgtcctggctatccccaactaccactaccgagacgacggcctgaagatctgggcggccattgagagctttgtctcagaaatcgtgggctactattatcccagtgacgcatctgtgcagcaggattcggagctgcaggcctggactggcgagatttttgctcaggcgttcctgggccgggaaagctcaggtttcccaagccggctgtgcaccccaggagagatggtgaagttcctcactgcaatcatcttcaattgctctgcccagcacgctgctgtcaacagtgggcagcatgactttggggcctggatgcccaatgctccatcatccatgaggcagcccccaccccagaccaaggggaccaccaccctgaagacttacctagacaccctccctgaagtgaacatcagctgtaacaacctcctcctcttctggttggttagccaagaacccaaggaccagaggcccctgggcacctacccagatgagcacttcacagaggaggccccgaggcggagcatcgccgccttccagagccgcctggcccagatctcaagggacatccaggagcggaaccagggtctggcactgccctacacctacctggaccctcccctcattgagaacagcgtctccatctaa

SEQ ID NO: 12 - DNA, 2136 bp, Artificial Sequence
  Synthetic
atggccgtgtacagactgtgcgtgaccaccggaccttacctgagagccggcacactggacaacatcagcgtgacactcgtgggcacatgtggcgagagccctaagcagagactggacagaatgggcagagacttcgcccctggcagcgtgcaaaagtacaaagtgcggtgtacagccgagctgggcgaactgctgctgctgagagtgcacaaagagcgctacgccttcttcagaaaggacagctggtactgcagccggatctgcgtgacagaacctgacggctccgtgtctcacttcccctgctatcagtggatcgagggctactgcaccgtggaactgaggcctggaaccgccagaaccatctgccaggattctctgcccctgctgctggaccacagaaccagagaactgcgggccagacaagagtgctaccggtggaagatctacgccccaggcttcccttgcatggtggacgtgaacagcttccaagagatggaaagcgacaagaagttcgccctgaccaagaccaccacctgtgtggatcagggcgacagcagcggcaacagatacctgcctggcttccccatgaagatcgacatccccagcctgatgtacatggaacccaacgtgcggtacagcgccaccaagacaatcagcctgctgttcaacgctatccccgccagcctgggcatgaagctgagaggactgctggatagaaaaggcagctggaagaaactggacgatatgcagaacatcttctggtgccacaagaccttcaccaccaaatacgtgaccgagcattggtgcgaggaccacttcttcggctaccagtacctgaacggcgtgaaccccgtgatgctgcactgcatctctagcctgcctagcaagctgcccgtgaccaacgatatggtggccccactgctgggccaagacacatgcctgcagaccgagctggaacggggcaatatcttcctggccgactactggattctggccgaggctcctacacactgcctgaatggcagacagcagtacgtggccgctcctctgtgtctgctttggctgtctcctcagggtgctctggtgcctctggccattcagctgtctcagacccctggacctgacagccccatcttcctgccaacagacagcgagtgggattggctgctggccaaaacctgggtccgaaacagcgagttcctggtgcacgagaacaatacccacttcctgtgcacccatctgctgtgcgaggcctttgccatggccacacttagacagctgcccctgtgtcaccccatctacaagctgctgctccctcacaccagatacaccctgcaagtgaacacaatcgccagagccacactgctgaaccccgaaggcctggtggatcaagtgaccagcatcggcagacagggcctgatctacctgatgagcacaggactggcccacttcacctacaccaatttctgcctgccagactctctgagagccagaggcgtgctggctatccccaactaccactacagagatgacggcctgaagatctgggccgccatcgagagctttgtgtccgagatcgtgggctattactacccctccgatgccagcgtgcagcaggattctgaactgcaggcttggaccggcgagatcttcgcccaggcatttctgggaagagagagcagcggcttccctagcagactgtgtacaccaggcgagatggtcaagttcctgaccgccatcatcttcaactgcagcgcccagcatgccgccgtgaattctggccagcatgatttcggagcctggatgcccaatgctcccagctctatgagacagcctcctccacagaccaagggcaccaccacactgaaaacctacctggacaccctgccagaagtgaacatcagctgcaacaatctgctgctgttttggctggtgtcccaagagcctaaggaccagaggccactgggcacataccccgatgagcacttcacagaggaagcccctcggagatctatcgccgcctttcagagtcggctggcccagattagccgggacatccaagagagaaaccagggcctcgctctgccctacacctatctcgatcctccactgatcgagaacagcgtgtccatctga

SEQ ID NO: 13 - DNA, 2532 bp, Homo sapiens
atgccacgcggggcattcagaccctgtttgccggcgctgtatttcgctttcctgacctgccctactccagagcagagaatgagtggaacccaggctcctgatatccatctgggtgagccagccagagggaccggctgtgtcagaggcaagcaaacaagtattagagtgcaagactgtgggcggagagaggaagcccgagccgccagcagggagcttcggagagagaaagcccaggaacatcccagagagagctgggcccatcctcagccctacccagccccgcagcccctagccctccgcccagaaacccagccctgtccggcgtgccgctcttctcctccaggccggctgctgctgcggccagcgttgccggggcatcccttcctccttcccatcatggcagtgtaccgcctgtgtgtgaccactggtccctacctgagggccggcacactggacaacatctctgtcacactggtgggcacgtgtggtgaaagccccaagcagcggctagatcgaatgggcagggacttcgcccctggatcggtacagaagtacaaggtgcgttgcacagcggagctgggtgagctcttgctgctgcgtgtacacaaggagcgctacgctttcttccgcaaggactcttggtactgtagccgcatctgtgtcaccgaaccggatggtagtgtatcccacttcccctgctatcagtggattgaaggctactgcaccgtggagctgaggccaggaacagcaagaactatttgtcaggactctcttcccctcctcctggatcacaggacacgggagctccgggcccgacaagaatgctaccgctggaagatctatgcccctggcttcccctgcatggtagacgtcaacagctttcaggagatggagtcagacaagaaatttgccttgacaaagacgacaacttgtgtagaccagggtgacagcagtgggaatcggtacctgcccggcttccccatgaaaattgacatcccatccctgatgtacatggagcccaatgttcgatactcagccaccaagacgatctcgctgctcttcaatgccatccctgcgtccttgggaatgaagcttcgagggctgttggatcgcaagggctcctggaagaagctggatgacatgcagaacatcttctggtgccataagaccttcacgacaaagtatgtcacagagcactggtgtgaagatcacttctttgggtaccagtacctgaatggtgtcaatcccgtcatgctccactgcatctctagcttgcccagcaagctgcctgtcaccaatgacatggtggcccccttgctgggacaggacacatgcctgcagacagagctagagagggggaacatcttcctagcggactactggatcctggcggaggcccccacccactgcctaaacggccgccagcagtacgtggccgccccactgtgcctgctgtggctcagcccccagggggcgctggtgcccttggccatccagctcagccagacccccgggcctgacagccccatcttcctgcccactgactccgaatgggactggctgctggccaagacgtgggtgcgcaactctgagttcctggtgcacgaaaacaacacgcactttctgtgcacgcatttgctgtgcgaggccttcgccatggccacgctgcgccagctgccgctctgccaccccatctacaagctcctactcccccacactcgatacacgctgcaggtgaacaccatcgcgagggccacgctgctcaaccccgagggcctcgtggaccaggtcacgtccatcgggaggcaaggcctcatctacctcatgagcacgggcctggcccacttcacctacaccaatttctgccttccggacagcctgcgggcccgcggcgtcctggctatccccaactaccactaccgagacgacggcctgaagatctgggcggccattgagagctttgtctcagaaatcgtgggctactattatcccagtgacgcatctgtgcagcaggattcggagctgcaggcctggactggcgagatttttgctcaggcgttcctgggccgggaaagctcaggtttcccaagccggctgtgcaccccaggagagatggtgaagttcctcactgcaatcatcttcaattgctctgcccagcacgctgctgtcaacagtgggcagcatgactttggggcctggatgcccaatgctccatcatccatgaggcagcccccaccccagaccaaggggaccaccaccctgaagacttacctagacaccctccctgaagtgaacatcagctgtaacaacctcctcctcttctggttggttagccaagaacccaaggaccagaggcccctgggcacctacccagatgagcacttcacagaggaggccccgaggcggagcatcgccgccttccagagccgcctggcccagatctcaagggacatccaggagcggaaccagggtctggcactgccctacacctacctggaccctcccctcattgagaacagcgtctccatctaa

SEQ ID NO: 14 - DNA, 2532 bp, Artificial Sequence
  Synthetic
atgcctagaggcgccttcagaccttgtctgcccgctctgtacttcgccttcctgacatgccctacacctgagcagagaatgagcggcacacaggcccctgatatccacctgggagaacctgccagaggcacaggctgtgttagaggcaagcagaccagcatcagagtgcaggactgcggcagaagagaggaagccagagccgcctctagagagctgagaagagagaaggcccaagagcaccccagagagtcttgggctcaccctcagccttatcctgctcctcaacctctggctctgaggcccgaaacacagccatgtcctgcctgtagaagcagccctcctggtagactgctgctgaggcctgctttgcctggacacccttttctgctgcccatcatggccgtgtacagactgtgcgtgaccaccggaccttacctgagagccggcacactggacaacatcagcgtgacactcgtgggcacatgtggcgagagccctaagcagagactggacagaatgggcagagacttcgcccctggcagcgtgcaaaagtacaaagtgcggtgtacagccgagctgggcgaactgctgcttctgagagtgcacaaagagcgctacgccttcttcagaaaggacagctggtactgcagccggatctgcgtgacagaacctgacggctccgtgtctcacttcccctgctatcagtggatcgagggctactgcaccgtggaactcagacctggcaccgccagaaccatctgccaggattctctgcccctgctgctggaccacagaaccagagaactgagagccagacaagagtgctaccggtggaagatctacgccccaggcttcccttgcatggtggacgtgaacagcttccaagagatggaaagcgacaagaagttcgccctgaccaagaccaccacctgtgtggatcagggcgacagcagcggcaacagatacctgcctggcttccccatgaagatcgacatccccagcctgatgtacatggaacccaacgtgcggtacagcgccaccaagacaatcagcctgctgttcaacgctatccccgccagcctgggcatgaagctgagaggactgctggatagaaaaggcagctggaagaaactggacgatatgcagaacatcttctggtgccacaagaccttcaccaccaaatacgtgaccgagcattggtgcgaggaccacttcttcggctaccagtacctgaacggcgtgaaccccgtgatgctgcactgcatctctagcctgcctagcaagctgcccgtgaccaacgatatggtggccccactgctgggccaagacacatgcctgcagaccgagctggaacggggcaatatcttcctggccgactactggattctggccgaggctcctacacactgcctgaatggcagacagcagtacgtggccgctcctctgtgtctgctttggctgtctcctcagggtgctctggtgcccctggctattcagctgtctcagacccctggacctgacagccccatcttcctgccaacagacagcgagtgggattggctgctggccaaaacctgggtccgaaacagcgagttcctggtgcacgagaacaatacccacttcctgtgcacccatctgctgtgcgaggcctttgccatggccacacttagacagctgcccctgtgtcaccccatctacaagctgctgctgccccacaccagatacaccctgcaagtgaacacaatcgccagagccacactgctgaaccccgaaggcctggtggatcaagtgacctccattggcaggcagggcctgatctacctgatgagcacaggactggcccacttcacctacaccaatttctgcctgcctgatagcctgcgcgctagaggcgttctggccattcctaactaccactacagagatgacggcctgaagatctgggccgccatcgagagctttgtgtccgagatcgtgggctattactacccctccgatgccagcgtgcagcaggattctgaactgcaggcttggaccggcgagatcttcgcccaggcatttctgggaagagagagcagcggcttccctagcagactgtgtacaccaggcgagatggtcaagttcctgaccgccatcatcttcaactgcagcgcccagcatgccgccgtgaattctggccagcatgatttcggagcctggatgcccaatgctcccagctctatgagacagcctcctccacagaccaagggcaccaccacactgaaaacctacctggataccctgccagaagtgaacatcagctgcaacaacctgctcctgttctggctggtgtcccaagagcctaaggatcagaggcccctgggcacataccccgatgagcactttacagaagaggccccaagacggtctatcgccgcctttcaatcccggctggcccagattagccgggacatccaagagagaaaccagggcctcgctctgccttacacctatctggaccctccactgatcgagaacagcgtgtccatctga

SEQ ID NO: 15 - DNA, 477 bp, Homo sapiens
atgatgcggttcatgctattattcagccggcagggaaaactgcggctgcaaaaatggtacctggccacttcggacaaggaacggaagaagatggtgcgcgagctcatgcaggttgtcctggctcgaaagcccaagatgtgcagcttcctggagtggagggacctcaaagttgtctataagagatatgccagcctctacttctgctgcgccatcgagggccaagacaatgagctcatcacactggagctgatccaccgatacgtggagctcttagacaaatactttggcagtgtgtgcgagctggacatcatcttcaactttgagaaggcctacttcatcctggatgagtttttgatggggggggatgtccaggacacctccaagaagagtgtgctgaaagccatcgagcaggctgacctactgcaagaggaggatgagtcgccacggagtgtgctggaggagatgggtttggcatag

SEQ ID NO: 16 - DNA, 477 bp, Artificial Sequence
  Synthetic
atgatgcggttcatgctgctgttcagcagacagggcaagctgcggctgcagaagtggtatctggccaccagcgacaaagaacggaagaagatggtccgagagctgatgcaggtcgtgctggccagaaagcccaagatgtgcagcttcctggaatggcgggacctgaaggtggtgtacaagagatacgccagcctgtacttctgctgcgccatcgaaggccaggacaacgagctgatcaccctggaactgatccacagatacgtggaactgctggacaagtacttcggcagcgtgtgcgagctggacatcatcttcaacttcgagaaggcctactttatcctggacgagttcctgatgggcggcgacgtgcaggacaccagcaagaaatctgtgctgaaggccatcgagcaggccgacctgctgcaagaagaggacgagagccctagaagcgtgctggaagaaatgggcctcgcctga

SEQ ID NO: 17 - DNA, 1770 bp, Homo sapiens
atgttacatctgcaccattcttgtttgtgtttcaggagctggctgccagcgatgctcgctgtactgctaagtttggcaccatcagcttccagcgacatttccgcctcccgaccgaacatccttcttctgatggcggacgaccttggcattggggacattggctgctatggcaacaacaccatgaggactccgaatattgaccgccttgcagaggacggcgtgaagctgacccaacacatctctgccgcatctttgtgcaccccaagcagagccgccttcctcacgggcagataccctgtgcgatcagggatggtttccagcattggttaccgtgttcttcagtggaccggagcatctggaggtcttccaacaaatgagacaacttttgcaaaaatactgaaagagaaaggctatgccactggactcattggaaaatggcatctgggtctcaactgtgagtcagccagtgatcattgccaccaccctctccatcatggctttgaccatttctacggaatgcctttctccttgatgggtgattgcgcccgctgggaactctcagagaagcgtgtcaacctggaacaaaaactcaacttcctcttccaagtcctggccttggttgccctcacactggtagcagggaagctcacacacctgatacccgtctcgtggatgccggtcatctggtcagccctttcggccgtcctcctcctcgcaagctcctattttgtgggtgctctgattgtccatgccgattgctttctgatgagaaaccacaccatcacggagcagcccatgtgcttccaaagaacgacaccccttattctgcaggaggttgcgtcctttctcaaaaggaataagcatgggcctttcctcctctttgtttcctttctacacgttcacatccctcttatcactatggagaacttcctcgggaagagtctccacgggctgtatggggacaacgtagaggagatggactggatggtaggacggatccttgacactttggacgtggagggtttgagcaacagcaccctcatttattttacgtcggatcacggcggttccctagagaatcaacttggaaacacccagtatggtggctggaatggaatttataaaggtgggaagggcatgggaggatgggaaggtgggatccgcgtgcccgggatcttccgctggcccggggtgctcccggccggccgagtgattggcgagcccacgagtctgatggacgtgttccccaccgtggtccggctggcgggcggcgaggtgccccaggacagagtgattgacggccaagaccttctgcccttgctcctggggacagcccaacactcagaccacgagttcctgatgcattattgtgagaggtttctgcacgcagccaggtggcatcaacgggacagaggaacaatgtggaaagtccactttgtgacgcctgtgttccagccagagggagccggtgcctgctatggaagaaaggtctgcccgtgctttggggaaaaagtagtccaccacgatccacctttgctctttgacctctcaagagacccttctgagacccacatcctcacaccagcctcagagcccgtgttctatcaggtgatggaacgagtccagcaggcggtgtgggaacaccagcggacactcagcccagttcctctgcagctggacaggctgggcaacatctggagaccgtggctgcagccctgctgtggcccgttccccctctgctggtgccttagggaagatgacccacaataa

SEQ ID NO: 18 - DNA, 1770 bp, Artificial Sequence
  Synthetic
atgctgcatctgcaccacagctgcctgtgcttcagatcttggctgcctgccatgctggccgtgctgttgtctcttgcccctagcgccagcagcgatatcagcgcctccagacctaacatcctgctgctgatggccgacgatctcggcatcggcgatatcggctgctacggcaacaacaccatgcggacccctaacatcgacagactggccgaggatggcgtgaagctgacacagcatattagcgccgccagcctgtgcaccccttctagagctgcatttctgaccggcagataccctgtgcggagcggcatggtgtctagcatcggctatagagtgctgcagtggacaggcgcttctggcggccttcctaccaacgagacaaccttcgccaaaatcctgaaagagaagggctacgccaccggcctgatcggaaaatggcacctgggactgaattgcgagagcgccagcgaccactgtcaccatcctctgcaccatggcttcgaccacttctacggcatgcccttcagcctgatgggcgattgtgccagatgggagctgagcgagaagagagtgaacctggaacagaagctgaacttcctgttccaagtgctggccctggtggccctgacactggttgctggcaaactgacccatctgatccccgtgtcctggatgcccgtgatttggagcgctctgtctgctgtgctgctgctggctagcagctattttgtgggcgccctgatcgtgcacgccgactgctttctgatgcggaaccacaccatcaccgagcagcccatgtgcttccagagaacaacccctctgatcctgcaagaggtggccagcttcctgaagcggaacaagcacggcccatttctgctgttcgtgtccttcctccacgtgcacatccctctgattacgatggaaaacttcctgggcaagagcctgcacggcctgtacggcgataacgtggaagagatggactggatggtcggaagaatcctggacaccctggacgtggaaggcctgagcaatagcaccctgatctacttcacctccgaccacggcggcagcctggaaaatcagctgggcaatacccagtacggcggctggaacggcatctacaaaggcggcaaaggcatgggcggatgggaaggcggaattagagtgcccggcatcttcagatggcctggcgttttgcctgccggaagagtgatcggcgagcccacctctctgatggacgtgttccctacagttgtgcggctggctggcggagaagtgcctcaggatagagtgattgacggccaggacctgctgcctctgttgctgggaacagcccagcacagcgatcacgagttcctcatgcactactgcgagcggtttctgcacgccgccagatggcaccagcgagatagaggcacaatgtggaaggtgcacttcgtgacccctgtgttccagcctgaaggtgccggcgcttgttacggcagaaaagtgtgcccttgcttcggcgagaaggtggtgcatcacgatcctccactgctgtttgacctgagcagggaccctagcgagacacacattctgacaccagccagcgagcccgtgttctaccaagtgatggaaagggtgcagcaggccgtgtgggagcaccagagaactctgtctccagtgcctctgcagctggatcggctgggcaacatttggaggccttggctccagccttgctgcggcccttttcctctgtgctggtgcctgagagaagatgaccctcagtga

SEQ ID NO: 19 - DNA, 729 bp, Homo sapiens
atgagcaatccgcggtctttggaagaggagaaatatgatatgtcaggtgcccgcctggccctaatactgtgtgtcaccaaagcccgggaaggttccgaagaagacctggatgctctggaacacatgtttcggcagctgagattcgaaagcaccatgaaaagagaccccactgccgagcaattccaggaagagctggaaaaattccagcaggccatcgattcccgggaagatcccgtcagttgtgccttcgtggtactcatggctcacgggagggaaggcttcctcaagggagaagatggggagatggtcaagctggagaatctcttcgaggccctgaacaacaagaactgccaggccctgcgagctaagcccaaggtgtacatcatacaggcctgtcgaggagaacaaagggaccccggtgaaacagtaggtggagatgagattgtgatggtcatcaaagacagcccacaaaccatcccaacatacacagatgccttgcacgtttattccacggtagagggatacatcgcctaccgacatgatcagaaaggctcatgctttatccagaccctggtggatgtgttcacgaagaggaaaggacatatcttggaacttctgacagaggtgacccggcggatggcagaagcagagctggttcaagaaggaaaagcaaggaaaacgaaccctgaaatccaaagcaccctccggaaacggctgtatctgcagtag

SEQ ID NO: 20 - DNA, 729 bp, Artificial Sequence
  Synthetic
atgagcaaccccagaagcctggaagaagagaagtacgacatgagcggcgccagactggccctgatcctgtgtgtgacaaaggccagagagggcagcgaagaggatctggatgccctggaacacatgttccggcagctgagattcgagagcaccatgaagagagatcccaccgccgagcagttccaagaggaactggaaaagttccagcaggccatcgacagcagagaagatcccgtgtcctgcgccttcgtggtgctgatggctcacggcagagagggatttctgaaaggcgaggacggcgagatggtcaagctggaaaatctgttcgaggccctgaacaacaagaactgtcaggccctgcgggccaagcctaaggtgtacatcatccaggcctgcagaggcgagcaaagagatcctggcgaaaccgtcggcggcgacgagatcgtgatggtcatcaaggacagccctcagacaatccccacctacacagatgccctgcacgtgtacagcaccgtggaaggctatatcgcctaccggcacgatcagaagggcagctgcttcatccagacactggtggacgtgttcaccaagcggaagggccacattctcgagctgctgaccgaagtgaccagaagaatggccgaggccgagctggtgcaagagggcaaagccagaaagacaaaccccgagatccagagcaccctgcggaagagactgtacctgcagtga

SEQ ID NO: 21 - DNA, 1590 bp, Homo sapiens
atgggctcgtctcgggcaccctggatggggcgtgtgggtgggcacgggatgatggcactgctgctggctggtctcctcctgccagggaccttggctaagagcattggcaccttctcagacccttgtaaggaccccacgcgtatcacctcccctaacgacccctgcctcactgggaagggtgactccagcggtttcagtagctacagtggctccagcagttctggcagctccatttccagtgccagaagctctggtggtggctccagtggtagctccagcggatccagcattgcccagggtggttctgcaggatcttttaagccaggaacggggtattcccaggtcagctactcctccggatctggctctagtctacaaggtgcatccggttcctcccagctggggagcagcagctctcactcgggaaacagcggctctcactcgggaagcagcagctctcattcgagcagcagcagcagctttcagttcagcagcagcagcttccaagtagggaatggctctgctctgccaaccaatgacaactcttaccgcggaatactaaacccttcccagcctggacaaagctcttcctcttcccagacctttggggtatccagcagtggccaaagcgtcagctccaaccagcgtccctgtagttcggacatccccgactctccctgcagtggagggcccatcgtctcgcactccggcccctacatccccagctcccactctgtgtcagggggtcagaggcctgtggtggtggtggtggaccagcacggttctggtgcccctggagtggttcaaggtcccccctgtagcaatggtggccttccaggcaagccctgtcccccaatcacctctgtagacaaatcctatggtggctacgaggtggtgggtggctcctctgacagttatctggttccaggcatgacctacagtaagggtaaaatctaccctgtgggctacttcaccaaagagaaccctgtgaaaggctctccaggggtcccttcctttgcagctgggccccccatctctgagggcaaatacttctccagcaaccccatcatccccagccagtcggcagcttcctcggccattgcattccagccagtggggactggtggggtccagctctgtggaggcggctccacgggctccaagggaccctgctctccctccagttctcgagtccccagcagttctagcatttccagcagctccggtttaccctaccatccctgcggcagtgcttcccagagcccctgctccccaccaggcaccggctccttcagcagcagctccagttcccaatccagtggcaaaatcatccttcagccttgtggcagcaagtccagctcttctggtcacccttgcatgtctgtctcctccttgacactgactgggggccccgatggctctccccatcctgatccctccgctggtgccaagccctgtggctccagcagtgctggaaagatcccctgccgctccatccgggatatcctagcccaagtgaagcctctggggccccagctagctgaccctgaagttttcctaccccaaggagagttactcaacagtccataa

SEQ ID NO: 22 - DNA, 1590 bp, Artificial Sequence
  Synthetic
atgggcagttctagagccccttggatgggcagagttggcggccatggaatgatggctctgctgctggctggactgctgctccctggaacactggccaagagcatcggcaccttcagcgatccatgcaaggaccccaccagaatcacaagccccaacgatccttgcctgaccggcaagggcgatagcagcggctttagcagctacagcggcagctctagctctggcagcagcatcagcagcgccagatcttctggcggaggcagctctggttctagctccggatcttctatcgcccaaggcggctctgccggctcttttaagcctggcacaggctacagccaggtgtcctactctagcggctctggaagttctctgcagggcgcctctggatcttctcagctgggatctagcagcagccactccggcaattctggctctcactctggctccagcagctcccactctagcagctccagctccttccagttcagctcctccagcttccaagtcggcaatggaagcgccctgcctaccaacgacaacagctatcggggcattctgaacccctctcagcctggccaaagcagcagcagctctcagacatttggcgtgtccagcagcggccagtccgtgtctagcaatcagaggccttgcagcagcgacatccctgactctccatgttctggcggccctatcgtgtctcacagcggaccttacatcccttccagccactctgtgtctggcggacaaagacctgtggtggtggtcgtggatcagcatggaagcggagcacctggtgttgtgcagggccctccttgttctaatggcggcctgcctggaaagccctgtcctcctatcaccagcgtggacaagagctacggcggctatgaggttgtcggcggctccagcgattcttacctggtgcctggcatgacctacagcaagggcaagatctaccccgtgggctacttcaccaaagaaaaccccgtgaagggcagccctggcgtgccatcttttgccgctggacctcctatctccgagggcaagtacttcagcagcaaccctatcatccccagccagagcgccgcctcttctgccattgcttttcagcctgttggcaccggtggcgtgcaactttgtggcggaggaagcacaggcagcaagggaccttgtagcccatccagctctagagtgcccagctcctctagcatctcttccagctccggcctgccatatcacccttgtggatctgccagccagtctccttgcagccctcctggaacaggcagcttcagttcctctagctcctctcagagcagcggcaagatcatcctgcagccttgtggcagcaagtctagctccagcggacacccttgtatgagcgtgtcctctctgacactgacaggcggccctgatggaagccctcatcctgatccatctgccggcgctaagccatgtggaagctctagcgccggaaagatcccctgcagatccatcagagacattctggcccaagtgaagcctctgggccctcagcttgctgaccccgaagtgtttctgcctcaaggcgagctgctgaacagcccctaa

SEQ ID NO: 23 - DNA, 1152 bp, Homo sapiens
atgttttggacgtttaaagaatggttctggttggaaagattctggcttcctccaacaataaagtggtcagatcttgaggatcacgatggactcgtctttgtaaaaccttctcatttatacgtgacaattccatatgcttttctcttgctgattatcagacgtgtatttgaaaaatttgttgcttcacctctagcaaaatcatttggcattaaagagacagttcgaaaggttacaccaaatactgtcttagagaattttttcaaacattccacaaggcaaccattgcaaactgatatttatggactggcaaagaagtgtaacttgacggagcgccaggtggaaagatggtttaggagtcggcggaatcaagagaggccttccaggctgaagaaattccaggaagcttgctggagatttgcattttacttaatgatcactgttgctggaattgcgtttctttatgataaaccttggctatatgacttatgggaggtttggaatggctatcccaaacagcccctgctgccatcccagtactggtactacattttagaaatgagtttttattggtctctgttatttagacttggctttgatgtcaagagaaaggattttctagctcatatcatccaccacctggctgctattagtctgatgagcttctcttggtgtgctaattatattcgcagtgggaccctcgtgatgattgtacacgatgtggctgacatttggctggagtctgctaagatgttttcttatgctggatggacgcagacctgtaacaccctgtttttcatcttctccaccatatttttcatcagccgcctcattgtttttcctttctggattttatattgcacgctgatcttgcctatgtatcacctcgagcctttcttttcatacatcttcctcaacctacagctcatgatcttgcaggtccttcacctttactggggttattacatcttgaagatgctcaacagatgtatattcatgaagagcatccaggatgtgaggagtgatgacgaggattatgaagaggaagaggaagaggaagaagaagaggctaccaaaggcaaagagatggattgtttaaagaacggcctcagggctgagaggcacctcattcccaatggccagcatggccattag

SEQ ID NO: 24 - DNA, 1152 bp, Artificial Sequence
  Synthetic
atgttctggaccttcaaagagtggttctggctggaaagattctggctgcctcctaccatcaagtggagcgacctggaagatcacgacggcctggtgttcgtgaagcccagccacctgtacgtgacaatcccttacgccttcctgctgctgatcatccggcgggtgttcgagaagttcgtggcctctcctctggccaaatccttcggcatcaaagaaaccgtgcggaaagtgacccctaataccgtgctggaaaacttcttcaagcacagcaccagacagcccctgcagaccgatatctatggcctggccaagaagtgcaacctgaccgagagacaggtggaacggtggttcagaagccggcggaatcaagagaggcccagcagactgaagaaattccaagaagcctgttggcgcttcgccttctacctgatgatcaccgtggccggaatcgcctttctgtacgacaagccctggctgtacgacctgtgggaagtgtggaacggctaccccaaacagcctctgctgcccagccagtattggtactacatcctggaaatgagcttctactggtccctgctgttcagactgggcttcgacgtgaagcggaaggactttctggcccacatcatccaccacctggccgccattagcctgatgtccttttcttggtgcgccaactacattcggagcggcaccctggtcatgattgtgcacgatgtggccgacatctggctcgagagcgccaagatgtttagctacgccggctggacccagacctgcaacaccctgttctttatcttcagcaccatctttttcatctcccggctgatcgtgttccccttctggattctgtactgcaccctgatcctgcctatgtaccacctggaacctttcttcagctacatcttcctgaacctgcagctgatgatcctgcaggtcctgcacctgtactggggctactatatcctgaagatgctgaaccggtgcatctttatgaagtccatccaggacgtgcggagcgacgacgaggactacgaggaagaagaggaagaggaagaagaagaggccaccaagggcaaagagatggactgcctgaagaacggcctgcgggccgagaggcacctgattcctaatggacagcacggccactga

SEQ ID NO: 25 - DNA, 1275 bp, Homo sapiens
atgaccctgcgacctggaacaatgcggctggcctgcatgttctcttccatcctgctgttcggagctgcaggcctcctcctcttcatcagcctgcaggaccctacggagctcgccccccagcaggtgccaggaataaagttcaacatcaggccaaggcagccccaccacgacctcccaccaggcggctcccaggatggtgacttgaaggaacccacagagagggtcactcgggacttatccagtggggccccgaggggccgcaacctgccagcgcctgaccagcctcaacccccgctgcagaggggaacccgtctgcggctccgccagcgccgtcgccgtctgctcatcaagaaaatgccagctgcggcgaccatcccggccaacagctcggacgcgcccttcatccggccgggacccgggacgctggatggccgctgggtcagcctgcaccggagccagcaggagcgcaagcgggtgatgcaggaggcctgcgccaagtaccgggcgagcagcagccgccgggccgtcacgccccgccacgtgtcccgtatcttcgtggaggaccgccaccgcgtgctctactgcgaggtgcccaaggccggctgctccaattggaagcgggtgctcatggtgctggccggcctggcctcgtccactgccgacatccagcacaacaccgtccactatggcagcgctctcaagcgcctggacaccttcgaccgccagggtatcttgcaccgtctcagcacctacaccaagatgctctttgtccgcgagcccttcgagaggctggtgtccgccttccgcgacaagtttgagcaccccaacagctactatcacccggtcttcggcaaggccatcctggcccggtaccgcgccaatgcctctcgggaggccctgcggaccggctctggggtgcgttttcccgagttcgtccagtacctgctggacgtgcaccggcccgtggggatggacattcactgggaccatgtcagccggctctgcagcccctgcctcatcgactacgatttcgtaggcaagttcgagagcatggaggacgatgccaacttcttcctgagcctcatccgcgcgccgcggaacctgaccttcccccggttcaaggaccggcactcgcaggaggcgcggaccacagcgaggatcgcccaccagtacttcgcccaactctcggccctgcaaaggcagcgcacctacgacttctactacatggattacctgatgttcaactattccaagccctttgcagatctgtactga

SEQ ID NO: 26 - DNA, 1275 bp, Artificial Sequence
  Synthetic
atgacactgaggcccggcacaatgagactggcctgcatgttcagcagcatcctgctgtttggagccgccggactgctgctgttcatcagtctgcaggaccctacagagctggcccctcagcaagtgcctggcatcaagttcaacatcagacccagacagccccaccacgatcttccacctggtggatctcaggacggcgacctgaaagaacccaccgagagagtgaccagggatctgtctagcggagcccctagaggcagaaacctgcctgctcctgatcagcctcagcctccactgcagagaggcaccagactgagactgcggcagagaagaagaaggctgctgatcaagaagatgcctgccgccgctacaatccccgccaattcttctgacgcccctttcatcagacctggacctggcacactggacggcagatgggtttccctgcacagatctcagcaagagcggaagagagtgatgcaagaggcctgcgccaagtacagagccagcagctctagaagggccgtgacacccagacatgtgtcccggatcttcgtggaagatagacaccgggtgctgtactgcgaggtgccaaaggccggctgcagcaattggaagagggtgctgatggtgctggccggcctggcttcttctacagccgacatccagcacaacaccgtgcactatggcagcgccctgaagcggctggacacctttgatagacagggcatcctgcaccggctgagcacctacaccaagatgctgttcgtgcgcgagcccttcgagagactggtgtccgccttcagagacaagttcgagcaccccaacagctactatcaccccgtgttcggcaaggccatcctggccagatatagagccaacgcctctagagaggccctgagaacaggctctggcgtgcggtttcctgagttcgtgcagtacctgctggacgtgcacagacccgtcggaatggacatccactgggaccacgtgtccagactgtgcagcccttgcctgatcgactacgacttcgtgggcaagtttgagagcatggaagatgacgccaacttctttctgtccctgattcgggcccctcggaacctgacattccccagattcaaggaccggcacagccaagaggccagaaccacagccagaatcgcccaccagtacttcgcccagctgtctgccctgcagagacagcggacctacgatttctactacatggactacctgatgttcaactacagcaagcccttcgccgacctgtactga

SEQ ID NO: 27 - DNA, 636 bp, Homo sapiens
atggccaacgcggggctgcagctgttgggcttcattctcgccttcctgggatggatcggcgccatcgtcagcactgccctgccccagtggaggatttactcctatgccggcgacaacatcgtgaccgcccaggccatgtacgaggggctgtggatgtcctgcgtgtcgcagagcaccgggcagatccagtgcaaagtctttgactccttgctgaatctgagcagcacattgcaagcaacccgtgccttgatggtggttggcatcctcctgggagtgatagcaatctttgtggccaccgttggcatgaagtgtatgaagtgcttggaagacgatgaggtgcagaagatgaggatggctgtcattgggggtgcgatatttcttcttgcaggtctggctattttagttgccacagcatggtatggcaatagaatcgttcaagaattctatgaccctatgaccccagtcaatgccaggtacgaatttggtcaggctctcttcactggctgggctgctgcttctctctgccttctgggaggtgccctactttgctgttcctgtccccgaaaaacaacctcttacccaacaccaaggccctatccaaaacctgcaccttccagcgggaaagactacgtgtga

SEQ ID NO: 28 - DNA, 636 bp, Artificial Sequence
  Synthetic
atggccaatgctggactgcagctgctgggcttcatcctggcctttctcggatggatcggcgccatcgtgtctacagctttgcctcagtggcggatctacagctacgccggcgacaatatcgtgacagcccaggctatgtacgaaggcctgtggatgagctgcgtgtcccagtctacaggccagatccagtgcaaggtgttcgacagcctgctgaacctgagcagcacactgcaggctacaagagccctgatggtcgtgggaatcctgctgggagtgatcgccatctttgtggccaccgtgggcatgaagtgtatgaagtgcctggaagatgacgaggtgcagaaaatgcggatggccgtgatcggcggagccatctttctgctggctggactggctatcctggtggccactgcttggtacggcaaccggatcgtgcaagagttctacgaccccatgacacccgtgaacgccagatacgagtttggacaggccctgttcacaggatgggccgctgcttctctgtgtctgcttggcggagcactgctgtgctgcagctgtcccagaaagaccacaagctaccccacacctcggccttatcctaagcctgctcctagcagcggcaaggactacgtgtaa

SEQ ID NO: 29 - DNA, 297 bp, Homo sapiens
atgatacctggaggcttatctgaggccaaacccgccactccagaaatccaggagattgttgataaggttaaaccacagcttgaagaaaaaacaaatgagacttacggaaaattggaagctgtgcagtataaaactcaagttgttgctggaacaaattactacattaaggtacgagcaggtgataataaatatatgcacttgaaagtattcaaaagtcttcccggacaaaatgaggacttggtacttactggataccaggttgacaaaaacaaggatgacgagctgacgggcttttag

SEQ ID NO: 30 - DNA, 297 bp, Artificial Sequence
  Synthetic
atgattcctggcggcctgtctgaggccaaacctgccacacctgagatccaagagatcgtggacaaagtgaagccccagctggaagaaaagaccaacgagacatacggcaagctggaagccgtgcagtacaagacacaggtggtggccggcaccaactactacatcaaagtgcgcgctggcgacaacaagtacatgcacctgaaggtgttcaagagcctgcctggccagaacgaggatctggtgctgacaggctaccaggtggacaagaacaaggacgacgagctgaccggcttctga

SEQ ID NO: 31 - DNA, 1596 bp, Homo sapiens
atgctgcccatcacagaccgcctgctgcacctcctggggctggagaagacggcgttccgcatatacgcggtgtccacccttctcctcttcctgctcttcttcctgttccgcctgctgctgcggttcctgaggctctgcaggagcttctacatcacctgccgccggctgcgctgcttcccccagcctccccggcgcaactggctgctgggccacctgggcatgtaccttccaaatgaggcgggccttcaagatgagaagaaggtactggacaacatgcaccatgtactcttggtatggatgggacctgtcctgccgctgttggttctggtgcaccctgattacatcaaaccccttttgggagcctcagctgccatcgcccccaaggatgacctcttctatggcttcctaaaaccttggctaggggatgggctgctgctcagcaaaggtgacaagtggagccggcaccgtcgcctgctgacacccgccttccactttgacatcctgaagccttacatgaagatcttcaaccagagcgctgacattatgcatgctaaatggcggcatctggcagagggctcagcggtctcccttgatatgtttgagcatatcagcctcatgaccctggacagtcttcagaaatgtgtcttcagctacaacagcaactgccaagagaagatgagtgattatatctccgctatcattgaactgagcgctctgtctgtccggcgccagtatcgcttgcaccactacctcgacttcatttactaccgctcggcggatgggcggaggttccggcaggcctgtgacatggtgcaccacttcaccactgaagtcatccaggaacggcggcgggcactgcgtcagcagggggccgaggcctggcttaaggccaagcaggggaagaccttggactttattgatgtgctgctcctggccagggatgaagatggaaaggaactgtcagacgaggatatccgagccgaagcagacaccttcatgtttgagggtcacgacacaacatccagtgggatctcttggatgctgttcaatttggcaaagtatccggaataccaggagaaatgccgagaagagattcaggaagtcatgaaaggccgggagctggaggagctggagtgggacgatctgactcagctgccctttacaactatgtgcattaaggagagcctgcgccagtacccacctgtcactcttgtctctcgccaatgcacggaggacatcaagctcccagatgggcgcatcatccccaaaggaatcatctgcttggtcagcatctatggaacccaccacaaccccacagtgtggcctgactccaaggtgtacaacccctaccgctttgacccggacaacccacagcagcgctctccactggcctatgtgcccttctctgcaggacccaggaattgcatcggacagagcttcgccatggccgagttgcgcgtggttgtggcactaacactgctacgtttccgcctgagcgtggaccgaacgcgcaaggtgcggcggaagccggagctcatactgcgcacggagaacgggctctggctcaaggtggagccgctgcctccgcgggcctga

SEQ ID NO: 32 - DNA, 1596 bp, Artificial Sequence
  Synthetic
atgctgcccatcaccgacagactgctgcatctgctgggcctcgagaaaaccgccttcagaatctacgccgtgtctaccctgctgctgttcctgctgtttttcctgttccggctgctgctgagattcctgcggctgtgccggtccttctacatcacctgtcggcggctgagatgcttccctcagcctcctagaagaaactggctgctgggacacctgggcatgtacctgcctaatgaagccggcctgcaggacgagaagaaagtgctggacaacatgcaccacgtgctgctcgtgtggatgggacctgttctgcctctgctggtgctggtgcaccccgactatatcaagccactgctgggagcctctgccgctatcgcccctaaggacgatctgttctacggcttcctgaagccttggctcggagatggcctgctgctcagcaaaggcgacaagtggtccagacacagacggctgctgacccctgccttccacttcgacattctgaagccctacatgaagatctttaaccagagcgccgacatcatgcacgccaagtggcgacatctggccgagggatctgctgtgtccctggacatgttcgagcacatcagcctgatgaccctggacagcctgcagaaatgcgtgttcagctacaacagcaactgccaagagaagatgagcgactacatcagcgccatcatcgagctgagcgctctgtctgtgcggcggcagtatagactgcaccactacctggacttcatctactacagaagcgccgacggcagacggttcagacaggcttgtgacatggtgcatcacttcaccaccgaagtgatccaagagcggcggagagcactgagacagcaaggtgcagaagcctggctgaaggccaagcagggcaagaccctggatttcatcgatgtgctgctgctcgccagagatgaggacggcaaagagctgtccgacgaggacattagagccgaggccgacaccttcatgtttgagggccacgataccaccagctccggcatcagctggatgctgttcaacctggccaagtatcccgagtatcaagagaagtgccgggaagagattcaagaagtgatgaagggccgcgagctggaagaactggaatgggacgatctcacccagctgcctttcaccaccatgtgcatcaaagagagcctgcggcagtaccctcctgtgacactggtgtccagacagtgcaccgaggacatcaagctgcccgatggcagaatcatccccaagggcatcatctgcctggtgtctatctacggcacccaccacaatcctaccgtgtggcccgatagcaaggtgtacaacccctacagattcgaccccgacaatccccagcagagaagccctctggcctacgtgccattttccgccggacctagaaactgcatcggccagtcttttgccatggccgagctgagagtggtggtggctctgaccctgctgaggttcagactgagcgtggaccggaccagaaaagtgcggagaaagcccgagctgatcctgagaaccgagaacggcctgtggctgaaagtggaacccctgccacctagagcctga

SEQ ID NO: 33 - DNA, 693 bp, Homo sapiens
atgactaccaacgcgggccccttgcacccatactggcctcagcacctaagactggacaactttgtacctaatgaccgccccacctggcatatactggctggcctcttctctgtcacaggggtcttagtcgtgaccacatggctgttgtcaggtcgtgctgcggttgtcccattggggacttggcggcgactgtccctgtgctggtttgcagtgtgtgggttcattcacctggtgatcgagggctggttcgttctctactacgaagacctgcttggagaccaagccttcttatctcaactctggaaagagtatgccaagggagacagccgatacatcctgggtgacaacttcacagtgtgcatggaaaccatcacagcttgcctgtggggaccactcagcctgtgggtggtgatcgcctttctccgccagcatcccctccgcttcattctacagcttgtggtctctgtgggccagatctatggggatgtgctctacttcctgacagagcaccgcgacggattccagcacggagagctgggccaccctctctacttctggttttactttgtcttcatgaatgccctgtggctggtgctgcctggagtccttgtgcttgatgctgtgaagcacctcactcatgcccagagcacgctggatgccaaggccacaaaagccaagagcaagaagaactga

SEQ ID NO: 34 - DNA, 693 bp, Artificial Sequence
  Synthetic
atgaccaccaatgccggacctctgcacccttactggcctcagcatctgcggctggacaacttcgtgcccaacgacagacctacctggcacattctggccggcctgttttctgtgaccggcgtgctggtggtcaccacatggttgctttctggcagagccgccgtggtgcctcttggaacttggagaaggctgagcctgtgttggttcgccgtgtgcggctttatccacctggtcatcgaaggatggttcgtgctgtactacgaggacctgctgggcgatcaggcctttctgagccagctgtggaaagagtacgccaagggcgacagccggtacatcctgggcgataacttcaccgtgtgcatggaaaccatcaccgcctgtctgtggggccctctgtctctgtgggttgtgatcgccttcctgagacagcaccctctgcggttcattctgcagctggtggtgtccgtgggccagatctatggcgacgtgctgtatttcctgaccgagcacagagatggcttccagcatggcgaactgggacaccctctgtacttttggttctacttcgtgttcatgaatgccctgtggctggtgctgcctggcgttctggttctggacgccgtgaaacacctgacacacgcccagtctaccctggacgccaaagccacaaaggccaagagcaagaagaactga

SEQ ID NO: 35 - DNA, 945 bp, Homo sapiens
atggggctcctggactcggagccgggtagtgtcctaaacgtagtgtccacggcactcaacgacacggtagagttctaccgctggacctggtccatcgcagataagcgtgtggaaaattggcctctgatgcagtctccttggcctacactaagtataagcactctttatctcctgtttgtgtggctgggtccaaaatggatgaaggaccgagaaccttttcagatgcgtctagtgctcattatctataattttgggatggttttgcttaacctctttatcttcagagagttattcatgggatcatataatgcgggatatagctatatttgccagagtgtggattattctaataatgttcatgaagtcaggatagctgctgctctgtggtggtactttgtatctaaaggagttgagtatttggacacagtgttttttattctgagaaagaaaaacaaccaagtttctttccttcatgtgtatcatcactgtacgatgtttaccttgtggtggattggaattaagtgggttgcaggaggacaagcattttttggagcccagttgaattcctttatccatgtgattatgtactcatactatgggttaactgcatttggcccatggattcagaaatatctttggtggaaacgatacctgactatgttgcaactgattcaattccatgtgaccattgggcacacggcactgtctctttacactgactgccccttccccaaatggatgcactgggctctaattgcctatgcaatcagcttcatatttctctttcttaacttctacattcggacatacaaagagcctaagaaaccaaaagctggaaaaacagccatgaatggtatttcagcaaatggtgtgagcaaatcagaaaaacaactcatgatagaaaatggaaaaaagcagaaaaatggaaaagcaaaaggagattaa

SEQ ID NO: 36 - DNA, 945 bp, Artificial Sequence
  Synthetic
atgggcctgctggattctgagcctggcagcgtgctgaacgtggtgtctacagccctgaacgacaccgtcgagttctaccggtggacctggtctatcgccgacaagagagtggaaaactggcccctgatgcagagcccctggcctacactgagcatcagcacactgtacctgctgttcgtgtggctgggccccaagtggatgaaggacagagaacccttccagatgcggctggtgctgatcatctacaacttcggcatggtgctgctgaacctgttcatcttccgcgagctgttcatgggcagctacaacgccggctacagctacatctgtcagagcgtggactacagcaacaacgtgcacgaagtgcggattgccgctgctctgtggtggtacttcgtgtccaaaggcgtggaatacctggacaccgtgttcttcatcctgcggaagaaaaacaaccaggtgtccttcctgcacgtgtaccaccattgcaccatgttcaccctgtggtggatcggcatcaaatgggtggcaggcggccaggccttttttggcgcccagctgaacagctttatccacgtgatcatgtacagctactacggcctgaccgccttcggaccctggattcagaaatacctctggtggaagcgctacctgaccatgctgcagctgatccagttccacgtgaccatcggacacactgccctgagcctgtacaccgactgtccatttccaaagtggatgcactgggccctgatcgcctacgctatctcctttatcttcctgttcctgaacttctacatccggacctacaaagagcccaagaagcccaaggccggcaagaccgccatgaatggcattagcgcaaacggcgtgtccaagagcgagaagcagctgatgatcgagaacggcaagaagcagaaaaacggcaaggccaagggcgactga

SEQ ID NO: 37 - DNA, 12186 bp, Homo sapiens
atgtctactctcctggaaaacatctttgccataattaatcttttcaagcaatattcaaaaaaagataaaaacactgacacattgagtaaaaaagagctgaaggaacttctggaaaaggaatttcggcaaatcctgaagaatccagatgacccagatatggttgatgtcttcatggatcacttggatatagaccacaacaagaaaattgacttcactgagtttcttctgatggtattcaagttggctcaagcatattatgagtctaccagaaaagagaatttaccgatatcaggacacaagcacagaaagcacagtcatcatgataaacatgaagataataaacaggaagaaaacaaagaaaacagaaaaagaccctcaagtctggaaagaagaaacaatagaaaagggaataagggaagatccaagagcccaagagaaacaggggggaaaaggcatgaatctagttctgaaaaaaaagaaagaaaaggatattcacctactcatagagaagaagaatatggaaaaaaccatcataactcaagtaaaaaagagaaaaacaagactgaaaatactagattaggagacaataggaagaggctaagtgaaagacttgaagagaaagaagacaatgaagaaggagtatatgattatgaaaatacaggaagaatgactcaaaaatggatacaatcaggccatattgccacatattacacaatccaggatgaagcctatgacaccactgatagtctattagaagaaaacaaaatatatgaaagatcaaggtcatctgatggcaaatcatcatctcaagtgaacaggtcaagacatgaaaatacaagccaggtaccattgcaggagtccaggacaagaaagcgtaggggatccagagttagccaggacagggacagtgagggacactcagaagactctgagaggcactctgggtcggcttccagaaaccatcatggatctgcgtgggagcagtcaagagatggctccagacaccccaggtcccatgatgaagacagagccagtcatgggcactctgcagacagctccagacaatcaggcactcgtcacgcagagacttcctctcgtggacagactgcatcatcccatgaacaggcaagatcaagtccaggagaaagacatggatccggccaccagcagtcagcagacagctccagacactcagccactgggcgcgggcaagcttcatctgcagtcagcgatcgtggacaccgggggtctagcggtagtcaggccagtgacagtgagggacattcagaaaactcagacacacaatcagtgtcaggccacggaaaggctgggctgagacagcagagccaccaagagtccacacgtggccggtcaggggaacggtctggacgttcagggtcttccctctaccaggtgagcactcatgaacagcctgactctgcccatggacggaccgggaccagcactggaggaagacaaggatcgcaccacgagcaggcacgagacagctccaggcattcagcgtcccaagagggtcaggacaccattcgtggacacccggggtcaagcagaggaggaaggcagggatcccaccacgagcaatcggtaaataggtctggacactcaggttcccatcacagccacaccacatcccagggaaggtctgatgcctcccatgggcagtcaggatccagaagtgcaagcagacaaacacgaaatgaggaacaatcaggagacggcaccaggcactcagggtcacgtcatcatgaagcttcctctcaggctgacagctctagacactcacaggtgggccagggacaatcatcggggcccaggacaagtaggaaccagggatccagtgttagccaggacagtgacagtcagggacactcagaagactctgagaggtggtctgggtctgcttccagaaaccatcatggatctgctcaggagcagtcaagagatggctccagacaccccaggtcccatcacgaagacagagctggtcatgggcactctgcagacagctccagaaaatcaggcactcgtcacacacagaattcctctagtggacaggctgcgtcatcccatgaacaggcaagatcaagtgcaggagaaagacatggatcccgccaccagctccagtcagcagacagctccagacactcaggcactgggcacggacaagcttcatctgcagtcagagacagtggacaccgagggtccagtggtagtcaggccactgacagtgagggacattcagaagactcagacacacagtcagtgtcaggccatggacaggctggtcaccatcagcagagccaccaagagtccgcacgtgaccggtcaggggaaaggtctcgacgttcagggtctttcctctaccaggtgagcactcataaacagtctgagtcctcccatggatggacagggcccagcactggagtaagacaaggatcccaccatgagcaggcacgagacaactccaggcactcagcatcccaagatggtcaggacaccattcgtggacacccggggtcaagcagaagaggaaggcaggggtcccaccacgagcaatcggtagataggtctggacactcagggtcccatcacagccacaccacatcccagggaaggtctgatgcctcccgtgggcagtcaggatccagaagtgcaagcagaacaacacgtaatgaggaacaatcaagagacggctccaggcactcagggtcacgtcaccatgaagcttcctctcatgccgacatctctagacactcacaggcaggccagggacaatcagaggggtccaggacaagcaggcgccagggatccagtgttagccaggacagtgacagtgagggacattcagaagactctgagaggtggtctgggtctgcttccagaaaccatcgtggatctgctcaggagcagtcaagacatggctccagacaccccaggtcccatcacgaagacagagccggtcacgggcactctgcagacagctccagacaatcaggaactcctcacgcagagacttcctctggtggacaggctgcgtcatcccatgaacaggcaagatcaagtccaggagaaagacacggatcccgccaccagcagtcagcagacagctccagacactcaggcattccgcgcagacaagcttcatctgcagtcagagacagtggacactgggggtccagtggtagtcaggccagtgatagtgagggacattcagaggagtcagacacacagtcagtgtcaggccatggacaggatgggccccatcagcagagccaccaagagtccgcacgtgactggtcagggggaaggtctggacgttcagggtctttcatctaccaggtgagcactcatgaacagtctgagtctgcccatgggcggaccaggaccagcactggacgaagacaaggatcccaccacgagcaggcacgagacagctccaggcactcagcgtcccaagagggtcaggacaccattcgtgcacacccggggtcaaggagaggaggaaggcagggatcccaccatgagcaatcggtagatagatctggacactcagggtcccatcacagccacaccacatcccagggaaggtctgatgcctcccatgggcagtcaggatccagaagtgcaagcagacaaactcgtaaggacaaacaatcaggagacggctccaggcactcagggtcacgtcaccatgaagctgcctcttgggctgacagctctagacactcacaggtgggacaggaacaatcatcggggtccaggacaagcaggcaccagggatccagtgttagccaggacagtgacagtgagagacactcagacgactccgagaggttgtctgggtctgcttccagaaaccatcatggatcttctcgggagcagtcaagagatggctccagacaccctgggttccatcaagaagacagagccagtcacgggcactctgcagacagctccagacaatcaggcactcatcacacagagtcttcctctcatggacaggctgtgtcatcccatgaacaggcaagatcaagtccaggagaaagacatggatcccgccaccagcagtcagcagacagctccagacactcaggcattgggcacagacaagcttcatctgcagtcagagacagtggacaccgagggtccagtggtagtcaggtcactaacagtgagggacattcagaagactcagacacacagtcagtgtcagcccacggacaagctgggccccatcagcagagccacaaagagtccgcacgtggccagtcaggggaaagctctggacgttcaaggtctttcctctaccaggtgagctctcatgaacagtctgagtccacacacggacagactgcacccagcactggaggaagacaaggatcccgccatgagcaggcacgaaacagctctaggcactcagcatcccaagacggtcaggacaccattcgtggacacccggggtcaagcagaggaggaaggcagggatcctaccacgagcaatcagtagataggtctggacactcagggtaccatcacagccacaccacaccccagggaaggtctgatgcctcccatgggcagtcaggacccagaagtgcaagcaggcaaacaagaaatgaggaacaatcaggagacggctccaggcactcagggtcacgtcaccatgaaccttccactcgggccggcagctctagacactcacaggtgggccagggagaatcagcggggtccaagacaagcaggcgccagggatccagtgttagtcaggacagggacagtgagggacactcagaagactctgagaggcggtctgagtcggcttccagaaaccattatggatctgctcgggagcagtcaagacatggctccaggaaccccaggtcccatcaagaagatagagccagtcatgggcactctgcagagagctccagacaatcaggcactcgtcatgcagagacttcctctggtggacaggctgcatcatcccaggaacaggcaaggtcaagtccaggagaaagacatggatcccgccaccagcagtcagcagacagctccacagactcaggcactgggcgcagacaagattcatctgtagtcggagacagtggaaaccgagggtccagtggtagccaggccagtgacagcgagggacactcagaagagtcagacacacagtcagtgtcagcccacggacaggctgggccccatcagcagagccaccaagagtccacacgtggccagtcaggggaaaggtctggacgttcagggtctttcctctaccaggtgagcactcatgaacagtctgagtccgcccatggacgcacagggcccagcactggaggaagacaaagatcccgccacgagcaggcacgagacagctccaggcactcagcgtcccaagagggtcaggacaccattcgtggacacccagggtcaagcagaggaggaaggcagggatcccactatgagcaatcggtagatagttctggacactcagggtctcatcacagccacaccacgtcccaggaaaggtctgatgtctcccgtgggcagtcaggatccagaagtgtcagcagacaaacacgtaatgagaaacaatcaggagacggctccaggcactcagggtcgcgtcaccatgaagcttcctctcgggccgacagctctagacactcgcaggtgggccagggacaatcatcagggcccaggacaagcaggaaccagggatccagtgttagccaggacagtgacagtcagggacactcagaagactctgagaggtggtctgggtctgcttccagaaaccatcttggatctgcttgggagcagtcaagagatggctccagacaccctgggtcccatcacgaagacagagccggtcacgggcactctgcagacagctccagacaatcaggcactcgtcacacagagtcttcctctcgtggacaggctgcgtcatcccatgaacaggcaagatcaagtgcaggagaaagacatggatcccaccaccagctccagtcagcagacagctccagacactcaggcattgggcatggacaagcttcatctgcagtcagagacagtggacaccgagggtacagtggtagtcaggccagtgacagtgagggacattcagaagactcagacacacagtcagtgtcagcacagggaaaagctgggccccatcagcagagccacaaagagtccgcacgtggccagtcaggggaaagctctggacgttcagggtctttcctctaccaggtgagcactcatgaacagtctgagtccacccatggacagtctgcgcccagcactggaggaagacaaggatcccattatgatcaggcacaagacagctccaggcactcagcatcccaagagggtcaggacaccattcgtggacacccggggccaagcagaggaggaagacaggggtcccaccaagagcaatcggtagataggtctggacactcagggtctcatcacagccacaccacatcccagggaaggtctgatgcctcccgtgggcagtcaggatccagaagtgcaagcagaaaaacatatgacaaggaacaatcaggagatggctctaggcactcagggtcgcatcatcatgaagcttcctcttgggccgacagctctagacactcactggtgggccagggacaatcatcagggcccaggacaagcaggccccggggatccagtgttagccaggacagtgacagtgagggacactcagaagattctgagaggcggtctgggtctgcgtccagaaaccatcatggatctgctcaggagcagtcaagagatggctccagacaccccaggtcccatcacgaagacagagccggtcatgggcactctgcagagagctccagacaatcaggcactcatcatgcagagaattcctctggtggacaggctgcatcatcccatgaacaggcaagatcaagtgcaggagagagacacggatcccaccaccagcagtcagcagacagctccagacactcaggcattgggcacggacaagcttcatctgcagtcagagacagtggacaccgagggtccagtggtagtcaggccagtgacagtgagggacattcagaagactcagacacacagtcagtgtcagcccacggacaggctgggccccatcagcagagccaccaagagtccacacgtggccggtcagcaggaaggtctggacgttcagggtctttcctctaccaggtgagcactcatgaacagtctgagtccgcccatggacggaccgggaccagcactggaggaagacaaggatcccaccacaagcaggcacgagacagctccaggcactcaacgtcccaagagggtcaggacaccattcatggacacccggggtcaagcagtggaggaaggcagggatcccactacgagcaattggtagatagatctggacactcagggtctcatcacagccacaccacatcccagggaaggtctgatgcctcccatgggcactcaggatccagaagtgcaagcagacaaactcgtaacgatgaacaatcaggagacggctccaggcactcagggtcgcgtcaccatgaagcttcctctcgggccgacagctctggacactcgcaggtgggccagggacaatcagaggggcccaggacaagcaggaactggggatccagttttagccaggacagtgacagtcagggacactcagaagactctgagaggtggtctgggtctgcttccagaaaccatcatggatctgctcaggagcagctaagagatggctccagacaccccaggtcccatcaagaagacagagctggtcatgggcactctgcagacagctccagacaatcaggcactcgtcacacacagacttcctctggtggacaggctgcatcatcccatgaacaggcaagatcaagtgcaggagaaagacatggatcccaccaccagcagtcagcagacagctccagacactcaggcattgggcacggacaagcttcatctgcagtcagagacagtggacaccgagggtacagtggtagtcaggccagtgacaatgagggacattcagaagactcagacacacagtcagtgtcagcccacggacaggctgggtcccatcagcagagccaccaagagtccgcacgtggccggtcaggggaaacgtctggacattcaggatctttcctctaccaggtgagcactcatgaacagtctgagtcctcccatggatggacggggcccagcactagaggaagacaaggatcccgccatgagcaggcacaagacagctccaggcactcagcatcccaagacggtcaggacaccattcgtggacacccggggtcaagcagaggaggaaggcaggggtaccaccacgagcattcggtagatagctctggacactcagggtcccatcacagccacaccacatcccagggaaggtctgatgcctcccgtgggcagtcaggatccagaagtgcaagcagaacaacacgtaatgaggaacaatcaggagacggctccaggcactcagggtcgcgtcaccatgaagcttccactcatgccgacatctctagacactcacaggcagtccagggacaatcagaggggtccaggagaagcaggcgccagggatccagtgtgagccaggacagtgacagtgagggacattcagaagactctgagaggtggtctgggtctgcttccagaaaccatcatggatctgctcaggagcagctaagagatggctccagacaccccaggtcccatcaagaagacagagctggtcatgggcactctgcagacagctccagacaatcaggcactcgtcacacacagacttcctctggtggacaggctgcatcatcccatgaacaggcaagatcaagtgcaggagaaagacatggatcccaccaccagcagtcagcagacagctccagacactcaggcattgggcacggacaagcttcatctgcagtcagagacagtggacaccgagggtacagtggtagtcaggccagtgacaatgagggacattcagaagactcagacacacagtcagtgtcagcccacggacaggctgggtcccatcagcagagccaccaagagtccgcacgtggccggtcaggggaaacgtctggacattcaggatctttcctctaccaggtgagcactcatgaacagtctgagtcctcccatggatggacggggcccagcactagaggaagacaaggatcccgccatgagcaggcacaagacagctccaggcactcagcatcccaatacggtcaggacaccattcgtggacacccggggtcaagcagaggaggaaggcaggggtaccaccacgagcattcggtagatagctctggacactcagggtcccatcacagccacaccacatcccagggaaggtctgatgcctcccgtgggcagtcaggatccagaagtgcaagcagaacaacacgtaatgaggaacaatcaggagacagctccaggcactcagtgtcacgtcaccatgaagcttccactcatgccgacatctctagacactcacaggcagtccagggacaatcagaggggtccaggagaagcaggcgccagggatccagtgtgagccaggacagtgacagtgagggacattcagaagactctgagaggtggtctgggtctgcttccagaaaccatcgtggatctgttcaggagcagtcaaggcacggctccagacaccccaggtcccatcacgaagacagagccggtcacgggcactctgcagaccgctccagacaatcaggcactcgtcacgcagagacttcctctggtggacaggctgcatcatcccatgaacaggcaagatcaagtccaggagagagacacggatcccgccaccagcagtcagcagacagctccagacactcaggcattccgcgtggacaagcttcatctgcagtcagagacagtagacactgggggtccagtggtagtcaggccagtgatagtgagggacattcagaagagtcagacacacagtcagtgtcaggccatggacaggctgggccccatcagcagagccaccaagagtccgcacgtgaccggtcagggggaaggtctggacgttcagggtctttcctctaccaggtgagcactcatgaacagtctgagtctgcccatgggcggaccaggaccagcactggacgaagacaaggatcccaccacgagcaggcacgagacagctccaggcactcagcgtcccaagagggtcaggacaccattcgtggacacccggggtcaagcagaagaggaaggcagggatcccactacgagcaatcggtagataggtctggacactcagggtcccatcacagccacaccacatcccagggaaggtctgatgcctcccgtgggcagtcaggatccagaagtgccagcagacaaactcgtaatgacgaacaatcaggagatggctccaggcactcatggtcgcatcaccatgaagcttccactcaggcggacagctctagacactcacagtccggccagggacaatcagcggggcccaggacaagcaggaaccagggatccagtgttagccaggacagtgacagtcagggacactcagaagactctgagaggtggtctgggtctgcttccagaaaccatcgtggatctgctcaggagcagtcaagagatggctccagacaccccacgtcccatcacgaagacagagccggtcacgggcactctgcagagagctccagacaatcaggcactcatcatgcagagaattcctctggtggacaggctgcatcatcccatgaacaggcaagatcaagtgcaggagagagacatggatcccaccaccagcagtcagcagacagctccagacactcaggcattgggcacggacaagcttcatctgcagtcagagacagtggacaccgagggtccagtggtagtcaggccagtgacagtgagggacattcagaagactcagacacacagtcagtgtcagcccacggacaggctgggccccatcagcagagccaccaagagtccacacgtggccggtcagcaggaaggtctggacgttcagggtctttcctctaccaggtgagcactcatgaacagtctgagtctgcccatggacgggctgggcccagtactggaggaagacaaggatcccgccacgagcaggcacgagacagctccaggcactcagcgtcccaagagggtcaggacaccattcgtggacacccggggtcaaggagaggaggaagacagggatcctaccacgagcaatcggtagataggtctggacactcagggtcccatcacagccacaccacatcccagggaaggtctgatgcctcccatgggcagtcaggatccagaagtgcaagcagagaaacacgtaatgaggaacagtcaggagacggctccaggcactcagggtcgcgtcaccatgaagcttccactcaggctgacagctctagacactcacagtccggccagggtgaatcagcggggtccaggagaagcaggcgccagggatccagtgttagccaggacagtgacagtgaggcatacccagaggactctgagaggcgatctgagtctgcttccagaaaccatcatggatcttctcgggagcagtcaagagatggctccagacaccccggatcctctcaccgcgatacagccagtcatgtacagtcttcacctgtacagtcagactctagtaccgctaaggaacatggtcactttagtagtctttcacaagattctgcgtatcactcaggaatacagtcacgtggcagtcctcacagttctagttcttatcattatcaatctgagggcactgaaaggcaaaaaggtcaatcaggtttagtttggagacatggcagctatggtagtgcagattatgattatggtgaatccgggtttagacactctcagcacggaagtgttagttacaattccaatcctgttgttttcaaggaaagatctgatatctgtaaagcaagtgcgtttggtaaagatcatccaaggtattatgcaacgtatattaataaggacccaggtttatgtggccattctagtgatatatcgaaacaactgggatttagtcagtcacagagatactattactatgagtaa

SEQ ID NO: 38 - DNA, 12186 bp, Artificial Sequence
  Synthetic
atgtctacactgctggaaaacatcttcgccatcatcaacctgttcaagcagtacagcaagaaggacaagaacaccgacacactgagcaagaaagagctgaaagaactgctcgagaaagagttccggcagatcctgaagaaccccgacgatcccgatatggtggacgtgttcatggaccacctggacatcgaccacaacaagaagatcgacttcaccgagtttctgctgatggtgttcaagctggcccaggcctactacgagagcaccagaaaagagaacctgcctatcagcggccacaagcacagaaagcacagccaccacgacaagcacgaggacaacaagcaagaagagaacaaagagaatcggaagcggcccagcagcctggaaagacggaacaacagaaagggcaacaagggcagaagcaagagccctagagagacaggcggcaagagacacgagagcagctccgagaagaaagagagaaagggctacagccccacacacagagaggaagagtacggcaagaaccaccacaacagcagcaaaaaagagaagaacaagaccgagaacacccggctgggcgacaatagaaagagactgagcgagcggctggaagagaaagaggacaacgaggaaggcgtgtacgactacgagaataccggccggatgacccagaagtggattcagtctggccacattgccacctactacaccatccaggacgaggcctacgacaccaccgatagcctgctggaagaaaacaagatctacgagcggagcagaagcagcgacggcaagagcagcagccaagtgaacagaagccggcacgagaacacaagccaggtgccactgcaagagagccggacaagaaagcggagaggcagcagagtgtcccaggatagagatagcgagggccacagcgaggatagcgagagacattctggcagcgccagcagaaatcaccacggatctgcctgggagcagagcagagatggctctagacaccctagaagccacgacgaggatagagcctctcacggccactctgccgattctagcagacagtctggcaccagacacgccgagacaagctctagaggacagacagccagctctcacgagcaggctagatcttccccaggcgaaagacacggctctggacatcagcagtctgccgatagttctagacacagcgccacaggcagaggacaggcctcttctgctgtgtccgatagaggccacagaggcagctctggaagccaggcctctgattctgagggccactccgagaacagcgatacccagtctgtgtctggacacggcaaagccggactgagacagcagagccaccaagagagcactagaggcagaagcggcgagagaagcggcagatctggcagctctctgtaccaggtgtccacacacgaacagcctgatagcgcccacggcagaactggaacatctactggtggcagacagggctctcaccacgaacaggccagagattcttccagacactccgccagccaagaaggccaggataccattagaggacaccccggatctagcagaggcggaaggcagggatctcatcatgagcagtccgtgaacagatccggccatagcggaagccaccacagccacacaacatcccagggaagatccgatgccagccacggccaatctggaagcagaagcgcctccagacagaccagaaatgaggaacagagcggcgacggcacaaggcacagcggatctagacatcacgaagccagcagccaggccgatagcagcagacactctcaagttggccagggccagtctagcggccccagaacatctagaaatcagggcagctccgtgtctcaggactccgatagtcagggccattctgaggattctgagcgttggagcggcagcgcctctagaaaccatcatggctctgcccaagagcagtctcgcgacggaagcagacatccacgcagccaccatgaagatagagccggccatggacatagcgccgacagctctagaaagagcggaaccagacacacccagaactcctctagcggacaggccgcatcttcacatgagcaggcaagaagctctgccggcgaaaggcatggttccagacatcagctgcagagcgccgactcttctagacattccggaacaggacatggccaggccagcagcgctgttagagattctggccatagaggctcaagcggctctcaggccaccgattccgaaggacactccgaagattctgacacccagagtgtctctggccacggacaggcaggacatcaccagcagtctcaccaagagtccgccagagatagatctggcgagagatccagaagatccggcagcttcctgtatcaagtctctacccacaagcagagcgagtctagccacggctggacaggaccttctacaggcgttagacagggcagtcaccatgagcaagcccgggacaattctaggcatagcgcctctcaggacggccaggacactataagaggccatcctggctctagccgcagaggtagacagggaagccatcacgaacagtctgtggatagaagcggccattccggcagccatcactctcacacaactagccaggggagatctgacgcatctagaggccagtctggatctagaagcgccagccggaccactaggaacgaggaacagtctagagatggcagcaggcactccggcagtagacatcatgaggcctctagccatgccgacatctcaagacattctcaggccggacagggccagagcgaaggctctagaacatctcggagacagggatctagcgtcagccaggacagcgacagcgaggggcattctgaagatagcgaacgttggagtggctccgcctctcggaatcatagaggaagcgcccaagaacagagcagacacggatcaaggcaccctcgctcacaccatgaggaccgtgctggtcatggacattccgctgattcctctaggcagagcggcacacctcacgccgaaacatctagcggaggacaagccgctagcagccatgaacaagctagaagctctcctggggagagacatggaagtagacaccagcagagcgctgatagcagccggcattctggaatccctagaaggcaggctagctccgccgtcagagattccggacattggggttctagtggttcccaggctagcgactctgaagggcacagcgaagagtccgatacacagagcgttagcggacatggacaggatggacctcaccaacaaagccatcaagaatctgctagagattggagcggaggaagatctgggagaagcggaagcttcatctaccaagtttcaacccacgagcaatccgagtccgctcacggaagaaccagaacaagcaccggcagaaggcaaggttcccatcacgagcaagccagagactctagccggcactctgcctctcaagagggccaagatacaatcagggctcaccccggtagtagaagaggcggtcgacaggggtcacatcatgaacaaagcgtggacagatctggacacagcggctcccaccactctcataccacatctcaaggccgctccgacgcctctcatggtcaatctgggtctagatctgccagcaggcagacccggaaagataagcaatctggcgacggctccagacatagtggaagcaggcatcatgaagccgccagctgggctgatagctctaggcattctcaagtgggacaagagcaaagctccggcagcaggacaagcagacaccagggaagctctgtgtcccaagacagcgattccgagcggcattccgatgactctgagagactgtccggaagcgcatcccggaatcaccatggaagcagccgggaacaaagtcgcgacggttcaagacacccaggcttccatcaagaggatagggctagtcacggtcacagcgctgattctagtaggcagtccggaactcaccacaccgaatctagcagtcacggccaggctgtgtcctctcatgaacaggctcgttctagccctggcgaacggcacggatctcgacaccaacaatcagccgactccagcagacatagcggcattggacacagacaggcatcctctgccgttagagacagcggacatagagggtcctctggctcccaagtgacaaactcagaggggcactcagaggattccgacacacagtcagtgtctgcccatggccaagctggaccacaccagcaatcccacaaagaaagtgccaggggacagagcggagagtccagcggcaggtctagaagtttcctttaccaagtgtccagtcatgagcaatctgagagcacccacggccagacagccccttctaccggcggcagacaagggtctagacacgaacaagcccggaactcctcaaggcattctgctagccaggacggacaagacacaatccggggacatcctggtagttcacgcggaggcaggcaggggtcctaccatgaacaatcagtggatcggagcggacacagtggctaccatcatagccatacaacccctcagggacgctctgatgcttctcatgggcagtcaggcccaagaagtgcttcccggcagactcggaatgaagaacaaagcggagatggctcccggcacagtggatcacgccatcatgaaccatctaccagagccggcagctccaggcatagccaagttggacaaggcgagagcgccggctccaaaaccagtcgtagacaaggctccagcgtttcccaggatcgggacagtgaaggccactctgaggactccgagagaagaagcgagtccgcctcaagaaatcattacggcagcgctagagagcagtcccggcacggttctaggaaccccagaagtcaccaagaggacagagccagtcatggccacagtgctgaaagttctcgccagtccggcactaggcatgccgaaaccagtagtggtggacaggctgcaagtagtcaagaacaggcaagatcaagcccaggcgagcgccatggttctaggcaccaacagtctgctgacagcagcacagatagcggaacaggcagacggcaggatagctctgttgtcggcgatagcggcaataggggcagtagtggatctcaggcttctgacagcgaaggccatagcgaagaaagcgacactcagagcgtgtccgcacacggtcaagcaggccctcatcaacaatcacaccaagaatccacacggggacagtccggggaacgtagcggacgttctgggagttttctctatcaagtgtcaacgcatgaacagtccgagagtgcccatggaagaaccggacctagtactggtggaaggcagaggtccagacatgaacaggccagggacagcagcagacattcagcctctcaagaaggacaggacacgattcgcggccatccgggaagtagtcgaggtggacgccaaggctctcactatgagcagagcgttgacagctccgggcactctggttctcaccattctcacactacaagccaagaaagatccgacgtgtccaggggccaaagtggcagtagatctgtgtcaaggcagacgagaaacgagaagcagtcaggcgacggatctcggcactcaggttcaaggcaccatgaagctagcagcagagccgattcctccaggcactcacaagtcggacagggacaaagcagcggccctaggacttccagaaaccagggatcatccgttagccaagacagtgactcccaggggcatagtgaagattccgaacgttggagcggatctgcctccagaaatcacctgggaagcgcttgggagcaatcaagagatggttctcggcatcccggctctcatcacgaggacagagcaggacatggacactccgcagatagctccagacagagcggcactagacataccgagagcagttcaagaggccaggccgcaagttcacacgaacaggctaggtcctctgcaggcgagagacatggcagccaccatcaactgcagtccgctgactcatcccgccactctggaattggacatggtcaagcctccagcgcagtccgcgattctgggcacagaggatattctggctcacaagcctccgactcagagggacatagcgaggactctgatacacagtctgtcagcgcccagggaaaagctgggccacatcaacagagtcacaaagagtctgctcgcgggcagtctggcgaaagtagcggaagaagtgggagctttctgtaccaagtttctacacatgagcagagtgaaagcacacacggacagtcagctcctagcactggcggtcgtcaaggctcccattatgatcaggcccaggactcttctcggcacagcgcttcacaagaggggcaagacactattaggggacaccctggaccatccagaggtggccggcaaggatcacatcaagagcaatcagtggacagaagcgggcactcaggcagtcaccacagccatacgacaagccaaggcagatctgatgcctccagagggcagagcggctctagaagtgccagcagaaagacctacgacaaagaacagtccggcgacggtagtagacattcaggctcacaccaccacgaagcctctagctgggccgacagtagtaggcactctcttgtcggccaaggacagtcctccggaccaagaacctctagaccaagagggtcaagtgtctcccaagactctgactccgaagggcattccgaggacagcgaaagaagatctggctctgccagtaggaaccaccacggaagtgcacaagaacaatcccgagatgggagcagacaccctcggagtcatcacgaagatcgcgccggacacggacattctgccgaatcatccaggcaaagtggaacccaccacgctgagaatagttctggcggccaggcagcaagtagccatgagcaagcaagatccagtgctggcgagcgtcacgggtcccatcatcagcaaagtgcagacagcagtcgtcacagcggaatcggacacgggcaagcaagttcagcagttagagactccggccaccgaggtagtagtggtagccaagctagcgatagtgaaggccattcagaagattcagatacccaaagcgtcagtgctcatggacaggccggtccacatcagcaatctcatcaagagagtacccggggcagatcagctgggagatccggtcgctctggatcattcttgtaccaagtctccacgcacgagcagtcagaaagcgctcatggcaggacaggcacttcaaccggtggacggcagggctcccatcacaaacaagcacgcgatagtagcaggcacagcacgagccaagagggacaagataccatccacggacaccctggaagctctagcggaggcagacagggtagccattatgagcagctggtggatcgctctggtcactcagggtcacaccatagccacactacttctcaggggagaagcgacgcttcccacgggcattctgggagtagatccgcaagccgccagacaagaaatgatgagcaaagtggcgacggaagtcgccattctggctcaagacatcacgaggctagctctagggccgatagctctggacatagtcaagtcggccaggggcaatccgagggacctagaacaagcagaaattggggcagcagcttcagccaagactcagattctcagggtcacagcgaggactccgaacgttggagcggcagtgccagcagaaatcatcatggaagcgcccaagagcagctgagagatggcagtagacaccccagatcacaccaagaggatcgcgcaggacatgggcacagtgccgattcctctagacagtccgggactagacacacacagacatcttcaggcggccaagccgcatcaagtcatgagcaagctaggtctagcgcaggcgagaggcacggttctcatcatcaacagagcgccgattcaagccggcacagcggtataggccatggacaggcttctagcgcagtcagagatagcggccacaggggctattccggatcacaggcaagcgacaatgagggtcatagcgaagatagtgatacccagtccgtttctgctcacggacaggctggaagccaccaacagtcccatcaagaaagcgccagaggaagatcaggcgaaacctctggccacagcggttcattcctctatcaagtttccactcacgaacaaagcgaaagcagccacggatggactggcccatccacaagaggcagacaaggcagcaggcatgagcaggctcaggattcaagcagacacagcgcatcccaggatggacaggataccatacggggccatccaggctcaagtagaggcggccgtcagggatatcaccacgagcacagtgttgattcttccgggcatagtggctcacatcactcccatacaacttcccaaggccgaagcgacgccagtagaggacaaagtggttcaagatccgcctccaggaccacacgcaacgaggaacaatcaggcgacgggtctagacactccggctctagacaccacgaagcttctacccacgccgacatcagtagacatagtcaggccgtgcagggacagtctgaaggcagtagaagatcacgcaggcaaggctcctcagttagtcaagattcagacagcgagggacacagtgaagattctgagcgttggagtggctcagccagccgcaatcatcacggctccgctcaagaacaacttcgagatggtagtcggcacccgagaagtcatcaagaagatagagctggacacggccactccgctgacagtagtagacagtctggaacacggcacacccagacaagtagtggcggacaagcagcatcatcccacgagcaagctagaagttcagccggcgagcggcatggatcacaccaccagcaatctgctgactcctcaagacacagtggaattggtcacggccaagccagtagtgccgtccgagatagtggacataggggatacagcggtagccaggcatccgataacgagggacactcagaagatagcgacacccaaagcgtttcagcccatggccaggcaggatctcaccaacagagtcaccaagaaagcgctcggggcagatctggggagacatctggccattccggatcatttctctaccaagtgtctacccacgaacagtctgaaagctctcacggctggaccgggccaagcacaagaggacgtcaaggatcaagacacgagcaggcccaggatagctcaagacatagcgccagccagtatggccaggacacaattcgagggcaccctggttcctcaagaggcggacgacaaggctaccaccacgaacactctgtcgattccagcggccactccggatctcatcacagccacaccaccagtcaggggcgatctgatgctagtagaggccaatcaggctcccgcagcgcttccagaaccaccagaaacgaagaacaatctggggatagctcacggcacagcgtgtcaagacaccatgaggccagcacacacgccgacatttccagacatagccaggcagttcaggggcaaagcgaaggttccagaaggtctagacgacaggggagttcagttagccaggattccgattcagagggccattccgaggactccgaacgttggagcggaagcgcctctcgcaatcataggggctctgtgcaagaacagtcaaggcatggctctcggcacccacgatctcaccatgaggatagagcaggacacggtcattcagccgacagaagcagacaaagcggaaccaggcatgccgagacatcatccggtggacaggccgcttcaagccatgaacaggcacgatcttcacctggcgagcgacatggtagcagacatcaacagtccgcagacagctctcgccacagtggtatccctaggggccaagcctcttcagccgttcgtgacagtaggcattggggctcatcaggctctcaagcaagtgatagcgaggggcactccgaagaatccgatactcaatctgttagcggccacggccaggccggtcctcaccaacaatcccaccaagagagcgctcgagatagatccggcggcagaagtggacgatctggttcatttctgtatcaagtctcaacacacgaacaatcagaatctgcccacggccgcaccagaacctcaactggtcgtcggcaaggcagccatcatgaacaggcaagagatagctcccgccacagcgctagccaagaaggccaagacactatcagagggcaccccggaagttccagaagaggaaggcaaggcagtcactacgagcagagtgtggatagatcagggcattcagggtcccatcactcccacaccacctcacaaggccgcagtgatgcatctaggggacaatctggcagtcggagtgcatctagacaaacccggaacgacgagcagagcggagatggaagcagacactcttggagccaccatcacgaggcctccacacaggctgactcttcacgccattctcagtctggtcaaggccaaagcgctggccccaggaccagtagaaatcaaggttctagtgtcagtcaggactcagacagccaaggccactccgaggatagtgaacgttggagcggttccgcaagcagaaatcacagaggctccgcacaagagcagtccagagatggctccaggcatcctacctcacaccacgaggatagagctggtcacgggcatagcgccgaaagttccagacaatctggaactcatcacgccgagaattccagcggaggccaggctgcatcttcccacgaacaagcaagatcatccgctggcgagaggcatggaagtcatcaccagcaatcagcagatagcagtcgccactcaggcatcggtcacggacaagcttcatctgccgtcagagactcaggccacaggggatctagcggttctcaggcaagcgatagcgaaggacattctgaggactcagacacacagagcgtttccgctcatgggcaagctggtccccatcaacagtcacatcaagagtctaccagaggccgcagcgccggaagaagtggtagatctggaagctttctttatcaagtgtctactcacgagcaatccgaaagtgcccatggcagagcaggaccatcaacaggtggtcgccaggggtctagacatgagcaggctagagactcctctaggcactctgcttcccaagaaggccaggacacaatccgtggacacccaggctctcgaagaggtggtagacaaggatcttaccacgagcagtccgtcgatagatcaggccacagtggcagtcatcattcccataccaccagccagggacgtagcgacgctagtcacggacaaagtggcagcagaagcgctagcagagagactcgcaacgaagaacagtctggcgacggctcaaggcactctggatcaaggcatcacgaagcctctactcaggccgatagctcccgacattctcagagcggtcaaggcgaatccgctggatcacgacgatctagacggcaagggtccagtgtgtcacaggattcagactctgaggcttaccctgaggacagtgaacgcagatccgagagcgcaagtcggaaccaccatggctccagccgcgagcaaagtcgagatggatctcgtcacccaggcagctcccacagagatacagctagccatgtgcagtctagccccgtgcagagcgactcttccacagccaaagagcacggacacttctccagtctgagtcaggattctgcctaccacagcggcatccagagtagaggatctccccacagcagctcctcttatcactaccagtccgagggcactgagcggcagaaaggacaatccggacttgtgtggcggcacggctcttatggcagcgctgactacgattacggcgagtctggctttaggcacagccagcatggcagcgtgtcctacaacagcaaccccgtggtgtttaaagagcgcagcgatatctgcaaggcctccgcctttggaaaggatcaccctcggtactacgccacctacatcaacaaggaccctggactgtgtggccactcctccgatatcagcaagcagctgggattttcccagagccagcggtactactactacgaatga

SEQ ID NO: 39 - DNA, 7176 bp, Homo sapiens
atgaccgacctcttgagaagtgttgtcaccgtaattgatgttttctacaaatacaccaagcaagatggggagtgtggcacactgagcaagggtgaactaaaggaacttctggagaaagagcttcatccagttctgaagaacccagatgatccagacacagtggatgtcatcatgcatatgctggatcgagatcatgacagaagattggactttactgagtttcttttgatgatattcaagctgactatggcctgcaacaaggtcctcagcaaagaatactgcaaagcttcagggtcaaagaagcataggcgtggtcaccgacaccaagaagaagaaagtgaaacagaagaggatgaagaggatacaccaggacataaatcaggttacagacattcaagttggagtgagggagaggagcatggatatagttctgggcactcaaggggaactgtgaaatgtagacatgggtccaactccaggaggctaggaagacaaggtaatttatccagctctgggaaccaagagggatctcagaaaagataccacaggtccagctgtggtcattcatggagtggtggcaaagacagacatggttccagctctgtagaactgagagaaagaataaacaagtcacacattagcccttctagggaatctggggaggagtatgaatctggatctggatcaaacagttgggaaaggaaaggtcatggtggtctgtcatgtggattggagactagtgggcatgaatcaaactctactcagtcaagaattagagaacaaaagcttgggtctagctgttcaggttcaggagacagtgggaggcgaagtcatgcatgtggttatagcaattcaagtgggtgtggaaggccacaaaatgcttcaagttcttgtcagtcacatagatttggagggcaaggaaatcaatttagctatattcagtcaggctgtcagtcaggaattaagggaggacaaggccatggctgtgtctcaggaggtcagccctctggatgtggtcaacctgagtctaacccctgtagtcagtcctatagtcagagaggatatggagctagagaaaatggtcaaccacagaactgtggaggacaatggagaacaggctcaagtcagtcctcttgctgtggacaatatgggtctggaggtagccagtcttgtagtaatggtcaacatgaatatggttcctgtggccgcttttcaaactcttctagttcaaatgaattttccaaatgtgatcaatatgggtctggttcaagtcagtctactagctttgaacaacatggaacaggcttgagtcagtcctctgggttcgaacaacatgtatgtggctcaggtcaaacttgtggccagcatgagtctacatcaagtcaatccttgggctatgaccagcatgggtctagctcaggtaagacatctggctttggacaacatgggtctggctcaggtcagtcctctggctttggacaatgtgggtcaggctcaggtcagtcctctggctttggacagcatgggtctgtctcaggacaatcctctggttttggacagcatgggtctgtctcaggacaatcctctggttttggacaacatgagtctagatcacgtcagtctagctatggccaacatggttctggctcaagtcaatcatctggctatggccaatatgggtctagagagacatctggctttggacaacatgggttgggctcaggtcaatccactggctttggccaatatggatcgggctcaggtcagtcctctggctttggacaacatgggtctggctcaggacaatcctctggctttggacaacatgagtctagatcaggtcagtctagttatggccaacacagttctggctcaagtcagtcatctggctatggccaacatgggtctagacagacatctggctttggacaacatgggtcaggctcaagtcaatccactggctttggccaatatggatcaggctcaggtcagtcctctggctttggacaacatgtttctggctcaggacaatcctctggttttggacaacatgagtctagatcaggtcattctagctatggccaacatggttttggctcaagtcaatcatctggctatggtcaacatgggtcaagttcaggacagacatctggatttggacaacacgagttaagctcaggtcagtcttccagctttggccaacatggatcaggctcaggtcagtcctctggctttggacaacatgggtctggctcaggacaatcctctggctttggacaacatgagtctagatcaggtcagtctagctatggccaacacagttctggctcaagtcagtcatctggctatggccaacatgggtctagacagacatctggctttggacaacatgggtcaggctcaagtcaatccactggctttggccaatatggatcaggctcaggtcagtccgctggctttggacaacatgggtctggctcaggacaatcctctggctttggacagcatgagtctagatcacatcagtccagctatggccaacatggttctggctcaagtcaatcatctggctatggtcaacatgggtcaagttcgggacagacatctggctttggacaacacaggtcaagctcaggtcaatactctggctttggacaacatggatcaggctcaggtcagtccagtggctttggacaacatgggactggctcaggacaatactctggttttggacaacatgagtctagatcacatcagtctagctatggccaacatggttctggctcaagtcagtcatctggctatggtcaacatgggtcaagttcaggacagacttttggatttggacaacacaggtcaggctcaggtcaatcctctggctttggccaacatggatcaggctcaggtcagtcctctggctttggacaacatgagtcaggctcaggaaaatcctctggctttggacagcatgagtctagatcaagtcagtctaattatggccaacatggttctggctcaagtcagtcatctggctatggtcaacatgggtctagttcaggacagacaactggctttggacaacacaggtcaagctcaggccaatactcaggctttggacaacatggatcaggctcagatcagtcctctggctttggacaacatgggactggttcaggacaatcctctggttttggacaatatgagtctagatcacgtcagtctagctatggccaacatggttctggctcaagtcaatcatctggctatggtcaacatgggtcaaattcaggacagacatctggatttggacaacacaggccaggctcaggtcagtcctctggctttggccaatatggatcgggctcaggtcagtcttctggctttggacaacatgggtcaggcacaggtaaatcctctggctttgcacagcatgagtacagatcaggtcagtctagctatggccaacatggtactggctccagtcaatcatctggctgtggccaacatgagtctggctcaggtccaaccacaagttttggacagcatgtgtctggctcagacaatttctctagttctggacaacatatatctgactcaggtcagtccactggatttggccaatatggttcaggctcaggtcaatcaactggcttgggccagggtgaatctcaacaagtagagtcaggatccacagttcatgggagacaggaaactactcatggtcagacaataaataccactagacatagccagtctggtcaaggacaatccacacagacagggtccagggtaactagaagacgaagatctagccaaagtgagaacagtgacagtgaagtgcactcaaaggtctcacacagacattcagaacacattcacacacaagctggatctcactacccaaagtcaggatccacagttcgcagaagacaaggaactactcatggacagagaggagataccactagacatggccattctggtcatggacagtctacacagacaggttccagaacatctggaagacagagatttagccacagtgatgccactgacagtgaagtgcactcaggggtctcacatagaccacactcacaagaacaaactcacagccaagctggatctcaacatggagagtcagaatccacagttcatgagagacatgaaactacttatggacagacaggagaggccactggacatggccactctggtcatggacagtccacacagagagggtccaggacaactggaagaaggggatctggccatagtgagtccagtgacagtgaagtgcactcagggggctcacacagaccacaatcacaagaacaaactcatggccaagccggatctcaacatggagagtcaggatccacagttcatgggagacacggaactactcatggacagacaggagataccactagacatgcccactatcatcatggaaaatccacacagagagggtccagtacaactggaagaaggggatctggccacagtgagtccagtgacagtgaagtgcactcagggggctcgcacacacattcaggacacactcacggccaaagtggatctcaacatggagagtcagaatccataattcatgacagacacagaattactcatggacagacaggagataccactagacattcctactctggtcatgaacaaaccacacagacagggtccaggacaactggaagacagagaactagccacagtgagtccactgacagtgaagtgcactcagggggctcacacagaccacactcacgagaacacacttacggccaagccggatctcaacatgaagagccagaattcacagttcatgagagacacggaactactcatggacagataggagataccactggacattcccactctggtcatggacagtccacacagagagggtccaggacaactggaagacagagatctagccacagtgagtccagtgacagtgaagtgcactcaggggtctcacacacacatacaggacacactcatggtcaagctggatctcaacatggacagtcagaatccatagttcctgagagacatggaactactcatggacagacaggagataccactagacatgcccactatcatcatggattaaccacacagacagggtccaggactactggaagaaggggatctggccacagtgagtacagtgacagtgaagggtactcaggagtctcacatacacattcaggacacactcatggccaagccagatctcaacatggagagtcagaatccatagttcatgagagacatggaactatacatggacagacaggcgataccaccagacatgcccactctggtcatggacagtccacacagacagggtccaggaccactggaagaaggtcatctggccacagtgagtacagtgacagtgaagggcactcagggttctcacaaagaccacactcacgaggacacactcacggccaggctggatctcaacatggagagtcagaatccatagttgacgagagacatggaactactcatggacagacaggagataccagtggacattctcaatctggtcatggacagtccacacagtcaggatccagtacaactggaagaaggagatctggccacagtgagtccagtgacagtgaagtgcactcagggggctcacatacacattcaggacacacacacagccaagccaggtctcaacatggagagtcagaatccacagttcacaagagacaccaaactactcatggacagacaggagataccactgaacatggccaccctagtcatggacaaaccatacagacagggtccaggacaactggaagaaggggatctggccacagtgagtacagtgacagtgaagggccctcaggggtctcacacacacattcaggacacactcacggtcaagctggatctcactatccagagtcaggatcctcagttcatgagagacacggaactactcatggacaaacagcagataccactagacatggccactctggtcatggacagtccacacagagagggtccaggacaactggaagaagggcatctggccacagtgagtacagtgacagtgaagggcactcaggggtctcacacacacattcaggacacgctcatggccaagccggatctcaacatggagagtcaggatcctcagttcatgagagacacggaactactcatggacagacaggagataccactagacatgctcactctggtcatggacagtccacacagagagggtcaaggacagctggaagaaggggatctggccacagtgagtccagtgacagtgaagtgcactcaggggtctcacacacacattcaggacacacttatggccaagccagatctcaacatggagagtcaggatctgccattcacgggagacagggaactatacatggacagacaggagataccactagacatggccagtctggtcatggacagtccacacagacaggttccaggacaactggaagacaaagatctagtcacagtgagtccagtgatagtgaagtgcactcagaggcctcacccacacattcaggacacactcacagccaagccggatctcgacatggacagtcaggatcctcaggtcatgggagacagggaactactcatggacagacaggagataccactagacatgcccactatggttatggacaatccacacagagagggtccaggacaactggaagaaggggatctggccacagtgagtccagtgacagtgaagtgcactcatggggctcacacacacattcaggacacattcagggccaagctggatctcaacaaagacagccaggatccacagttcatgggagactggaaactactcatggacagacaggagataccactagacatggccattctggttatggacaatccacacagacaggttccagatctagtagagcaagtcattttcagtcacatagtagtgaaaggcaaaggcatggatcaagtcaggtttggaaacatggcagctatggacctgcagaatatgactatgggcacactgggtatgggccttctggtggcagcagaaaaagcatcagtaattctcacctttcatggtcaacagacagcactgcaaacaagcaactgtctagacattga

SEQ ID NO: 40 - DNA, 681 bp, Homo sapiens
atggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaa

SEQ ID NO: 41 - DNA, 681 bp, Artificial Sequence
  Synthetic
atggattggggcaccctgcagacaattctcggcggcgtgaacaagcacagcaccagcatcggaaagatctggctgaccgtgctgttcatcttccgcatcatgatcctggtggtggccgccaaagaagtgtggggcgacgaacaggccgacttcgtgtgtaataccctgcagcctggatgcaagaacgtgtgctacgaccactacttccccatcagccacatcagactgtgggccctgcagctgatcttcgtgtctacaccagctctgctggtggccatgcacgtggcctatagacggcacgagaagaagcggaagttcatcaagggcgagatcaagtccgagttcaaggacatcgaggaaatcaagacccagaaagtgcggatcgagggcagcctgtggtggacctataccagcagcatcttcttccgcgtgatcttcgaggccgcctttatgtacgtgttctacgtgatgtacgacggcttcagcatgcagcggctggtcaagtgtaacgcctggccttgtcctaacaccgtggactgcttcgtgtcccggcctaccgagaaaaccgtgttcaccgtgtttatgatcgccgtgtccggcatctgcatcctgctgaatgtgaccgagctgtgctacctgctgatccggtactgtagcggcaagagcaagaaacccgtgtga

SEQ ID NO: 42 - DNA, 813 bp, Homo sapiens
atggactggaagacactccaggccctactgagcggtgtgaacaagtactccacagcgttcgggcgcatctggctgtccgtggtgttcgtcttccgggtgctggtatacgtggtggctgcagagcgcgtgtggggggatgagcagaaggactttgactgcaacaccaagcagcccggctgcaccaacgtctgctacgacaactacttccccatctccaacatccgcctctgggccctgcagctcatcttcgtcacatgcccctcgctgctggtcatcctgcacgtggcctaccgtgaggagcgggagcgccggcaccgccagaaacacggggaccagtgcgccaagctgtacgacaacgcaggcaagaagcacggaggcctgtggtggacctacctgttcagcctcatcttcaagctcatcattgagttcctcttcctctacctgctgcacactctctggcatggcttcaatatgccgcgcctggtgcagtgtgccaacgtggccccctgccccaacatcgtggactgctacattgcccgacctaccgagaagaaaatcttcacctacttcatggtgggcgcctccgccgtctgcatcgtactcaccatctgtgagctctgctacctcatctgccacagggtcctgcgaggcctgcacaaggacaagcctcgagggggttgcagcccctcgtcctccgccagccgagcttccacctgccgctgccaccacaagctggtggaggctggggaggtggatccagacccaggcaataacaagctgcaggcttcagcacccaacctgacccccatctga

SEQ ID NO: 43 - DNA, 813 bp, Artificial Sequence
  Synthetic
atggactggaaaaccctgcaggctctgctgagcggcgtgaacaagtacagcaccgccttcggcagaatctggctgagcgtggtgttcgtgttcagagtgctggtgtatgtggtggccgccgaaagagtttggggcgacgagcagaaggacttcgactgcaacacaaagcagcccggctgcaccaacgtgtgctacgacaactacttccccatcagcaacatccggctgtgggccctgcagctgatcttcgtgacatgtcctagcctgctggtcatcctgcacgtggcctacagagaggaacgcgagagaaggcacagacagaaacacggcgatcagtgcgccaagctgtacgacaatgccggcaaaaagcacggcggcctgtggtggacatacctgttcagcctgatcttcaagctgattatcgagttcctgttcctgtacctgctgcacaccctgtggcacggcttcaacatgcctagactggtgcagtgtgccaacgtggccccttgtcctaacatcgtggactgctacattgcccggcctaccgagaagaagatctttacctacttcatggtcggagccagcgccgtgtgtatcgtgctgaccatttgcgagctgtgctacctgatctgccaccgggttctgagaggcctgcacaaggataagcctagaggcggctgtagccctagcagctctgcctctagagccagcacctgtagatgccaccacaagctggtggaagccggcgaagtcgatcctgatcctggcaacaacaagctgcaggccagcgctcctaatctgacccctatctga

SEQ ID NO: 44 - DNA, 801 bp, Homo sapiens
atgaactgggcatttctgcagggcctgctgagtggcgtgaacaagtactccacagtgctgagccgcatctggctgtctgtggtgttcatctttcgtgtgctggtgtacgtggtggcagcggaggaggtgtgggacgatgagcagaaggactttgtctgcaacaccaagcagcccggctgccccaacgtctgctatgacgagttcttccccgtgtcccacgtgcgcctctgggccctacagctcatcctggtcacgtgcccctcactgctcgtggtcatgcacgtggcctaccgcgaggaacgcgagcgcaagcaccacctgaaacacgggcccaatgccccgtccctgtacgacaacctgagcaagaagcggggcggactgtggtggacgtacttgctgagcctcatcttcaaggccgccgtggatgctggcttcctctatatcttccaccgcctctacaaggattatgacatgccccgcgtggtggcctgctccgtggagccttgcccccacactgtggactgttacatctcccggcccacggagaagaaggtcttcacctacttcatggtgaccacagctgccatctgcatcctgctcaacctcagtgaagtcttctacctggtgggcaagaggtgcatggagatcttcggccccaggcaccggcggcctcggtgccgggaatgcctacccgatacgtgcccaccatatgtcctctcccagggagggcaccctgaggatgggaactctgtcctaatgaaggctgggtcggccccagtggatgcaggtgggtatccataa

SEQ ID NO: 45 - DNA, 801 bp, Artificial Sequence
  Synthetic
atgaactgggcctttctgcagggactgctgagcggcgtgaacaagtacagcaccgtgctgagcagaatctggctgagcgtggtgttcatcttcagagtgctggtgtacgtggtggccgccgaagaagtttgggacgacgagcagaaagacttcgtgtgcaacaccaagcagcctggctgccccaacgtgtgctacgacgagttcttccccgtgtctcatgtgcggctgtgggccctgcaactgatcctggtcacatgtcctagcctgctggtggtcatgcacgtggcctacagagaggaacgcgagagaaagcaccacctgaagcacggccctaatgctcccagcctgtacgacaacctgagcaagaaacgcggcggactgtggtggacatacctgctgagcctgatcttcaaggccgccgtggatgccggcttcctgtacatcttccaccggctgtacaaggactacgacatgcccagagtggtggcctgcagcgtggaaccttgtcctcacaccgtggactgctacatcagcagacccaccgagaagaaggtgttcacctacttcatggtcaccaccgccgccatctgcatcctgctgaatctgtccgaggtgttctacctcgtgggcaagagatgcatggaaatcttcggccccagacacagacggcccagatgcagagaatgcctgcctgacacctgtcctccatacgtgctgtctcaaggcggacaccccgaggatggcaatagcgtgttgatgaaggccggcagcgctcctgttgatgccggcggatatccttga

SEQ ID NO: 46 - DNA, 786 bp, Homo sapiens
atggattgggggacgctgcacactttcatcgggggtgtcaacaaacactccaccagcatcgggaaggtgtggatcacagtcatctttattttccgagtcatgatcctcgtggtggctgcccaggaagtgtggggtgacgagcaagaggacttcgtctgcaacacactgcaaccgggatgcaaaaatgtgtgctatgaccactttttcccggtgtcccacatccggctgtgggccctccagctgatcttcgtctccaccccagcgctgctggtggccatgcatgtggcctactacaggcacgaaaccactcgcaagttcaggcgaggagagaagaggaatgatttcaaagacatagaggacattaaaaagcagaaggttcggatagaggggtcgctgtggtggacgtacaccagcagcatctttttccgaatcatctttgaagcagcctttatgtatgtgttttacttcctttacaatgggtaccacctgccctgggtgttgaaatgtgggattgacccctgccccaaccttgttgactgctttatttctaggccaacagagaagaccgtgtttaccatttttatgatttctgcgtctgtgatttgcatgctgcttaacgtggcagagttgtgctacctgctgctgaaagtgtgttttaggagatcaaagagagcacagacgcaaaaaaatcaccccaatcatgccctaaaggagagtaagcagaatgaaatgaatgagctgatttcagatagtggtcaaaatgcaatcacaggtttcccaagctaa

SEQ ID NO: 47 - DNA, 786 bp, Artificial Sequence
  Synthetic
atggattggggcaccctgcacacctttatcggcggcgtgaacaagcacagcaccagcatcggcaaagtgtggatcaccgtgatcttcatcttccgcgtgatgatcctggtggtggccgctcaagaagtgtggggcgacgagcaagaggacttcgtgtgcaataccctgcagcctggatgcaagaacgtgtgctacgaccacttcttccccgtgtctcacatcagactgtgggccctgcagctgatcttcgtgtctacaccagctctgctggtggccatgcacgtggcctactacagacacgagacaacccggaagttcagacggggcgagaagcggaacgacttcaaggacatcgaggacatcaagaagcagaaagtccggatcgagggcagcctgtggtggacctataccagcagcatcttcttccggatcatcttcgaggccgcctttatgtacgtgttctacttcctgtacaacggctaccatctgccttgggtgctgaagtgcggcatcgacccctgtcctaacctggtggactgcttcatcagcagacccaccgagaaaaccgtgttcaccatcttcatgatcagcgccagcgtgatctgcatgctgctgaatgtggccgagctgtgctacctgctgctcaaagtgtgcttccggcggtccaagagagcccagacacagaagaatcaccccaatcacgccctgaaagagtccaagcagaacgagatgaacgagctgatctccgacagcggccagaacgccatcacaggctttccaagctga

SEQ ID NO: 48 - DNA, 999 bp, Homo sapiens
atgctgctgctggctgccgccttcctcgtggccttcgtgctgctgctgtacatggtgtctccgctcatcagccccaagcccctcgccctgcccggggcgcatgtggtggttacaggaggttccagtggcatcgggaagtgcattgctatcgagtgctataaacaaggagcttttataactctggttgcacgaaatgaggataagctgctgcaggcaaagaaagaaattgaaatgcactctattaatgacaaacaggtggtgctttgcatatcagttgatgtatctcaagactataaccaagtagagaatgtcataaaacaagcacaggagaaactgggtccagtggacatgctggtaaattgtgcaggaatggcagtgtcaggaaaatttgaagatcttgaagttagtacctttgaaaggttaatgagcatcaattacctgggcagcgtgtaccccagccgggccgtgatcaccaccatgaaggagcgccgggtgggcaggatcgtgtttgtgtcctcccaggcaggacagttgggattattcggtttcacagcctactctgcatccaagtttgccataaggggattggcagaagctttgcagatggaggtgaagccatataatgtctacatcacagttgcttacccaccagacacagacacacctggctttgccgaagaaaacagaacaaagcctttggagactcgacttatttcagagaccacatctgtgtgcaaaccagaacaggtggccaaacaaattgttaaagatgccatacaaggaaatttcaacagttcccttggctcagatgggtacatgctctcggccctgacctgtgggatggctccagtaacttctattactgaggggctccagcaggtggtcaccatgggccttttccgcactattgctttgttttaccttggaagttttgacagcatagttcgtcgctgcatgatgcagagagaaaaatctgaaaatgcagacaaaactgcctaa

SEQ ID NO: 49 - DNA, 999 bp, Artificial Sequence
  Synthetic
atgctgcttctggccgctgctttcctggtggcctttgtgctgctgctgtacatggtgtccccactgatcagccccaagcctcttgcacttcctggcgctcacgttgtcgtgacaggcggatcttctggcatcggcaagtgtatcgccatcgagtgctataagcagggcgccttcatcacactggtggccagaaacgaggacaagctgctgcaggccaagaaagaaatcgagatgcacagcatcaacgacaagcaggtcgtgctgtgtatcagcgtggacgtgtcccaggactacaaccaggtggaaaacgtgatcaagcaggcccaagagaagctgggccccgtggacatgctggttaattgtgccggaatggccgtgtccggcaagttcgaggatctggaagtgtccaccttcgagcggctgatgagcatcaactacctgggcagcgtgtaccccagcagagccgtgatcaccaccatgaaggagcggagagtcggccggatcgtgtttgtgtctagtcaggctggacagctgggcctgtttggctttacagcctacagcgccagcaagttcgccatcagaggactggctgaagccctgcagatggaagtgaagccctacaacgtgtacatcaccgtggcctatcctccagacaccgacacacctggcttcgccgaggaaaacaggaccaagccactggaaacccggctgatctccgagacaaccagcgtgtgcaagcctgaacaggtggccaagcagatcgtgaaggatgccatccagggcaacttcaacagcagcctgggctccgatggctacatgctgtctgccctgacatgtggcatggcccctgtgacctctatcaccgagggactccagcaggttgtgaccatgggactgttccggacaatcgccctgttctatctgggcagcttcgacagcatcgtgcggcggtgtatgatgcagagagagaagtccgagaacgccgacaagaccgcctaa

SEQ ID NO: 50 - DNA, 1935 bp, Homo sapiens
atgagtcgacagtttagttccaggtctgggtaccgaagtggagggggcttcagctctggctctgctgggatcatcaactaccagcgcaggaccaccagcagctccacacgccgcagtggaggaggtggtgggagattttcaagctgtggtggtggtggtggtagctttggtgctggtggtggatttggaagtcggagtcttgttaaccttggtggcagtaaaagcatctccataagtgtggctagaggaggtggacgtggtagtggctttggtggtggttatggtggtggtggctttggtggtggtggctttggtggtggtggctttggtggaggtggcattgggggtggtggctttggtggttttggcagtggtggtggtggttttggtggaggtggctttgggggtggtggatatgggggtggttatggtcctgtctgccctcctggtggcatacaagaagtcactatcaaccagagccttcttcagcccctcaatgtggagattgaccctgagatccaaaaggtgaagtctcgagaaagggagcaaatcaagtcactcaacaaccaatttgcctccttcattgacaaggtgaggttcctggagcagcagaaccaggtactgcaaacaaaatgggagctgctgcagcaggtagatacctccactagaacccataatttagagccctactttgagtcattcatcaacaatctccgaaggagagtggaccaactgaagagtgatcaatctcggttggattcggaactgaagaacatgcaggacatggtggaggattaccggaacaagtatgaggatgaaatcaacaagcggacaaatgcagagaatgaatttgtgaccatcaagaaggatgtggatggtgcttatatgaccaaggtggaccttcaggccaaacttgacaacttgcagcaggaaattgatttccttacagcactctaccaagcagagttgtctcagatgcagactcaaatcagtgaaactaatgtcatcctctctatggacaacaaccgcagtctcgacctggacagcatcattgctgaggtcaaggcccagtacgaggatatagcccagaagagcaaagctgaggccgagtccttgtaccagagcaagtatgaagagctgcagatcactgctggcagacatggggatagtgtgagaaattcaaagatagaaatttctgagctgaatcgtgtgatccagagacttagatctgaaatcgacaatgtcaagaagcagatctccaacttgcagcagtccatcagtgatgcagagcagcgtggcgagaatgccctcaaggatgccaagaacaagctgaatgacctggaggatgccctgcagcaggccaaggaagacctggcccgcctgctgcgcgactaccaggagctgatgaacacaaagctggccctggatctggagattgccacctacaggaccctcctggagggagaagaaagcaggatgtctggagaatgtgccccgaacgtgagtgtgtctgtgagcacaagccacaccaccatcagtggaggtggcagccgaggaggtggcggcggtggctacggctctggaggtagcagctatggctccggaggtggtagctatggttctggaggtggcggcggcggcggccgtggcagctatggctccggaggtagcagctacggctccggaggtggcagctatggctctggaggtggcggcggcggccatggcagctacggctccggaagcagcagtgggggctacagaggtggctctggaggcggcggcggcggcagctctggcggccggggctctggcggcgggagctctggaggctccataggaggccggggatccagctctgggggtgtcaagtcctctggtggcagttccagcgtgaagtttgtttctaccacttattccggagtaaccagataa

SEQ ID NO: 51 - DNA, 1935 bp, Artificial Sequence
  Synthetic
atgagccggcagttcagcagcagaagcggctacagatctggcggcggattttcttctggctccgccggcatcatcaactaccagagaagaaccaccagcagcagcaccagaagaagtggcggaggcggcggaagattttctagttgtggtggcggtggcggaagctttggagcaggcggaggatttggcagcagatccctggttaatctcggcggcagcaagagcatcagcatctctgttgctagaggcggaggcagaggctctggatttggaggcggatatggtggtggtggctttggcggcggtggtttcggcggaggcggatttggcggcggaggaattggaggcggtggctttggaggatttggaagcggaggtggtggattcggtggcggtggattcggaggtggcggttatggtggcggctatggacctgtttgtcctcctggcggcatccaagaagtgaccatcaaccagtctctgctgcagcccctgaacgtggaaatcgaccccgagatccagaaagtgaagtcccgcgagagagagcagatcaagagcctgaacaaccagttcgccagctttatcgacaaagtgcggttcctggaacagcagaaccaggtgctgcagaccaagtgggagctgctgcaacaggtggacaccagcacacggacccacaacctggaaccttacttcgagagcttcatcaacaacctgcggcggagagtggaccagctgaagtccgatcagagcagactggacagcgagctgaagaacatgcaggacatggtggaagattaccggaacaaatacgaggacgagatcaacaagcggaccaacgccgagaacgagttcgtgacaatcaagaaagacgtggacggcgcctacatgaccaaggtggacctgcaggccaagctggacaacctgcagcaagaaatcgacttcctgacagccctgtaccaggccgagctgagccagatgcagacacagatcagcgagacaaacgtgatcctgagcatggacaacaacagaagcctggacctggacagcatcattgccgaagtgaaggcccagtacgaggatatcgcccagaagtctaaggccgaggccgagagcctgtatcagtctaagtatgaggaactgcagatcaccgccggcagacacggcgatagcgtgcggaatagcaagatcgagatctccgagctgaacagagtgatccagcggctgagaagcgagatcgacaacgtgaagaagcagatcagcaatctgcagcagtccatcagcgacgccgagcagagaggcgaaaacgccctgaaggacgccaagaacaagctgaacgacctggaagatgccctgcagcaggccaaagaggacctggctagactgctgcgggactaccaagagctgatgaacaccaagctggccctggatctggaaatcgccacctacagaaccctgctggaaggcgaggaatccagaatgagcggcgagtgtgcccctaacgtgtcagtgtctgtgtccaccagccacaccacaatctctggcggaggttcaagaggtggtggcggcggtggatatggaagcggcggaagctcttatggatctggtggcggcagttacggatctggcggaggtggcggcggaggtagaggatcttatggaagtggcggcagcagctatggttcaggcggagggtcttatggttctggcggcggaggcggtggacacggaagttatggaagcggatctagcagcggcggctaccgcggaggatcaggcggaggcggaggtggaagttctggtggaagaggttctggcggaggaagctctggcggatctatcggcggaagaggatcttctagcggcggagtgaaaagcagcggaggctccagcagcgtgaagttcgtgtccacaacatacagcggcgtgacccggtaa

SEQ ID NO: 52 - DNA, 1920 bp, Homo sapiens
atgagttgtcagatctcttgcaaatctcgaggaagaggaggaggtggaggaggattccggggcttcagcagcggctcagctgtggtgtctggtggaagccggagatcaacttccagcttctcctgcttgagccgccatggtggtggtggcgggggcttcggtggaggcggctttggcagtcggagtcttgttggccttggagggaccaagagcatctccattagtgtggctggaggaggtggtggctttggcgccgctggtggatttggtggcagaggaggtggttttggaggcggcagcagctttggaggtggcagcggcttcagtggtggtggtttcggtggaggcggctttggtggaggccgctttggaggttttgggggccctggtggtgttggaggtttagggggtcctggtggctttgggcctggaggataccctggtggcatccacgaagtctctgtcaaccagagcctcctgcagcctctcaacgtgaaagttgacccagagatccagaatgtgaaggcccaagagcgtgagcagatcaaaactctcaacaacaaatttgcctccttcattgacaaggtgcggttcttggagcagcagaaccaggtgttacagaccaaatgggagctgctacaacaaatgaatgttggcacccgccccatcaacctggagcccatcttccaggggtatatcgacagcctcaagagatatctggatgggctcactgcagaaagaacatcacagaattcagagctgaataacatgcaggatcttgtggaggattataagaagaagtatgaggatgaaatcaataagcgcacagctgctgagaatgattttgtgacgcttaaaaaggacgtggacaatgcctacatgataaaggtggagttgcagtccaaggtggacctgctgaaccaggaaattgagtttctgaaagttctctatgatgcggagatatcccagatacatcagagtgtcactgacaccaacgtcatcctctccatggacaacagccgcaacctggacttggatagcatcatcgccgaggtcaaggcccagtatgaggagatcgcccagaggagcaaggaagaagcggaggccctgtaccacagcaagtatgaggagctccaggtgactgtcgggagacatggagacagcctgaaagagatcaagatagagatcagcgagctgaaccgcgtgatccagaggctgcagggggagatcgcacatgtgaagaagcagtgtaagaatgtgcaagatgccatcgcagatgccgagcagcgtggggagcatgccctcaaggatgccaggaacaagttgaatgacctggaggaggccctgcagcaggccaaggaggacttggcgcggctgctgcgtgactaccaggagctgatgaacgtgaagctggccctagatgtggagatcgccacctaccgcaaactgctggagggcgaggagtgcaggatgtctggagacctcagcagcaatgtgactgtgtctgtgacaagcagcaccatttcatcaaatgtggcatccaaggctgcctttggaggttctggaggtagagggtccagttccggaggaggatacagctctggaagcagcagttatggctctggaggccgacagtctggctccagaggcggtagtggaggaggaggttctatctctggaggaggatatggctctggcggtggttctggaggaagatacggatctggtggtggctctaagggagggtccatctctggaggaggatatggctctggaggtggaaaacacagctctggaggtggctctagaggaggctccagctctggaggaggatatggctctggaggtgggggttctagctctgtaaagggtagctcaggtgaagcttttggttccagcgtgaccttctcttttagataa

SEQ ID NO: 53 - DNA, 1920 bp, Artificial Sequence
  Synthetic
atgagctgccagatcagctgcaagagcagaggaagaggtggcggcggaggcggctttagaggattttcttctggctccgccgtggtgtctggcggcagcagaagaagcacaagcagcttcagctgcctgagcagacatggtggcggtggcggaggatttggaggcggtggttttggcagcagatctctcgttggcctcggcggcacaaagtccatctctatttctgtggctggcggaggtggcggatttggtgctgctggtggatttggcggaagaggcggaggtttcggcggaggaagttcttttggcggaggctccggattttcaggcggtggcttcggaggcggtggctttggtggtggaagattcggcggttttggaggccctggcggagttggaggtcttggaggaccaggcggattcggacctggtggatatcctggcggcattcacgaggtgtccgtgaaccagtctctgctgcagcccctgaacgtgaaggtggaccccgagatccagaatgtgaaggcccaagagagagagcagatcaagaccctgaacaacaagttcgccagctttatcgacaaagtgcggttcctggaacagcagaaccaggtgctgcagaccaagtgggaactgctccagcagatgaacgtgggcaccagacctatcaacctggaacctatcttccagggctacatcgacagcctgaagagatacctggatggcctgaccgccgagagaaccagccagaatagcgagctgaacaatatgcaggacctggtcgaggactacaagaagaagtacgaggacgagatcaacaagcggacagccgccgagaacgacttcgtgaccctgaagaaagacgtggacaacgcctacatgatcaaggtggaactgcagagcaaagtggacctgctgaatcaagagatcgagttcctgaaggtgctgtacgacgccgagatctcccagatccaccagagcgtgaccgacaccaacgtgatcctgagcatggacaacagccggaacctggacctggacagcatcattgccgaagtgaaagcccagtacgaagagatcgcccagcggagcaaagaggaagccgaggctctgtaccacagcaaatacgaggaactgcaagtgaccgtgggcagacacggcgactccctgaaagagatcaagatcgagatcagcgaactgaacagagtgatccagcggctgcagggcgaaatcgcccacgtgaagaaacagtgcaagaacgtgcaggacgccattgccgatgccgagcaaaggggagaacacgccctgaaggacgcccggaacaagctgaacgatctggaagaggctctgcagcaggctaaagaggacctggccagactgctgcgggactaccaagagctgatgaatgtgaaactggccctggacgtggaaatcgccacctacagaaagctgctggaaggcgaggaatgccggatgagcggagatctgagcagcaacgtgacagtgtctgtgaccagctccaccatcagctccaacgtggccagcaaggccgcctttggaggtagcggtggaagaggatctagttccggcggagggtacagcagcggctctagctcttatggaagcggcggcagacagagcggatctagaggcggaagcggaggcggaggatcaatttctggtggcggatatggctctggcggcggttctggtggtagatacggttcaggtggcgggtctaaaggcggctctatatcaggcggcggatacggatcaggcggaggcaaacactcttctggcggaggtagtagaggcggcagttcaagcggcggtggttatggatctggcggaggcggatctagcagcgtgaaaggatctagcggcgaggcctttggcagctccgtgaccttcagcttcagatga

SEQ ID NO: 54 - DNA, 1872 bp, Homo sapiens
atgagctgcagacagttctcctcgtcctacttgagccgcagcggcgggggtggcgggggcggcctgggcagcgggggcagcataaggtcttcctacagccgcttcagctcctcagggggcggtggaggagggggccgattcagctcttctagtggctatggtgggggaagctctcgtgtctgtgggaggggaggcggtggcagttttggctacagctacggcggaggatctgggggtggttttagtgccagtagtttaggcggtggctttgggggtggttccagaggttttggtggtgcttctggaggaggctatagtagttctgggggttttggaggtggctttggtggtggttctggaggtggctttggtggtggctatgggagtgggtttggggggtttgggggctttggaggtggtgctggaggaggtgatggtggtattctgactgctaatgagaagagcaccatgcaggaactcaattctcggctggcctcttacttggataaggtgcaggctctagaggaggccaacaacgacctggagaataagatccaggattggtacgacaagaagggacctgctgctatccagaagaactactccccttattataacactattgatgatctcaaggaccagattgtggacctgacagtgggcaacaacaaaactctcctggacattgacaacactcgcatgacactggatgacttcaggataaagtttgagatggagcaaaacctgcggcaaggagtggatgctgacatcaatggcctgcggcaggtgctggacaatctgaccatggagaagtctgacctggagatgcagtatgagactctgcaggaggagctgatggccctcaagaagaatcataaggaggagatgagtcagctgactgggcagaacagtggagatgtcaatgtggagataaacgttgctcctggcaaagatctcaccaagaccctcaatgacatgcgtcaggagtatgagcagctcattgctaagaacagaaaggacatcgagaatcaatatgagactcagataacccagatcgagcatgaggtatccagtagtggtcaggaggtgcagtccagtgccaaggaggtgacccagctccggcacggtgtccaggagttggagattgagctgcagtctcagctcagcaagaaagcagctctggagaagagcttggaagacacgaagaaccgctactgtggccagctgcagatgatccaggagcagatcagtaacttggaggcccagatcactgacgtccggcaagagatcgagtgccagaatcaggaatacagccttctgctcagcattaagatgcggctggagaaggaaatcgagacctaccacaacctccttgagggaggccaggaagactttgaatcctccggagctggaaaaattggccttggaggtcgaggaggaagtggaggcagttatggaagaggatccaggggaggaagtggaggcagctatggtggaggaggaagtggaggtggctatggtggaggaagtgggtccaggggaggaagtggaggcagctacggtggaggaagtggttctggaggaggtagtggaggtggctatggtggaggaagtggaggtggccatagcggaggaagtggaggtggtcatagtggaggaagtgggggcaactatggaggaggaagtggctctggaggaggaagtgggggtggctatggtggaggaagtgggtccaggggaggaagtggaggcagccatggtggaggaagtggttttggaggtgaaagtggaggcagctacggaggcggtgaagaagcgagtggaagtggtggcggctacggaggaggaagcggaaaatcatcccattcctag

SEQ ID NO: 55 - DNA, 1872 bp, Artificial Sequence
  Synthetic
atgagctgcagacagttcagcagcagctacctgagcagatctggtggtggcggaggcggaggacttggatctggcggatctatcagaagctcctacagccggtttagcagctctggcggaggtggcggcggtggcagattttcttctagctctggatacggcggaggcagcagcagagtttgtggaagaggcggcggaggaagcttcggctactcttatggcggtggttctggcggcggattttctgccagttctctcggaggcggttttggaggcggctccagaggatttggcggagcttcaggtggcggatactctagcagtggtggcttcggcggtggatttggtggtggtagtggtggtggattcggaggcggctatggctctggctttggcggcttcggaggtttcggcggaggtgcaggcggaggcgacggtggtattctgaccgccaatgagaagtccaccatgcaagagctgaacagcagactggcctcctacctggacaaagtgcaggccctggaagaagccaacaacgacctggaaaacaagatccaggattggtacgacaagaagggccctgccgccatccagaagaactacagcccctactacaacaccatcgacgacctgaaggaccagatcgtggatctgaccgtgggcaacaacaagaccctgctggacatcgacaacacccggatgaccctggacgacttccggatcaagttcgagatggaacagaacctgagacagggcgttgacgccgacatcaacggactcagacaggtgctggacaacctgaccatggaaaagtccgatctggaaatgcagtacgagacactgcaagaggaactgatggccctgaagaaaaaccacaaagaagagatgagccagctgaccggccagaactctggcgacgtgaacgtggaaatcaacgtggcccctggcaaggacctgaccaagacactgaacgacatgcggcaagagtacgagcagctgatcgccaagaaccggaaggacatcgagaaccagtatgagacacagatcacccagatcgagcacgaggtgtcctctagcggacaagaggtgcagagcagcgccaaagaagtgacccagctcagacacggcgtgcaagaactggaaatcgagctgcagtcccagctgagcaagaaggccgctctggaaaagagcctggaagataccaagaacagatactgcggccagctgcagatgatccaagagcagatcagcaacctggaagcccagatcaccgacgtccggcaagagatcgagtgccagaatcaagagtacagcctgctgctgtccatcaagatgcggctggaaaaagagattgagacataccacaacctgctggaaggcggccaagaggactttgagtctagcggagccggaaagatcggcctcggaggtagaggtggaagcggcggatcttatggcagaggctctagaggcggttccggcggaagttatggtggcggtggcagtggcggaggatatggcggaggatctggatctagaggcggcagcggaggctcttatggtggtggaagtggaagcggtggtggtagcggcggaggctacggtggcggatctggcggaggacattctggtggttcaggcggagggcatagtggtggctctggtggcaattatggcggcggtagcggatcaggcggtggctccggcggtggttatggcggaggaagcggaagtagaggcggctctggtggatctcacggcggtggatcaggatttggaggcgaatctggcggctcatatggcggcggagaagaagcctctggaagcggaggcggctacggcggtggctctggaaaaagctctcacagctga

SEQ ID NO: 56 - DNA, 1755 bp, Homo sapiens
atgtctgttcgatacagctcaagcaagcactactcttcctcccgcagtggaggaggaggaggaggaggaggatgtggaggaggaggaggagtgtcatccctaagaatttctagcagcaaaggctcccttggtggaggatttagctcaggggggttcagtggtggctcttttagccgtgggagctctggtgggggctgctttgggggctcatcaggtggctatggaggattaggaggttttggtggaggtagctttcgtggaagctatggaagtagcagctttggtgggagttatggaggcagctttggagggggcagtttcggaggtggcagctttggtgggggcagctttggtggaggcggctttggtggaggcggctttggaggaggctttggtggtggatttggaggagatggtggccttctctctggaaatgaaaaagtaaccatgcagaatctgaatgaccgcctggcttcctacttggacaaagttcgggctctggaagaatcaaactatgagctggaaggcaaaatcaaggagtggtatgaaaagcatggcaactcacatcagggggagcctcgtgactacagcaaatactacaaaaccatcgatgaccttaaaaatcagattctcaacctaacaactgataatgccaacatcctgcttcagatcgacaatgccaggctggcagctgatgacttcaggctgaagtatgagaatgaggtagctctgcgccagagcgtggaggctgacatcaacggcctgcgtagggtgctggatgagctgaccctgaccaaggctgacctggagatgcaaattgagagcctgactgaagagctggcctatctgaagaagaaccacgaggaggaaatgaaagaccttcgaaatgtgtccactggtgatgtgaatgtggaaatgaatgctgccccgggtgttgatctgactcaacttctgaataacatgagaagccaatatgaacaacttgctgaacaaaaccgcaaagatgctgaagcctggttcaatgaaaagagcaaggaactgactacagaaattgataataacattgaacagatatccagctataaatctgagattactgaattgagacgtaatgtacaagctctggagatagaactacagtcccaactggccttgaaacaatccctggaagcctccttggcagaaacagaaggtcgctactgtgtgcagctctcacagattcaggcccagatatccgctctggaagaacagttgcaacagattcgagctgaaaccgagtgccagaatactgaataccaacaactcctggatattaagatccgactggagaatgaaattcaaacctaccgcagcctgctagaaggagagggaagttccggaggcggcggacgcggcggcggaagtttcggcggcggctacggcggcggaagctccggcggcggaagctccggcggcggccacggcggcggccacggcggcagttccggcggcggctacggaggcggaagctccggcggcggaagctccggcggcggctacgggggcggaagctccagcggcggccacggcggcagttccagcggcggctacggtggtggcagttccggcggcggcggcggcggctacgggggcggcagctccggcggcggcagcagctccggcggcggatacggcggcggcagctccagcggaggccacaagtcctcctcttccgggtccgtgggcgagtcttcatctaagggaccaagatactaa

SEQ ID NO: 57 - DNA, 1755 bp, Artificial Sequence
  Synthetic
atgagcgtgcggtacagcagcagcaagcactacagcagctctagatctggtggtggtggcggaggcggtggatgtggcggtggcggcggagttagtagcctgagaatcagctctagcaagggctctctcggcggaggctttagctctggcggattttctggcggcagcttcagcagaggatctagcggcggaggatgttttggcggctctagcggaggatatggcggcctcggaggatttggaggcggctcttttagaggcagctacggctccagcagcttcggcggatcttacggcggttcttttggcggaggaagcttcggaggcggatcatttggtggtggctcatttggcggcggtggctttggaggtggtggttttggtggcggatttggcggtggttttggcggagatggtggcctgctgagcggcaacgagaaagtgaccatgcagaacctgaacgacagactggccagctacctggacaaagtgcgcgccctggaagagagcaactacgagctggaaggcaagatcaaagagtggtacgagaagcacggcaacagccaccagggcgagcctagagactactccaagtactacaagaccatcgacgacctgaagaaccagatcctgaacctgaccaccgacaacgccaacatcctgctgcagatcgacaatgccagactggctgccgacgacttccggctgaagtacgagaatgaggtggccctgagacagagcgtggaagccgatatcaacggcctgagaagagtgctggacgagctgaccctgaccaaggccgatctggaaatgcagattgagagcctgaccgaggaactggcctatctgaagaagaaccacgaggaagagatgaaggacctgcggaacgtgtccaccggcgacgtgaacgtggaaatgaatgctgctccgggcgtcgacctgacacagctgctgaacaacatgcggagccagtacgagcagctggccgagcagaatagaaaggatgccgaggcctggttcaacgagaagtccaaagagctgaccacagagatcgataacaacatcgagcagatcagcagctacaagagcgagatcaccgagctgcggagaaacgtgcaggccctggaaatcgagctgcagtctcagctggccctgaagcagtctctggaagcctctctggccgaaaccgagggcagatactgtgtgcagctgagccagattcaggcccagattagcgccctcgaggaacagctgcagcagatccgggctgaaaccgagtgccagaacaccgaataccagcaactgctggacatcaagatccggctggaaaacgagatccagacctacagaagcctgctcgaaggcgagggaagttcaggtggtggtggaagaggtggcggaagttttggaggcggttatggcggaggatcatctggcggaggttcaagcggaggtggacatggtggcggccatggtggatcttcaggcggcggatacggcggtggaagttccggcggaggtagtagtggcggtggctatggtggcggatcttcttctggtggccatggcggtagtagcagcggaggctacggcggagggtctagtggcggcggaggtggtggctatggcggaggcagttcaggcggaggctcatccagtggtggtggatacggcggaggatcttctagcggcggacacaagtctagcagctctggctctgtgggcgagagcagctccaagggacctagatactga

SEQ ID NO: 58 - DNA, 1197 bp, Homo sapiens
atgatgtggctgcttttaacaacaacttgtttgatctgtggaactttaaatgctggtggattccttgatttggaaaatgaagtgaatcctgaggtgtggatgaatactagtgaaatcatcatctacaatggctaccccagtgaagagtatgaagtcaccactgaagatgggtatatactccttgtcaacagaattccttatgggcgaacacatgctaggagcacaggtccccggccagttgtgtatatgcagcatgccctgtttgcagacaatgcctactggcttgagaattatgctaatggaagccttggattccttctagcagatgcaggttatgatgtatggatgggaaacagtcggggaaacacttggtcaagaagacacaaaacactctcagagacagatgagaaattctgggcctttagttttgatgaaatggccaaatatgatctcccaggagtaatagacttcattgtaaataaaactggtcaggagaaattgtatttcattggacattcacttggcactacaatagggtttgtagccttttccaccatgcctgaactggcacaaagaatcaaaatgaattttgccttgggtcctacgatctcattcaaatatcccacgggcatttttaccaggttttttctacttccaaattccataatcaaggctgtttttggtaccaaaggtttctttttagaagataagaaaacgaagatagcttctaccaaaatctgcaacaataagatactctggttgatatgtagcgaatttatgtccttatgggctggatccaacaagaaaaatatgaatcagagtcgaatggatgtgtatatgtcacatgctcccactggttcatcagtacacaacattctgcatataaaacagctttaccactctgatgaattcagagcttatgactggggaaatgacgctgataatatgaaacattacaatcagagtcatccccctatatatgacctgactgccatgaaagtgcctactgctatttgggctggtggacatgatgtcctcgtaacaccccaggatgtggccaggatactccctcaaatcaagagtcttcattactttaagctattgccagattggaaccactttgattttgtctggggcctcgatgcccctcaacggatgtacagtgaaatcatagctttaatgaaggcatattcctaa

SEQ ID NO: 59 - DNA, 1197 bp, Artificial Sequence
  Synthetic
atgatgtggctgctgctgaccaccacctgtctgatctgcggcactctgaatgccggcggattcctggacctggaaaacgaagtgaaccccgaagtgtggatgaacaccagcgagatcatcatctacaacggctaccccagcgaggaatatgaagtgaccaccgaggacggctacatcctgctggtcaacagaatcccctacggcagaacccacgccagaagcacaggacctagacctgtggtgtacatgcagcacgccctgttcgccgacaatgcctactggctggaaaactacgccaacggcagcctgggattcctgctggccgatgccggatacgatgtgtggatgggcaacagcagaggcaacacctggtccagaaggcacaagaccctgagcgagacagacgagaagttctgggccttcagcttcgacgagatggccaaatacgacctgcctggcgtgatcgacttcatcgtgaacaagaccggccaagagaagctgtacttcatcggccacagcctgggcaccaccatcggctttgtggcctttagcaccatgcctgagctggcccagcggatcaagatgaactttgccctgggacctaccatctccttcaagtaccccaccggaatcttcacccggttctttctgctgcccaacagcatcatcaaggccgtgttcggaacaaagggattcttcttggaggacaaaaagaccaagatcgccagcaccaagatctgcaacaacaagatcctgtggctgatctgcagcgagttcatgtctctgtgggccggcagcaacaagaaaaacatgaaccagagccggatggacgtgtacatgtctcacgcccctacaggcagcagcgtgcacaacatcctgcacatcaagcagctgtaccacagcgacgagttccgggcctacgactggggaaacgacgccgacaacatgaagcactacaaccagtctcaccctcctatctacgacctgaccgccatgaaggtgccaaccgctatttgggctggcggccatgatgtgctggtcacacctcaggatgtggccagaatcctgcctcagatcaagagcctgcactacttcaagctgctgcccgactggaaccacttcgactttgtgtggggactcgacgcccctcagagaatgtacagcgagattatcgccctgatgaaggcctacagctga

SEQ ID NO: 60 - DNA, 939 bp, Homo sapiens
atgtcttatcagaaaaagcagcccacccctcagcccccagtggactgcgtgaagacctctggcggcggtggcggtggcggcggcagcggcggtggtggctgcggcttcttcggcggcggcggctcagggggcggtagcagcggttctggctgcggctactccggcggcggtggctactctggcggcggctgcggcgggggctcctccggcggcgggggcgggggcggcattggaggctgcggagggggctccggtgggagcgtcaagtactccggaggcggcggctcctccggcgggggctctggctgtttctccagcggtgggggcggctccggctgcttctcctccggtggcggcggctcctccgggggcggctccggctgcttctccagcggtgggggcggctcctccgggggcggctccggctgcttctcctccggcggcggcggcttctcgggccaggcggtccagtgccagagctacggaggcgtctctagcggcggctcctccgggggcggctccggctgcttctccagcggcgggggcggcggctctgtctgcggctactctggcggcggctctggctgcggcggaggctcctctggcggcagcggctccggctacgtctcctcgcagcaggtcactcagacctcgtgcgcgccccagccgagttacggaggggggtcgtccggcggcggcggcagcggcggaagcggctgcttctccagcggcgggggcggcgggagctccggctgcggcggcggctcctccgggattggcagcggctgcatcatcagtggcgggggctccgtctgcggaggtggttcctctggaggcggcggcggcggctcctccgtgggtggctccgggagtggcaagggcgtcccgatctgccaccagacccagcagaagcaggcgcctacctggccgtccaaatag

SEQ ID NO: 61 - DNA, 939 bp, Artificial Sequence
  Synthetic
atgagctaccagaagaagcagcccacacctcagcctccagtggactgcgtgaaaacatctggtggtggtggcggaggcggtggatctggcggcggaggatgtggattttttggaggcggaggttccggcggaggcagctctggatctggatgtggttatagcggtggcggtggctactcaggcggcggatgtggcggaggatcttcaggcggcggaggcggaggtggaattggaggttgtggtggtggttctggcggaagcgtgaagtatagcggaggtggcggatcttctggcggaggctctggctgtttttctagcggaggcggaggcagcggctgttttagtagtggtggcggcggtagttcaggtggcggcagtggatgttttagctctggcggaggtggaagctctggcggcggaagcggatgtttttcaagtggcggtggtggctttagcggccaggctgttcagtgtcagtcttacggcggagtgtccagcggcggttctagtggcggcggttctggatgctttagttcaggcggtggcggcggatctgtgtgtggatacagtggcggtggaagtggatgcggaggcggatcaagcggaggatctggctctggctatgtgtccagccagcaagtgacccagacaagctgtgcccctcagccttcttatggtggcgggtctagtggtggcggtggatcaggcggatctggctgttttagttctggtggcggaggcggcagttctggatgtggtggtggatctagcggcatcggcagcggctgcattattagcggcggtggtagtgtttgcggcggagggtcatctggtggcggcggtggcggttcttctgttggtggatctggatctggcaagggcgtgcccatctgtcaccagacacagcagaaacaggcccctacctggcctagcaagtga

SEQ ID NO: 62 - DNA, 1560 bp, Homo sapiens
atgattccggtgtcgctggtggtggtggtggtgggtggctggactgtcgtctacctgaccgacttggtgctgaagtcatctgtctattttaaacattcttatgaagactggctggaaaacaacggactgagcatctcccctttccacataagatggcaaactgctgttttcaatcgtgccttttacagttggggacggcggaaagcaaggatgctttaccaatggttcaattttggaatggtgtttggcgtaattgccatgtttagctcattttttctccttggaaaaacgctgatgcagactttggcacaaatgatggctgactctccctcttcttattcttcctcctcttcttcctcttcctcctcttcttcctcttcctcttcttcatcttcttcctcttcctcgcttcacaatgaacaggtgttacaagttgtggttcctggtataaatttacccgtcaatcaactgacctatttcttcacggcagttctcattagtggtgttgtacatgaaattggacatgggatagcagctattagggaacaagttcgatttaatggctttgggatttttctcttcattatttatcctggagcatttgttgatctgttcaccactcatttgcaacttatatcgccagtccagcagctaaggatattttgtgcaggtatctggcataattttgtccttgcactcttgggtattttagctcttgttctcctcccagtaattctcttgccattttactacactggagttggggtgctcatcactgaagttgctgaggactctcctgccattggacccagaggcctttttgtgggagaccttgtcacccatctacaggattgtcctgttactaatgtgcaagattggaatgaatgtttagataccatcgcctatgagccccaaattggttactgtataagtgcatcaactttacagcagttaagtttcccagttagagcatacaaacgactagatggttcaactgaatgctgtaacaatcacagcctcacagatgtgtgcttttcctacagaaataattttaataagcgtttgcatacatgtcttcctgcccggaaagcagttgaagcaactcaagtttgcagaaccaataaagactgtaaaaaaagctcaagttcaagtttctgtataataccttctttggaaactcacactcgcttaataaaagtaaaacacccacctcagattgatatgttatacgtaggacatcctctgcatcttcactacacagtgagcatcaccagttttatcccacgttttaactttctaagcatagatctgccagtggttgtggagacatttgtcaagtacctgatttccctctcaggagctctggctattgttaatgcagtaccctgctttgctttggatggacaatggattctaaactctttcttggatgccacccttacctcagtgattggagacaatgatgtcaaagatctaatagggtttttcatcttgctgggtggcagtgtacttttggctgccaatgtgaccctgggactctggatggttacagcacggtaa

SEQ ID NO: 63 - DNA, 1560 bp, Artificial Sequence
  Synthetic
atgatccccgtgtctctggtggtggtcgttgttggcggatggaccgtggtgtacctgaccgacctggtgctgaagtccagcgtgtacttcaagcacagctacgaggactggctggaaaacaacggcctgagcatcagccccttccacatcagatggcagaccgccgtgttcaaccgggccttttacagctggggtagaagaaaggccagaatgctgtaccagtggttcaacttcggcatggtgttcggcgtgatcgctatgttcagctcattcttcctgctgggcaagaccctgatgcagacactggcccagatgatggccgactctcctagcagctacagcagcagctccagctcctctagcagctcctccagctcaagcagttccagtagctctagctccagcagcctgcacaacgaacaggtgctgcaggttgtggtgcccggcatcaacctgcctgtgaaccagctgacctacttcttcaccgccgtgctgatctctggcgtggtgcacgaaatcggacacggaatcgccgccatcagagaacaagtgcggttcaacggcttcggcatcttcctgttcatcatctaccctggcgccttcgtggacctgttcaccacacatctgcagctgatctccccagtgcagcagctgagaatcttctgcgccggcatctggcacaacttcgtgctggctctgctgggaatcctggctctggttctgctgcccgttatcctgctgcctttctactataccggcgtgggagtgctgatcaccgaggtggcagaggattctcctgccatcggacctagaggcctgttcgtgggcgatctggttacccatctgcaggactgccccgtgaccaacgtgcaggattggaacgagtgcctggacacaatcgcctacgagccccagatcggctactgcatctctgcctctacactgcagcaactgagcttccctgtgcgggcctacaagagactggatggcagcaccgagtgctgtaacaaccacagcctgaccgatgtgtgcttcagctaccggaacaacttcaacaagcggctgcacacctgtctgcccgccagaaaagctgtggaagccacacaagtgtgccggaccaacaaggactgcaagaagtcctcaagctcctccttctgcatcatccccagcctggaaacccacaccagactgatcaaagtgaagcaccctcctcagatcgacatgctgtacgtgggacaccctctgcatctgcactacaccgtgtccatcaccagctttatccccagattcaacttcctcagcatcgatctgcccgtggtggtggaaaccttcgtgaagtacctgatcagcctgtctggcgccctggccatcgtgaatgccgtgccttgttttgccctggacggccagtggattctgaacagcttcctggacgccacactgaccagcgtgatcggcgacaacgacgtgaaggatctgatcggcttcttcatcctgctcggcggctctgtgctgctggccgctaatgttacactcggcctgtggatggtcaccgccagataa

SEQ ID NO: 64 - DNA, 1401 bp, Homo sapiens
atgccgggtgactcctccccggggactctcccactctgggacgcgtcactctccccacccctgggtccggaccccggcggcttctcgcgcgcgagccacgcgggggacaagtcgcggccacctgctccggagctggggagcccgggcgccgtccgcccgcgcgtcggttcgtgtgccccgggccccatggagctgcgggtcagcaacaccagctgcgagaacggttccctgctccacctctactgctcctcccaagaagtcctgtgccagattgtcaatgacctcagccctgaggtgcccagcaatgccacctttcacagctggcaggaaagaatcaggcagaactatggcttctacatcggcctgggcctggcattcctgtctagcttcctcatcggcagcagcgtcatcctcaagaagaaaggcctcttgcgactcgtggccacgggagccactcgagctgtggatggaggcttcggctacctgaaagatgcaatgtggtgggctggatttctcaccatggctgctggagaagttgccaactttggagcctacgcatttgcacctgcaacagtcgtcacgcctctgggagcgctgagtgtcctcataagtgccatcctctcctcatatttcctgagggagagtctgaacctgctggggaagctgggctgtgtgatctgtgtggccggaagcacagtgatggtgatacatgctcctgaggaagagaaggtcactaccatcatggagatggcttccaagatgaaagacacagggttcatcgtgtttgctgtgcttctgctggtgtcatgcctcatcctcatctttgtcattgccccacgttacgggcaaaggaatatcctcatctacatcatcatctgctctgtgatcggggccttctctgtggctgctgtcaaggggctgggcatcaccatcaagaacttcttccaggggctgccagttgtccggcacccgctcccctacatcctgtccctcatcctggcactgtccctcagcactcaggtcaacttcctcaacagagcactggacattttcaacacttccctggtgttccccatctactacgtgttcttcaccacggtggtcgttacctcgtccatcatcctcttcaaggagtggtacagcatgtctgctgtggacattgcaggcaccctctcgggctttgtcaccatcatcttgggcgtgttcatgctgcatgctttcaaagacctggacatcagctgcgccagcttgccccacatgcacaaaaacccacccccttctcccgccccggaacccactgttattagactggaagacaagaacgtccttgtggacaatatagaacttgccagcacctcatcaccagaagagaaacccaaagtatttataatccattcctga

SEQ ID NO: 65 - DNA, 1401 bp, Artificial Sequence
  Synthetic
atgcctggcgatagctctcctggaacactgcctctgtgggacgcctctttgtctcctcctctgggacctgatcctggcggcttttctagagcctctcacgccggcgataagagcagacctcctgctcctgaactgggatctcctggcgccgttagacctagagtgggatcttgtgcccctggacctatggaactgcgggtgtccaataccagctgcgagaatggctctctgctgcacctgtactgcagcagccaagaggtgctgtgccagatcgtgaatgatctgagccctgaggtgcccagcaacgccacatttcacagctggcaagagcggatcagacagaactacggcttctacatcggcctgggcctcgccttcctgagcagctttctgatcggcagcagcgtgatcctgaagaagaagggcctgctgagactggtggccacaggcgctacaagagctgtggatggcggattcggctacctgaaggacgctatgtggtgggccggcttcctgacaatggctgctggcgaggtggccaattttggcgcctatgcctttgctcctgccaccgtggttacacctctgggagcactgagcgtgctgatctccgctatcctgtccagctacttcctgagagagagcctgaacctgctgggcaagctgggctgtgtgatttgtgtggccggctccaccgtgatggtcattcatgcccctgaagaggaaaaagtgaccaccatcatggaaatggccagcaagatgaaggacaccggcttcatcgtgttcgccgtgctgctgctggtgtcctgcctgattctgatcttcgtgatcgcccctagatacggccagcggaacatcctgatctacatcatcatctgctccgtgatcggcgccttttctgtggccgctgtgaaaggcctgggaatcaccatcaagaatttcttccagggcctgcctgtcgtgcggcaccctctgccttatatcctgtctctgattctggccctgagcctgtctacccaagtgaactttctgaacagagccctggacatcttcaacacaagcctggtgttccccatctactacgtgttcttcaccacagtggtcgtgaccagctccatcatcctgttcaaagaatggtacagcatgagcgccgtggacattgccggcacactgagcggctttgtgacaatcatcctgggcgtgttcatgctgcacgccttcaaggacctggatatcagctgcgccagcctgcctcacatgcacaagaaccctccaccttctccagctccagagcctaccgtgatccggctggaagataagaacgtgctggtggacaacatcgagctggcctccacaagcagccccgaagagaagcccaaggtgttcatcatccactcttga

SEQ ID NO: 66 - DNA, 1344 bp, Homo sapiens
atgccgggtgactcctccccggggactctcccactctgggacgcgtcactctccccacccctgggtccggaccccggcggcttctcgcgcgcgagccacgcgggggacaagtcgcggccacctgctccggagctggggagcccgggcgccgtccgcccgcgcgtcggttcgtgtgccccgggccccatggagctgcgggtcagcaacaccagctgcgagaacggttccctgctccacctctactgctcctcccaagaagtcctgtgccagattgtcaatgacctcagccctgaggtgcccagcaatgccacctttcacagctggcaggaaagaatcaggcagaactatggcttctacatcggcctgggcctggcattcctgtctagcttcctcatcggcagcagcgtcatcctcaagaagaaaggcctcttgcgactcgtggccacgggagccactcgagctgtggctgctggagaagttgccaactttggagcctacgcatttgcacctgcaacagtcgtcacgcctctgggagcgctgagtgtcctcataagtgccatcctctcctcatatttcctgagggagagtctgaacctgctggggaagctgggctgtgtgatctgtgtggccggaagcacagtgatggtgatacatgctcctgaggaagagaaggtcactaccatcatggagatggcttccaagatgaaagacacagggttcatcgtgtttgctgtgcttctgctggtgtcatgcctcatcctcatctttgtcattgccccacgttacgggcaaaggaatatcctcatctacatcatcatctgctctgtgatcggggccttctctgtggctgctgtcaaggggctgggcatcaccatcaagaacttcttccaggggctgccagttgtccggcacccgctcccctacatcctgtccctcatcctggcactgtccctcagcactcaggtcaacttcctcaacagagcactggacattttcaacacttccctggtgttccccatctactacgtgttcttcaccacggtggtcgttacctcgtccatcatcctcttcaaggagtggtacagcatgtctgctgtggacattgcaggcaccctctcgggctttgtcaccatcatcttgggcgtgttcatgctgcatgctttcaaagacctggacatcagctgcgccagcttgccccacatgcacaaaaacccacccccttctcccgccccggaacccactgttattagactggaagacaagaacgtccttgtggacaatatagaacttgccagcacctcatcaccagaagagaaacccaaagtatttataatccattcctga

SEQ ID NO: 67 - DNA, 1344 bp, Artificial Sequence
  Synthetic
atgcctggcgatagctctcctggaacactgcctctgtgggacgcctctttgtctcctcctctgggacctgatcctggcggcttttctagagcctctcacgccggcgataagagcagacctcctgctcctgaactgggatctcctggcgccgttagacctagagtgggatcttgtgcccctggacctatggaactgcgggtgtccaataccagctgcgagaatggctctctgctgcacctgtactgcagcagccaagaggtgctgtgccagatcgtgaatgatctgagccctgaggtgcccagcaacgccacatttcacagctggcaagagcggatcagacagaactacggcttctacatcggcctgggcctcgccttcctgagcagctttctgatcggcagcagcgtgatcctgaagaagaagggcctgctgagactggtggccacaggcgctacaagagctgttgctgctggcgaggtggccaattttggcgcctatgcctttgctcctgccaccgtggttacacctctgggagcactgagcgtgctgatctccgctatcctgtccagctacttcctgagagagagcctgaacctgctgggcaagctgggctgtgtgatttgtgtggccggctccaccgtgatggtcattcatgcccctgaagaggaaaaagtgaccaccatcatggaaatggccagcaagatgaaggacaccggcttcatcgtgttcgccgtgctgctgctggtgtcctgcctgattctgatcttcgtgatcgcccctagatacggccagcggaacatcctgatctacatcatcatctgctccgtgatcggcgccttttctgtggccgctgtgaaaggcctgggaatcaccatcaagaatttcttccagggcctgcctgtcgtgcggcaccctctgccttatatcctgtctctgattctggccctgagcctgtctacccaagtgaactttctgaacagagccctggacatcttcaacacaagcctggtgttccccatctactacgtgttcttcaccacagtggtcgtgaccagctccatcatcctgttcaaagaatggtacagcatgagcgccgtggacattgccggcacactgagcggctttgtgacaatcatcctgggcgtgttcatgctgcacgccttcaaggacctggatatcagctgcgccagcctgcctcacatgcacaagaaccctccaccttctccagctccagagcctaccgtgatccggctggaagataagaacgtgctggtggacaacatcgagctggcctccacaagcagccccgaagagaagcccaaggtgttcatcatccacagctga

SEQ ID NO: 68 - DNA, 1122 bp, Homo sapiens
atggaaccagcagttagcgagccaatgagagaccaagtcgcacggactcatttgacagaggacactcccaaagtgaatgctgacatagaaaaggttaaccagaatcaggccaagagatgcacagtgatcggtggctctggattcctggggcagcacatggtggagcagttgctggcaagaggatatgctgtcaatgtatttgatatccagcaagggtttgataatccccaggtgcggttctttctgggtgacctctgcagccgacaggatctgtacccagctctgaaaggtgtaaacacagttttccactgtgcgtcacccccaccatccagtaacaacaaggagctcttttatagagtgaattacattggcaccaagaatgtcattgaaacttgcaaagaggctggggttcagaaactcattttaaccagcagtgccagtgtcatctttgagggcgtcgatatcaagaatggaactgaagaccttccctatgccatgaaacccattgactactacacagagactaagatcttacaggagagggcagttctgggcgccaacgatcctgagaagaatttcttaaccacagccatccgccctcatggcattttcggcccaagggacccgcagttggtacccatcctcatcgaggcagccaggaacggcaagatgaagttcgtgattggaaatgggaagaacttggtggacttcacctttgtggagaacgtggtccatggacacatcctggcggcagagcagctctcccgagactcgacactgggtgggaaggcatttcacatcaccaatgatgagcccatccctttctggacattcctgtctcgcatcctgacaggcctcaattatgaggcccccaagtaccacatcccctactgggtggcctactacctggccctcctgctatccctgctggtgatggtgatcagtcctgtcatccagctgcagcccaccttcacacccatgcgggtcgcactggctggcacattccactactacagctgcgagagagccaaaaaggccatgggctaccagccactagtgaccatggatgatgctatggagaggaccgtgcagagctttcgccacctgcggagggtcaagtga

SEQ ID NO: 69 - DNA, 1122 bp, Artificial Sequence
  Synthetic
atggaacctgccgtgtccgagcctatgagagatcaggtggccagaacacacctgaccgaggacacccctaaagtgaacgccgacatcgagaaagtgaaccagaaccaggccaagagatgcaccgtgatcggcggctctggatttctgggccagcacatggttgaacagctgctggccagaggctacgccgtgaacgtgttcgatatccagcagggcttcgacaaccctcaagtgcggttcttcctgggcgacctgtgcagcagacaggatctgtaccctgctctgaagggcgtgaacaccgtgttccactgtgcctctccacctccaagcagcaacaacaaagaactgttctaccgcgtgaactacatcggcaccaagaacgtgatcgagacatgcaaagaggccggcgtccagaagctgatcctgacatctagcgccagcgtgatcttcgaaggcgtggacatcaagaacggcaccgaggatctgccctacgccatgaagcccatcgactactacaccgagacaaagatcctgcaagagcgggccgtgctgggcgccaatgatcccgagaagaatttcctgaccaccgccatcagaccccacggcatctttggccctagagatcctcagctggtgcccatcctgattgaggccgccagaaacggcaagatgaagtttgtgatcggcaacgggaagaacctggtggacttcaccttcgtggaaaacgtggtgcacggccacattctggctgccgagcagctgagcagagattctacactcggcggcaaggccttccacattaccaacgacgagcctattcctttctggaccttcctgagcagaatcctgaccggcctgaactacgaggcccctaagtatcacatcccctactgggtcgcctactatctggccctgctgctgtctctgctggtcatggttatcagccccgtgatccagctgcagcccaccttcacaccaatgagagtggccctggccggcaccttccactactactcttgcgagagggccaagaaagccatgggctaccagcctctggtcaccatggacgacgccatggaaagaaccgtgcagagcttccggcacctgagaagagtgaagtga

SEQ ID NO: 70 - DNA, 972 bp, Homo sapiens
atgagtgcggtgtgcggtggagcggcgcggatgctgcggacgccgggacgccacggctacgccgccgagttctccccgtacctgccgggccgcctggcctgcgccaccgcgcagcactacggcatcgcgggctgtggaaccctactaatattggatccagatgaagctgggctaaggctttttagaagctttgactggaatgatggtttgtttgatgtgacttggagtgagaacaacgaacatgtcctcatcacctgtagtggcgatggctcgctgcagctctgggacactgccaaagctgcagggccactgcaagtctataaagaacacgctcaggaggtgtatagtgttgattggagccaaaccagaggtgaacagcttgtggtgtctggctcatgggatcaaactgtcaaattgtgggatccaactgttggaaagtctctgtgcacctttagaggccatgaaagtattatttatagcacaatctggtctccccacatccctggttgttttgcttcagcctcaggtgatcagactctgagaatatgggatgtgaaggcagcaggagtaagaatcgtgattcctgcacatcaggcagaaatcttgagttgtgactggtgtaaatacaatgagaatttgctggtgaccggggcggttgactgtagtttgagaggctgggacttaaggaatgtacgacaaccagtgtttgaacttcttggtcatacctatgctattaggagggtgaaattttcaccatttcatgcttctgtgctggcctcttgctcgtatgattttactgtaagattctggaacttttcaaagcctgactctcttcttgaaacagtggagcatcatacagagtttacttgtggtttagacttcagtcttcagagccccactcaggtggctgactgttcttgggatgaaacaataaagatctatgaccctgcttgtcttactattcctgcttga

SEQ ID NO: 71 - DNA, 972 bp, Artificial Sequence
  Synthetic
atgtctgctgtgtgtggcggagccgccagaatgctgagaacacctggcagacatggctacgccgccgagttctctccttacctgcctggaaggctggcctgtgccacagctcagcactatggaatcgccggctgtggaaccctgctgattctggaccctgatgaggccggactgcggctgttcagaagcttcgattggaacgacggcctgttcgatgtgacttggagcgagaacaacgaacacgtgctgatcacctgtagcggcgacggatctctgcagctgtgggatacagccaaagccgccggacctctgcaggtctacaaagaacatgcccaagaggtgtacagcgtggactggtcccagacaagaggcgagcagctggtggtgtctggcagctgggatcagacagtgaaactgtgggaccccaccgtgggcaagagcctgtgtacctttagaggccacgagagcatcatctacagcaccatctggtccccacacatccctggctgttttgcctctgcctctggcgatcagaccctgagaatctgggatgtgaaagccgctggcgtgcggatcgtgattcctgctcatcaggccgagatcctgagctgcgactggtgcaagtacaacgagaacctgctcgtgacaggcgccgtggattgctctctgagaggctgggacctgagaaacgtgcggcagcctgtgtttgagctgctgggccacacatacgccatcagaagagtgaagttcagccccttccacgcctccgtgctggcctcttgcagctacgatttcaccgtgcggttctggaacttcagcaagcccgacagcctgctggaaaccgtggaacaccacaccgagttcacctgtggcctggactttagcctgcagagccctacacaggtggccgactgttcttgggacgagacaatcaagatctacgaccccgcctgcctgacaatccctgcttaa

SEQ ID NO: 72 - DNA, 1602 bp, Homo sapiens
atggcttttgcaaatctgcggaaagtgctcatcagtgacagcctggacccttgctgccggaagatcttgcaagatggagggctgcaggtggtggaaaagcagaaccttagcaaagaggagctgatagcggagctgcaggactgtgaaggccttattgttcgctctgccaccaaggtgaccgctgatgtcatcaacgcagctgagaaactccaggtggtgggcagggctggcacaggtgtggacaatgtggatctggaggccgcaacaaggaagggcatcttggttatgaacacccccaatgggaacagcctcagtgccgcagaactcacttgtggaatgatcatgtgcctggccaggcagattccccaggcgacggcttcgatgaaggacggcaaatgggagcggaagaagttcatgggaacagagctgaatggaaagaccctgggaattcttggcctgggcaggattgggagagaggtagctacccggatgcagtcctttgggatgaagactatagggtatgaccccatcatttccccagaggtctcggcctcctttggtgttcagcagctgcccctggaggagatctggcctctctgtgatttcatcactgtgcacactcctctcctgccctccacgacaggcttgctgaatgacaacacctttgcccagtgcaagaagggggtgcgtgtggtgaactgtgcccgtggagggatcgtggacgaaggcgccctgctccgggccctgcagtctggccagtgtgccggggctgcactggacgtgtttacggaagagccgccacgggaccgggccttggtggaccatgagaatgtcatcagctgtccccacctgggtgccagcaccaaggaggctcagagccgctgtggggaggaaattgctgttcagttcgtggacatggtgaaggggaaatctctcacgggggttgtgaatgcccaggcccttaccagtgccttctctccacacaccaagccttggattggtctggcagaagctctggggacactgatgcgagcctgggctgggtcccccaaagggaccatccaggtgataacacagggaacatccctgaagaatgctgggaactgcctaagccccgcagtcattgtcggcctcctgaaagaggcttccaagcaggcggatgtgaacttggtgaacgctaagctgctggtgaaagaggctggcctcaatgtcaccacctcccacagccctgctgcaccaggggagcaaggcttcggggaatgcctcctggccgtggccctggcaggcgccccttaccaggctgtgggcttggtccaaggcactacgcctgtactgcaggggctcaatggagctgtcttcaggccagaagtgcctctccgcagggacctgcccctgctcctattccggactcagacctctgaccctgcaatgctgcctaccatgattggcctcctggcagaggcaggcgtgcggctgctgtcctaccagacttcactggtgtcagatggggagacctggcacgtcatgggcatctcctccttgctgcccagcctggaagcgtggaagcagcatgtgactgaagccttccagttccacttctaa

SEQ ID NO: 73 - DNA, 1602 bp, Artificial Sequence
  Synthetic
atggccttcgccaacctgagaaaggtgctgatctccgactctctggacccctgctgcagaaagattctgcaggatggcggcctgcaggtcgtggaaaagcagaacctgagcaaagaggaactgatcgccgagctgcaggattgcgagggactgatcgtcagatccgccaccaaagtgaccgccgacgtgatcaatgccgccgagaaactgcaggttgtgggcagagctggaaccggcgtggacaatgtggatctggaagccgccaccagaaagggcatcctggtcatgaacacccctaacggcaacagcctgtctgccgccgaactgacctgcggaatgatcatgtgcctggccagacagatccctcaggccacagcctctatgaaggacggcaagtgggagcgcaagaaattcatgggcaccgagctgaacggcaagaccctgggaatcctcggcctgggcagaatcggaagagaagtggccacacggatgcagagcttcggcatgaagaccatcggctacgaccccatcatcagccctgaagtgtctgccagctttggcgtgcagcagctgcctctggaagagatctggcccctgtgcgacttcatcaccgtgcacacacctctgctgcctagcacaaccggcctgctgaacgacaacacattcgcccagtgcaagaaaggcgtgcgggtcgtgaattgtgccagaggcggaattgtggacgaaggcgctctgctgagagccctgcaatctggtcaatgtgctggcgccgctctggacgtgttcacagaggaacctccaagagacagagccctggtggaccacgagaacgtgatcagctgtcctcacctgggcgcctctacaaaagaggcccagagccgttgcggagaagagatcgctgtgcagttcgtggacatggtcaagggcaagagcctgaccggcgtcgtgaatgctcaggccctgacaagcgcttttagccctcacaccaagccttggatcggactggctgaagccctgggcacactgatgagagcatgggccggatctccaaagggcaccatccaagtgatcacccagggcaccagcctgaagaacgccggcaattgtctgagccctgccgtgattgtgggactgctgaaagaggccagcaagcaggccgatgtgaacctggttaacgccaagctgctggtcaaagaggccggactgaacgtcaccacctctcactctccagctgctccaggcgagcaaggctttggagaatgtctgctggctgtggctcttgctggcgctccttatcaggcagttggactggtgcagggcacaacccctgttctgcaaggactgaatggcgccgtgttcagacctgaggtgcccctgagaagggatctgccactgctgctgttcagaacccagaccagcgatcctgccatgctgcctacaatgattggcctgttggccgaggccggcgtcagactgctgagctatcagacaagcctggtgtccgacggcgagacatggcacgtgatgggaatctctagcctgctgccaagcctggaagcttggaagcagcacgtgaccgaggccttccagttccacttctga

SEQ ID NO: 74 - DNA, 1017 bp, Homo sapiens
atggagcagcttcgcgccgccgcccgtctgcagattgttctgggccacctcggccgcccctcggccggggctgtcgtagctcatcccacttcagggactatttcctctgccagtttccatcctcaacaattccagtatactctggataataacgttctaaccctggaacagagaaaattttatgaagaaaatgggtttctagtaatcaaaaatcttgtacctgatgccgatattcaacgctttcggaatgagtttgaaaaaatctgcagaaaggaggtgaaaccattaggattaacagtaatgagagatgtgaccatttcgaaatccgaatatgctccaagtgagaagatgatcacgaaggtccaggatttccaggaagataaggagctcttcagatactgcactctccccgagattctgaaatatgtggagtgcttcactggacctaatattatggccatgcacacaatgttgataaacaaacctccagattctggcaagaagacgtcccgtcaccccctgcaccaggacctgcactatttccccttcaggcccagcgatctcatcgtttgcgcctggacggcgatggagcacatcagccggaacaacggctgtctggttgtgctcccaggcacacacaagggctccctgaagccccacgattaccccaagtgggaggggggagttaacaaaatgttccacgggatccaggactacgaggaaaacaaggcccgggtgcacctggtgatggagaagggcgacactgttttcttccatcctttgctcatccacggatctggtcagaataaaacccagggattccggaaggcaatttcctgccatttcgccagtgccgattgccactacattgacgtgaagggcaccagtcaagaaaacatcgagaaggaagttgtaggaatagcacataaattctttggagctgaaaatagcgtgaacttgaaggatatttggatgtttcgagctcgacttgtgaaaggagaaagaaccaatctttga

SEQ ID NO: 75 - DNA, 1017 bp, Artificial Sequence
  Synthetic
atggaacagctgagagccgctgccagactgcagattgtgctgggacatctgggcagaccatctgctggtgctgtggtggcccatcctacaagcggcacaattagcagcgccagcttccatcctcagcagttccagtacaccctggacaacaacgtgctgaccctggaacagcggaagttctacgaagagaacggcttcctggtcatcaagaacctggtgcctgacgccgacatccagcggttcagaaacgagttcgagaagatctgccggaaagaagtgaagcccctgggcctgaccgtgatgagagatgtgaccatcagcaagagcgagtacgcccctagcgagaagatgatcaccaaggtgcaggacttccaagaggacaaagagctgttccggtactgcaccctgcctgagatcctgaaatacgtggaatgcttcacaggccccaacattatggccatgcacaccatgctgatcaacaagcctccagacagcggcaagaaaaccagcagacaccctctgcaccaggacctgcactacttcccattcagacccagcgacctgatcgtgtgtgcctggacagccatggaacacatcagccggaacaatggctgcctggtggtgctgcctggaacacacaagggaagcctgaagcctcacgactaccccaagtgggaaggcggcgtgaacaagatgttccacggcatccaggactatgaagagaacaaggcccgggtgcacctggtcatggaaaagggcgacaccgtgttctttcacccactgctgatccacggcagcggccagaacaagacccagggctttagaaaggccatcagctgccacttcgccagcgccgattgccactacatcgacgtgaagggcaccagccaagagaacatcgagaaagaagtcgtgggaatcgcccacaagttctttggcgccgagaacagcgtgaacctgaaggatatctggatgttccgggccagactggtcaagggcgagcgcacaaatctgtga

SEQ ID NO: 76 - DNA, 1599 bp, Homo sapiens
atggaagaacaggtgttcaagggggacccggacacccctcactccatctccttctcgggcagtggattcctctccttctaccaggcgggggctgtggacgccctgcgggacctggccccccggatgctggaaacagcccaccgctttgcggggacatcggcaggtgctgtgatcgccgccctggccatctgcgggattgaaatggatgagtatctcagagtcctcaacgtgggtgtggccgaggtgaagaaatccttcctggggcccttgtccccgtcctgtaagatggtgcagatgatgaggcagtttctgtaccgggtcctgcccgaggactcctacaaggtcaccacggggaagctccatgtgagcctcacccgcttaacggacggggagaatgtggtggtttcagagttcacgtccaaggaggagctcattgaggccctatactgcagctgcttcgtcccggtgtactgtggcctcatccccccgacttaccgcggtgtgaggtacatcgatgggggcttcacgggcatgcagccctgtgccttctggaccgacgccatcaccatctccaccttcagtgggcagcaggacatctgtccccgggactgcccggccatcttccacgacttccgcatgttcaactgctccttccagttctccctggagaacatcgccaggatgacccacgcattgttccccccggacctggtgatcctgcacgattactactaccgagggtacgaggatgcagttttgtacttgaggcggctgaatgctgtttatcttaattcttcctccaagagagtgattttcccccgggtggaagtgtactgccagatagaactcgcccttggcaatgagtgccctgaacgcagtcaaccaagccttcgagcacggcaggccagtctggaaggagccacacaacctcacaaggagtgggttcccaaaggggatggaaggggcagccatggtccgcctgtgtcccaacctgtgcagacacttgaattcacatgcgagtcacctgtttcagcaccagtctctccacttgagcagccacctgcacagccactggcctcttcaactccactttctctaagtggcatgccacctgtatcattcccagctgtgcacaagccacccagctccacacctggttcatcactgcccaccccaccacctggactgtcacctctgtcacctcagcagcaggtacaaccgtctggatcaccagccagatccctacactctcaggcacccacttcacccaggccatccctggggccttcaactgtgggggcacctcaaacactgccccgaagttctctttcagccttccctgctcagccacctgtggaggaactaggccaagaacagccccaagctgtagctcttcttgtctcttcaaaaccaaaaagcgccgtgcctctggttcatgtgaaggaaaccgtcagcaagccttatgtaacggagagccctgctgaagactcaaactgggtgaataaggtcttcaagaagaacaagcaaaagacaagtggcaccagaaaaggcttcccaagacattcgggatccaaaaaaccaagcagcaaagtgcagtga

SEQ ID NO: 77 - DNA, 1599 bp, Artificial Sequence
  Synthetic
atggaagaacaggtgttcaagggcgaccccgacacacctcacagcatcagctttagcggcagcggcttcctgagcttctatcaggctggtgccgtggacgccctgagagatcttgcccctagaatgctggaaacagcccacagattcgccggcacatctgctggcgctgtgattgccgctctggccatctgtggcatcgagatggacgagtacctgcgggtgctgaatgtgggcgtcgccgaagtgaagaagtcctttctgggccctctgagccccagctgcaagatggtgcagatgatgcggcagttcctgtacagagtgctgcccgaggacagctacaaagtgaccacaggcaagctgcacgtgtccctgaccagactgaccgacggcgagaatgtggtggtgtccgagttcaccagcaaagaggaactgatcgaggccctgtactgcagctgcttcgtgcctgtgtactgcggactgatccctccaacctatagaggcgtgcggtacatcgatggcggcttcactggaatgcagccctgcgccttttggaccgacgccatcaccatcagcaccttctctggccagcaggacatctgccccagagattgccctgccatcttccacgacttccggatgttcaactgcagcttccagttcagcctggaaaacattgcccggatgacccacgctctgttcccacctgatctggtcatcctgcacgactactactaccggggctacgaggatgccgtgctgtacctgagaaggctgaacgccgtgtacctgaacagcagcagcaagagagtgatcttccccagagtggaagtgtactgccagatcgagctggccctgggcaatgagtgtcccgagagatctcagccaagcctgagagccagacaggcttctctggaaggcgccacacagccccacaaagaatgggttcccaaaggcgacggcagaggctctcatggacctccagtttctcagcccgtgcagaccctggaattcacatgcgagagccctgtgtcagcccctgtgtctcctctggaacagcctcctgctcagcctctggcctctagcacacctctgtctctgtctggcatgcctcctgtgtcctttccagccgtgcacaagcctccaagcagcacccctggatctagcctgcctacacctcctccaggactgagcccactgtctcctcagcagcaggttcagcctagcggatctccagccagaagcctgcattctcaggcccctacaagccccagaccaagcctgggaccttctacagttggagcccctcagacactgcccagaagcagcctttctgctttccccgctcagccacctgtggaagaactgggacaagagcagcctcaggctgtggcactgctggtgtctagcaagcctaagtctgccgtgcctctggtgcacgtgaaagaaaccgtgtccaagccttacgtgaccgagtctcctgccgaggactccaactgggtcaacaaagtgttcaagaagaacaagcagaaaaccagcggcacccggaagggcttccctagacactctggcagcaagaaacccagctccaaggtgcagtga

SEQ ID NO: 78 - DNA, 1314 bp, Homo sapiens
atggtgcagatgatgaggcagtttctgtaccgggtcctgcccgaggactcctacaaggtcaccacggggaagctccatgtgagcctcacccgcttaacggacggggagaatgtggtggtttcagagttcacgtccaaggaggagctcattgaggccctatactgcagctgcttcgtcccggtgtactgtggcctcatccccccgacttaccgcggtgtgaggtacatcgatgggggcttcacgggcatgcagccctgtgccttctggaccgacgccatcaccatctccaccttcagtgggcagcaggacatctgtccccgggactgcccggccatcttccacgacttccgcatgttcaactgctccttccagttctccctggagaacatcgccaggatgacccacgcattgttccccccggacctggtgatcctgcacgattactactaccgagggtacgaggatgcagttttgtacttgaggcggctgaatgctgtttatcttaattcttcctccaagagagtgattttcccccgggtggaagtgtactgccagatagaactcgcccttggcaatgagtgccctgaacgcagtcaaccaagccttcgagcacggcaggccagtctggaaggagccacacaacctcacaaggagtgggttcccaaaggggatggaaggggcagccatggtccgcctgtgtcccaacctgtgcagacacttgaattcacatgcgagtcacctgtttcagcaccagtctctccacttgagcagccacctgcacagccactggcctcttcaactccactttctctaagtggcatgccacctgtatcattcccagctgtgcacaagccacccagctccacacctggttcatcactgcccaccccaccacctggactgtcacctctgtcacctcagcagcaggtacaaccgtctggatcaccagccagatccctacactctcaggcacccacttcacccaggccatccctggggccttcaactgtgggggcacctcaaacactgccccgaagttctctttcagccttccctgctcagccacctgtggaggaactaggccaagaacagccccaagctgtagctcttcttgtctcttcaaaaccaaaaagcgccgtgcctctggttcatgtgaaggaaaccgtcagcaagccttatgtaacggagagccctgctgaagactcaaactgggtgaataaggtcttcaagaagaacaagcaaaagacaagtggcaccagaaaaggcttcccaagacattcgggatccaaaaaaccaagcagcaaagtgcagtga

SEQ ID NO: 79 - DNA, 1314 bp, Artificial Sequence
  Synthetic
atggtgcagatgatgcggcagttcctgtacagagtgctgcccgaggacagctacaaagtgaccacaggcaagctgcacgtgtccctgaccagactgaccgacggcgagaatgtggtggtgtccgagttcaccagcaaagaggaactgatcgaggccctgtactgcagctgcttcgtgcctgtgtactgcggactgatccctccaacctatagaggcgtgcggtacatcgatggcggcttcactggaatgcagccctgcgccttttggaccgacgccatcaccatcagcaccttctctggccagcaggacatctgccccagagattgccctgccatcttccacgacttccggatgttcaactgcagcttccagttcagcctggaaaacattgcccggatgacccacgctctgttcccacctgatctggtcatcctgcacgactactactaccggggctacgaggatgccgtgctgtacctgagaaggctgaacgccgtgtacctgaacagcagcagcaagagagtgatcttccccagagtggaagtgtactgccagatcgagctggccctgggcaatgagtgccctgagagatctcagccaagcctgagagccagacaggcttctctggaaggcgccacacagccccacaaagaatgggttcccaaaggcgacggcagaggctctcatggacctccagtttctcagcccgtgcagaccctggaattcacatgcgagagccctgtgtcagcccctgtgtctcctctggaacagcctcctgctcagcctctggcctctagcacacctctgtctctgtctggcatgcctcctgtgtcctttccagccgtgcacaagcctccaagcagcacccctggatctagcctgcctacacctcctccaggactgagcccactgtctcctcagcagcaggttcagcctagcggatctccagccagaagcctgcattctcaggcccctacaagccccagaccatctctgggaccttctacagtgggagcccctcagacactgcctagaagcagcctgagtgctttccccgctcagccacctgtggaagaactgggacaagagcagcctcaggctgtggcactgctggtgtctagcaagcctaagtctgccgtgcctctggtgcacgtgaaagaaaccgtgtccaagccttacgtgaccgagtctcctgccgaggactccaactgggtcaacaaggtgttcaagaagaacaagcagaaaaccagcggcacccggaagggcttccctagacactctggcagcaagaaacccagctccaaggtgcagtga

SEQ ID NO: 80 - DNA, 1341 bp, Homo sapiens
atggtgcagatgatgaggcagtttctgtaccgggtcctgcccgaggactcctacaaggtcaccacggggaagctccatgtgagcctcacccgcttaacggacggggagaatgtggtggtttcagagttcacgtccaaggaggagctcattgaggccctatactgcagctgcttcgtcccggtgtactgtggcctcatccccccgacttaccgcggtgtgtgggcatttctcactctgcccccacagaggtacatcgatgggggcttcacgggcatgcagccctgtgccttctggaccgacgccatcaccatctccaccttcagtgggcagcaggacatctgtccccgggactgcccggccatcttccacgacttccgcatgttcaactgctccttccagttctccctggagaacatcgccaggatgacccacgcattgttccccccggacctggtgatcctgcacgattactactaccgagggtacgaggatgcagttttgtacttgaggcggctgaatgctgtttatcttaattcttcctccaagagagtgattttcccccgggtggaagtgtactgccagatagaactcgcccttggcaatgagtgccctgaacgcagtcaaccaagccttcgagcacggcaggccagtctggaaggagccacacaacctcacaaggagtgggttcccaaaggggatggaaggggcagccatggtccgcctgtgtcccaacctgtgcagacacttgaattcacatgcgagtcacctgtttcagcaccagtctctccacttgagcagccacctgcacagccactggcctcttcaactccactttctctaagtggcatgccacctgtatcattcccagctgtgcacaagccacccagctccacacctggttcatcactgcccaccccaccacctggactgtcacctctgtcacctcagcagcaggtacaaccgtctggatcaccagccagatccctacactctcaggcacccacttcacccaggccatccctggggccttcaactgtgggggcacctcaaacactgccccgaagttctctttcagccttccctgctcagccacctgtggaggaactaggccaagaacagccccaagctgtagctcttcttgtctcttcaaaaccaaaaagcgccgtgcctctggttcatgtgaaggaaaccgtcagcaagccttatgtaacggagagccctgctgaagactcaaactgggtgaataaggtcttcaagaagaacaagcaaaagacaagtggcaccagaaaaggcttcccaagacattcgggatccaaaaaaccaagcagcaaagtgcagtga

SEQ ID NO: 81 - DNA, 1341 bp, Artificial Sequence
  Synthetic
atggtgcagatgatgcggcagttcctgtacagagtgctgcccgaggacagctacaaagtgaccacaggcaagctgcacgtgtccctgaccagactgaccgacggcgagaatgtggtggtgtccgagttcaccagcaaagaggaactgatcgaggccctgtactgcagctgcttcgtgcctgtgtactgcggactgatccctccaacctacagaggcgtgtgggcctttctgacactgcctcctcagcggtatatcgacggcggctttaccggaatgcagccctgtgccttttggaccgacgccatcaccatcagcaccttctctggccagcaggacatctgccccagagattgccctgccatcttccacgacttccggatgttcaactgcagcttccagttcagcctggaaaacattgcccggatgacccacgctctgttcccacctgatctggtcatcctgcacgactactactaccggggctacgaggatgccgtgctgtacctgagaaggctgaacgccgtgtacctgaacagcagcagcaagagagtgatcttccccagagtggaagtgtactgccagatcgagctggccctgggcaatgagtgccctgagagatctcagccaagcctgagagccagacaggcttctctggaaggcgccacacagccccacaaagaatgggtgccaaaaggcgacggcagaggctctcatggacctccagtttctcagcccgtgcagaccctggaattcacatgcgagagccctgtgtcagcccctgtgtctcctctggaacagcctcctgctcagcctctggcctctagcacacctctgtctctgtctggcatgcctcctgtgtcctttccagccgtgcacaagcctccaagcagcacccctggatctagcctgcctacacctcctccaggactgagcccactgtctccacagcaacaggtgcagcctagcggatctccagccagaagcctgcattctcaggcccctacaagccccagaccatctctgggaccttctacagtgggagcccctcagacactgccaagaagcagcctgtcagctttccccgctcagccacctgtggaagaactgggacaagagcagcctcaggctgtggcactgctggtgtctagcaagcctaagtctgccgtgcctctggtgcacgtgaaagaaaccgtgtccaagccttacgtgaccgagtctcctgccgaggactccaactgggtcaacaaggtgttcaagaagaacaagcagaaaaccagcggcacccggaagggcttccctagacactctggcagcaagaaacccagctccaaggtgcagtga

SEQ ID NO: 82 - DNA, 426 bp, Homo sapiens
atgaatgccagaggacttggatctgagctaaaggacagtattccagttactgaactttcagcaagtggaccttttgaaagtcatgatcttcttcggaaaggtttttcttgtgtgaaaaatgaacttttgcctagtcatccccttgaattatcagaaaaaaatttccagctcaaccaagataaaatgaatttttccacactgagaaacattcagggtctatttgctccgctaaaattacagatggaattcaaggcagtgcagcaggttcagcgtcttccatttctttcaagctcaaatctttcactggatgttttgaggggtaatgatgagactattggatttgaggatattcttaatgatccatcacaaagcgaagtcatgggagagccacacttgatggtggaatataaacttggtttactgtaa

SEQ ID NO: 83 - DNA, 426 bp, Artificial Sequence
  Synthetic
atgaatgccagaggcctgggcagcgagctgaaggattctatccctgtgaccgagctgagcgccagcggaccttttgagtctcacgacctgctgcggaagggcttcagctgcgtgaagaatgagctgctgccttctcaccctctggaactgagcgagaagaacttccagctgaaccaggacaagatgaacttcagcaccctgcggaacatccagggcctgtttgcccctctgaagctgcagatggagttcaaggccgtgcagcaggttcagagactgccctttctgagcagcagcaacctgagcctggatgtgctgagaggcaacgacgagacaatcggcttcgaggacatcctgaacgaccccagccagagcgaagtgatgggagagcctcacctgatggtcgagtacaagctgggcctgctgtga

SEQ ID NO: 84 - DNA, 1113 bp, Homo sapiens
atggacgcccccaggcaggtggtcaactttgggcctggtcccgccaagctgccgcactcagtgttgttagagatacaaaaggaattattagactacaaaggagttggcattagtgttcttgaaatgagtcacaggtcatcagattttgccaagattattaacaatacagagaatcttgtgcgggaattgctagctgttccagacaactataaggtgatttttctgcaaggaggtgggtgcggccagttcagtgctgtccccttaaacctcattggcttgaaagcaggaaggtgtgctgactatgtggtgacaggagcttggtcagctaaggccgcagaagaagccaagaagtttgggactataaatatcgttcaccctaaacttgggagttatacaaaaattccagatccaagcacctggaacctcaacccagatgcctcctacgtgtattattgcgcaaatgagacggtgcatggtgtggagtttgactttatacccgatgtcaagggagcagtactggtttgtgacatgtcctcaaacttcctgtccaagccagtggatgtttccaagtttggtgtgatttttgctggtgcccagaagaatgttggctctgctggggtcaccgtggtgattgtccgtgatgacctgctggggtttgccctccgagagtgcccctcggtcctggaatacaaggtgcaggctggaaacagctccttgtacaacacgcctccatgtttcagcatctacgtcatgggcttggttctggagtggattaaaaacaatggaggtgccgcggccatggagaagcttagctccatcaaatctcaaacaatttatgagattattgataattctcaaggattctacgtttgtccagtggagccccaaaatagaagcaagatgaatattccattccgcattggcaatgccaaaggagatgatgctttagaaaaaagatttcttgataaagctcttgaactcaatatgttgtccttgaaagggcataggtctgtgggaggcatccgggcctctctgtataatgctgtcacaattgaagacgttcagaagctggccgccttcatgaaaaaatttttggagatgcatcagctatga

SEQ ID NO: 85 - DNA, 1113 bp, Artificial Sequence
  Synthetic
atggatgcccctagacaggtggtcaacttcggacctggacctgccaagctgcctcactctgtgctgctggaaatccagaaagagctgctggactacaaaggcgtgggcatcagcgtgctcgagatgagccacagaagcagcgacttcgccaagatcatcaacaacaccgagaacctcgtgcgggaactgctggccgtgcctgacaactacaaagtgatcttcctgcaaggcggcggatgcggccagttttctgctgtgcctctgaacctgatcggcctgaaggccggaagatgcgccgattatgtggtcacaggcgcttggagcgccaaggccgctgaggaagccaagaagttcggcaccatcaacatcgtgcaccccaagctgggcagctacacaaagatccccgatcctagcacctggaatctgaaccccgatgccagctacgtgtactactgcgccaacgagacagtgcacggcgtggaatttgacttcatccccgatgtgaagggcgccgtgctcgtgtgtgatatgagcagcaacttcctgagcaagcccgtggacgtgtccaagttcggcgtgatctttgccggcgctcagaaaaacgtgggatctgccggcgtgaccgtggtcattgtcagagatgatctgctgggcttcgccctgagagaatgcccttctgtgctcgagtacaaggtgcaggccggcaacagcagcctgtacaatacccctccatgcttcagcatctacgtgatgggcctcgtgctggaatggatcaagaacaatggcggagccgccgctatggaaaagctgagcagcatcaagagccagaccatctacgagatcatcgacaatagccagggcttctacgtgtgccccgtggaaccccagaaccggtccaagatgaacatccccttccggatcggcaacgccaaaggcgacgatgccctggaaaagcggttcctggataaggccctggaactgaatatgctgagcctgaagggccacagatccgtcggaggaatcagagccagcctgtataacgccgtgaccatcgaggacgtgcagaaactggctgcctttatgaagaaattcctcgagatgcaccagctgtga

SEQ ID NO: 86 - DNA, 942 bp, Homo sapiens
atggcggccctcacagacctctcatttatgtatcgctggttcaagaactgcaatctggttggcaacctctcagagaagtacgtcttcatcacaggctgtgactctggcttcgggaacctgctggccaaacagctggttgatcggggcatgcaggtgctggctgcttgcttcactgaggagggatcccagaaacttcagcgggatacctcctatcggctgcagaccaccctactggatgtcaccaagagcgaaagcatcaaggcggcggcccagtgggtgagggacaaagtgggcgaacaaggcctctgggccctggtgaacaatgctggtgtgggcctgcccagtggtcccaacgaatggctgaccaaggatgactttgtgaaggtgattaatgtgaacctggtgggactgatcgaagtgacccttcacatgctgcccatggtcaagagagcccggggcagggttgtcaacatgtccagctctggtggtcgtgtggctgtcattggtggtggctactgcgtctccaagtttggcgttgaggccttctctgacagcataaggcgtgagctctactactttggggtgaaagtctgcatcattgagccagggaactatcggacagccattctcggcaaggagaacctggagtcacgcatgcgaaagctttgggagaggctgcctcaggagacccgggacagctacggagaggattatttccgcatctatactgacaagttaaaaaacataatgcaggtggcagagcccagagtcagagatgtcatcaacagcatggagcatgctattgtttcccggagccctcgcatccgctacaaccctggcctggatgccaaactcctctacatccctctggctaagttgcccacccctgtgacagatttcatcctaagccggtaccttccaaggccagcggacagtgtctaa

SEQ ID NO: 87 - DNA, 942 bp, Artificial Sequence
  Synthetic
atggccgctctgaccgacctgagcttcatgtaccggtggttcaagaactgcaacctcgtgggcaacctgagcgagaaatacgtgttcatcaccggctgcgacagcggcttcggaaatctgctggctaagcagctggtggacagaggcatgcaggttctggccgcctgctttacagaggaaggctcccagaagctgcagcgggacacaagctacagactgcagaccacactgctggacgtgaccaagagcgagagcatcaaagccgccgctcagtgggtccgagacaaagtgggagaacaaggactgtgggccctcgtgaacaatgctggcgttggactgcctagcggccccaatgagtggctgaccaaggacgacttcgtgaaagtgatcaacgtgaaccttgtgggcctgatcgaagtgaccctgcacatgctgcccatggtcaagcgagccagaggcagagtggtcaacatgagcagctctggcggaagagtggccgtgatcggcggaggctattgcgtgtccaagtttggcgtggaagccttcagcgacagcatcagaagagagctgtactacttcggagtgaaagtgtgcatcatcgagcccggcaactaccggacagccatcctgggcaaagagaacctggaaagccggatgcggaagctgtgggagagactgccccaagagacaagagacagctacggcgaggactacttccggatctacaccgacaagctgaagaacatcatgcaggtcgccgagcctagagtgcgggacgtgatcaactctatggaacacgccatcgtgtctcggagccccagaatcagatacaaccctggcctggacgccaagctgctgtatatccctctggccaaactgcccacacctgtgaccgacttcatcctgagcagatacctgcctagacctgccgacagcgtgtga

SEQ ID NO: 88 - DNA, 1125 bp, Homo sapiens
atggatgacctctgtgaagcaaatggcacttttgccatcagcttatttaaaatattgggggaagaggacaactcaagaaacgtattcttctctcccatgagcatctcctctgccctggccatggtcttcatgggggcaaagggaagcactgcagcccagatgtcccaggcactttgtttatacaaagacggagatattcaccgaggtttccagtcacttctcagtgaagttaacagaactggcactcagtacttgcttagaactgccaacagactctttggagaaaagacgtgtgatttccttccagactttaaagaatactgtcagaagttctatcaggcagagctggaggagttgtcctttgctgaagacactgaagagtgcaggaagcatataaatgactgggtggcagagaagactgaaggtaagatttcagaggtactggatgctgggacagtcgatcccctgacaaagctggtccttgtgaatgccatttatttcaagggaaagtggaatgagcaatttgacagaaagtacacaaggggaatgctctttaaaaccaacgaggaaaaaaagacagtgcagatgatgtttaaggaagctaagtttaaaatggggtatgcggatgaggtacacacccaggtcctggagctgccctatgtggaagaggagctgagcatggtcattctgcttcccgatgacaacacggacctcgccgtggtggaaaaagcacttacatatgagaaattcaaagcctggacaaattcagaaaagttgacaaaaagtaaggttcaagttttccttcccagattaaagctggaggagagttatgacttggagcctttccttcgaagattaggaatgatcgatgcttttgacgaagccaaggcagacttttctggaatgtcaactgagaagaatgtgcctctgtccaaggttgcccacaagtgcttcgtggaggtcaatgaggaaggcacagaggctgccgcagccactgctgtggtcaggaattcccggtgcagcagaatggagccaagattctgtgcagaccacccttttcttttcttcatcaggcaccacaaaaccaactgcatcttgttctgtggcaggttctcttctccgtaa

SEQ ID NO: 89 - DNA, 1125 bp, Artificial Sequence
  Synthetic
atggacgatctgtgcgaggccaatggcaccttcgccatcagcctgttcaagatcctgggcgaagaggacaacagccggaacgtgttcttcagccccatgagcatcagcagcgccctggccatggtgtttatgggcgccaagggatctaccgccgctcagatgtctcaggccctgtgcctgtacaaggacggcgatatccacagaggcttccagagcctgctgagcgaagtgaacagaaccggcacacagtacctgctgcggaccgccaatagactgttcggcgagaaaacctgcgacttcctgcctgacttcaaagagtactgccagaagttctaccaggccgagctggaagaactgagcttcgccgaggataccgaggaatgccggaagcacatcaacgactgggtcgccgagaaaacagagggcaagatcagcgaggtgctggacgccggaacagtggaccctctgacaaagctggtgctggtcaacgccatctacttcaaaggcaagtggaacgagcagttcgaccggaagtacacccggggcatgctgtttaagaccaacgaagagaagaaaaccgtgcagatgatgttcaaagaggccaagttcaagatgggctacgccgacgaggtgcacacacaggttctggaactgccctacgtggaagaggaactgagcatggtcatcctgctgcctgacgacaacaccgatctggccgtggtggaaaaggccctgacctacgagaagttcaaggcctggaccaacagcgagaagctgaccaagagcaaggtgcaagtgttcctgccacggctgaaactggaagagagctacgacctggaacctttcctgcggagactgggcatgatcgacgccttcgatgaggccaaggccgactttagcggcatgagcaccgagaagaatgtgcccctgtctaaggtggcccacaagtgcttcgtggaagtgaacgaggaaggcacagaagccgccgctgctacagccgtcgtcagaaatagccggtgcagccggatggaaccccggttttgtgccgaccatccttttctgttcttcatccggcaccacaagaccaactgcatcctgttctgcggcagattcagcagcccttga

SEQ ID NO: 90 - DNA, 1932 bp, Homo sapiens
atgctgcttggagcctctctggtgggggtgctgctgttctccaagctggtgctgaaactgccctggacccaggtgggattctccctgttgttcctctacttgggatctggcggctggcgcttcatccgggtcttcatcaagaccatcaggcgcgatatctttggcggcctggtcctcctgaaggtgaaggcaaaggtgcgacagtgcctgcaggagcggcggacagtgcccattttgtttgcctctaccgttcggcgccaccccgacaagacggccctgatcttcgagggcacagatacccactggaccttccgccagctggatgagtactcaagcagtgtagccaacttcctgcaggcccggggcctggcctcgggcgatgtggctgccatcttcatggagaaccgcaatgagttcgtgggcctatggctgggcatggccaagctcggtgtggaggcagccctcatcaacaccaacctgcggcgggatgctctgctccactgcctcaccacctcgcgcgcacgggcccttgtctttggcagcgaaatggcctcagccatctgtgaggtccatgccagcctggacccctcgctcagcctcttctgctctggctcctgggagcccggtgcggtgcctccaagcacagaacacctggaccctctgctgaaagatgctcccaagcaccttcccagttgccctgacaagggcttcacagataaactgttctacatctacacatccggcaccacagggctgcccaaggccgccatcgtggtgcacagcaggtattaccgcatggctgccctggtgtactatggattccgcatgcggcccaacgacatcgtctatgactgcctccccctctaccactcagcaggaaacatcgtgggaatcggccagtgcctgctgcatggcatgacggtggtgattcggaagaagttctcagcctcccggttctgggacgattgtatcaagtacaactgcacgattgtgcagtacattggtgaactgtgccgctacctcctgaaccagccaccgcgggaggcagaaaaccagcaccaggttcgcatggcactaggcaatggcctccggcagtccatctggaccaacttttccagccgcttccacataccccaggtggctgagttctacggggccacagagtgcaactgtagcctgggcaacttcgacagccaggtgggggcctgtggtttcaatagccgcatcctgtccttcgtgtaccccatccggttggtacgtgtcaacgaggacaccatggagctgatccgggggcccgacggcgtctgcattccctgccagccaggtgagccgggccagctggtgggccgcatcatccagaaagaccccctgcgccgcttcgatggctacctcaaccagggcgccaacaacaagaagattgccaaggatgtcttcaagaagggggaccaggcctaccttactggtgatgtgctggtgatggacgagctgggctacctgtacttccgagaccgcactggggacacgttccgctggaaaggtgagaacgtgtccaccaccgaggtggaaggcacactcagccgcctgctggacatggctgacgtggccgtgtatggtgtcgaggtgccaggaaccgagggccgggccggaatggctgctgtggccagccccactggcaactgtgacctggagcgctttgctcaggtcttggagaaggaactgcccctgtatgcgcgccccatcttcctgcgcctcctgcctgagctgcacaaaacaggaacctacaagttccagaagacagagctacggaaggagggctttgacccggctattgtgaaagacccgctgttctatctagatgcccagaagggccgctacgtcccgctggaccaagaggcctacagccgcatccaggcaggcgaggagaagctgtga

SEQ ID NO: 91 - DNA, 1932 bp, Artificial Sequence
  Synthetic
atgctgctgggagcctctcttgtgggcgtgctgctgtttagcaagctggtgctgaagctgccctggacacaagtgggcttcagcctgctgtttctgtacctcggatctggcggctggcggttcatccgggtgttcatcaagaccatccggcgggatatcttcggcggactggtgctgctgaaagtgaaggccaaagtgcggcagtgcctgcaagagcggagaaccgtgcctatcctgtttgccagcacagtgcggagacaccccgataagacagccctgatcttcgagggcaccgacacacactggaccttcagacagctggacgagtacagcagcagcgtggccaatttcctgcaggccagaggactggctagcggagatgtggccgccatcttcatggaaaaccggaacgagttcgtcggcctgtggctcggaatggccaagctgggagttgaggccgctctgatcaacaccaacctgagaagagatgccctgctgcactgcctgaccacctctagagctagagccctggtgttcggcagcgaaatggccagcgccatctgtgaagtgcacgcctctctcgatcctagcctgagcctgttttgttccggctcttgggaacctggcgccgtgcctccatctacagaacatctcgaccctctgctgaaggacgcccctaaacatctgcctagctgccccgacaagggcttcaccgacaagctgttctacatctacaccagcggcaccaccggactgcctaaagccgctattgtggtgcacagccggtactacagaatggccgcactggtgtactacggcttccggatgaggcccaacgacatcgtgtacgactgcctgcctctgtaccacagcgccggcaatatcgttggcatcggccagtgtctgctgcacggcatgacagtcgtgatccggaagaagttcagcgccagcagattctgggacgactgcatcaagtacaactgcaccatcgtccagtacatcggcgagctgtgcagatacctgctgaaccagcctcctagagaggccgagaaccagcaccaagtgcggatggcccttggcaatggcctgagacagagcatctggaccaacttcagcagccggtttcacatccctcaggtggccgagttttacggcgccaccgagtgtaattgcagcctgggcaacttcgacagccaagtgggagcctgcggcttcaacagcagaatcctgagcttcgtgtaccccatccggctcgtcagagtgaacgaggacaccatggaactgatcagaggccccgatggcgtgtgcatcccttgtcaacctggcgaacctggccagctcgtgggcagaattatccagaaggacccactgcggagattcgacggctacctgaatcagggcgccaacaacaagaaaatcgccaaggacgtgttcaagaagggcgaccaggcctacctgactggcgacgtgctggttatggatgagctgggctacctgtacttccgggacagaaccggcgacaccttcagatggaagggcgagaatgtgtccaccaccgaggtggaaggcaccctgtctagactgctggatatggctgacgtggccgtgtacggcgtggaagtgcctggaactgaaggcagagctggcatggctgctgtggctagccctaccggcaattgcgacctggaaagattcgcccaggtgctggaaaaagagctgcccctgtacgccagacctatcttcctgagactgctgcccgagctgcacaagaccggcacctacaagttccagaaaaccgagctgcggaaagagggcttcgaccctgctatcgtgaaggaccctctgttctacctggacgcccagaaaggcagatacgtgcccctggatcaagaggcctactccagaatccaggccggcgaagagaagctgtga

SEQ ID NO: 92 - DNA, 777 bp, Homo sapiens
atgtcagcttaccctaaaagctacaatccgttcgacgacgacggggaggacgaaggcgcccggccggccccttggagggacgcccgagacctccccgacgggcccgacgcgcccgcggacaggcagcagtacttgcggcaggaggtcctccgcagggctgaggccacggccgccagcaccagcaggtccctggccctcatgtacgagtccgagaaggttggggtcgcctcttccgaggagctcgcccgtcagcgaggagtcctggagcgcacagagaagatggtggacaagatggaccaagatttgaagatcagccagaaacacatcaatagcattaagagcgtgtttggggggctggtcaattacttcaaatccaaaccagtagagaccccacctgaacagaatggcaccctcacctcccagcccaacaacagattgaaagaagctataagtacaagtaaagaacaggaagcaaagtaccaggccagccacccaaaccttagaaagctggatgatacagaccctgtccccagaggggctggttctgccatgagtactgatgcttacccaaagaacccacaccttcgagcctatcaccagaagatcgacagcaacctagatgagctgtccatgggactgggtcgtctgaaggacatagccctggggatgcagacagaaattgaggagcaagatgacattcttgaccggctgacaaccaaagtggacaagttagatgtcaacataaaaagcacagaaagaaaagttcgacaactctga

SEQ ID NO: 93 - DNA, 777 bp, Artificial Sequence
  Synthetic
atgagcgcttaccccaagagctacaaccccttcgacgacgacggcgaggatgaaggtgctagacctgctccttggcgggacgctagagatctgcctgatggacctgatgctcccgccgacagacagcagtacctgagacaagaagtgctgcggagagccgaagccacagccgcctctacatctagatctctggccctgatgtacgagagcgagaaagtgggcgtcgccagctctgaagaactggccagacagagaggcgtgctggaacggacagagaagatggtggacaagatggaccaggacctgaagatcagccagaagcacatcaacagcatcaagagcgtgttcggcggcctggtcaactacttcaagagcaagcccgtggaaacccctcctgagcagaatggcaccctgaccagccagcctaacaaccggctgaaagaggccatcagcaccagcaaagagcaagaggccaagtaccaggccagccatccaaacctgagaaagctggacgacacagaccccgtgcctagaggtgctggaagcgccatgagcaccgacgcctatcctaagaaccctcacctgagagcctaccaccagaagatcgacagcaacctggacgagctgtctatgggcctgggcagactgaaggatatcgccctgggaatgcagaccgagatcgaggaacaggacgacatcctggaccggctgaccaccaaggtggacaaactggacgtgaacatcaagtccaccgagcggaaagtgcggcagctgtaa

SEQ ID NO: 94 - DNA, 2568 bp, Homo sapiens
atggggagcgatcgggcccgcaagggcggagggggcccgaaggacttcggcgcgggactcaagtacaactcccggcacgagaaagtgaatggcttggaggaaggcgtggagttcctgccagtcaacaacgtcaagaaggtggaaaagcatggcccggggcgctgggtggtgctggcagccgtgctgatcggcctcctcttggtcttgctggggatcggcttcctggtgtggcatttgcagtaccgggacgtgcgtgtccagaaggtcttcaatggctacatgaggatcacaaatgagaattttgtggatgcctacgagaactccaactccactgagtttgtaagcctggccagcaaggtgaaggacgcgctgaagctgctgtacagcggagtcccattcctgggcccctaccacaaggagtcggctgtgacggccttcagcgagggcagcgtcatcgcctactactggtctgagttcagcatcccgcagcacctggtggaggaggccgagcgcgtcatggccgaggagcgcgtagtcatgctgcccccgcgggcgcgctccctgaagtcctttgtggtcacctcagtggtggctttccccacggactccaaaacagtacagaggacccaggacaacagctgcagctttggcctgcacgcccgcggtgtggagctgatgcgcttcaccacgcccggcttccctgacagcccctaccccgctcatgcccgctgccagtgggccctgcggggggacgccgactcagtgctgagcctcaccttccgcagctttgaccttgcgtcctgcgacgagcgcggcagcgacctggtgacggtgtacaacaccctgagccccatggagccccacgccctggtgcagttgtgtggcacctaccctccctcctacaacctgaccttccactcctcccagaacgtcctgctcatcacactgataaccaacactgagcggcggcatcccggctttgaggccaccttcttccagctgcctaggatgagcagctgtggaggccgcttacgtaaagcccaggggacattcaacagcccctactacccaggccactacccacccaacattgactgcacatggaacattgaggtgcccaacaaccagcatgtgaaggtgcgcttcaaattcttctacctgctggagcccggcgtgcctgcgggcacctgccccaaggactacgtggagatcaacggggagaaatactgcggagagaggtcccagttcgtcgtcaccagcaacagcaacaagatcacagttcgcttccactcagatcagtcctacaccgacaccggcttcttagctgaatacctctcctacgactccagtgacccatgcccggggcagttcacgtgccgcacggggcggtgtatccggaaggagctgcgctgtgatggctgggccgactgcaccgaccacagcgatgagctcaactgcagttgcgacgccggccaccagttcacgtgcaagaacaagttctgcaagcccctcttctgggtctgcgacagtgtgaacgactgcggagacaacagcgacgagcaggggtgcagttgtccggcccagaccttcaggtgttccaatgggaagtgcctctcgaaaagccagcagtgcaatgggaaggacgactgtggggacgggtccgacgaggcctcctgccccaaggtgaacgtcgtcacttgtaccaaacacacctaccgctgcctcaatgggctctgcttgagcaagggcaaccctgagtgtgacgggaaggaggactgtagcgacggctcagatgagaaggactgcgactgtgggctgcggtcattcacgagacaggctcgtgttgttgggggcacggatgcggatgagggcgagtggccctggcaggtaagcctgcatgctctgggccagggccacatctgcggtgcttccctcatctctcccaactggctggtctctgccgcacactgctacatcgatgacagaggattcaggtactcagaccccacgcagtggacggccttcctgggcttgcacgaccagagccagcgcagcgcccctggggtgcaggagcgcaggctcaagcgcatcatctcccaccccttcttcaatgacttcaccttcgactatgacatcgcgctgctggagctggagaaaccggcagagtacagctccatggtgcggcccatctgcctgccggacgcctcccatgtcttccctgccggcaaggccatctgggtcacgggctggggacacacccagtatggaggcactggcgcgctgatcctgcaaaagggtgagatccgcgtcatcaaccagaccacctgcgagaacctcctgccgcagcagatcacgccgcgcatgatgtgcgtgggcttcctcagcggcggcgtggactcctgccagggtgattccgggggacccctgtccagcgtggaggcggatgggcggatcttccaggccggtgtggtgagctggggagacggctgcgctcagaggaacaagccaggcgtgtacacaaggctccctctgtttcgggactggatcaaagagaacactggggtatag

SEQ ID NO: 95 - DNA, 2568 bp, Artificial Sequence
  Synthetic
atgggcagcgacagagccagaaaaggtggcggcggacccaaggattttggcgccggactgaagtacaacagccggcacgagaaagtgaacggcctggaagaaggcgtggaatttctgcccgtgaacaacgtgaagaaggtggaaaagcacggccctggcagatgggttgtgctggctgctgttctgatcggcctgctgctggtgctgctcggcattggatttctcgtgtggcatctgcagtaccgggatgtgcgggtgcagaaggtgttcaacggctacatgcggatcaccaacgagaacttcgtggacgcctacgagaacagcaacagcaccgagtttgtgtccctggccagcaaagtgaaggacgccctgaagctgctgtactccggcgtgccatttctgggcccctaccacaaagaaagcgccgtgacagcctttagcgagggcagcgtgatcgcctactattggagcgagttcagcatccctcagcacctggtggaagaggccgaaagagtgatggccgaggaacgggttgtgatgctgcctcctagagccagatctctgaagtccttcgtggtcaccagcgtggtggcctttccaaccgatagcaagaccgtgcagcggacccaggacaacagctgtagctttggactgcacgccagaggcgtcgagctgatgagattcaccacacctggcttccctgacagcccctatcctgctcatgctagatgccagtgggctctgagaggcgacgctgattctgtgctgagcctgaccttcagaagcttcgacctggcctcctgtgatgagaggggcagcgatctggtcaccgtgtacaacacactgagccccatggaaccccacgctctggttcagctgtgtggcacataccctccaagctacaatctgaccttccacagctcccagaacgtgctgctgatcaccctgatcacaaacaccgagagaaggcaccctggcttcgaggccacattcttccagctgcctagaatgagcagctgcggcggcagactgagaaaggcccagggcaccttcaacagcccttactaccctggccactatcctccaaacatcgactgcacctggaacatcgaggtgcccaacaaccagcatgtgaaagtgcggttcaagttcttctacctgctggaacctggcgtgcccgctggcacatgtcctaaggactacgtggaaatcaacggcgagaagtactgcggcgagcggagccagtttgtcgtgaccagcaactccaacaagatcaccgtgcggttccacagcgaccagagctataccgataccggcttcctggccgagtacctgagctacgatagcagcgatccttgtcctggccagttcacctgtagaaccggcagatgcatccggaaagaactgagatgtgacggctgggccgattgcaccgatcacagcgacgagctgaattgcagctgtgatgccggccaccagtttacctgcaagaacaagttctgcaagcccctgttctgggtctgcgacagcgtgaacgattgcggcgacaactctgatgagcagggctgctcttgtcccgctcagaccttcaggtgcagcaacggcaagtgcctgagcaagagccagcagtgcaatggcaaggacgactgcggagatggctccgatgaagccagctgtcccaaagtgaatgtggtcacatgcaccaagcacacctaccggtgcctgaatggcctgtgtctgagcaagggcaaccccgagtgtgacggcaaagaggattgcagcgacggctccgacgagaaggattgcgattgtggcctgcggagcttcaccagacaggccagagttgttggcggcaccgatgccgatgaaggcgaatggccttggcaggtttcactgcatgccctcggacagggccatatttgtggcgccagcctgatcagccccaactggcttgtttctgccgctcactgctacatcgacgaccggggcttcagatacagcgaccctactcagtggaccgccttcctgggactgcatgaccagtctcagagaagcgctccaggcgtgcaagagcggagactgaagcggatcatctctcaccctttcttcaacgacttcaccttcgactacgatatcgccctgctcgagctggaaaagcccgccgagtactcttctatggtccgacctatctgcctgcctgacgcctctcatgtgtttccagccggcaaggccatctgggttacaggctggggacacacacagtatggcggaacaggcgccctgatcctgcagaaaggcgagatccgcgtgatcaaccagaccacatgcgagaacctgctgcctcagcagatcacccctcggatgatgtgcgtgggctttctgtctggcggagtggattcctgtcaaggcgattctggcggccctctgtctagcgtggaagccgatggcagaatctttcaggccggcgttgtgtcctggggagatggatgtgcccagaggaacaagcccggcgtgtacacaagactgcctctgttcagagactggatcaaagaaaacaccggcgtgtga

SEQ ID NO: 96 - DNA, 1752 bp, Homo sapiens
atgcctttaaggaagatgaagatccctttcctcctactgttctttctgtgggaagccgagagccacgcagcatcaaggccgaacatcatcctggtgatggctgacgacctcggcattggagatcctgggtgctatgggaacaaaactatcaggactcccaatatcgaccggttggccagtgggggagtgaaactcactcagcacctggcagcatcaccgctgtgcacaccaagcagggcagccttcatgactggccggtaccctgtccgatcaggaatggcatcttggtcccgcactggagttttcctcttcacagcctcttcgggaggacttcccaccgatgagattacctttgctaagcttctgaaggatcaaggttattcaacagcactgatagggaaatggcaccttgggatgagctgtcacagcaagactgacttctgtcaccaccctttacatcacggcttcaattatttctatgggatctctttgaccaatctgagagactgcaagcccggagagggcagtgtcttcaccacgggcttcaagaggctggtcttcctccccctgcagatcgtcggggtcaccctccttacccttgctgcactcaattgtctggggctactccacgtgcctctaggcgtttttttcagccttctcttcctagcagccctaatcctgacccttttcttgggcttccttcattacttccggcccctgaactgcttcatgatgaggaactacgagatcattcagcagcccatgtcctatgacaatctcacccagaggctaacggtggaggcggcccagttcatacagcggaacactgagactccgttcctgcttgtcttgtcctacctccacgtgcacacagccctgttctccagcaaagactttgctggcaaaagtcaacacggagtctacggggatgctgttgaggaaatggactggagtgtggggcagatcttgaaccttctggatgagctgagattggctaatgataccctcatctacttcacatcggaccagggagcacatgtagaagaagtgtcttccaaaggagaaattcatggcggaagtaatgggatctataaaggaggaaaagcaaacaactgggaaggaggtatccgggttccaggcatccttcgttggcccagggtgatacaggctggccagaagattgatgagcccactagcaacatggacatatttcctacagtagccaagctggctggagctcccttgcctgaggacaggatcattgatggacgtgatctgatgcccctgcttgaaggaaaaagccaacgctccgatcatgagtttctcttccattactgcaacgcctacttaaatgctgtgcgctggcaccctcagaacagcacatccatctggaaggcctttttcttcacccccaacttcaaccccgtgggttccaacggatgctttgccacacacgtgtgcttctgtttcgggagttatgtcacccatcacgacccacctttactctttgatatttccaaagatcccagagagagaaacccactaactccagcatccgagccccggttttatgaaatcctcaaagtcatgcaggaagctgcggacagacacacccagaccctgccagaggtgcccgatcagttttcatggaacaactttctttggaagccctggcttcagctgtgctgtccttccaccggcctgtcttgccagtgtgatagagaaaaacaggataagagactgagccgctag

SEQ ID NO: 97 - DNA, 1752 bp, Artificial Sequence
  Synthetic
atgcccctgcggaagatgaagatccccttcctgctgctgttcttcctgtgggaagccgaatctcacgccgccagcagacccaacatcatcctggtcatggccgacgatctcggcatcggcgatcctggctgttacggcaacaagaccatcagaacccctaacatcgaccggctggcaagcggcggagtgaaactgacacaacacctggccgcctctccactgtgtacaccttctagagccgccttcatgaccggcagatatcctgtgcgctctggcatggccagctggtctagaacaggcgtgttcctgttcaccgccagctctggtggactgcccaccgatgagatcaccttcgccaagctgctgaaggaccagggctacagcacagccctgatcggaaaatggcacctgggcatgagctgccacagcaagaccgatttctgccaccatcctctgcaccacggcttcaactacttctacggcatcagcctgaccaacctgcgggattgcaaacctggcgagggcagcgtgttcaccaccggctttaagcggctggtgttcctgcctctgcagatcgtgggagtgaccctgctgacactggccgctctgaattgtctgggactgctgcatgtgcccctgggcgtgttcttcagcctgctgtttctggctgccctgattctgaccctgttcctgggcttcctgcactacttcagacccctgaactgcttcatgatgcggaactacgagatcatccagcagcctatgagctacgacaatctgacccagcggctgaccgtggaagccgctcagttcatccagcggaataccgagacacctttcctgctggtgctgagctacctgcacgtccacactgccctgttcagcagcaaggatttcgccggcaagtctcagcatggcgtgtacggggatgccgtggaagagatggattggagcgtgggccagatcctgaacctgctggacgaactgcggctggccaacgacaccctgatctacttcacctctgatcagggcgcccacgtggaagaagtgtctagcaagggcgagatccacggcggcagcaacggaatctacaaaggcggcaaggccaacaactgggaaggcggcattagagtgcccggcatcctgagatggcccagagtgattcaggccggccagaagatcgacgagcccaccagcaacatggacatcttccctaccgtggccaagctggctggtgctccactgcctgaggacagaatcatcgacggcagagatctgatgcctctgctggaaggcaagagccagagaagcgaccacgagtttctgttccactactgcaacgcctacctgaacgccgtgcgttggcaccctcagaacagcacctctatctggaaggcctttttcttcacgcccaacttcaaccccgtgggctccaatggctgcttcgccactcacgtgtgcttttgcttcggcagctacgtgacccaccacgatcctccactgctgttcgacatcagcaaggaccccagagagcggaaccctctgacaccagcctctgagccccggttttacgagattctgaaagtgatgcaagaggccgccgaccggcacacacaaacactgccagaagtgcccgaccagttcagctggaacaattttctgtggaagccctggctgcagctgtgctgcccttctaccggactgagctgtcagtgcgacagagagaagcaggacaagcggctgagcagatga

SEQ ID NO: 98 - DNA, 1854 bp, Homo sapiens
atggcttttccccatcggccggacgcccctgagctgcctgacttctccatgctgaagaggctggctcgagaccagctcatctatctgctggagcagcttcctggaaaaaaggatttattcattgaggcagatctcatgagccctttggatcgaattgccaatgtctccatcctgaagcaacacgaagtagacaagctatacaaggtggagaacaagccagccctcagctccaatgaacaattgtgcttcttggtcagaccccgcatcaagaatatgcgatacattgccagtcttgtcaatgctgacaaattggctggccgaactcgcaaatacaaagtgatcttcagccctcaaaagttctatgcgtgtgagatggtgcttgaggaagagggaatctatggagatgtgagctgtgatgaatgggccttctctttgctgcctcttgatgtggatctgctgagcatggaactaccagaatttttcagggattactttctggaaggagatcagcgttggatcaacactgtagctcaggccttacaccttctcagcactctctatggaccctttccaaactgctatggaattggcaggtgcgccaagatggcatatgaattgtggaggaacctggaggaggaggaggatggcgaaaccaagggccgaaggccagagattggacatatctttctcttggacagagatgtggactttgtgacagcactttgctcccaagtggtttatgagggcctagtagatgacaccttccgcatcaagtgtgggagtgtcgactttggcccagaagtcacatcctctgacaagagcctgaaggtgctactcaatgccgaggacaaggtgtttaatgagattcggaacgagcacttctccaatgtctttggcttcttgagccagaaggcccggaacttgcaggcccagtatgatcgccggagaggcatggacattaagcagatgaagaatttcgtgtcccaggagctcaagggcctgaaacaggagcaccgcctgctgagtctccatattggggcctgtgaatccatcatgaagaagaaaaccaagcaggatttccaggagctaatcaagactgagcatgcactgctagaggggttcaacatccgggagagcaccagctacattgaggaacacatagaccggcaggtgtcgcctatagaaagcctgcgcctcatgtgccttttgtccatcactgagaatggtttgatccccaaggattaccgatctctgaaaacacagtatctgcagagctatggccctgagcacctgctaaccttctccaatctgcgaagagctgggctcctaacggagcaggcccccggggacaccctcacagccgtggagagtaaagtgagcaagctggtgaccgacaaggctgcaggaaagattactgatgccttcagttctctggccaagaggagcaattttcgtgccatcagcaaaaagctgaatttgatcccacgtgtggacggcgagtatgatctgaaagtgccccgagacatggcttacgtcttcggtggtgcttatgtgcccctgagctgccgaatcattgagcaggtgctagagcggcgaagctggcagggccttgatgaggtggtacggctgctcaactgcagtgactttgcattcacagatatgactaaggaagacaaggcttccagtgagtccctgcgcctcatcttggtggtgttcttgggtggttgtacattctctgagatctcagccctccggttcctgggcagagagaaaggctacaggttcattttcctgacgacagcagtcacaaacagcgctcgccttatggaggccatgagtgaggtgaaagcctga

SEQ ID NO: 99 - DNA, 1854 bp, Artificial Sequence
  Synthetic
atggcctttccacacagacctgatgctcccgagctgcccgacttctccatgctgaaaagactggcccgggaccagctgatctacctgctggaacagctgcccggcaagaaggacctgttcatcgaggccgatctgatgagccctctggacagaatcgccaacgtgtccatcctgaagcagcacgaggtggacaagctgtacaaggtggaaaacaagcccgctctgagcagcaacgagcagctgtgctttctcgtgcggcccagaatcaagaacatgcggtatatcgccagcctggtcaacgccgataagctggccggcagaacccggaagtacaaagtgatcttcagcccgcaaaagttctacgcctgcgagatggtgctggaagaggaaggcatctacggcgacgtgtcctgtgatgagtgggccttttctctgctgcccctggatgtggacctgctgagcatggaactgcccgagttcttccgggactacttcctggaaggcgaccagcggtggatcaacacagttgcccaagctctgcatctgctgtctaccctgtacggcccctttcctaactgctacggcatcggcagatgcgccaagatggcctatgagctgtggcggaacctggaagaagaagaggacggcgagacaaaaggcagaaggcctgagatcggccacatctttctgctggaccgcgacgtggacttcgtgacagccctgtgtagccaggtggtgtacgaaggcctggtggacgacaccttcaggatcaagtgtggcagcgtggactttggccccgaagtgaccagcagcgacaagtctctgaaggtgctgctgaacgccgaggacaaggtgttcaacgagatccggaacgagcacttcagcaacgtgttcggcttcctgagccagaaggccagaaatctgcaggcccagtacgatcggagaagaggcatggacatcaagcagatgaagaacttcgtgtcccaagagctgaagggcctgaagcaagagcacagactgctgagcctgcacatcggagcctgcgagtccatcatgaagaaaaagaccaagcaggacttccaagaactgatcaagaccgagcacgccctgctcgagggcttcaacatcagagagagcaccagctacatcgaggaacacatcgacagacaggtgtccccaatcgagagcctgagactgatgtgcctgctgtccatcaccgagaacggactgatccccaaggactacagaagcctgaaaacccagtacctgcagagctacggccccgaacatctgctgaccttctccaatctgcggagagccggactgctgacagaacaggctcctggcgatacactgaccgccgtggaaagcaaggtgtccaagctggtcaccgataaggccgctggcaagatcaccgatgcctttagcagcctggccaagcggagcaacttccgggccatcagcaagaagctgaatctgatccctagagtggatggcgagtacgacctgaaagtgcccagagacatggcttatgtgttcggcggagcctacgtgcccctgagctgcagaatcatcgagcaggtcctggaacggcggtcttggcagggactcgatgaggttgtccggctgctgaattgcagcgacttcgccttcaccgacatgaccaaagaggataaggccagctctgagagcctgcggctgatcctggtggtgtttctcggcggctgtaccttcagcgagatcagcgccctgagattcctgggaagagagaagggctaccgcttcatcttcctgaccaccgccgtgaccaatagcgccagactgatggaagccatgagcgaagtgaaagcctga

SEQ ID NO: 100 - DNA, 1428 bp, Homo sapiens
atggggatgtgggcatcgctggacgctttgtgggagatgccggccgagaagcgtatcttcggggccgtgctgctcttttcctggacagtgtatctttgggagaccttcctagcacagcggcagagaaggatatataaaacaacaactcatgtaccaccggagttaggacagatcatggattctgaaacatttgagaaatctcgactctatcaactggataaaagcactttcagcttctggtcaggactctattcagagactgaaggcactcttattcttctctttggaggaataccttatctctggagactttctggacggttctgtggttatgctggctttggaccagaatatgagatcactcagtccctggtgtttctgctgttggctacacttttcagtgcattgactggtttgccatggagtctttataatacttttgtgatagaagaaaaacatggcttcaatcaacagactttggggttcttcatgaaagatgcaatcaagaaatttgttgtgactcagtgtattttgttgcctgtgtcttcacttctactttacattattaaaattgggggtgactatttttttatttatgcctggctgttcacattagttgtgtctctggttcttgtcacaatctatgctgattatattgcccctttatttgacaaattcacacctctgcctgagggaaagcttaaagaagaaattgaagtaatggcaaagagtattgactttcctttgacgaaggtgtatgttgtggaaggatctaaacgctcttcccacagcaatgcttatttttatggcttcttcaagaacaagcgaatagttttgtttgacactctactagaagagtactctgtactaaacaaagacatccaggaggattctggcatggaaccccgcaatgaggaagaagggaacagtgaagaaataaaagctaaagttaaaaataagaaacaaggatgtaaaaatgaggaggtactcgctgtactaggccatgaactggggcactggaagttgggacatacagtcaaaaatatcattattagccagatgaattctttcctgtgtttttttttatttgctgtattaattggtcgaaaggagctttttgctgcatttggtttttatgatagccaacccactcttattggactattgatcatcttccagtttattttttcaccttacaatgaggttctttctttttgcctaacagtcctaagccgcagatttgagtttcaagctgatgcatttgccaagaaacttgggaaggctaaagacttatattctgctttaatcaaacttaacaaagataacttgggattccctgtttctgactggttgttctcaatgtggcattattctcatcctccactgctagagagacttcaagctttgaaaactatgaagcaacactga

SEQ ID NO: 101 - DNA, 1428 bp, Artificial Sequence
  Synthetic
atgggaatgtgggcctctctggatgccctgtgggaaatgcctgccgagaagagaatcttcggcgccgtgctgctgttcagctggacagtgtatctgtgggagacatttctggcccagcggcagcggagaatctacaagaccacaacacacgtgccacctgagctgggacagatcatggacagcgagacattcgagaagtcccggctgtaccagctggataagagcaccttcagcttttggagcggcctgtacagcgaaaccgagggcacactgatcctgctgtttggcggcatcccttacctgtggcggctgagcggaagattctgtggctatgccggcttcggccccgagtacgagattacccagagcctggtgttcctgctgctggccacactgtttagcgccctgacaggactgccttggagcctgtacaacaccttcgtgatcgaggaaaagcacggcttcaaccagcagaccctgggcttctttatgaaggacgccatcaagaaattcgtggtcacccagtgcatcctgctgcctgtgtcatctctgctgctgtacatcatcaagatcggcggcgactacttcttcatctacgcctggctgttcaccctggtggtgtctctggtgctggtcacaatctacgccgactatatcgcccctctgttcgacaagttcacccctctgcctgagggcaagctgaaagaagagatcgaagtgatggccaagagcatcgacttcccactgaccaaggtgtacgtggtggaaggcagcaagagaagcagccacagcaacgcctacttctacggcttcttcaagaacaagcggatcgtgctgttcgataccctgctggaagagtacagcgtgctgaacaaggacatccaagaggacagcggcatggaaccccggaatgaggaagagggcaacagcgaagagatcaaggccaaagtgaagaacaagaagcagggctgcaagaacgaagaggtgctggccgtgctgggacacgaactcggacattggaaactgggccacaccgtgaagaatatcatcatcagccagatgaacagcttcctgtgcttcttcctgtttgccgtgctgatcggccggaaagaactgttcgccgccttcggcttctacgacagccagcctacactgatcggactgctgatcatcttccagttcatcttcagcccctacaacgaggtgctgagcttctgcctgacagtgctgtccagaagattcgagttccaagccgacgccttcgccaagaagctgggcaaagccaaggacctgtactctgccctgatcaagctgaacaaagataacctgggctttcccgtgtccgactggctgtttagcatgtggcactactctcaccctccactgctggaaagactgcaggccctgaaaaccatgaagcagcactga

SEQ ID NO: 102 - protein, 2595 aa, Homo sapiens
MASLFHQLQILVWKNWLGVKRQPLWTLVLILWPVIIFIILAITRTKFPPTAKPTCYLAPRNLPSTGFFPFLQTLLCDTDSKCKDTPYGPQDLLRRKGIDDALFKDSEILRKSSNLDKDSSLSFQSTQVPERRHASLATVFPSPSSDLEIPGTYTFNGSQVLARILGLEKLLKQNSTSEDIRRELCDSYSGYIVDDAFSWTFLGRNVFNKFCLSNMTLLESSLQELNKQFSQLSSDPNNQKIVFQEIVRMLSFFSQVQEQKAVWQLLSSFPNVFQNDTSLSNLFDVLRKANSVLLVVQKVYPRFATNEGFRTLQKSVKHLLYTLDSPAQGDSDNITHVWNEDDGQTLSPSSLAAQLLILENFEDALLNISANSPYIPYLACVRNVTDSLARGSPENLRLLQSTIRFKKSFLRNGSYEDYFPPVPEVLKSKLSQLRNLTELLCESETFSLIEKSCQLSDMSFGSLCEESEFDLQLLEAAELGTEIAASLLYHDNVISKKVRDLLTGDPSKINLNMDQFLEQALQMNYLENITQLIPIIEAMLHVNNSADASEKPGQLLEMFKNVEELKEDLRRTTGMSNRTIDKLLAIPIPDNRAEIISQVFWLHSCDTNITTPKLEDAMKEFCNLSLSERSRQSYLIGLTLLHYLNIYNFTYKVFFPRKDQKPVEKMMELFIRLKEILNQMASGTHPLLDKMRSLKQMHLPRSVPLTQAMYRSNRMNTPQGSFSTISQALCSQGITTEYLTAMLPSSQRPKGNHTKDFLTYKLTKEQIASKYGIPINSTPFCFSLYKDIINMPAGPVIWAFLKPMLLGRILYAPYNPVTKAIMEKSNVTLRQLAELREKSQEWMDKSPLFMNSFHLLNQAIPMLQNTLRNPFVQVFVKFSVGLDAVELLKQIDELDILRLKLENNIDIIDQLNTLSSLTVNISSCVLYDRIQAAKTIDEMEREAKRLYKSNELFGSVIFKLPSNRSWHRGYDSGNVFLPPVIKYTIRMSLKTAQTTRSLRTKIWAPGPHNSPSHNQIYGRAFIYLQDSIERAIIELQTGRNSQEIAVQVQAIPYPCFMKDNFLTSVSYSLPIVLMVAWVVFIAAFVKKLVYEKDLRLHEYMKMMGVNSCSHFFAWLIESVGFLLVTIVILIIILKFGNILPKTNGFILFLYFSDYSFSVIAMSYLISVFFNNTNIAALIGSLIYIIAFFPFIVLVTVENELSYVLKVFMSLLSPTAFSYASQYIARYEEQGIGLQWENMYTSPVQDDTTSFGWLCCLILADSFIYFLIAWYVRNVFPGTYGMAAPWYFPILPSYWKERFGCAEVKPEKSNGLMFTNIMMQNTNPSASPEYMFSSNIEPEPKDLTVGVALHGVTKIYGSKVAVDNLNLNFYEGHITSLLGPNGAGKTTTISMLTGLFGASAGTIFVYGKDIKTDLHTVRKNMGVCMQHDVLFSYLTTKEHLLLYGSIKVPHWTKKQLHEEVKRTLKDTGLYSHRHKRVGTLSGGMKRKLSISIALIGGSRVVILDEPSTGVDPCSRRSIWDVISKNKTARTIILSTHHLDEAEVLSDRIAFLEQGGLRCCGSPFYLKEAFGDGYHLTLTKKKSPNLNANAVCDTMAVTAMIQSHLPEAYLKEDIGGELVYVLPPFSTKVSGAYLSLLRALDNGMGDLNIGCYGISDTTVEEVFLNLTKESQKNSAMSLEHLTQKKIGNSNANGISTPDDLSVSSSNFTDRDDKILTRGERLDGFGLLLKKIMAILIKRFHHTRRNWKGLIAQVILPIVFVTTAMGLGTLRNSSNSYPEIQISPSLYGTSEQTAFYANYHPSTEALVSAMWDFPGIDNMCLNTSDLQCLNKDSLEKWNTSGEPITNFGVCSCSENVQECPKFNYSPPHRRTYSSQVIYNLTGQRVENYLISTANEFVQKRYGGWSFGLPLTKDLRFDITGVPANRTLAKVWYDPEGYHSLPAYLNSLNNFLLRVNMSKYDAARHGIIMYSHPYPGVQDQEQATISSLIDILVALSILMGYSVTTASFVTYVVREHQTKAKQLQHISGIGVTCYWVTNFIYDMVFYLVPVAFSIGIIAIFKLPAFYSENNLGAVSLLLLLFGYATFSWMYLLAGLFHETGMAFITYVCVNLFFGINSIVSLSVVYFLSKEKPNDPTLELISETLKRIFLIFPQFCFGYGLIELSQQQSVLDFLKAYGVEYPNETFEMNKLGAMFVALVSQGTMFFSLRLLINESLIKKLRLFFRKFNSSHVRETIDEDEDVRAERLRVESGAAEFDLVQLYCLTKTYQLIHKKIIAVNNISIGIPAGECFGLLGVNGAGKTTIFKMLTGDIIPSSGNILIRNKTGSLGHVDSHSSLVGYCPQEDALDDLVTVEEHLYFYARVHGIPEKDIKETVHKLLRRLHLMPFKDRATSMCSYGTKRKLSTALALIGKPSILLLDEPSSGMDPKSKRHLWKIISEEVQNKCSVILTSHSMEECEALCTRLAIMVNGKFQCIGSLQHIKSRFGRGFTVKVHLKNNKVTMETLTKFMQLHFPKTYLKDQHLSMLEYHVPVTAGGVANIFDLLETNKTALNITNFLVSQTTLEEVFINFAKDQKSYETADTSSQGSTISVDSQDDQMES

SEQ ID NO: 103 - protein, 2277 aa, Homo sapiens
MFTYIKIITSGDSDNITHVWNEDDGQTLSPSSLAAQLLILENFEDALLNISANSPYIPYLACVRNVTDSLARGSPENLRLLQSTIRFKKSFLRNGSYEDYFPPVPEVLKSKLSQLRNLTELLCESETFSLIEKSCQLSDMSFGSLCEESEFDLQLLEAAELGTEIAASLLYHDNVISKKVRDLLTGDPSKINLNMDQFLEQALQMNYLENITQLIPIIEAMLHVNNSADASEKPGQLLEMFKNVEELKEDLRRTTGMSNRTIDKLLAIPIPDNRAEIISQVFWLHSCDTNITTPKLEDAMKEFCNLSLSERSRQSYLIGLTLLHYLNIYNFTYKVFFPRKDQKPVEKMMELFIRLKEILNQMASGTHPLLDKMRSLKQMHLPRSVPLTQAMYRSNRMNTPQGSFSTISQALCSQGITTEYLTAMLPSSQRPKGNHTKDFLTYKLTKEQIASKYGIPINSTPFCFSLYKDIINMPAGPVIWAFLKPMLLGRILYAPYNPVTKAIMEKSNVTLRQLAELREKSQEWMDKSPLFMNSFHLLNQAIPMLQNTLRNPFVQVFVKFSVGLDAVELLKQIDELDILRLKLENNIDIIDQLNTLSSLTVNISSCVLYDRIQAAKTIDEMEREAKRLYKSNELFGSVIFKLPSNRSWHRGYDSGNVFLPPVIKYTIRMSLKTAQTTRSLRTKIWAPGPHNSPSHNQIYGRAFIYLQDSIERAIIELQTGRNSQEIAVQVQAIPYPCFMKDNFLTSVSYSLPIVLMVAWVVFIAAFVKKLVYEKDLRLHEYMKMMGVNSCSHFFAWLIESVGFLLVTIVILIIILKFGNILPKTNGFILFLYFSDYSFSVIAMSYLISVFFNNTNIAALIGSLIYIIAFFPFIVLVTVENELSYVLKVFMSLLSPTAFSYASQYIARYEEQGIGLQWENMYTSPVQDDTTSFGWLCCLILADSFIYFLIAWYVRNVFPGTYGMAAPWYFPILPSYWKERFGCAEVKPEKSNGLMFTNIMMQNTNPSASPEYMFSSNIEPEPKDLTVGVALHGVTKIYGSKVAVDNLNLNFYEGHITSLLGPNGAGKTTTISMLTGLFGASAGTIFVYGKDIKTDLHTVRKNMGVCMQHDVLFSYLTTKEHLLLYGSIKVPHWTKKQLHEEVKRTLKDTGLYSHRHKRVGTLSGGMKRKLSISIALIGGSRVVILDEPSTGVDPCSRRSIWDVISKNKTARTIILSTHHLDEAEVLSDRIAFLEQGGLRCCGSPFYLKEAFGDGYHLTLTKKKSPNLNANAVCDTMAVTAMIQSHLPEAYLKEDIGGELVYVLPPFSTKVSGAYLSLLRALDNGMGDLNIGCYGISDTTVEEVFLNLTKESQKNSAMSLEHLTQKKIGNSNANGISTPDDLSVSSSNFTDRDDKILTRGERLDGFGLLLKKIMAILIKRFHHTRRNWKGLIAQVILPIVFVTTAMGLGTLRNSSNSYPEIQISPSLYGTSEQTAFYANYHPSTEALVSAMWDFPGIDNMCLNTSDLQCLNKDSLEKWNTSGEPITNFGVCSCSENVQECPKFNYSPPHRRTYSSQVIYNLTGQRVENYLISTANEFVQKRYGGWSFGLPLTKDLRFDITGVPANRTLAKVWYDPEGYHSLPAYLNSLNNFLLRVNMSKYDAARHGIIMYSHPYPGVQDQEQATISSLIDILVALSILMGYSVTTASFVTYVVREHQTKAKQLQHISGIGVTCYWVTNFIYDMVFYLVPVAFSIGIIAIFKLPAFYSENNLGAVSLLLLLFGYATFSWMYLLAGLFHETGMAFITYVCVNLFFGINSIVSLSVVYFLSKEKPNDPTLELISETLKRIFLIFPQFCFGYGLIELSQQQSVLDFLKAYGVEYPNETFEMNKLGAMFVALVSQGTMFFSLRLLINESLIKKLRLFFRKFNSSHVRETIDEDEDVRAERLRVESGAAEFDLVQLYCLTKTYQLIHKKIIAVNNISIGIPAGECFGLLGVNGAGKTTIFKMLTGDIIPSSGNILIRNKTGSLGHVDSHSSLVGYCPQEDALDDLVTVEEHLYFYARVHGIPEKDIKETVHKLLRRLHLMPFKDRATSMCSYGTKRKLSTALALIGKPSILLLDEPSSGMDPKSKRHLWKIISEEVQNKCSVILTSHSMEECEALCTRLAIMVNGKFQCIGSLQHIKSRFGRGFTVKVHLKNNKVTMETLTKFMQLHFPKTYLKDQHLSMLEYHVPVTAGGVANIFDLLETNKTALNITNFLVSQTTLEEVFINFAKDQKSYETADTSSQGSTISVDSQDDQMES

SEQ ID NO: 104 - protein, 349 aa, Homo sapiens
MAAEEEEVDSADTGERSGWLTGWLPTWCPTSISHLKEAEEKMLKCVPCTYKKEPVRISNGNKIWTLKFSHNISNKTPLVLLHGFGGGLGLWALNFGDLCTNRPVYAFDLLGFGRSSRPRFDSDAEEVENQFVESIEEWRCALGLDKMILLGHNLGGFLAAAYSLKYPSRVNHLILVEPWGFPERPDLADQDRPIPVWIRALGAALTPFNPLAGLRIAGPFGLSLVQRLRPDFKRKYSSMFEDDTVTEYIYHCNVQTPSGETAFKNMTIPYGWAKRPMLQRIGKMHPDIPVSVIFGARSCIDGNSGTSIQSLRPHSYVKTIAILGAGHYVYADQPEEFNQKVKEICDTVD

SEQ ID NO: 105 - protein, 508 aa, Homo sapiens
MELEVRRVRQAFLSGRSRPLRFRLQQLEALRRMVQEREKDILTAIAADLCKSEFNVYSQEVITVLGEIDFMLENLPEWVTAKPVKKNVLTMLDEAYIQPQPLGVVLIIGAWNYPFVLTIQPLIGAIAAGNAVIIKPSELSENTAKILAKLLPQYLDQDLYIVINGGVEETTELLKQRFDHIFYTGNTAVGKIVMEAAAKHLTPVTLELGGKSPCYIDKDCDLDIVCRRITWGKYMNCGQTCIAPDYILCEASLQNQIVWKIKETVKEFYGENIKESPDYERIINLRHFKRILSLLEGQKIAFGGETDEATRYIAPTVLTDVDPKTKVMQEEIFGPILPIVPVKNVDEAINFINEREKPLALYVFSHNHKLIKRMIDETSSGGVTGNDVIMHFTLNSFPFGGVGSSGMGAYHGKHSFDTFSHQRPCLLKSLKREGANKLRYPPNSQSKVDWGKFFLLKRFNKEKLGLLLLTFLGIVAAVLVKKYQAVLRRKALLIFLVVHRLRWSSKQR

SEQ ID NO: 106 - protein, 701 aa, Homo sapiens
MATYKVRVATGTDLLSGTRDSISLTIVGTQGESHKQLLNHFGRDFATGAVGQYTVQCPQDLGELIIIRLHKERYAFFPKDPWYCNYVQICAPNGRIYHFPAYQWMDGYETLALREATGKTTADDSLPVLLEHRKEEIRAKQDFYHWRVFLPGLPSYVHIPSYRPPVRRHRNPNRPEWNGYIPGFPILINFKATKFLNLNLRYSFLKTASFFVRLGPMALAFKVRGLLDCKHSWKRLKDIRKIFPGKKSVVSEYVAEHWAEDTFFGYQYLNGVNPGLIRRCTRIPDKFPVTDDMVAPFLGEGTCLQAELEKGNIYLADYRIMEGIPTVELSGRKQHHCAPLCLLHFGPEGKMMPIAIQLSQTPGPDCPIFLPSDSEWDWLLAKTWVRYAEFYSHEAIAHLLETHLIAEAFCLALLRNLPMCHPLYKLLIPHTRYTVQINSIGRAVLLNEGGLSAKGMSLGVEGFAGVMVRALSELTYDSLYLPNDFVERGVQDLPGYYYRDDSLAVWNALEKYVTEIITYYYPSDAAVEGDPELQSWVQEIFKECLLGRESSGFPRCLRTVPELIRYVTIVIYTCSAKHAAVNTGQMEFTAWMPNFPASMRNPPIQTKGLTTLETFMDTLPDVKTTCITLLVLWTLSREPDDRRPLGHFPDIHFVEEAPRRSIEAFRQRLNQISHDIRQRNKCLPIPYYYLDPVLIENSISI

SEQ ID NO: 107 - protein, 711 aa, Homo sapiens
MAVYRLCVTTGPYLRAGTLDNISVTLVGTCGESPKQRLDRMGRDFAPGSVQKYKVRCTAELGELLLLRVHKERYAFFRKDSWYCSRICVTEPDGSVSHFPCYQWIEGYCTVELRPGTARTICQDSLPLLLDHRTRELRARQECYRWKIYAPGFPCMVDVNSFQEMESDKKFALTKTTTCVDQGDSSGNRYLPGFPMKIDIPSLMYMEPNVRYSATKTISLLFNAIPASLGMKLRGLLDRKGSWKKLDDMQNIFWCHKTFTTKYVTEHWCEDHFFGYQYLNGVNPVMLHCISSLPSKLPVTNDMVAPLLGQDTCLQTELERGNIFLADYWILAEAPTHCLNGRQQYVAAPLCLLWLSPQGALVPLAIQLSQTPGPDSPIFLPTDSEWDWLLAKTWVRNSEFLVHENNTHFLCTHLLCEAFAMATLRQLPLCHPIYKLLLPHTRYTLQVNTIARATLLNPEGLVDQVTSIGRQGLIYLMSTGLAHFTYTNFCLPDSLRARGVLAIPNYHYRDDGLKIWAAIESFVSEIVGYYYPSDASVQQDSELQAWTGEIFAQAFLGRESSGFPSRLCTPGEMVKFLTAIIFNCSAQHAAVNSGQHDFGAWMPNAPSSMRQPPPQTKGTTTLKTYLDTLPEVNISCNNLLLFWLVSQEPKDQRPLGTYPDEHFTEEAPRRSIAAFQSRLAQISRDIQERNQGLALPYTYLDPPLIENSVSI

SEQ ID NO: 108 - protein, 843 aa, Homo sapiens
MPRGAFRPCLPALYFAFLTCPTPEQRMSGTQAPDIHLGEPARGTGCVRGKQTSIRVQDCGRREEARAASRELRREKAQEHPRESWAHPQPYPAPQPLALRPETQPCPACRSSPPGRLLLRPALPGHPFLLPIMAVYRLCVTTGPYLRAGTLDNISVTLVGTCGESPKQRLDRMGRDFAPGSVQKYKVRCTAELGELLLLRVHKERYAFFRKDSWYCSRICVTEPDGSVSHFPCYQWIEGYCTVELRPGTARTICQDSLPLLLDHRTRELRARQECYRWKIYAPGFPCMVDVNSFQEMESDKKFALTKTTTCVDQGDSSGNRYLPGFPMKIDIPSLMYMEPNVRYSATKTISLLFNAIPASLGMKLRGLLDRKGSWKKLDDMQNIFWCHKTFTTKYVTEHWCEDHFFGYQYLNGVNPVMLHCISSLPSKLPVTNDMVAPLLGQDTCLQTELERGNIFLADYWILAEAPTHCLNGRQQYVAAPLCLLWLSPQGALVPLAIQLSQTPGPDSPIFLPTDSEWDWLLAKTWVRNSEFLVHENNTHFLCTHLLCEAFAMATLRQLPLCHPIYKLLLPHTRYTLQVNTIARATLLNPEGLVDQVTSIGRQGLIYLMSTGLAHFTYTNFCLPDSLRARGVLAIPNYHYRDDGLKIWAAIESFVSEIVGYYYPSDASVQQDSELQAWTGEIFAQAFLGRESSGFPSRLCTPGEMVKFLTAIIFNCSAQHAAVNSGQHDFGAWMPNAPSSMRQPPPQTKGTTTLKTYLDTLPEVNISCNNLLLFWLVSQEPKDQRPLGTYPDEHFTEEAPRRSIAAFQSRLAQISRDIQERNQGLALPYTYLDPPLIENSVSI

SEQ ID NO: 109 - protein, 158 aa, Homo sapiens
MMRFMLLFSRQGKLRLQKWYLATSDKERKKMVRELMQVVLARKPKMCSFLEWRDLKVVYKRYASLYFCCAIEGQDNELITLELIHRYVELLDKYFGSVCELDIIFNFEKAYFILDEFLMGGDVQDTSKKSVLKAIEQADLLQEEDESPRSVLEEMGLA

SEQ ID NO: 110 - protein, 589 aa, Homo sapiens
MLHLHHSCLCFRSWLPAMLAVLLSLAPSASSDISASRPNILLLMADDLGIGDIGCYGNNTMRTPNIDRLAEDGVKLTQHISAASLCTPSRAAFLTGRYPVRSGMVSSIGYRVLQWTGASGGLPTNETTFAKILKEKGYATGLIGKWHLGLNCESASDHCHHPLHHGFDHFYGMPFSLMGDCARWELSEKRVNLEQKLNFLFQVLALVALTLVAGKLTHLIPVSWMPVIWSALSAVLLLASSYFVGALIVHADCFLMRNHTITEQPMCFQRTTPLILQEVASFLKRNKHGPFLLFVSFLHVHIPLITMENFLGKSLHGLYGDNVEEMDWMVGRILDTLDVEGLSNSTLIYFTSDHGGSLENQLGNTQYGGWNGIYKGGKGMGGWEGGIRVPGIFRWPGVLPAGRVIGEPTSLMDVFPTVVRLAGGEVPQDRVIDGQDLLPLLLGTAQHSDHEFLMHYCERFLHAARWHQRDRGTMWKVHFVTPVFQPEGAGACYGRKVCPCFGEKVVHHDPPLLFDLSRDPSETHILTPASEPVFYQVMERVQQAVWEHQRTLSPVPLQLDRLGNIWRPWLQPCCGPFPLCWCLREDDPQ

SEQ ID NO: 111 - protein, 242 aa, Homo sapiens
MSNPRSLEEEKYDMSGARLALILCVTKAREGSEEDLDALEHMFRQLRFESTMKRDPTAEQFQEELEKFQQAIDSREDPVSCAFVVLMAHGREGFLKGEDGEMVKLENLFEALNNKNCQALRAKPKVYIIQACRGEQRDPGETVGGDEIVMVIKDSPQTIPTYTDALHVYSTVEGYIAYRHDQKGSCFIQTLVDVFTKRKGHILELLTEVTRRMAEAELVQEGKARKTNPEIQSTLRKRLYLQ

SEQ ID NO: 112 - protein, 529 aa, Homo sapiens
MGSSRAPWMGRVGGHGMMALLLAGLLLPGTLAKSIGTFSDPCKDPTRITSPNDPCLTGKGDSSGFSSYSGSSSSGSSISSARSSGGGSSGSSSGSSIAQGGSAGSFKPGTGYSQVSYSSGSGSSLQGASGSSQLGSSSSHSGNSGSHSGSSSSHSSSSSSFQFSSSSFQVGNGSALPTNDNSYRGILNPSQPGQSSSSSQTFGVSSSGQSVSSNQRPCSSDIPDSPCSGGPIVSHSGPYIPSSHSVSGGQRPVVVVVDQHGSGAPGVVQGPPCSNGGLPGKPCPPITSVDKSYGGYEVVGGSSDSYLVPGMTYSKGKIYPVGYFTKENPVKGSPGVPSFAAGPPISEGKYFSSNPIIPSQSAASSAIAFQPVGTGGVQLCGGGSTGSKGPCSPSSSRVPSSSSISSSSGLPYHPCGSASQSPCSPPGTGSFSSSSSSQSSGKIILQPCGSKSSSSGHPCMSVSSLTLTGGPDGSPHPDPSAGAKPCGSSSAGKIPCRSIRDILAQVKPLGPQLADPEVFLPQGELLNSP

SEQ ID NO: 113 - protein, 383 aa, Homo sapiens
MFWTFKEWFWLERFWLPPTIKWSDLEDHDGLVFVKPSHLYVTIPYAFLLLIIRRVFEKFVASPLAKSFGIKETVRKVTPNTVLENFFKHSTRQPLQTDIYGLAKKCNLTERQVERWFRSRRNQERPSRLKKFQEACWRFAFYLMITVAGIAFLYDKPWLYDLWEVWNGYPKQPLLPSQYWYYILEMSFYWSLLFRLGFDVKRKDFLAHIIHHLAAISLMSFSWCANYIRSGTLVMIVHDVADIWLESAKMFSYAGWTQTCNTLFFIFSTIFFISRLIVFPFWILYCTLILPMYHLEPFFSYIFLNLQLMILQVLHLYWGYYILKMLNRCIFMKSIQDVRSDDEDYEEEEEEEEEEATKGKEMDCLKNGLRAERHLIPNGQHGH

SEQ ID NO: 114 - protein, 424 aa, Homo sapiens
MTLRPGTMRLACMFSSILLFGAAGLLLFISLQDPTELAPQQVPGIKFNIRPRQPHHDLPPGGSQDGDLKEPTERVTRDLSSGAPRGRNLPAPDQPQPPLQRGTRLRLRQRRRRLLIKKMPAAATIPANSSDAPFIRPGPGTLDGRWVSLHRSQQERKRVMQEACAKYRASSSRRAVTPRHVSRIFVEDRHRVLYCEVPKAGCSNWKRVLMVLAGLASSTADIQHNTVHYGSALKRLDTFDRQGILHRLSTYTKMLFVREPFERLVSAFRDKFEHPNSYYHPVFGKAILARYRANASREALRTGSGVRFPEFVQYLLDVHRPVGMDIHWDHVSRLCSPCLIDYDFVGKFESMEDDANFFLSLIRAPRNLTFPRFKDRHSQEARTTARIAHQYFAQLSALQRQRTYDFYYMDYLMFNYSKPFADLY

SEQ ID NO: 115 - protein, 211 aa, Homo sapiens
MANAGLQLLGFILAFLGWIGAIVSTALPQWRIYSYAGDNIVTAQAMYEGLWMSCVSQSTGQIQCKVFDSLLNLSSTLQATRALMVVGILLGVIAIFVATVGMKCMKCLEDDEVQKMRMAVIGGAIFLLAGLAILVATAWYGNRIVQEFYDPMTPVNARYEFGQALFTGWAAASLCLLGGALLCCSCPRKTTSYPTPRPYPKPAPSSGKDYV

SEQ ID NO: 116 - protein, 98 aa, Homo sapiens
MIPGGLSEAKPATPEIQEIVDKVKPQLEEKTNETYGKLEAVQYKTQVVAGTNYYIKVRAGDNKYMHLKVFKSLPGQNEDLVLTGYQVDKNKDDELTGF

SEQ ID NO: 117 - protein, 531 aa, Homo sapiens
MLPITDRLLHLLGLEKTAFRIYAVSTLLLFLLFFLFRLLLRFLRLCRSFYITCRRLRCFPQPPRRNWLLGHLGMYLPNEAGLQDEKKVLDNMHHVLLVWMGPVLPLLVLVHPDYIKPLLGASAAIAPKDDLFYGFLKPWLGDGLLLSKGDKWSRHRRLLTPAFHFDILKPYMKIFNQSADIMHAKWRHLAEGSAVSLDMFEHISLMTLDSLQKCVFSYNSNCQEKMSDYISAIIELSALSVRRQYRLHHYLDFIYYRSADGRRFRQACDMVHHFTTEVIQERRRALRQQGAEAWLKAKQGKTLDFIDVLLLARDEDGKELSDEDIRAEADTFMFEGHDTTSSGISWMLFNLAKYPEYQEKCREEIQEVMKGRELEELEWDDLTQLPFTTMCIKESLRQYPPVTLVSRQCTEDIKLPDGRIIPKGIICLVSIYGTHHNPTVWPDSKVYNPYRFDPDNPQQRSPLAYVPFSAGPRNCIGQSFAMAELRVVVALTLLRFRLSVDRTRKVRRKPELILRTENGLWLKVEPLPPRA

SEQ ID NO: 118 - protein, 230 aa, Homo sapiens [sequence omitted]

SEQ ID NO: 119 - protein, 314 aa, Homo sapiens
MGLLDSEPGSVLNVVSTALNDTVEFYRWTWSIADKRVENWPLMQSPWPTLSISTLYLLFVWLGPKWMKDREPFQMRLVLIIYNFGMVLLNLFIFRELFMGSYNAGYSYICQSVDYSNNVHEVRIAAALWWYFVSKGVEYLDTVFFILRKKNNQVSFLHVYHHCTMFTLWWIGIKWVAGGQAFFGAQLNSFIHVIMYSYYGLTAFGPWIQKYLWWKRYLTMLQLIQFHVTIGHTALSLYTDCPFPKWMHWALIAYAISFIFLFLNFYIRTYKEPKKPKAGKTAMNGISANGVSKSEKQLMIENGKKQKNGKAKGD

SEQ ID NO: 120 - protein, 4061 aa, Homo sapiens
MSTLLENIFAIINLFKQYSKKDKNTDTLSKKELKELLEKEFRQILKNPDDPDMVDVFMDHLDIDHNKKIDFTEFLLMVFKLAQAYYESTRKENLPISGHKHRKHSHHDKHEDNKQEENKENRKRPSSLERRNNRKGNKGRSKSPRETGGKRHESSSEKKERKGYSPTHREEEYGKNHHNSSKKEKNKTENTRLGDNRKRLSERLEEKEDNEEGVYDYENTGRMTQKWIQSGHIATYYTIQDEAYDTTDSLLEENKIYERSRSSDGKSSSQVNRSRHENTSQVPLQESRTRKRRGSRVSQDRDSEGHSEDSERHSGSASRNHHGSAWEQSRDGSRHPRSHDEDRASHGHSADSSRQSGTRHAETSSRGQTASSHEQARSSPGERHGSGHQQSADSSRHSATGRGQASSAVSDRGHRGSSGSQASDSEGHSENSDTQSVSGHGKAGLRQQSHQESTRGRSGERSGRSGSSLYQVSTHEQPDSAHGRTGTSTGGRQGSHHEQARDSSRHSASQEGQDTIRGHPGSSRGGRQGSHHEQSVNRSGHSGSHHSHTTSQGRSDASHGQSGSRSASRQTRNEEQSGDGTRHSGSRHHEASSQADSSRHSQVGQGQSSGPRTSRNQGSSVSQDSDSQGHSEDSERWSGSASRNHHGSAQEQSRDGSRHPRSHHEDRAGHGHSADSSRKSGTRHTQNSSSGQAASSHEQARSSAGERHGSRHQLQSADSSRHSGTGHGQASSAVRDSGHRGSSGSQATDSEGHSEDSDTQSVSGHGQAGHHQQSHQESARDRSGERSRRSGSFLYQVSTHKQSESSHGWTGPSTGVRQGSHHEQARDNSRHSASQDGQDTIRGHPGSSRRGRQGSHHEQSVDRSGHSGSHHSHTTSQGRSDASRGQSGSRSASRTTRNEEQSRDGSRHSGSRHHEASSHADISRHSQAGQGQSEGSRTSRRQGSSVSQDSDSEGHSEDSERWSGSASRNHRGSAQEQSRHGSRHPRSHHEDRAGHGHSADSSRQSGTPHAETSSGGQAASSHEQARSSPGERHGSRHQQSADSSRHSGIPRRQASSAVRDSGHWGSSGSQASDSEGHSEESDTQSVSGHGQDGPHQQSHQESARDWSGGRSGRSGSFIYQVSTHEQSESAHGRTRTSTGRRQGSHHEQARDSSRHSASQEGQDTIRAHPGSRRGGRQGSHHEQSVDRSGHSGSHHSHTTSQGRSDASHGQSGSRSASRQTRKDKQSGDGSRHSGSRHHEAASWADSSRHSQVGQEQSSGSRTSRHQGSSVSQDSDSERHSDDSERLSGSASRNHHGSSREQSRDGSRHPGFHQEDRASHGHSADSSRQSGTHHTESSSHGQAVSSHEQARSSPGERHGSRHQQSADSSRHSGIGHRQASSAVRDSGHRGSSGSQVTNSEGHSEDSDTQSVSAHGQAGPHQQSHKESARGQSGESSGRSRSFLYQVSSHEQSESTHGQTAPSTGGRQGSRHEQARNSSRHSASQDGQDTIRGHPGSSRGGRQGSYHEQSVDRSGHSGYHHSHTTPQGRSDASHGQSGPRSASRQTRNEEQSGDGSRHSGSRHHEPSTRAGSSRHSQVGQGESAGSKTSRRQGSSVSQDRDSEGHSEDSERRSESASRNHYGSAREQSRHGSRNPRSHQEDRASHGHSAESSRQSGTRHAETSSGGQAASSQEQARSSPGERHGSRHQQSADSSTDSGTGRRQDSSVVGDSGNRGSSGSQASDSEGHSEESDTQSVSAHGQAGPHQQSHQESTRGQSGERSGRSGSFLYQVSTHEQSESAHGRTGPSTGGRQRSRHEQARDSSRHSASQEGQDTIRGHPGSSRGGRQGSHYEQSVDSSGHSGSHHSHTTSQERSDVSRGQSGSRSVSRQTRNEKQSGDGSRHSGSRHHEASSRADSSRHSQVGQGQSSGPRTSRNQGSSVSQDSDSQGHSEDSERWSGSASRNHLGSAWEQSRDGSRHPGSHHEDRAGHGHSADSSRQSGTRHTESSSRGQAASSHEQARSSAGERHGSHHQLQSADSSRHSGIGHGQASSAVRDSGHRGYSGSQASDSEGHSEDSDTQSVSAQGKAGPHQQSHKESARGQSGESSGRSGSFLYQVSTHEQSESTHGQSAPSTGGRQGSHYDQAQDSSRHSASQEGQDTIRGHPGPSRGGRQGSHQEQSVDRSGHSGSHHSHTTSQGRSDASRGQSGSRSASRKTYDKEQSGDGSRHSGSHHHEASSWADSSRHSLVGQGQSSGPRTSRPRGSSVSQDSDSEGHSEDSERRSGSASRNHHGSAQEQSRDGSRHPRSHHEDRAGHGHSAESSRQSGTHHAENSSGGQAASSHEQARSSAGERHGSHHQQSADSSRHSGIGHGQASSAVRDSGHRGSSGSQASDSEGHSEDSDTQSVSAHGQAGPHQQSHQESTRGRSAGRSGRSGSFLYQVSTHEQSESAHGRTGTSTGGRQGSHHKQARDSSRHSTSQEGQDTIHGHPGSSSGGRQGSHYEQLVDRSGHSGSHHSHTTSQGRSDASHGHSGSRSASRQTRNDEQSGDGSRHSGSRHHEASSRADSSGHSQVGQGQSEGPRTSRNWGSSFSQDSDSQGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYQVSTHEQSESSHGWTGPSTRGRQGSRHEQAQDSSRHSASQYGQDTIRGHPGSSRGGRQGYHHEHSVDSSGHSGSHHSHTTSQGRSDASRGQSGSRSASRTTRNEEQSGDSSRHSVSRHHEASTHADISRHSQAVQGQSEGSRRSRRQGSSVSQDSDSEGHSEDSERWSGSASRNHRGSVQEQSRHGSRHPRSHHEDRAGHGHSADRSRQSGTRHAETSSGGQAASSHEQARSSPGERHGSRHQQSADSSRHSGIPRGQASSAVRDSRHWGSSGSQASDSEGHSEESDTQSVSGHGQAGPHQQSHQESARDRSGGRSGRSGSFLYQVSTHEQSESAHGRTRTSTGRRQGSHHEQARDSSRHSASQEGQDTIRGHPGSSRRGRQGSHYEQSVDRSGHSGSHHSHTTSQGRSDASRGQSGSRSASRQTRNDEQSGDGSRHSWSHHHEASTQADSSRHSQSGQGQSAGPRTSRNQGSSVSQDSDSQGHSEDSERWSGSASRNHRGSAQEQSRDGSRHPTSHHEDRAGHGHSAESSRQSGTHHAENSSGGQAASSHEQARSSAGERHGSHHQQSADSSRHSGIGHGQASSAVRDSGHRGSSGSQASDSEGHSEDSDTQSVSAHGQAGPHQQSHQESTRGRSAGRSGRSGSFLYQVSTHEQSESAHGRAGPSTGGRQGSRHEQARDSSRHSASQEGQDTIRGHPGSRRGGRQGSYHEQSVDRSGHSGSHHSHTTSQGRSDASHGQSGSRSASRETRNEEQSGDGSRHSGSRHHEASTQADSSRHSQSGQGESAGSRRSRRQGSSVSQDSDSEAYPEDSERRSESASRNHHGSSREQSRDGSRHPGSSHRDTASHVQSSPVQSDSSTAKEHGHFSSLSQDSAYHSGIQSRGSPHSSSSYHYQSEGTERQKGQSGLVWRHGSYGSADYDYGESGFRHSQHGSVSYNSNPVVFKERSDICKASAFGKDHPRYYATYINKDPGLCGHSSDISKQLGFSQSQRYYYYE

SEQ ID NO: 121 - protein, 2391 aa, Homo sapiens
MTDLLRSVVTVIDVFYKYTKQDGECGTLSKGELKELLEKELHPVLKNPDDPDTVDVIMHMLDRDHDRRLDFTEFLLMIFKLTMACNKVLSKEYCKASGSKKHRRGHRHQEEESETEEDEEDTPGHKSGYRHSSWSEGEEHGYSSGHSRGTVKCRHGSNSRRLGRQGNLSSSGNQEGSQKRYHRSSCGHSWSGGKDRHGSSSVELRERINKSHISPSRESGEEYESGSGSNSWERKGHGGLSCGLETSGHESNSTQSRIREQKLGSSCSGSGDSGRRSHACGYSNSSGCGRPQNASSSCQSHRFGGQGNQFSYIQSGCQSGIKGGQGHGCVSGGQPSGCGQPESNPCSQSYSQRGYGARENGQPQNCGGQWRTGSSQSSCCGQYGSGGSQSCSNGQHEYGSCGRFSNSSSSNEFSKCDQYGSGSSQSTSFEQHGTGLSQSSGFEQHVCGSGQTCGQHESTSSQSLGYDQHGSSSGKTSGFGQHGSGSGQSSGFGQCGSGSGQSSGFGQHGSVSGQSSGFGQHGSVSGQSSGFGQHESRSRQSSYGQHGSGSSQSSGYGQYGSRETSGFGQHGLGSGQSTGFGQYGSGSGQSSGFGQHGSGSGQSSGFGQHESRSGQSSYGQHSSGSSQSSGYGQHGSRQTSGFGQHGSGSSQSTGFGQYGSGSGQSSGFGQHVSGSGQSSGFGQHESRSGHSSYGQHGFGSSQSSGYGQHGSSSGQTSGFGQHELSSGQSSSFGQHGSGSGQSSGFGQHGSGSGQSSGFGQHESRSGQSSYGQHSSGSSQSSGYGQHGSRQTSGFGQHGSGSSQSTGFGQYGSGSGQSAGFGQHGSGSGQSSGFGQHESRSHQSSYGQHGSGSSQSSGYGQHGSSSGQTSGFGQHRSSSGQYSGFGQHGSGSGQSSGFGQHGTGSGQYSGFGQHESRSHQSSYGQHGSGSSQSSGYGQHGSSSGQTFGFGQHRSGSGQSSGFGQHGSGSGQSSGFGQHESGSGKSSGFGQHESRSSQSNYGQHGSGSSQSSGYGQHGSSSGQTTGFGQHRSSSGQYSGFGQHGSGSDQSSGFGQHGTGSGQSSGFGQYESRSRQSSYGQHGSGSSQSSGYGQHGSNSGQTSGFGQHRPGSGQSSGFGQYGSGSGQSSGFGQHGSGTGKSSGFAQHEYRSGQSSYGQHGTGSSQSSGCGQHESGSGPTTSFGQHVSGSDNFSSSGQHISDSGQSTGFGQYGSGSGQSTGLGQGESQQVESGSTVHGRQETTHGQTINTTRHSQSGQGQSTQTGSRVTRRRRSSQSENSDSEVHSKVSHRHSEHIHTQAGSHYPKSGSTVRRRQGTTHGQRGDTTRHGHSGHGQSTQTGSRTSGRQRFSHSDATDSEVHSGVSHRPHSQEQTHSQAGSQHGESESTVHERHETTYGQTGEATGHGHSGHGQSTQRGSRTTGRRGSGHSESSDSEVHSGGSHRPQSQEQTHGQAGSQHGESGSTVHGRHGTTHGQTGDTTRHAHYHHGKSTQRGSSTTGRRGSGHSESSDSEVHSGGSHTHSGHTHGQSGSQHGESESIIHDRHRITHGQTGDTTRHSYSGHEQTTQTGSRTTGRQRTSHSESTDSEVHSGGSHRPHSREHTYGQAGSQHEEPEFTVHERHGTTHGQIGDTTGHSHSGHGQSTQRGSRTTGRQRSSHSESSDSEVHSGVSHTHTGHTHGQAGSQHGQSESIVPERHGTTHGQTGDTTRHAHYHHGLTTQTGSRTTGRRGSGHSEYSDSEGYSGVSHTHSGHTHGQARSQHGESESIVHERHGTIHGQTGDTTRHAHSGHGQSTQTGSRTTGRRSSGHSEYSDSEGHSGFSQRPHSRGHTHGQAGSQHGESESIVDERHGTTHGQTGDTSGHSQSGHGQSTQSGSSTTGRRRSGHSESSDSEVHSGGSHTHSGHTHSQARSQHGESESTVHKRHQTTHGQTGDTTEHGHPSHGQTIQTGSRTTGRRGSGHSEYSDSEGPSGVSHTHSGHTHGQAGSHYPESGSSVHERHGTTHGQTADTTRHGHSGHGQSTQRGSRTTGRRASGHSEYSDSEGHSGVSHTHSGHAHGQAGSQHGESGSSVHERHGTTHGQTGDTTRHAHSGHGQSTQRGSRTAGRRGSGHSESSDSEVHSGVSHTHSGHTYGQARSQHGESGSAIHGRQGTIHGQTGDTTRHGQSGHGQSTQTGSRTTGRQRSSHSESSDSEVHSEASPTHSGHTHSQAGSRHGQSGSSGHGRQGTTHGQTGDTTRHAHYGYGQSTQRGSRTTGRRGSGHSESSDSEVHSWGSHTHSGHIQGQAGSQQRQPGSTVHGRLETTHGQTGDTTRHGHSGYGQSTQTGSRSSRASHFQSHSSERQRHGSSQVWKHGSYGPAEYDYGHTGYGPSGGSRKSISNSHLSWSTDSTANKQLSRH

SEQ ID NO: 122 - protein, 226 aa, Homo sapiens
MDWGTLQTILGGVNKHSTSIGKIWLTVLFIFRIMILVVAAKEVWGDEQADFVCNTLQPGCKNVCYDHYFPISHIRLWALQLIFVSTPALLVAMHVAYRRHEKKRKFIKGEIKSEFKDIEEIKTQKVRIEGSLWWTYTSSIFFRVIFEAAFMYVFYVMYDGFSMQRLVKCNAWPCPNTVDCFVSRPTEKTVFTVFMIAVSGICILLNVTELCYLLIRYCSGKSKKPV

SEQ ID NO: 123 - protein, 270 aa, Homo sapiens
MDWKTLQALLSGVNKYSTAFGRIWLSVVFVFRVLVYVVAAERVWGDEQKDFDCNTKQPGCTNVCYDNYFPISNIRLWALQLIFVTCPSLLVILHVAYREERERRHRQKHGDQCAKLYDNAGKKHGGLWWTYLFSLIFKLIIEFLFLYLLHTLWHGFNMPRLVQCANVAPCPNIVDCYIARPTEKKIFTYFMVGASAVCIVLTICELCYLICHRVLRGLHKDKPRGGCSPSSSASRASTCRCHHKLVEAGEVDPDPGNNKLQASAPNLTPI

SEQ ID NO: 124 - protein, 266 aa, Homo sapiens
MNWAFLQGLLSGVNKYSTVLSRIWLSVVFIFRVLVYVVAAEEVWDDEQKDFVCNTKQPGCPNVCYDEFFPVSHVRLWALQLILVTCPSLLVVMHVAYREERERKHHLKHGPNAPSLYDNLSKKRGGLWWTYLLSLIFKAAVDAGFLYIFHRLYKDYDMPRVVACSVEPCPHTVDCYISRPTEKKVFTYFMVTTAAICILLNLSEVFYLVGKRCMEIFGPRHRRPRCRECLPDTCPPYVLSQGGHPEDGNSVLMKAGSAPVDAGGYP

SEQ ID NO: 125 - protein, 261 aa, Homo sapiens
MDWGTLHTFIGGVNKHSTSIGKVWITVIFIFRVMILVVAAQEVWGDEQEDFVCNTLQPGCKNVCYDHFFPVSHIRLWALQLIFVSTPALLVAMHVAYYRHETTRKFRRGEKRNDFKDIEDIKKQKVRIEGSLWWTYTSSIFFRIIFEAAFMYVFYFLYNGYHLPWVLKCGIDPCPNLVDCFISRPTEKTVFTIFMISASVICMLLNVAELCYLLLKVCFRRSKRAQTQKNHPNHALKESKQNEMNELISDSGQNAITGFPS

SEQ ID NO: 126 - protein, 332 aa, Homo sapiens
MLLLAAAFLVAFVLLLYMVSPLISPKPLALPGAHVVVTGGSSGIGKCIAIECYKQGAFITLVARNEDKLLQAKKEIEMHSINDKQVVLCISVDVSQDYNQVENVIKQAQEKLGPVDMLVNCAGMAVSGKFEDLEVSTFERLMSINYLGSVYPSRAVITTMKERRVGRIVFVSSQAGQLGLFGFTAYSASKFAIRGLAEALQMEVKPYNVYITVAYPPDTDTPGFAEENRTKPLETRLISETTSVCKPEQVAKQIVKDAIQGNFNSSLGSDGYMLSALTCGMAPVTSITEGLQQVVTMGLFRTIALFYLGSFDSIVRRCMMQREKSENADKTA

SEQ ID NO: 127 - protein, 644 aa, Homo sapiens
MSRQFSSRSGYRSGGGFSSGSAGIINYQRRTTSSSTRRSGGGGGRFSSCGGGGGSFGAGGGFGSRSLVNLGGSKSISISVARGGGRGSGFGGGYGGGGFGGGGFGGGGFGGGGIGGGGFGGFGSGGGGFGGGGFGGGGYGGGYGPVCPPGGIQEVTINQSLLQPLNVEIDPEIQKVKSREREQIKSLNNQFASFIDKVRFLEQQNQVLQTKWELLQQVDTSTRTHNLEPYFESFINNLRRRVDQLKSDQSRLDSELKNMQDMVEDYRNKYEDEINKRTNAENEFVTIKKDVDGAYMTKVDLQAKLDNLQQEIDFLTALYQAELSQMQTQISETNVILSMDNNRSLDLDSIIAEVKAQYEDIAQKSKAEAESLYQSKYEELQITAGRHGDSVRNSKIEISELNRVIQRLRSEIDNVKKQISNLQQSISDAEQRGENALKDAKNKLNDLEDALQQAKEDLARLLRDYQELMNTKLALDLEIATYRTLLEGEESRMSGECAPNVSVSVSTSHTTISGGGSRGGGGGGYGSGGSSYGSGGGSYGSGGGGGGGRGSYGSGGSSYGSGGGSYGSGGGGGGHGSYGSGSSSGGYRGGSGGGGGGSSGGRGSGGGSSGGSIGGRGSSSGGVKSSGGSSSVKFVSTTYSGVTR

SEQ ID NO: 128 - protein, 639 aa, Homo sapiens
MSCQISCKSRGRGGGGGGFRGFSSGSAVVSGGSRRSTSSFSCLSRHGGGGGGFGGGGFGSRSLVGLGGTKSISISVAGGGGGFGAAGGFGGRGGGFGGGSSFGGGSGFSGGGFGGGGFGGGRFGGFGGPGGVGGLGGPGGFGPGGYPGGIHEVSVNQSLLQPLNVKVDPEIQNVKAQEREQIKTLNNKFASFIDKVRFLEQQNQVLQTKWELLQQMNVGTRPINLEPIFQGYIDSLKRYLDGLTAERTSQNSELNNMQDLVEDYKKKYEDEINKRTAAENDFVTLKKDVDNAYMIKVELQSKVDLLNQEIEFLKVLYDAEISQIHQSVTDTNVILSMDNSRNLDLDSIIAEVKAQYEEIAQRSKEEAEALYHSKYEELQVTVGRHGDSLKEIKIEISELNRVIQRLQGEIAHVKKQCKNVQDAIADAEQRGEHALKDARNKLNDLEEALQQAKEDLARLLRDYQELMNVKLALDVEIATYRKLLEGEECRMSGDLSSNVTVSVTSSTISSNVASKAAFGGSGGRGSSSGGGYSSGSSSYGSGGRQSGSRGGSGGGGSISGGGYGSGGGSGGRYGSGGGSKGGSISGGGYGSGGGKHSSGGGSRGGSSSGGGYGSGGGGSSSVKGSSGEAFGSSVTFSFR

SEQ ID NO: 129 - protein, 623 aa, Homo sapiens
MSCRQFSSSYLSRSGGGGGGGLGSGGSIRSSYSRFSSSGGGGGGGRFSSSSGYGGGSSRVCGRGGGGSFGYSYGGGSGGGFSASSLGGGFGGGSRGFGGASGGGYSSSGGFGGGFGGGSGGGFGGGYGSGFGGFGGFGGGAGGGDGGILTANEKSTMQELNSRLASYLDKVQALEEANNDLENKIQDWYDKKGPAAIQKNYSPYYNTIDDLKDQIVDLTVGNNKTLLDIDNTRMTLDDFRIKFEMEQNLRQGVDADINGLRQVLDNLTMEKSDLEMQYETLQEELMALKKNHKEEMSQLTGQNSGDVNVEINVAPGKDLTKTLNDMRQEYEQLIAKNRKDIENQYETQITQIEHEVSSSGQEVQSSAKEVTQLRHGVQELEIELQSQLSKKAALEKSLEDTKNRYCGQLQMIQEQISNLEAQITDVRQEIECQNQEYSLLLSIKMRLEKEIETYHNLLEGGQEDFESSGAGKIGLGGRGGSGGSYGRGSRGGSGGSYGGGGSGGGYGGGSGSRGGSGGSYGGGSGSGGGSGGGYGGGSGGGHSGGSGGGHSGGSGGNYGGGSGSGGGSGGGYGGGSGSRGGSGGSHGGGSGFGGESGGSYGGGEEASGSGGGYGGGSGKSSHS

SEQ ID NO: 130 - protein, 584 aa, Homo sapiens
MSVRYSSSKHYSSSRSGGGGGGGGCGGGGGVSSLRISSSKGSLGGGFSSGGFSGGSFSRGSSGGGCFGGSSGGYGGLGGFGGGSFRGSYGSSSFGGSYGGSFGGGSFGGGSFGGGSFGGGGFGGGGFGGGFGGGFGGDGGLLSGNEKVTMQNLNDRLASYLDKVRALEESNYELEGKIKEWYEKHGNSHQGEPRDYSKYYKTIDDLKNQILNLTTDNANILLQIDNARLAADDFRLKYENEVALRQSVEADINGLRRVLDELTLTKADLEMQIESLTEELAYLKKNHEEEMKDLRNVSTGDVNVEMNAAPGVDLTQLLNNMRSQYEQLAEQNRKDAEAWFNEKSKELTTEIDNNIEQISSYKSEITELRRNVQALEIELQSQLALKQSLEASLAETEGRYCVQLSQIQAQISALEEQLQQIRAETECQNTEYQQLLDIKIRLENEIQTYRSLLEGEGSSGGGGRGGGSFGGGYGGGSSGGGSSGGGHGGGHGGSSGGGYGGGSSGGGSSGGGYGGGSSSGGHGGSSSGGYGGGSSGGGGGGYGGGSSGGGSSSGGGYGGGSSSGGHKSSSSGSVGESSSKGPRY

SEQ ID NO: 131 - protein, 398 aa, Homo sapiens
MMWLLLTTTCLICGTLNAGGFLDLENEVNPEVWMNTSEIIIYNGYPSEEYEVTTEDGYILLVNRIPYGRTHARSTGPRPVVYMQHALFADNAYWLENYANGSLGFLLADAGYDVWMGNSRGNTWSRRHKTLSETDEKFWAFSFDEMAKYDLPGVIDFIVNKTGQEKLYFIGHSLGTTIGFVAFSTMPELAQRIKMNFALGPTISFKYPTGIFTRFFLLPNSIIKAVFGTKGFFLEDKKTKIASTKICNNKILWLICSEFMSLWAGSNKKNMNQSRMDVYMSHAPTGSSVHNILHIKQLYHSDEFRAYDWGNDADNMKHYNQSHPPIYDLTAMKVPTAIWAGGHDVLVTPQDVARILPQIKSLHYFKLLPDWNHFDFVWGLDAPQRMYSEIIALMKAYS

SEQ ID NO: 132 - protein, 312 aa, Homo sapiens
MSYQKKQPTPQPPVDCVKTSGGGGGGGGSGGGGCGFFGGGGSGGGSSGSGCGYSGGGGYSGGGCGGGSSGGGGGGGIGGCGGGSGGSVKYSGGGGSSGGGSGCFSSGGGGSGCFSSGGGGSSGGGSGCFSSGGGGSSGGGSGCFSSGGGGFSGQAVQCQSYGGVSSGGSSGGGSGCFSSGGGGGSVCGYSGGGSGCGGGSSGGSGSGYVSSQQVTQTSCAPQPSYGGGSSGGGGSGGSGCFSSGGGGGSSGCGGGSSGIGSGCIISGGGSVCGGGSSGGGGGGSSVGGSGSGKGVPICHQTQQKQAPTWPSK

SEQ ID NO: 133 - protein, 519 aa, Homo sapiens
MIPVSLVVVVVGGWTVVYLTDLVLKSSVYFKHSYEDWLENNGLSISPFHIRWQTAVFNRAFYSWGRRKARMLYQWFNFGMVFGVIAMFSSFFLLGKTLMQTLAQMMADSPSSYSSSSSSSSSSSSSSSSSSSSSSSLHNEQVLQVVVPGINLPVNQLTYFFTAVLISGVVHEIGHGIAAIREQVRFNGFGIFLFIIYPGAFVDLFTTHLQLISPVQQLRIFCAGIWHNFVLALLGILALVLLPVILLPFYYTGVGVLITEVAEDSPAIGPRGLFVGDLVTHLQDCPVTNVQDWNECLDTIAYEPQIGYCISASTLQQLSFPVRAYKRLDGSTECCNNHSLTDVCFSYRNNFNKRLHTCLPARKAVEATQVCRTNKDCKKSSSSSFCIIPSLETHTRLIKVKHPPQIDMLYVGHPLHLHYTVSITSFIPRFNFLSIDLPVVVETFVKYLISLSGALAIVNAVPCFALDGQWILNSFLDATLTSVIGDNDVKDLIGFFILLGGSVLLAANVTLGLWMVTAR

SEQ ID NO: 134 - protein, 466 aa, Homo sapiens
MPGDSSPGTLPLWDASLSPPLGPDPGGFSRASHAGDKSRPPAPELGSPGAVRPRVGSCAPGPMELRVSNTSCENGSLLHLYCSSQEVLCQIVNDLSPEVPSNATFHSWQERIRQNYGFYIGLGLAFLSSFLIGSSVILKKKGLLRLVATGATRAVDGGFGYLKDAMWWAGFLTMAAGEVANFGAYAFAPATVVTPLGALSVLISAILSSYFLRESLNLLGKLGCVICVAGSTVMVIHAPEEEKVTTIMEMASKMKDTGFIVFAVLLLVSCLILIFVIAPRYGQRNILIYIIICSVIGAFSVAAVKGLGITIKNFFQGLPVVRHPLPYILSLILALSLSTQVNFLNRALDIFNTSLVFPIYYVFFTTVVVTSSIILFKEWYSMSAVDIAGTLSGFVTIILGVFMLHAFKDLDISCASLPHMHKNPPPSPAPEPTVIRLEDKNVLVDNIELASTSSPEEKPKVFIIHS

SEQ ID NO: 135 - protein, 447 aa, Homo sapiens
MPGDSSPGTLPLWDASLSPPLGPDPGGFSRASHAGDKSRPPAPELGSPGAVRPRVGSCAPGPMELRVSNTSCENGSLLHLYCSSQEVLCQIVNDLSPEVPSNATFHSWQERIRQNYGFYIGLGLAFLSSFLIGSSVILKKKGLLRLVATGATRAVAAGEVANFGAYAFAPATVVTPLGALSVLISAILSSYFLRESLNLLGKLGCVICVAGSTVMVIHAPEEEKVTTIMEMASKMKDTGFIVFAVLLLVSCLILIFVIAPRYGQRNILIYIIICSVIGAFSVAAVKGLGITIKNFFQGLPVVRHPLPYILSLILALSLSTQVNFLNRALDIFNTSLVFPIYYVFFTTVVVTSSIILFKEWYSMSAVDIAGTLSGFVTIILGVFMLHAFKDLDISCASLPHMHKNPPPSPAPEPTVIRLEDKNVLVDNIELASTSSPEEKPKVFIIHS

SEQ ID NO: 136 - protein, 373 aa, Homo sapiens
MEPAVSEPMRDQVARTHLTEDTPKVNADIEKVNQNQAKRCTVIGGSGFLGQHMVEQLLARGYAVNVFDIQQGFDNPQVRFFLGDLCSRQDLYPALKGVNTVFHCASPPPSSNNKELFYRVNYIGTKNVIETCKEAGVQKLILTSSASVIFEGVDIKNGTEDLPYAMKPIDYYTETKILQERAVLGANDPEKNFLTTAIRPHGIFGPRDPQLVPILIEAARNGKMKFVIGNGKNLVDFTFVENVVHGHILAAEQLSRDSTLGGKAFHITNDEPIPFWTFLSRILTGLNYEAPKYHIPYWVAYYLALLLSLLVMVISPVIQLQPTFTPMRVALAGTFHYYSCERAKKAMGYQPLVTMDDAMERTVQSFRHLRRVK

SEQ ID NO: 137 - protein, 323 aa, Homo sapiens
MSAVCGGAARMLRTPGRHGYAAEFSPYLPGRLACATAQHYGIAGCGTLLILDPDEAGLRLFRSFDWNDGLFDVTWSENNEHVLITCSGDGSLQLWDTAKAAGPLQVYKEHAQEVYSVDWSQTRGEQLVVSGSWDQTVKLWDPTVGKSLCTFRGHESIIYSTIWSPHIPGCFASASGDQTLRIWDVKAAGVRIVIPAHQAEILSCDWCKYNENLLVTGAVDCSLRGWDLRNVRQPVFELLGHTYAIRRVKFSPFHASVLASCSYDFTVRFWNFSKPDSLLETVEHHTEFTCGLDFSLQSPTQVADCSWDETIKIYDPACLTIPA

SEQ ID NO: 138 - protein, 533 aa, Homo sapiens
MAFANLRKVLISDSLDPCCRKILQDGGLQVVEKQNLSKEELIAELQDCEGLIVRSATKVTADVINAAEKLQVVGRAGTGVDNVDLEAATRKGILVMNTPNGNSLSAAELTCGMIMCLARQIPQATASMKDGKWERKKFMGTELNGKTLGILGLGRIGREVATRMQSFGMKTIGYDPIISPEVSASFGVQQLPLEEIWPLCDFITVHTPLLPSTTGLLNDNTFAQCKKGVRVVNCARGGIVDEGALLRALQSGQCAGAALDVFTEEPPRDRALVDHENVISCPHLGASTKEAQSRCGEEIAVQFVDMVKGKSLTGVVNAQALTSAFSPHTKPWIGLAEALGTLMRAWAGSPKGTIQVITQGTSLKNAGNCLSPAVIVGLLKEASKQADVNLVNAKLLVKEAGLNVTTSHSPAAPGEQGFGECLLAVALAGAPYQAVGLVQGTTPVLQGLNGAVFRPEVPLRRDLPLLLFRTQTSDPAMLPTMIGLLAEAGVRLLSYQTSLVSDGETWHVMGISSLLPSLEAWKQHVTEAFQFHF

SEQ ID NO: 139 - protein, 338 aa, Homo sapiens
MEQLRAAARLQIVLGHLGRPSAGAVVAHPTSGTISSASFHPQQFQYTLDNNVLTLEQRKFYEENGFLVIKNLVPDADIQRFRNEFEKICRKEVKPLGLTVMRDVTISKSEYAPSEKMITKVQDFQEDKELFRYCTLPEILKYVECFTGPNIMAMHTMLINKPPDSGKKTSRHPLHQDLHYFPFRPSDLIVCAWTAMEHISRNNGCLVVLPGTHKGSLKPHDYPKWEGGVNKMFHGIQDYEENKARVHLVMEKGDTVFFHPLLIHGSGQNKTQGFRKAISCHFASADCHYIDVKGTSQENIEKEVVGIAHKFFGAENSVNLKDIWMFRARLVKGERTNL

SEQ ID NO: 140 - protein, 532 aa, Homo sapiens
MEEQVFKGDPDTPHSISFSGSGFLSFYQAGAVDALRDLAPRMLETAHRFAGTSAGAVIAALAICGIEMDEYLRVLNVGVAEVKKSFLGPLSPSCKMVQMMRQFLYRVLPEDSYKVTTGKLHVSLTRLTDGENVVVSEFTSKEELIEALYCSCFVPVYCGLIPPTYRGVRYIDGGFTGMQPCAFWTDAITISTFSGQQDICPRDCPAIFHDFRMFNCSFQFSLENIARMTHALFPPDLVILHDYYYRGYEDAVLYLRRLNAVYLNSSSKRVIFPRVEVYCQIELALGNECPERSQPSLRARQASLEGATQPHKEWVPKGDGRGSHGPPVSQPVQTLEFTCESPVSAPVSPLEQPPAQPLASSTPLSLSGMPPVSFPAVHKPPSSTPGSSLPTPPPGLSPLSPQQQVQPSGSPARSLHSQAPTSPRPSLGPSTVGAPQTLPRSSLSAFPAQPPVEELGQEQPQAVALLVSSKPKSAVPLVHVKETVSKPYVTESPAEDSNWVNKVFKKNKQKTSGTRKGFPRHSGSKKPSSKVQ

SEQ ID NO: 141 - protein, 437 aa, Homo sapiens
MVQMMRQFLYRVLPEDSYKVTTGKLHVSLTRLTDGENVVVSEFTSKEELIEALYCSCFVPVYCGLIPPTYRGVRYIDGGFTGMQPCAFWTDAITISTFSGQQDICPRDCPAIFHDFRMFNCSFQFSLENIARMTHALFPPDLVILHDYYYRGYEDAVLYLRRLNAVYLNSSSKRVIFPRVEVYCQIELALGNECPERSQPSLRARQASLEGATQPHKEWVPKGDGRGSHGPPVSQPVQTLEFTCESPVSAPVSPLEQPPAQPLASSTPLSLSGMPPVSFPAVHKPPSSTPGSSLPTPPPGLSPLSPQQQVQPSGSPARSLHSQAPTSPRPSLGPSTVGAPQTLPRSSLSAFPAQPPVEELGQEQPQAVALLVSSKPKSAVPLVHVKETVSKPYVTESPAEDSNWVNKVFKKNKQKTSGTRKGFPRHSGSKKPSSKVQ

SEQ ID NO: 142 - protein, 446 aa, Homo sapiens
MVQMMRQFLYRVLPEDSYKVTTGKLHVSLTRLTDGENVVVSEFTSKEELIEALYCSCFVPVYCGLIPPTYRGVWAFLTLPPQRYIDGGFTGMQPCAFWTDAITISTFSGQQDICPRDCPAIFHDFRMFNCSFQFSLENIARMTHALFPPDLVILHDYYYRGYEDAVLYLRRLNAVYLNSSSKRVIFPRVEVYCQIELALGNECPERSQPSLRARQASLEGATQPHKEWVPKGDGRGSHGPPVSQPVQTLEFTCESPVSAPVSPLEQPPAQPLASSTPLSLSGMPPVSFPAVHKPPSSTPGSSLPTPPPGLSPLSPQQQVQPSGSPARSLHSQAPTSPRPSLGPSTVGAPQTLPRSSLSAFPAQPPVEELGQEQPQAVALLVSSKPKSAVPLVHVKETVSKPYVTESPAEDSNWVNKVFKKNKQKTSGTRKGFPRHSGSKKPSSKVQ

SEQ ID NO: 143 - protein, 141 aa, Homo sapiens
MNARGLGSELKDSIPVTELSASGPFESHDLLRKGFSCVKNELLPSHPLELSEKNFQLNQDKMNFSTLRNIQGLFAPLKLQMEFKAVQQVQRLPFLSSSNLSLDVLRGNDETIGFEDILNDPSQSEVMGEPHLMVEYKLGLL

SEQ ID NO: 144 - protein, 370 aa, Homo sapiens
MDAPRQVVNFGPGPAKLPHSVLLEIQKELLDYKGVGISVLEMSHRSSDFAKIINNTENLVRELLAVPDNYKVIFLQGGGCGQFSAVPLNLIGLKAGRCADYVVTGAWSAKAAEEAKKFGTINIVHPKLGSYTKIPDPSTWNLNPDASYVYYCANETVHGVEFDFIPDVKGAVLVCDMSSNFLSKPVDVSKFGVIFAGAQKNVGSAGVTVVIVRDDLLGFALRECPSVLEYKVQAGNSSLYNTPPCFSIYVMGLVLEWIKNNGGAAAMEKLSSIKSQTIYEIIDNSQGFYVCPVEPQNRSKMNIPFRIGNAKGDDALEKRFLDKALELNMLSLKGHRSVGGIRASLYNAVTIEDVQKLAAFMKKFLEMHQL

SEQ ID NO: 145 - protein, 313 aa, Homo sapiens
MAALTDLSFMYRWFKNCNLVGNLSEKYVFITGCDSGFGNLLAKQLVDRGMQVLAACFTEEGSQKLQRDTSYRLQTTLLDVTKSESIKAAAQWVRDKVGEQGLWALVNNAGVGLPSGPNEWLTKDDFVKVINVNLVGLIEVTLHMLPMVKRARGRVVNMSSSGGRVAVIGGGYCVSKFGVEAFSDSIRRELYYFGVKVCIIEPGNYRTAILGKENLESRMRKLWERLPQETRDSYGEDYFRIYTDKLKNIMQVAEPRVRDVINSMEHAIVSRSPRIRYNPGLDAKLLYIPLAKLPTPVTDFILSRYLPRPADSV

SEQ ID NO: 146 - protein, 374 aa, Homo sapiens
MDDLCEANGTFAISLFKILGEEDNSRNVFFSPMSISSALAMVFMGAKGSTAAQMSQALCLYKDGDIHRGFQSLLSEVNRTGTQYLLRTANRLFGEKTCDFLPDFKEYCQKFYQAELEELSFAEDTEECRKHINDWVAEKTEGKISEVLDAGTVDPLTKLVLVNAIYFKGKWNEQFDRKYTRGMLFKTNEEKKTVQMMFKEAKFKMGYADEVHTQVLELPYVEEELSMVILLPDDNTDLAVVEKALTYEKFKAWTNSEKLTKSKVQVFLPRLKLEESYDLEPFLRRLGMIDAFDEAKADFSGMSTEKNVPLSKVAHKCFVEVNEEGTEAAAATAVVRNSRCSRMEPRFCADHPFLFFIRHHKTNCILFCGRFSSP

SEQ ID NO: 147 - protein, 643 aa, Homo sapiens
MLLGASLVGVLLFSKLVLKLPWTQVGFSLLFLYLGSGGWRFIRVFIKTIRRDIFGGLVLLKVKAKVRQCLQERRTVPILFASTVRRHPDKTALIFEGTDTHWTFRQLDEYSSSVANFLQARGLASGDVAAIFMENRNEFVGLWLGMAKLGVEAALINTNLRRDALLHCLTTSRARALVFGSEMASAICEVHASLDPSLSLFCSGSWEPGAVPPSTEHLDPLLKDAPKHLPSCPDKGFTDKLFYIYTSGTTGLPKAAIVVHSRYYRMAALVYYGFRMRPNDIVYDCLPLYHSAGNIVGIGQCLLHGMTVVIRKKFSASRFWDDCIKYNCTIVQYIGELCRYLLNQPPREAENQHQVRMALGNGLRQSIWTNFSSRFHIPQVAEFYGATECNCSLGNFDSQVGACGFNSRILSFVYPIRLVRVNEDTMELIRGPDGVCIPCQPGEPGQLVGRIIQKDPLRRFDGYLNQGANNKKIAKDVFKKGDQAYLTGDVLVMDELGYLYFRDRTGDTFRWKGENVSTTEVEGTLSRLLDMADVAVYGVEVPGTEGRAGMAAVASPTGNCDLERFAQVLEKELPLYARPIFLRLLPELHKTGTYKFQKTELRKEGFDPAIVKDPLFYLDAQKGRYVPLDQEAYSRIQAGEEKL

SEQ ID NO: 148 - protein, 258 aa, Homo sapiens
MSAYPKSYNPFDDDGEDEGARPAPWRDARDLPDGPDAPADRQQYLRQEVLRRAEATAASTSRSLALMYESEKVGVASSEELARQRGVLERTEKMVDKMDQDLKISQKHINSIKSVFGGLVNYFKSKPVETPPEQNGTLTSQPNNRLKEAISTSKEQEAKYQASHPNLRKLDDTDPVPRGAGSAMSTDAYPKNPHLRAYHQKIDSNLDELSMGLGRLKDIALGMQTEIEEQDDILDRLTTKVDKLDVNIKSTERKVRQL

SEQ ID NO: 149 - protein, 855 aa, Homo sapiens [sequence omitted]

SEQ ID NO: 150 - protein, 583 aa, Homo sapiens
MPLRKMKIPFLLLFFLWEAESHAASRPNIILVMADDLGIGDPGCYGNKTIRTPNIDRLASGGVKLTQHLAASPLCTPSRAAFMTGRYPVRSGMASWSRTGVFLFTASSGGLPTDEITFAKLLKDQGYSTALIGKWHLGMSCHSKTDFCHHPLHHGFNYFYGISLTNLRDCKPGEGSVFTTGFKRLVFLPLQIVGVTLLTLAALNCLGLLHVPLGVFFSLLFLAALILTLFLGFLHYFRPLNCFMMRNYEIIQQPMSYDNLTQRLTVEAAQFIQRNTETPFLLVLSYLHVHTALFSSKDFAGKSQHGVYGDAVEEMDWSVGQILNLLDELRLANDTLIYFTSDQGAHVEEVSSKGEIHGGSNGIYKGGKANNWEGGIRVPGILRWPRVIQAGQKIDEPTSNMDIFPTVAKLAGAPLPEDRIIDGRDLMPLLEGKSQRSDHEFLFHYCNAYLNAVRWHPQNSTSIWKAFFFTPNFNPVGSNGCFATHVCFCFGSYVTHHDPPLLFDISKDPRERNPLTPASEPRFYEILKVMQEAADRHTQTLPEVPDQFSWNNFLWKPWLQLCCPSTGLSCQCDREKQDKRLSR

SEQ ID NO: 151 - protein, 617 aa, Homo sapiens
MAFPHRPDAPELPDFSMLKRLARDQLIYLLEQLPGKKDLFIEADLMSPLDRIANVSILKQHEVDKLYKVENKPALSSNEQLCFLVRPRIKNMRYIASLVNADKLAGRTRKYKVIFSPQKFYACEMVLEEEGIYGDVSCDEWAFSLLPLDVDLLSMELPEFFRDYFLEGDQRWINTVAQALHLLSTLYGPFPNCYGIGRCAKMAYELWRNLEEEEDGETKGRRPEIGHIFLLDRDVDFVTALCSQVVYEGLVDDTFRIKCGSVDFGPEVTSSDKSLKVLLNAEDKVFNEIRNEHFSNVFGFLSQKARNLQAQYDRRRGMDIKQMKNFVSQELKGLKQEHRLLSLHIGACESIMKKKTKQDFQELIKTEHALLEGFNIRESTSYIEEHIDRQVSPIESLRLMCLLSITENGLIPKDYRSLKTQYLQSYGPEHLLTFSNLRRAGLLTEQAPGDTLTAVESKVSKLVTDKAAGKITDAFSSLAKRSNFRAISKKLNLIPRVDGEYDLKVPRDMAYVFGGAYVPLSCRIIEQVLERRSWQGLDEVVRLLNCSDFAFTDMTKEDKASSESLRLILVVFLGGCTFSEISALRFLGREKGYRFIFLTTAVTNSARLMEAMSEVKA

SEQ ID NO: 152 - protein, 475 aa, Homo sapiens
MGMWASLDALWEMPAEKRIFGAVLLFSWTVYLWETFLAQRQRRIYKTTTHVPPELGQIMDSETFEKSRLYQLDKSTFSFWSGLYSETEGTLILLFGGIPYLWRLSGRFCGYAGFGPEYEITQSLVFLLLATLFSALTGLPWSLYNTFVIEEKHGFNQQTLGFFMKDAIKKFVVTQCILLPVSSLLLYIIKIGGDYFFIYAWLFTLVVSLVLVTIYADYIAPLFDKFTPLPEGKLKEEIEVMAKSIDFPLTKVYVVEGSKRSSHSNAYFYGFFKNKRIVLFDTLLEEYSVLNKDIQEDSGMEPRNEEEGNSEEIKAKVKNKKQGCKNEEVLAVLGHELGHWKLGHTVKNIIISQMNSFLCFFLFAVLIGRKELFAAFGFYDSQPTLIGLLIIFQFIFSPYNEVLSFCLTVLSRRFEFQADAFAKKLGKAKDLYSALIKLNKDNLGFPVSDWLFSMWHYSHPPLLERLQALKTMKQH

SEQ ID NO: 153 - DNA, 1098 bp, Homo sapiens
atggacgggcccgccgagccccagatcccgggcttgtgggacacctatgaagatgacatctcggaaatcagccagaagttgccaggtgaatacttccggtacaagggcgtccccttccccgtcggcctgtactcgctcgagagcatcagcttggcggagaacacccaagatgtgcgggacgacgacatctttatcatcacctaccccaagtcaggcacgacctggatgatcgagatcatctgcttaatcctgaaggaaggggatccatcctggatccgctccgtgcccatctgggagcgggcaccctggtgtgagaccattgtgggtgccttcagcctcccggaccagtacagcccccgcctcatgagctcccatcttcccatccagatcttcaccaaggccttcttcagctccaaggccaaggtgatctacatgggccgcaacccccgggacgttgtggtctccctctatcattactccaagatcgccgggcagttaaaggacccgggcacacccgaccagttcctgagggacttcctcaaaggcgaagtgcagtttggctcctggttcgaccacattaagggctggcttcggatgaagggcaaagacaacttcctatttatcacctacgaggagctgcagcaggacttacagggctccgtggagcgcatctgtgggttcctgggccgtccgctgggcaaggaggcactgggctccgtcgtggcacactcaaccttcagcgccatgaaggccaacaccatgtccaactacacgctgctgcctcccagcctgctggaccaccgtcgcggggccttcctccggaaaggggtctgcggcgactggaagaaccacttcacggtggcccagagcgaagccttcgatcgtgcctaccgcaagcagatgcgggggatgccgaccttcccctgggatgaagacccggaggaggacggcagcccagatcctgagcccagccctgagcctgagcccaagcccagccttgagcccaacaccagcctggagcgtgagcccagacccaactccagccccagccccagccccggccaggcctctgagaccccgcacccacgaccctcataa

SEQ ID NO: 154 - DNA, 1098 bp, Artificial Sequence
  Synthetic
atggatggacctgccgagcctcagattcctggcctgtgggatacctacgaggacgacatcagcgagatcagccagaagctgcccggcgagtacttcagatacaagggcgtgccatttcctgtgggcctgtacagcctggaatccatcagcctggccgagaacacacaggacgtgcgggacgacgacatcttcatcatcacataccccaagagcggcaccacctggatgatcgagatcatctgcctgatcctgaaagagggcgaccccagctggatcagaagcgtgccaatttgggagcgagccccttggtgcgagacaatcgtgggcgcattttccctgcctgaccagtacagccccagactgatgagcagccatctgccaatccagatcttcaccaaggcctttttcagcagcaaggccaaagtgatctacatgggcagaaaccccagagatgtggtggtgtccctgtaccactacagcaagatcgccggccagctgaaggaccccggaacacctgatcagtttctgcgggacttcctgaagggcgaagtgcagttcggcagttggttcgaccacatcaaaggctggctgcggatgaagggcaaagacaacttcctgttcatcacgtacgaggaactgcagcaggacctgcagggcagcgtggaaagaatctgcggctttctgggcagacccctgggaaaagaagccctgggatctgtcgtggcccacagcaccttttctgccatgaaggccaacaccatgagcaactacaccctgctgcctccaagcctgctggaccatagaagaggggcctttctgcggaagggcgtgtgtggcgattggaagaaccacttcacagtggcccagagcgaggccttcgacagagcctacagaaagcagatgcggggcatgcctacctttccatgggacgaagatcccgaagaggacggctctcctgatccagagccttctccagagccagaacctaagcctagcctggaacctaacacctctctggaaagagagcccagacctaacagcagcccctctccatctcctggacaggcctctgagacaccccatcctagacctagctga

SEQ ID NO: 155 - protein, 365 aa, Homo sapiens
MDGPAEPQIPGLWDTYEDDISEISQKLPGEYFRYKGVPFPVGLYSLESISLAENTQDVRDDDIFIITYPKSGTTWMIEIICLILKEGDPSWIRSVPIWERAPWCETIVGAFSLPDQYSPRLMSSHLPIQIFTKAFFSSKAKVIYMGRNPRDVVVSLYHYSKIAGQLKDPGTPDQFLRDFLKGEVQFGSWFDHIKGWLRMKGKDNFLFITYEELQQDLQGSVERICGFLGRPLGKEALGSVVAHSTFSAMKANTMSNYTLLPPSLLDHRRGAFLRKGVCGDWKNHFTVAQSEAFDRAYRKQMRGMPTFPWDEDPEEDGSPDPEPSPEPEPKPSLEPNTSLEREPRPNSSPSPSPGQASETPHPRPS

(19) United States
(12) Patent Application Publication
(10) Pub. No.: US 2023/0118087 A1
(43) Pub. Date: Apr. 20, 2023

(54) COMPOSITIONS AND METHODS FOR THE TREATMENT OF CONGENITAL ICHTHYOSES

(71) Applicant: Krystal Biotech, Inc., Pittsburgh, PA (US)

(72) Inventors: Suma KRISHNAN, Pittsburgh, PA (US); Pooja AGARWAL, Pittsburgh, PA (US)

(73) Assignee: Krystal Biotech, Inc., Pittsburgh, PA (US)

(21) Appl. No.: 17/639,878

(22) PCT Filed: Sep. 2, 2020
(86) PCT No.: PCT/US2020/049070

§ 371 Date: Mar. 2, 2022

Related U.S. Application Data
(60) Provisional application No. 62/895,045, filed on Sep. 3, 2019.

(51) Int. Cl. C12N 15/86; C07K 14/47; A61P 17/00

(52) U.S. Cl. CPC C12N 15/86; C07K 14/47; A61P 17/00; C12N 2710/16643; C12N 2710/16671

(57) ABSTRACT

The present disclosure provides recombinant nucleic acids comprising one or more polynucleotides encoding an ichthyosis-associated polypeptide; viruses comprising the recombinant nucleic acids; compositions comprising the recombinant nucleic acids and/or viruses; methods of their use; and articles of manufacture or kits thereof.

### CROSS REFERENCE TO RELATED APPLICATIONS

This application is a national stage application under 35 U.S.C. § 371 of International Application No. PCT/US2020/049070, filed internationally on Sep. 2, 2020, which claims the priority benefit of U.S. Provisional Application Ser. No. 62/895,045, filed Sep. 3, 2019, which is incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 761342001200SubSeqList.txt, date recorded: Sep. 19, 2022, size: 500,106 bytes).

### FIELD OF THE INVENTION

The present disclosure relates, in part, to recombinant nucleic acids, viruses, medicaments, pharmaceutical compositions, and methods of their use for treating subjects harboring loss-of-function mutations in, and/or pathogenic variants of, one or more ichthyosis-associated genes and/or for providing prophylactic, palliative, or therapeutic relief of one or more signs or symptoms of congenital ichthyosis, e.g., X-linked ichthyosis (XLI), epidermolytic ichthyoses (EI), ichthyosis vulgaris (IV), lamellar ichthyosis (LI), congenital ichthyosiform erythroderma (CIE), harlequin ichthyosis (HI), etc.

### BACKGROUND

Congenital ichthyoses are a heterogenous group of disorders manifesting at birth or infancy with visible scaling and/or thickening of the skin, which may be accompanied by variable degrees of redness (erythema), skin fragility, and/or blistering, as well as abnormalities of the hair, nails, and/or mucus membranes. Scaling and/or thickening of the outermost layer of the skin (hyperkeratosis) may be generalized or localized, may involve other organ systems (syndromic ichthyosis), or may be limited to skin and skin appendages (non-syndromic ichthyosis). The management of congenital ichthyoses is a life-long endeavor, which remains largely symptomatic. At present, disease treatment or management is generally supportive, and commonly focuses on skin lubrication and/or reducing scaling. Thus, there exists a clear need for novel treatment options for all forms of congenital ichthyoses. The present disclosure addresses this and other needs.

All references cited herein, including patent applications, patent publications, non-patent literature, and UniProtKB/Swiss-Prot Accession numbers are herein incorporated by reference in their entirety, as if each individual reference were specifically and individually indicated to be incorporated by reference.

### BRIEF SUMMARY

In some embodiments, provided herein are recombinant nucleic acids (e.g., recombinant herpes viral genomes) comprising the coding sequence of one or more ichthyosis-associated genes (e.g., a polynucleotide encoding a wild-type and/or functional ichthyosis-associated polypeptide) for use in viruses (e.g., herpes viruses), compositions, pharmaceutical formulations, medicaments, and/or methods useful for supplementing or treating ichthyosis-associated gene deficiencies in a subject in need thereof (e.g., a subject naturally harboring pathogenic variants of such gene(s)) and/or for providing prophylactic, palliative, or therapeutic relief of a wound, disorder, or disease of the skin in a subject having, or at risk of developing, one or more signs or symptoms of congenital ichthyosis (e.g., X-linked ichthyosis, lamellar ichthyosis, harlequin ichthyosis, etc.).

Certain aspects of the present disclosure relate to a recombinant herpes virus genome comprising one or more polynucleotides encoding an ichthyosis-associated polypeptide. In some embodiments, the recombinant herpes virus genome is replication competent. In some embodiments, the recombinant herpes virus genome is replication defective. In some embodiments that may be combined with any of the preceding embodiments, the recombinant herpes virus genome is selected from a recombinant herpes simplex virus genome, a recombinant varicella zoster virus genome, a recombinant human cytomegalovirus genome, a recombinant herpesvirus 6A genome, a recombinant herpesvirus 6B genome, a recombinant herpesvirus 7 genome, a recombinant Kaposi's sarcoma-associated herpesvirus genome, and any derivatives thereof. In some embodiments that may be combined with any of the preceding embodiments, the recombinant herpes virus genome is a recombinant herpes simplex virus genome. In some embodiments, the recombinant herpes simplex virus genome is a recombinant type 1 herpes simplex virus (HSV-1) genome, a recombinant type 2 herpes simplex virus (HSV-2) genome, or any derivatives thereof. In some embodiments, the recombinant herpes simplex virus genome is a recombinant type 1 herpes simplex virus (HSV-1) genome.

In some embodiments that may be combined with any of the preceding embodiments, the recombinant herpes simplex virus genome has been engineered to reduce or eliminate expression of one or more toxic herpes simplex virus genes. In some embodiments that may be combined with any of the preceding embodiments, the recombinant herpes simplex virus genome comprises an inactivating mutation. In some embodiments, the inactivating mutation is in a herpes simplex virus gene. In some embodiments, the inactivating mutation is a deletion of the coding sequence of the herpes simplex virus gene. In some embodiments, the herpes simplex virus gene is selected from Infected Cell Protein (ICP) 0, ICP4, ICP22, ICP27, ICP47, thymidine kinase (tk), Long Unique Region (UL) 41, and UL55. In some embodiments that may be combined with any of the preceding embodiments, the recombinant herpes simplex virus genome comprises an inactivating mutation in one or both copies of the ICP4 gene. In some embodiments that may be combined with any of the preceding embodiments, the recombinant herpes simplex virus genome comprises an inactivating mutation in the ICP22 gene. In some embodiments that may be combined with any of the preceding embodiments, the recombinant herpes simplex virus genome comprises an inactivating mutation in the UL41 gene. In some embodiments that may be combined with any of the preceding embodiments, the recombinant herpes simplex virus genome comprises an inactivating mutation in one or both copies of the ICP0 gene. In some embodiments that may be combined with any of the preceding embodiments, the recombinant herpes simplex virus genome comprises an inactivating mutation in the ICP27 gene. In some embodiments that may be combined with any of the preceding embodiments, the recombinant herpes simplex virus genome comprises an inactivating mutation in the UL55 gene. In some embodiments that may be combined with any of the preceding embodiments, the recombinant herpes simplex virus genome comprises an inactivating mutation in the Joint region. In some embodiments, the recombinant herpes simplex virus genome comprises a deletion of the Joint region. In some embodiments that may be combined with any of the preceding embodiments, the recombinant herpes simplex virus genome comprises the one or more polynucleotides encoding the ichthyosis-associated polypeptide within one or both copies of the ICP4 viral gene loci. In some embodiments that may be combined with any of the preceding embodiments, the recombinant herpes simplex virus genome comprises the one or more polynucleotides encoding the ichthyosis-associated polypeptide within the ICP22 viral gene locus. In some embodiments that may be combined with any of the preceding embodiments, the recombinant herpes simplex virus genome comprises the one or more polynucleotides encoding the ichthyosis-associated polypeptide within the UL41 viral gene locus.

In some embodiments that may be combined with any of the preceding embodiments, the ichthyosis-associated polypeptide is not a transglutaminase (TGM) polypeptide. In some embodiments, the ichthyosis-associated polypeptide is not a transglutaminase 1 (TGM1) polypeptide or a transglutaminase 5 (TGM5) polypeptide. In some embodiments that may be combined with any of the preceding embodiments, the ichthyosis-associated polypeptide is selected from an ATP-binding cassette sub-family A member 12 polypeptide (ABCA12), a 1-acylglycerol-3-phosphate O-acyltransferase ABHD5 polypeptide (ABHD5), an Aldehyde dehydrogenase family 3 member A2 polypeptide (ALDH3A2), an Arachidonate 12-lipoxygenase 12R-type polypeptide (ALOX12B), a Hydroperoxide isomerase ALOXE3 polypeptide (ALOXE3), an AP-1 complex subunit sigma-1A polypeptide (AP1S1), an Arylsulfatase E polypeptide (ARSE), a Caspase-14 polypeptide (CASP14), a Corneodesmosin polypeptide (CDSN), a Ceramide synthase 3 polypeptide (CERS3), a Carbohydrate sulfotransferase 8 polypeptide (CHST8), a Claudin-1 polypeptide (CLDN1), a Cystatin-A polypeptide (CSTA), a Cytochrome P450 4F22 polypeptide (CYP4F22), a 3-beta-hydroxysteroid-Delta(8),Delta(7)-isomerase polypeptide (EBP), an Elongation of very long chain fatty acids protein 4 polypeptide (ELOVL4), a Filaggrin polypeptide (FLG), a Filaggrin 2 polypeptide (FLG2), a Gap junction beta-2 polypeptide (GJB2), a Gap junction beta-3 polypeptide (GJB3), a Gap junction beta-4 polypeptide (GJB4), a Gap junction beta-6 polypeptide (GJB6), a 3-ketodihydrosphingosine reductase polypeptide (KDSR), a Keratin, type II cytoskeletal 1 polypeptide (KRT1), a Keratin, type II cytoskeletal 2 epidermal polypeptide (KRT2), a Keratin, type I cytoskeletal 9 polypeptide (KRT9), a Keratin, type I cytoskeletal 10 polypeptide (KRT10), a Lipase member N polypeptide (LIPN), a Loricrin polypeptide (LOR), a Membrane-bound transcription factor site-2 protease polypeptide (MBTPS2), a Magnesium transporter NIPA4 polypeptide (NIPAL4), a Sterol-4-alpha-carboxylate 3-dehydrogenase, decarboxylating polypeptide (NSDHL), a Peroxisomal targeting signal 2 receptor polypeptide (PEX7), a D-3-phosphoglycerate dehydrogenase polypeptide (PHGDH), a Phytanoyl-CoA dioxygenase, peroxisomal polypeptide (PHYH), Patatin-like phospholipase domain-containing protein 1 polypeptide (PNPLA1), a Proteasome maturation protein polypeptide (POMP), a Phosphoserine aminotransferase polypeptide (PSAT1), a Short-chain dehydrogenase/reductase family 9C member 7 polypeptide (SDR9C7), a Serpin B8 polypeptide (SERPINB8), a Long-chain fatty acid transport protein 4 polypeptide (SLC27A4), a Synaptosomal-associated protein 29 polypeptide (SNAP29), a Suppressor of tumorigenicity 14 protein polypeptide (ST14), a Steryl-sulfatase polypeptide (STS), a Sulfotransferase 2B1 polypeptide (SULT2B1), a Vacuolar protein sorting-associated protein 33B polypeptide (VPS33B), and a CAAX prenyl protease 1 homolog polypeptide (ZMPSTE24). In some embodiments that may be combined with any of the preceding embodiments, the ichthyosis-associated polypeptide is a human ichthyosis-associated polypeptide. In some embodiments that may be combined with any of the preceding embodiments, the ichthyosis-associated polypeptide comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from SEQ ID NOS: 102-152 or 155. In some embodiments that may be combined with any of the preceding embodiments, the ichthyosis-associated polypeptide is selected from ABCA12, ABHD5, ALDH3A2, ALOX12B, ALOXE3, AP1S1, ARSE, CASP14, CDSN, CERS3, CHST8, CLDN1, CSTA, CYP4F22, ELOVL4, KDSR, LIPN, MBTPS2, NIPAL4, PEX7, PHGDH, PHYH, PNPLA1, POMP, PSAT1, SDR9C7, SERPINB8, SLC27A4, SNAP29, ST14, STS, VPS33B, and ZMPSTE24. In some embodiments, the ichthyosis-associated polypeptide is selected from ABCA12, ABHD5, ALDH3A2, ALOX12B, ALOXE3, AP1S1, CASP14, CDSN, CERS3, CHST8, CLDN1, CSTA, CYP4F22, ELOVL4, KDSR, LIPN, NIPAL4, PEX7, PHGDH, PHYH, PNPLA1, POMP, PSAT1, SDR9C7, SERPINB8, SLC27A4, SNAP29, ST14, VPS33B, and ZMPSTE24. In some embodiments, the ichthyosis-associated polypeptide is selected from ARSE, MBTPS2, and STS. In some embodiments, the ichthyosis-associated polypeptide is STS.

In some embodiments that may be combined with any of the preceding embodiments, the recombinant herpes virus genome has reduced cytotoxicity when introduced into a target cell as compared to a corresponding wild-type herpes virus genome. In some embodiments, the target cell is a cell of the epidermis and/or dermis. In some embodiments, the target cell is a human cell.

Other aspects of the present disclosure relate to a herpes virus comprising any of the recombinant herpes virus genomes described herein. In some embodiments, the herpes virus is replication competent. In some embodiments, the herpes virus is replication defective. In some embodiments that may be combined with any of the preceding embodiments, the herpes virus has reduced cytotoxicity as compared to a corresponding wild-type herpes virus. In some embodiments that may be combined with any of the preceding embodiments, the herpes virus is selected from a herpes simplex virus, a varicella zoster virus, a human cytomegalovirus, a herpesvirus 6A, a herpesvirus 6B, a herpesvirus 7, and a Kaposi's sarcoma-associated herpesvirus. In some embodiments that may be combined with any of the preceding embodiments, the herpes virus is a herpes simplex virus. In some embodiments, the herpes simplex virus is a type 1 herpes simplex virus (HSV-1), a type 2 herpes simplex virus (HSV-2), or any derivatives thereof. In some embodiments, the herpes simplex virus is a type 1 herpes simplex virus (HSV-1).

Other aspects of the present disclosure related to a pharmaceutical composition comprising any of the recombinant herpes virus genomes and/or any of the herpes viruses described herein and a pharmaceutically acceptable excipient. In some embodiments, the pharmaceutical composition is suitable for topical, transdermal, subcutaneous, intradermal, oral, intranasal, intratracheal, sublingual, buccal, rectal, vaginal, inhaled, intravenous, intraarterial, intramuscular, intracardiac, intraosseous, intraperitoneal, transmucosal, intravitreal, subretinal, intraarticular, peri-articular, local, or epicutaneous administration. In some embodiments, the pharmaceutical composition is suitable for topical, transdermal, subcutaneous, intradermal, or transmucosal administration. In some embodiments, the pharmaceutical composition is suitable for topical, transdermal, or intradermal administration. In some embodiments, the pharmaceutical composition is suitable for topical administration.

Other aspects of the present disclosure relate to the use of any of the recombinant herpes virus genomes and/or herpes viruses described herein as a medicament.

Other aspects of the present disclosure relate to the use of any of the recombinant herpes virus genomes and/or herpes viruses described herein in a therapy.

Other aspects of the present disclosure relate to the use of any of the herpes viruses and/or pharmaceutical compositions described herein in the manufacture of a medicament for treating one or more forms of congenital ichthyosis.

Other aspects of the present disclosure relate to a method of providing prophylactic, palliative, or therapeutic relief to one or more signs or symptoms of congenital ichthyosis in a subject in need thereof comprising administering to the subject an effective amount of any of the herpes viruses and/or pharmaceutical compositions described herein. In some embodiments, the congenital ichthyosis is selected from harlequin ichthyosis (HI), autosomal recessive congenital ichthyosis (ARCI), lamellar ichthyosis (LI), congenital ichthyosiform erythroderma (CIE), Chanarin-Dorfman syndrome (CDS), Sjogren-Larsson syndrome (SLS), mental retardation, enteropathy, deafness, peripheral neuropathy, ichthyosis, and keratoderma (MEDNIK) syndrome, chondrodysplasia punctata 1 (CDPX1), chondrodysplasia punctata 2 (CDPX2), peeling skin syndrome (PSS), neonatal ichthyosis-sclerosing cholangitis (NISCH) syndrome, ichthyosis vulgaris, keratitis-ichthyosis-deafness syndrome (KID), palmoplantar keratoderma (PPK), palmoplantar keratoderma with sensorineural hearing loss (PPK/SNHL), epidermolytic palmoplantar keratoderma (EPPK), erythrokeratodermia variabilis (EKV), Clouston syndrome, progressive symmetric erythrokeratodermia, epidermolytic ichthyosis (EI), superficial epidermolytic ichthyosis (SEI), loricrin keratoderma, ichthyosis follicularis, alopecia, and photophobia (IFAP) syndrome, Olmsted syndrome, congenital hemidysplasia with ichthyosiform nevus and limb defects (CHILD) syndrome, Refsum disease, Neu-Laxova syndrome, keratosis linearis with ichthyosis congenita and sclerosing keratoderma (KLICK) syndrome, ichthyosis prematurity syndrome (IPS), cerebral dysgenesis, neuropathy, ichthyosis, and palmoplantar keratoderma (CEDNIK) syndrome, X-linked ichthyosis, arthrogryposis-renal dysfunction-cholestasis (ARC) syndrome, and restrictive dermopathy. See e.g., U.S. Pat. No. 10,525,090 which is incorporated herein by reference in its entirety for all purposes.

Other aspects of the present disclosure relate to a method of providing prophylactic, palliative, or therapeutic relief to one or more signs or symptoms of harlequin ichthyosis (HI) in a subject in need thereof comprising administering to the subject an effective amount of any of the herpes viruses and/or pharmaceutical compositions described herein. In some embodiments, the recombinant herpes virus genome comprises one or more polynucleotides encoding an ABCA12 polypeptide.

Other aspects of the present disclosure relate to a method of providing prophylactic, palliative, or therapeutic relief to one or more signs or symptoms of Chanarin-Dorfman syndrome (CDS) in a subject in need thereof comprising administering to the subject an effective amount of any of the herpes viruses and/or pharmaceutical compositions described herein. In some embodiments, the recombinant herpes virus genome comprises one or more polynucleotides encoding an ABHD5 polypeptide.

Other aspects of the present disclosure relate to a method of providing prophylactic, palliative, or therapeutic relief to one or more signs or symptoms of Sjogren-Larsson syndrome in a subject in need thereof comprising administering to the subject an effective amount of any of the herpes viruses and/or pharmaceutical compositions described herein. In some embodiments, the recombinant herpes virus genome comprises one or more polynucleotides encoding an ALDH3A2 polypeptide.

Other aspects of the present disclosure relate to a method of providing prophylactic, palliative, or therapeutic relief to one or more signs or symptoms of autosomal recessive congenital ichthyosis (ARCI) in a subject in need thereof comprising administering to the subject an effective amount of any of the herpes viruses and/or pharmaceutical compositions described herein. In some embodiments, the recombinant herpes virus genome comprises one or more polynucleotides encoding a polypeptide selected from ALOX12B, ALOXE3, CASP14, CERS3, CYP4F22, LIPN, NIPAL4, PNPLA1, SDR9C7, SLC27A4, ST14, and SULT2B1.

Other aspects of the present disclosure relate to a method of providing prophylactic, palliative, or therapeutic relief to one or more signs or symptoms of mental retardation, enteropathy, deafness, peripheral neuropathy, ichthyosis, and keratoderma (MEDNIK) syndrome in a subject in need thereof comprising administering to the subject an effective amount of any of the herpes viruses and/or pharmaceutical compositions described herein. In some embodiments, the recombinant herpes virus genome comprises one or more polynucleotides encoding an AP1S1 polypeptide.

Other aspects of the present disclosure relate to a method of providing prophylactic, palliative, or therapeutic relief to one or more signs or symptoms of chondrodysplasia punctata 1 (CDPX1) in a subject in need thereof comprising administering to the subject an effective amount of any of the herpes viruses and/or pharmaceutical compositions described herein. In some embodiments, the recombinant herpes virus genome comprises one or more polynucleotides encoding an ARSE polypeptide.

Other aspects of the present disclosure relate to a method of providing prophylactic, palliative, or therapeutic relief to one or more signs or symptoms of chondrodysplasia punctata 2 (CDPX2) in a subject in need thereof comprising administering to the subject an effective amount of any of the herpes viruses and/or pharmaceutical compositions described herein. In some embodiments, the recombinant herpes virus genome comprises one or more polynucleotides encoding an EBP polypeptide.

Other aspects of the present disclosure relate to a method of providing prophylactic, palliative, or therapeutic relief to one or more signs or symptoms of peeling skin syndrome (PSS) in a subject in need thereof comprising administering to the subject an effective amount of any of the herpes viruses and/or pharmaceutical compositions described herein. In some embodiments, the recombinant herpes virus genome comprises one or more polynucleotides encoding a polypeptide selected from CDSN, CHST8, CSTA, FLG2, and SERPINB8.

Other aspects of the present disclosure relate to a method of providing prophylactic, palliative, or therapeutic relief to one or more signs or symptoms of neonatal ichthyosis-sclerosing cholangitis (NISCH) syndrome in a subject in need thereof comprising administering to the subject an effective amount of any of the herpes viruses and/or pharmaceutical compositions described herein. In some embodiments, the recombinant herpes virus genome comprises one or more polynucleotides encoding a CLDN1 polypeptide.

Other aspects of the present disclosure relate to a method of providing prophylactic, palliative, or therapeutic relief to one or more signs or symptoms of ichthyosis vulgaris in a subject in need thereof comprising administering to the subject an effective amount of any of the herpes viruses and/or pharmaceutical compositions described herein. In some embodiments, the recombinant herpes virus genome comprises one or more polynucleotides encoding a FLG polypeptide.

Other aspects of the present disclosure relate to a method of providing prophylactic, palliative, or therapeutic relief to one or more signs or symptoms of keratitis-ichthyosis-deafness (KID) syndrome, Clouston syndrome, and/or palmoplantar keratoderma with sensorineural hearing loss (PPK/SNHL) in a subject in need thereof comprising administering to the subject an effective amount of any of the herpes viruses and/or pharmaceutical compositions described herein. In some embodiments, the recombinant herpes virus genome comprises one or more polynucleotides encoding a polypeptide selected from GJB2 and GJB6.

Other aspects of the present disclosure relate to a method of providing prophylactic, palliative, or therapeutic relief to one or more signs or symptoms of erythrokeratodermia variabilis (EKV) in a subject in need thereof comprising administering to the subject an effective amount of any of the herpes viruses and/or pharmaceutical compositions described herein. In some embodiments, the recombinant herpes virus genome comprises one or more polynucleotides encoding a polypeptide selected from GJB3 and GJB4.

Other aspects of the present disclosure relate to a method of providing prophylactic, palliative, or therapeutic relief to one or more signs or symptoms of progressive symmetric erythrokeratodermia in a subject in need thereof comprising administering to the subject an effective amount of any of the herpes viruses and/or pharmaceutical compositions described herein. In some embodiments, the recombinant herpes virus genome comprises one or more polynucleotides encoding a KDSR polypeptide.

Other aspects of the present disclosure relate to a method of providing prophylactic, palliative, or therapeutic relief to one or more signs or symptoms of epidermolytic ichthyosis (EI) and/or superficial epidermolytic ichthyosis (SEI) in a subject in need thereof comprising administering to the subject an effective amount of any of the herpes viruses and/or pharmaceutical compositions described herein. In some embodiments, the recombinant herpes virus genome comprises one or more polynucleotides encoding a polypeptide selected from KRT1, KRT2, and KRT10.

Other aspects of the present disclosure relate to a method of providing prophylactic, palliative, or therapeutic relief to one or more signs or symptoms of epidermolytic palmoplantar keratoderma (EPPK) in a subject in need thereof comprising administering to the subject an effective amount of any of the herpes viruses and/or pharmaceutical compositions described herein. In some embodiments, the recombinant herpes virus genome comprises one or more polynucleotides encoding a KRT9 polypeptide.

Other aspects of the present disclosure relate to a method of providing prophylactic, palliative, or therapeutic relief to one or more signs or symptoms of loricrin keratoderma in a subject in need thereof comprising administering to the subject an effective amount of any of the herpes viruses and/or pharmaceutical compositions described herein. In some embodiments, the recombinant herpes virus genome comprises one or more polynucleotides encoding a LOR polypeptide.

Other aspects of the present disclosure relate to a method of providing prophylactic, palliative, or therapeutic relief to one or more signs or symptoms of ichthyosis follicularis, alopecia, and photophobia (IFAP) syndrome in a subject in need thereof comprising administering to the subject an effective amount of any of the herpes viruses and/or pharmaceutical compositions described herein. In some embodiments, the recombinant herpes virus genome comprises one or more polynucleotides encoding a MBTPS2 polypeptide.

Other aspects of the present disclosure relate to a method of providing prophylactic, palliative, or therapeutic relief to one or more signs or symptoms of congenital hemidysplasia with ichthyosiform nevus and limb defects (CHILD) syndrome in a subject in need thereof comprising administering to the subject an effective amount of any of the herpes viruses and/or pharmaceutical compositions described herein. In some embodiments, the recombinant herpes virus genome comprises one or more polynucleotides encoding a NSDHL polypeptide.

Other aspects of the present disclosure relate to a method of providing prophylactic, palliative, or therapeutic relief to one or more signs or symptoms of Refsum disease in a subject in need thereof comprising administering to the subject an effective amount of any of the herpes viruses and/or pharmaceutical compositions described herein. In some embodiments, the recombinant herpes virus genome comprises one or more polynucleotides encoding a polypeptide selected from PEX7 and PHYH.

Other aspects of the present disclosure relate to a method of providing prophylactic, palliative, or therapeutic relief to one or more signs or symptoms of Neu-Laxova in a subject in need thereof comprising administering to the subject an effective amount of any of the herpes viruses and/or pharmaceutical compositions described herein. In some embodiments, the recombinant herpes virus genome comprises one or more polynucleotides encoding a polypeptide selected from PHGDH and PSAT1.

Other aspects of the present disclosure relate to a method of providing prophylactic, palliative, or therapeutic relief to one or more signs or symptoms of keratosis linearis with ichthyosis congenita and sclerosing keratoderma (KLICK) syndrome in a subject in need thereof comprising administering to the subject an effective amount of any of the herpes viruses and/or pharmaceutical compositions described herein. In some embodiments, the recombinant herpes virus genome comprises one or more polynucleotides encoding a POMP polypeptide.

Other aspects of the present disclosure relate to a method of providing prophylactic, palliative, or therapeutic relief to one or more signs or symptoms of ichthyosis prematurity syndrome (IPS) in a subject in need thereof comprising administering to the subject an effective amount of any of the herpes viruses and/or pharmaceutical compositions described herein. In some embodiments, the recombinant herpes virus genome comprises one or more polynucleotides encoding a SLC27A4 polypeptide.

Other aspects of the present disclosure relate to a method of providing prophylactic, palliative, or therapeutic relief to one or more signs or symptoms of cerebral dysgenesis, neuropathy, ichthyosis, and palmoplantar keratoderma (CEDNIK) syndrome in a subject in need thereof comprising administering to the subject an effective amount of any of the herpes viruses and/or pharmaceutical compositions described herein. In some embodiments, the recombinant herpes virus genome comprises one or more polynucleotides encoding a SNAP29 polypeptide.

Other aspects of the present disclosure relate to a method of providing prophylactic, palliative, or therapeutic relief to one or more signs or symptoms of X-linked ichthyosis in a subject in need thereof comprising administering to the subject an effective amount of any of the herpes viruses and/or pharmaceutical compositions described herein. In some embodiments, the recombinant herpes virus genome comprises one or more polynucleotides encoding a STS polypeptide.

Other aspects of the present disclosure relate to a method of providing prophylactic, palliative, or therapeutic relief to one or more signs or symptoms of arthrogryposis-renal dysfunction-cholestasis (ARC) syndrome in a subject in need thereof comprising administering to the subject an effective amount of any of the herpes viruses and/or pharmaceutical compositions described herein. In some embodiments, the recombinant herpes virus genome comprises one or more polynucleotides encoding a VPS33B polypeptide.

Other aspects of the present disclosure relate to a method of providing prophylactic, palliative, or therapeutic relief to one or more signs or symptoms of restrictive dermopathy in a subject in need thereof comprising administering to the subject an effective amount of any of the herpes viruses and/or pharmaceutical compositions described herein. In some embodiments, the recombinant herpes virus genome comprises one or more polynucleotides encoding a ZMPSTE24 polypeptide.

In some embodiments that may be combined with any of the preceding embodiments, the subject is a human. In some embodiments that may be combined with any of the preceding embodiments, the subject's genome comprises a pathogenic variant of an ichthyosis-associated gene. In some embodiments that may be combined with any of the preceding embodiments, the subject's genome comprises a loss-of-function mutation in an ichthyosis-associated gene.

In some embodiments that may be combined with any of the preceding embodiments, the herpes virus or pharmaceutical composition is administered topically, transdermally, subcutaneously, epicutaneously, intradermally, orally, sublingually, buccally, rectally, vaginally, intravenously, intraarterially, intramuscularly, intraosseously, intracardially, intraperitoneally, transmucosally, intravitreally, subretinally, intraarticularly, peri-articularly, locally, or via inhalation to the subject. In some embodiments, the herpes virus or pharmaceutical composition is administered topically, transdermally, subcutaneously, intradermally, or transmucosally to the subject. In some embodiments, the herpes virus or pharmaceutical composition is administered topically, transdermally, or intradermally to the subject. In some embodiments, the herpes virus or pharmaceutical composition is administered topically to the subject. In some embodiments that may be combined with any of the preceding embodiments, the skin of the subject is abraded or made more permeable prior to administration.

### BRIEF DESCRIPTION OF THE DRAWINGS

FIGS. 1A-1I show schematics of wild-type and modified herpes simplex virus genomes. FIG. 1A shows a wild-type herpes simplex virus genome. FIG. 1B shows a modified herpes simplex virus genome comprising deletions of the coding sequence of ICP4 (both copies), with an expression cassette containing a polynucleotide encoding an ichthyosis-associated polypeptide integrated at each of the ICP4 loci. FIG. 1C shows a modified herpes simplex virus genome comprising deletions of the coding sequences of ICP4 (both copies) and UL41, with an expression cassette containing a polynucleotide encoding an ichthyosis-associated polypeptide integrated at each of the ICP4 loci. FIG. 1D shows a modified herpes simplex virus genome comprising deletions of the coding sequences of ICP4 (both copies) and UL41, with an expression cassette containing a polynucleotide encoding an ichthyosis-associated polypeptide integrated at the UL41 locus. FIG. 1E shows a modified herpes simplex virus genome comprising deletions of the coding sequences of ICP4 (both copies) and ICP22, with an expression cassette containing a polynucleotide encoding an ichthyosis-associated polypeptide integrated at each of the ICP4 loci. FIG. 1F shows a modified herpes simplex virus genome comprising deletions of the coding sequences of ICP4 (both copies) and ICP22, with an expression cassette containing a polynucleotide encoding an ichthyosis-associated polypeptide integrated at the ICP22 locus. FIG. 1G shows a modified herpes simplex virus genome comprising deletions of the coding sequences of ICP4 (both copies), UL41, and ICP22, with an expression cassette containing a polynucleotide encoding an ichthyosis-associated polypeptide integrated at each of the ICP4 loci. FIG. 1H shows a modified herpes simplex virus genome comprising deletions of the coding sequences of ICP4 (both copies), UL41, and ICP22, with an expression cassette containing a polynucleotide encoding an ichthyosis-associated polypeptide integrated at the UL41 locus. FIG. 1I shows a modified herpes simplex virus genome comprising deletions of the coding sequences of ICP4 (both copies), UL41, and ICP22, with an expression cassette containing a polynucleotide encoding an ichthyosis-associated polypeptide integrated at the ICP22 locus.

FIGS. 2A-2G show in vitro assessments of HSV-TGM1 in immortalized and primary TGM1-deficient human ARCI keratinocytes grown in low calcium cell culture medium. FIG. 2A shows dose-dependent detection of human TGM1 DNA copies at increasing multiplicities of infection (MOIs) of HSV-TGM1 in immortalized keratinocytes, as assessed by qPCR. Data is presented as the average of two replicates ±SEM. FIG. 2B shows dose-dependent expression of human TGM1 transcripts at increasing MOIs of HSV-TGM1 in immortalized keratinocytes, as assessed by qRT-PCR. Data is presented as the average of two replicates ±SEM. FIG. 2C shows HSV-TGM1-mediated TGM1 protein expression in infected immortalized keratinocytes by western blot. FIG. 2D shows representative immunofluorescence images of human TGM1 protein expression upon HSV-TGM1 infection of immortalized keratinocytes. FIG. 2E shows representative immunofluorescence images of HSV-TGM1-dependent TGM1 enzymatic activity in immortalized keratinocytes. FIG. 2F shows HSV-TGM1-mediated TGM1 protein expression in infected primary cells by western blot analysis. FIG. 2G shows representative immunofluorescence images of human TGM1 protein expression upon HSV-TGM1 infection of primary cells. For these experiments, uninfected (mock) and HSV-mCherry infected (mCherry) cells were used as negative controls; normal primary keratinocytes (NPK) were used as a positive control. DAPI staining was used to visualize nuclei. GAPDH was used as a loading control. For western blot and immunofluorescence analyses, quantification of protein levels and fluorescence intensities are provided for each condition. Bar: 130 μm.

FIGS. 3A-3B show in vitro assessments of HSV-TGM1 in immortalized TGM1-deficient human ARCI keratinocytes grown in high calcium cell culture medium. FIG. 3A shows representative immunofluorescence images of human TGM1 protein expression upon HSV-TGM1 infection of immortalized keratinocytes. FIG. 3B shows representative immunofluorescence images of HSV-TGM1-dependent TGM1 enzymatic activity in immortalized keratinocytes. Uninfected (mock) cells were used as negative controls; normal primary keratinocytes (NPK) were used as a positive control. DAPI staining was used to visualize nuclei. Quantification of fluorescence intensities are provided for each condition. Bar: 130 μm.

FIG. 4 shows in vitro assessment of HSV-TGM1 in primary TGM1-deficient human ARCI keratinocytes grown in high calcium cell culture medium. Representative immunofluorescence images of human TGM1 protein expression upon HSV-TGM1 infection of primary keratinocytes. Uninfected (mock) cells were used as negative controls; normal primary keratinocytes (NPK) were used as a positive control. DAPI staining was used to visualize nuclei. Quantification of fluorescence intensities are provided for each condition. Bar: 130 μm.

FIGS. 5A-5B show viability assessments after HSV-TGM1 infection of primary TGM1-deficient human ARCI keratinocytes grown in low and high calcium cell culture medium. FIG. 5A shows representative brightfield images of primary keratinocytes, grown in low or high calcium cell culture medium, 48 hours after infection with HSV-TGM1 at the indicated MOIs. Uninfected (mock) cells were used as a negative control. FIG. 5B shows viability assessment of HSV-TGM1-infected primary LI patient keratinocytes at 48 h post-infection as determined by MTS Assay. For each condition, data is presented as the average of three separate experiments (with triplicate wells)±SEM. Bar: 370 μm.

FIGS. 6A-6D show in vivo evaluation of HSV-TGM1 via multiple routes of topical delivery to BALB/c mice. FIG. 6A shows representative hematoxylin and eosin (H&E)-stained samples harvested from tape stripped or acetone permeabilized BALB/c mouse skin treated topically with either HSV-TGM1 (low or high dose) or negative control (vehicle). FIG. 6B shows dose-dependent detection of human TGM1 DNA copies in mouse skin biopsies harvested 48 hours after permeabilization by tape stripping or acetone treatment and application of HSV-TGM1 (low or high dose) or negative control (vehicle), as assessed by qPCR. FIG. 6C shows dose-dependent expression of human TGM1 transcripts in mouse skin biopsies harvested 48 hours after permeabilization by tape stripping or acetone treatment and application of HSV-TGM1 (low or high dose) or negative control (vehicle), as assessed by qRT-PCR. For each vehicle control condition in the qPCR and qRT-PCR analysis, data is presented as the average of two tissue samples (two replicates tested/tissue sample)±SEM; for each HSV-TGM1 condition, data is presented as the average of four tissue samples (two replicates tested/tissue sample)±SEM. FIG. 6D shows representative immunofluorescence images of human TGM1, mouse loricrin, and mouse integrin alpha-6 protein localization in mouse skin biopsies harvested 48 hours after skin barrier disruption by acetone treatment or tape stripping and application of HSV-TGM1 (low or high dose) or negative control (vehicle). DAPI staining was used to visualize nuclei. Bar: 50 μm.

FIG. 7 shows in vivo evaluation of mouse TGM1 transcription upon HSV-TGM1 infection via multiple routes of topical delivery to BALB/c mice. Fold change of mouse TGM1 RNA copies in skin biopsies harvested 48 hours after permeabilization by tape stripping or acetone treatment and application of HSV-TGM1 (low or high dose) or vehicle alone relative to untreated control skin, as assessed by qPCR. For each vehicle condition, data is presented as the average of two tissue samples (two replicates tested/tissue sample)±SEM; for each HSV-TGM1 condition, data is presented as the average of four tissue samples (two replicates tested/tissue sample)±SEM. ns: not significant (p>0.05), as determined by two-tailed Student's T-test. Bar: 50 μm.

FIGS. 8A-8C show in vivo short-term pharmacokinetics of HSV-TGM1 upon topical delivery to BALB/c mice. FIG. 8A shows detection of human TGM1 DNA copies in skin biopsies harvested at the indicated timepoints from BALB/c mice treated topically with either HSV-TGM1 or negative control (vehicle). FIG. 8B shows detection of human TGM1 transcripts in skin biopsies harvested at the indicated timepoints from BALB/c mice treated topically with either HSV-TGM1 or negative control (vehicle). For each vehicle control condition in the qPCR and qRT-PCR analysis, data is presented as the average of two tissue samples (two replicates tested/tissue sample)±SEM; for each HSV-TGM1 condition, data is presented as the average of four tissue samples (two replicates tested/tissue sample)±SEM. FIG. 8C shows representative immunofluorescence images of human TGM1 and mouse loricrin protein localization in mouse skin biopsies harvested at the indicated timepoints from BALB/c mice treated topically with either HSV-TGM1 or negative control (vehicle). DAPI staining was used to visualize nuclei. Bar: 50 μm.

FIGS. 9A-9D show in vivo pharmacokinetics of HSV-TGM1 upon single and repeat topical delivery to BALB/c mice. FIG. 9A shows H&E-stained skin biopsies taken from BALB/c mice treated and harvested at the indicated timepoints after a single or repeat topical dose of either HSV-TGM1 or negative control (vehicle). FIG. 9B shows detection of human TGM1 DNA copies in skin biopsies taken from BALB/c mice treated and harvested at the indicated timepoints after a single or repeat topical dose of either HSV-TGM1 or negative control (vehicle). FIG. 9C shows detection of human TGM1 transcripts in skin biopsies taken from BALB/c mice treated and harvested at the indicated timepoints after a single or repeat topical dose of either HSV-TGM1 or negative control (vehicle). For each vehicle control condition in the qPCR and qRT-PCR analysis, data is presented as the average of two tissue samples (two replicates tested/tissue sample)±SEM; for each HSV-TGM1 condition, data is presented as the average of four or six tissue samples (two replicates tested/tissue sample)±SEM. FIG. 9D shows representative immunofluorescence images of human TGM1 and mouse loricrin protein colocalization in skin biopsies taken from BALB/c mice treated and harvested at the indicated timepoints after a single or repeat topical dose of either HSV-TGM1 or negative control (vehicle). DAPI staining was used to visualize nuclei. Bar: 50 μm.

### DETAILED DESCRIPTION

In some embodiments, the present disclosure relates to recombinant nucleic acids (e.g., recombinant herpes viral genomes) comprising one or more polynucleotides encoding one or more ichthyosis-associated polypeptides (e.g., encoding wild-type and/or functional ichthyosis-associated polypeptides), and/or use of these recombinant nucleic acids in viruses (e.g., herpes viruses), compositions, formulations, medicaments, and/or methods in order to supplement or treat endogenous ichthyosis-associated gene deficiencies (e.g., in a subject whose genome naturally harbors a pathogenic variant of the ichthyosis-associated gene(s)). Without wishing to be bound by theory, it is believed that the recombinant nucleic acids, viruses, compositions, formulations, medicaments, and methods described herein will help to treat the existing skin abnormalities in individuals suffering from congenital ichthyosis (such as individuals suffering from X-linked ichthyosis), as well as prevent or delay reformation of wounds or skin abnormalities in treated subjects.

The following description sets forth exemplary methods, parameters, and the like. It should be recognized, however, that such a description is not intended as a limitation on the scope of the present disclosure but is instead provided as a description of exemplary embodiments.

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Gene Transfer Vectors for Mammalian Cells (J. M. Miller and M. P. Calos, eds., 1987); PCR: The Polymerase Chain Reaction, (Mullis et al., eds., 1994); Short Protocols in Molecular Biology (Wiley and Sons, 1999).

### II. Definitions

Before describing the present disclosure in detail, it is to be understood that the present disclosure is not limited to particular compositions or biological systems, which can, of course, vary. It is also to be understood that the terminology used herein is for the purpose of describing particular embodiments only and is not intended to be limiting.

As used herein, the singular forms “a”, “an” and “the” include plural referents unless the content clearly dictates otherwise. Thus, for example, reference to “a molecule” optionally includes a combination of two or more such molecules, and the like.

As used herein, the term “and/or” may include any and all combinations of one or more of the associated listed items. For example, the term “a and/or b” may refer to “a alone”, “b alone”, “a or b”, or “a and b”; the term “a, b, and/or c” may refer to “a alone”, “b alone”, “c alone”, “a or b”, “a or c”, “b or c”, “a, b, or c”, “a and b”, “a and c” or, “b and c”, or “a, b, and c”; etc.

As used herein, the term “about” refers to the usual error range for the respective value readily known to the skilled person in this technical field. Reference to “about” a value or parameter herein includes (and describes) embodiments that are directed to that value or parameter per se.

It is understood that aspects and embodiments of the present disclosure include “comprising”, “consisting”, and “consisting essentially of” aspects and embodiments.

As used herein, the terms “polynucleotide”, “nucleic acid sequence”, “nucleic acid”, and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing non-nucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog, and inter-nucleotide modifications.

As used herein, a nucleic acid is “operatively linked” or “operably linked” when it is placed into a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation. Generally, “operatively linked” or “operably linked” means that the DNA or RNA sequences being linked are contiguous.

As used herein, the term “vector” refers to discrete elements that are used to introduce heterologous nucleic acids into cells for either expression or replication thereof. An expression vector includes vectors capable of expressing nucleic acids that are operatively linked with regulatory sequences, such as promoter regions, that are capable of effecting expression of such nucleic acids. Thus, an expression vector may refer to a DNA or RNA construct, such as a plasmid, a phage, recombinant virus, or other vector that, upon introduction into an appropriate host cell, results in expression of the nucleic acids. Appropriate expression vectors are well known to those of skill in the art and include those that are replicable in eukaryotic cells and those that remain episomal or those which integrate into the host cell genome.

As used herein, an “open reading frame” or “ORF” refers to a continuous stretch of nucleic acids, either DNA or RNA, that encode a protein or polypeptide. Typically, the nucleic acids comprise a translation start signal or initiation codon, such as ATG or AUG, and a termination codon.

As used herein, an “untranslated region” or “UTR” refers to untranslated nucleic acids at the 5′ and/or 3′ ends of an open reading frame. The inclusion of one or more UTRs in a polynucleotide may affect post-transcriptional regulation, mRNA stability, and/or translation of the polynucleotide.

As used herein, the term “transgene” refers to a polynucleotide that is capable of being transcribed into RNA and translated and/or expressed under appropriate conditions, after being introduced into a cell. In some embodiments, it confers a desired property to a cell into which it was introduced, or otherwise leads to a desired therapeutic or diagnostic outcome.

As used herein, the terms “polypeptide,” “protein,” and “peptide” are used interchangeably and may refer to a polymer of two or more amino acids.

As used herein, a “subject”, “host”, or an “individual” refers to any animal classified as a mammal, including humans, domestic and farm animals, and zoo, sports, or pet animals, such as dogs, horses, cats, cows, as well as animals used in research, such as mice, rats, hamsters, rabbits, and non-human primates, etc. In some embodiments, the mammal is human.

As used herein, the terms “pharmaceutical formulation” or “pharmaceutical composition” refer to a preparation which is in such a form as to permit the biological activity of the active ingredient(s) to be effective, and which contains no additional components which are unacceptably toxic to a subject to which the composition or formulation would be administered. “Pharmaceutically acceptable” excipients (e.g., vehicles, additives) are those which can reasonably be administered to a subject mammal to provide an effective dose of the active ingredient(s) employed.

As used herein, “cutaneous administration” or “cutaneously administering” refers to the delivery of a composition to a subject by contacting, directly or otherwise, a formulation comprising the composition to all (“systemic”) or a portion (“topical”) of the skin of a subject. The term encompasses several routes of administration including, but not limited to, topical and transdermal. Topical administration may be used as a means to deliver a composition to the epidermis or dermis of a subject, or to specific strata thereof.

As used herein, an “effective amount” is at least the minimum amount required to affect a measurable improvement or prevention of one or more symptoms of a particular disorder. An “effective amount” may vary according to factors such as the disease state, age, sex, and weight of the patient. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications used to treat symptoms of the disease, delaying the progression of the disease, and/or prolonging survival. An effective amount can be administered in one or more administrations. For purposes of the present disclosure, an effective amount of a recombinant nucleic acid, virus, and/or pharmaceutical composition is an amount sufficient to accomplish prophylactic or therapeutic treatment either directly or indirectly. As is understood in the clinical context, an effective amount of a recombinant nucleic acid, virus, and/or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, “treatment” refers to clinical intervention designed to alter the natural course of the individual or cell being treated during the course of clinical pathology. Desirable effects of treatment include decreasing the rate of disease/disorder/defect progression, ameliorating, or palliating the disease/disorder/defect state, and remission or improved prognosis. For example, an individual is successfully “treated” if one or more symptoms associated with congenital ichthyosis (e.g., X-linked ichthyosis, LI, CIE, HI, etc.) are mitigated or eliminated.

As used herein, the term “delaying progression of” a disease/disorder/defect refers to deferring, hindering, slowing, retarding, stabilizing, and/or postponing development of the disease/disorder/defect. This delay can be of varying lengths or time, depending on the history of the disease/disorder/defect and/or the individual being treated. As is evident to one of ordinary skill in the art, a sufficient or significant delay can, in effect, encompass prevention, in that the individual does not develop the disease.

### III. Recombinant Nucleic Acids

Certain aspects of the present disclosure relate to recombinant nucleic acids (e.g., isolated recombinant nucleic acids) comprising one or more (e.g., one or more, two or more, three or more, four or more, five or more, ten or more, etc.) polynucleotides encoding an ichthyosis-associated polypeptide (e.g., a human ichthyosis-associated polypeptide such as a Steryl-sulfatase polypeptide). In some embodiments, the present disclosure relates to recombinant nucleic acids (e.g., isolated recombinant nucleic acids) comprising one or more (e.g., one or more, two or more, three or more, four or more, five or more, ten or more, etc.) polynucleotides encoding two or more (e.g., two or more, three or more, four or more, five or more, etc.) ichthyosis-associated polypeptides. In some embodiments, the recombinant nucleic acid comprises one or more polynucleotides encoding two or more identical ichthyosis-associated polypeptides. In some embodiments, the recombinant nucleic acid comprises one or more polynucleotides encoding two or more different ichthyosis-associated polypeptides.

In some embodiments, the recombinant nucleic acid is a vector. In some embodiments, the recombinant nucleic acid is a viral vector. In some embodiments, the recombinant nucleic acid is a herpes viral vector. In some embodiments, the recombinant nucleic acid is a herpes simplex virus amplicon. In some embodiments, the recombinant nucleic acid is a recombinant herpes virus genome. In some embodiments, the recombinant nucleic acid is a recombinant herpes simplex virus genome. In some embodiments, the recombinant herpes simplex virus genome is a recombinant type 1 herpes simplex virus (HSV-1) genome.

Polynucleotides Encoding Ichthyosis-Associated Polypeptides

In some embodiments, the present disclosure relates to a recombinant nucleic acid comprising one or more polynucleotides comprising the coding sequence of an ichthyosis-associated gene. In some embodiments, an ichthyosis-associated gene is a wild-type and/or functional version of a gene that has been identified as comprising a pathogenic variant and/or loss-of-function mutation that is correlated with, causative of, or contributes to one or more forms of congenital ichthyosis (e.g., a pathogenic variant and/or loss-of function mutation in gene identified in a patient suffering from one or more of harlequin ichthyosis (HI), autosomal recessive congenital ichthyosis (ARCI), lamellar ichthyosis (LI), congenital ichthyosiform erythroderma (CIE), Chanarin-Dorfman syndrome (CDS), Sjogren-Larsson syndrome (SLS), mental retardation, enteropathy, deafness, peripheral neuropathy, ichthyosis, and keratoderma (MEDNIK) syndrome, chondrodysplasia punctata 1 (CDPX1), chondrodysplasia punctata 2 (CDPX2), peeling skin syndrome (PSS), neonatal ichthyosis-sclerosing cholangitis (NISCH) syndrome, ichthyosis vulgaris, keratitis-ichthyosis-deafness syndrome (KID), palmoplantar keratoderma (PPK), palmoplantar keratoderma with sensorineural hearing loss (PPK/SNHL), epidermolytic palmoplantar keratoderma (EPPK), erythrokeratodermia variabilis (EKV), Clouston syndrome, progressive symmetric erythrokeratodermia, epidermolytic ichthyosis (EI), superficial epidermolytic ichthyosis (SEI), loricrin keratoderma, ichthyosis follicularis, alopecia, and photophobia (IFAP) syndrome, Olmsted syndrome, congenital hemidysplasia with ichthyosiform nevus and limb defects (CHILD) syndrome, Refsum disease, Neu-Laxova syndrome, keratosis linearis with ichthyosis congenita and sclerosing keratoderma (KLICK) syndrome, ichthyosis prematurity syndrome (IPS), cerebral dysgenesis, neuropathy, ichthyosis, and palmoplantar keratoderma (CEDNIK) syndrome, X-linked ichthyosis, arthrogryposis-renal dysfunction-cholestasis (ARC) syndrome, and/or restrictive dermopathy). Genes harboring pathogenic variants and/or loss-of-function mutations that are correlated with, causative of, or contribute to one or more forms of congenital ichthyosis include, e.g., ABCA12, ABHD5, ALDH3A2, ALOX12B, ALOXE3, AP1S1, ARSE, CASP14, CDSN, CERS3, CHST8, CLDN1, CSTA, CYP4F22, EBP, ELOVL4, FLG, FLG2, GJB2, GJB3, GJB4, GJB6, KDSR, KRT1, KRT2, KRT9, KRT10, LIPN, LOR, MBTPS2, NIPAL4, NSDHL, PEX7, PHGDH, PHYH, PNPLA1, POMP, PSAT1, SDR9C7, SERPINB8, SLC27A4, SNAP29, ST14, STS, SULT2B1, VPS33B, and ZMPSTE24.

The coding sequence of any suitable ichthyosis-associated gene (including any isoform thereof) known in the art may be encoded by a polynucleotide of the present disclosure, including, for example, an ABCA12 gene (such as a human ABCA12 gene, e.g., as disclosed by NCBI Gene ID: 26154), an ABHD5 gene (such as a human ABHD5 gene, e.g., as disclosed by NCBI Gene ID: 51099), an ALDH3A2 gene (such as a human ALDH3A2 gene, e.g., as disclosed by NCBI Gene ID: 224), an ALOX12B gene (such as a human ALOX12B gene, e.g., as disclosed by NCBI Gene ID: 242), an ALOXE3 gene (such as a human ALOXE3 gene, e.g., as disclosed by NCBI Gene ID: 59344), an AP1S1 gene (such as a human AP1S1 gene, e.g., as disclosed by NCBI Gene ID: 1174), an ARSE gene (such as a human ARSE gene, e.g., as disclosed by NCBI Gene ID: 415), a CASP14 gene (such as a human CASP14 gene, e.g., as disclosed by NCBI Gene ID: 23581), a CDSN gene (such as a human CDSN gene, e.g., as disclosed by NCBI Gene ID: 1041), a CERS3 gene (such as a human CERS3 gene, e.g., as disclosed by NCBI Gene ID: 204219), a CHST8 gene (such as a human CHST8 gene, e.g., as disclosed by NCBI Gene ID: 64377), a CLDN1 gene (such as a human CLDN1 gene, e.g., as disclosed by NCBI Gene ID: 9076), a CSTA gene (such as a human CSTA gene, e.g., as disclosed by NCBI Gene ID: 1475), a CYP4F22 gene (such as a human CYP4F22 gene, e.g., as disclosed by NCBI Gene ID: 126410), an EBP gene (such as a human EBP gene, e.g., as disclosed by NCBI Gene ID: 10682), an ELOVL4 gene (such as a human ELOVL4 gene, e.g., as disclosed by NCBI Gene ID: 6785), a FLG gene (such as a human FLG gene, e.g., as disclosed by NCBI Gene ID: 2312), a FLG2 gene (such as a human FLG2 gene, e.g., as disclosed by NCBI Gene ID: 388698), a GJB2 gene (such as a human GJB2 gene, e.g., as disclosed by NCBI Gene ID: 2706), a GJB3 gene (such as a human GJB3 gene, e.g., as disclosed by NCBI Gene ID: 2707), a GJB4 gene (such as a human GJB4 gene, e.g., as disclosed by NCBI Gene ID: 127534), a GJB6 gene (such as a human GJB6 gene, e.g., as disclosed by NCBI Gene ID: 10804), a KDSR gene (such as a human KDSR gene, e.g., as disclosed by NCBI Gene ID: 2531), a KRT1 gene (such as a human KRT1 gene, e.g., as disclosed by NCBI Gene ID: 3848), a KRT2 gene (such as a human KRT2 gene, e.g., as disclosed by NCBI Gene ID: 3849), a KRT9 gene (such as a human KRT9 gene, e.g., as disclosed by NCBI Gene ID: 3857), a KRT10 gene (such as a human KRT10 gene, e.g., as disclosed by NCBI Gene ID: 3858), an LIPN gene (such as a human LIPN gene, e.g., as disclosed by NCBI Gene ID: 643418), an LOR gene (such as a human LOR gene, e.g., as disclosed by NCBI Gene ID: 4014), an MBTPS2 gene (such as a human MBTPS2 gene, e.g., as disclosed by NCBI Gene ID: 51360), an NIPAL4 gene (such as a human NIPAL4 gene, e.g., as disclosed by NCBI Gene ID: 348938), an NSDHL gene (such as a human NSDHL gene, e.g., as disclosed by NCBI Gene ID: 50814), a PEX7 gene (such as a human PEX7 gene, e.g., as disclosed by NCBI Gene ID: 5191), a PHGDH gene (such as a human PHGDH gene, e.g., as disclosed by NCBI Gene ID: 26227), a PHYH gene (such as a human PHYH gene, e.g., as disclosed by NCBI Gene ID: 5264), a PNPLA1 gene (such as a human PNPLA1 gene, e.g., as disclosed by NCBI Gene ID: 285848), a POMP gene (such as a human POMP gene, e.g., as disclosed by NCBI Gene ID: 51371), a PSAT1 gene (such as a human PSAT1 gene, e.g., as disclosed by NCBI Gene ID: 29968), an SDR9C7 gene (such as a human SDR9C7 gene, e.g., as disclosed by NCBI Gene ID: 121214), a SERPINB8 gene (such as a human SERPINB8 gene, e.g., as disclosed by NCBI Gene ID: 5271), an SLC27A4 gene (such as a human SLC27A4 gene, e.g., as disclosed by NCBI Gene ID: 10999), a SNAP29 gene (such as a human SNAP29 gene, e.g., as disclosed by NCBI Gene ID: 9342), an ST14 gene (such as a human ST14 gene, e.g., as disclosed by NCBI Gene ID: 6768), an STS gene (such as a human STS gene, e.g., as disclosed by NCBI Gene ID: 412), a SULT2B1 gene (such as a human SULT2B1 gene, e.g., as disclosed by NCBI Gene ID: 6820), a VPS33B gene (such as a human VPS33B gene, e.g., as disclosed by NCBI Gene ID: 26276), and a ZMPSTE24 gene (such as a human ZMPSTE24 gene, e.g., as disclosed by NCBI Gene ID: 10269). Methods of identifying ichthyosis-associated gene homologs/orthologs from additional species are known to one of ordinary skill in the art, including, for example, using a nucleic acid sequence alignment program such as the BLAST® blastn suite. In some embodiments, a polynucleotide of the preset disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of any of the ichthyosis-associated genes (and/or coding sequences thereof) described herein. In some embodiments, a polynucleotide of the present disclosure comprises the coding sequence of a human ichthyosis-associated gene.

In some embodiments, a polynucleotide of the present disclosure comprises a codon-optimized variant of the coding sequence of any of the ichthyosis-associated genes described herein or known in the art. In some embodiments, use a of a codon-optimized variant of the coding sequence of an ichthyosis-associated gene increases stability and/or yield of heterologous expression (RNA and/or protein) of the encoded polypeptide in a target cell, as compared to the stability and/or yield of heterologous expression of a corresponding, non-codon-optimized, wild-type sequence. Any suitable method known in the art for performing codon optimization of a sequence for expression in one or more target cells (e.g., one or more human cells) may be used, including, for example, by the methods described by Fath et al. (PLoS One. 2011 Mar. 3; 6(3): e17596).

In some embodiments, a polynucleotide of the present disclosure comprises the coding sequence of a human ABCA12 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NOS: 1-4. In some embodiments, a polynucleotide of the present disclosure comprises a sequence selected from SEQ ID NOS: 1-4. In some embodiments, a polynucleotide of the present disclosure comprises the sequence of SEQ ID NO: 1 or SEQ ID NO: 2.

In some embodiments, a polynucleotide of the present disclosure comprises a 5′ truncation, a 3′ truncation, or a fragment of the sequence of SEQ ID NO: 1 or SEQ ID NO: 2. In some embodiments, the 5′ truncation, 3′ truncation, or fragment of the sequence of SEQ ID NO: 1 or SEQ ID NO: 2 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 2000, at least 3000, at least 4000, at least 5000, at least 6000, at least 7000, but fewer than 7788 consecutive nucleotides of SEQ ID NO: 1 or SEQ ID NO: 2. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-7785 of SEQ ID NO: 1 or SEQ ID NO: 2. In some embodiments, a polynucleotide of the present disclosure comprises the sequence of nucleic acids 1-7785 of SEQ ID NO: 1 or SEQ ID NO: 2.

In some embodiments, a polynucleotide of the present disclosure comprises a 5′ truncation, a 3′ truncation, or a fragment of the sequence of SEQ ID NO: 3 or SEQ ID NO: 4. In some embodiments, the 5′ truncation, 3′ truncation, or fragment of the sequence of SEQ ID NO: 3 or SEQ ID NO: 4 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 2000, at least 3000, at least 4000, at least 5000, at least 6000, but fewer than 6834 consecutive nucleotides of SEQ ID NO: 3 or SEQ ID NO: 4. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-6831 of SEQ ID NO: 3 or SEQ ID NO: 4. In some embodiments, a polynucleotide of the present disclosure comprises the sequence of nucleic acids 1-6831 of SEQ ID NO: 3 or SEQ ID NO: 4.

In some embodiments, a polynucleotide of the present disclosure comprises the coding sequence of a human ABHD5 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 5 or SEQ ID NO: 6. In some embodiments, a polynucleotide of the present disclosure comprises the sequence of SEQ ID NO: 5 or SEQ ID NO: 6.

In some embodiments, a polynucleotide of the present disclosure comprises a 5′ truncation, a 3′ truncation, or a fragment of the sequence of SEQ ID NO: 5 or SEQ ID NO: 6. In some embodiments, the 5′ truncation, 3′ truncation, or fragment of the sequence of SEQ ID NO: 5 or SEQ ID NO: 6 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, but fewer than 1050 consecutive nucleotides of SEQ ID NO: 5 or SEQ ID NO: 6. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-1047 of SEQ ID NO: 5 or SEQ ID NO: 6. In some embodiments, a polynucleotide of the present disclosure comprises the sequence of nucleic acids 1-1047 of SEQ ID NO: 5 or SEQ ID NO: 6.

In some embodiments, a polynucleotide of the present disclosure comprises the coding sequence of a human ALDH3A2 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 7 or SEQ ID NO: 8. In some embodiments, a polynucleotide of the present disclosure comprises the sequence of SEQ ID NO: 7 or SEQ ID NO: 8.

In some embodiments, a polynucleotide of the present disclosure comprises a 5′ truncation, a 3′ truncation, or a fragment of the sequence of SEQ ID NO: 7 or SEQ ID NO: 8. In some embodiments, the 5′ truncation, 3′ truncation, or fragment of the sequence of SEQ ID NO: 7 or SEQ ID NO: 8 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, at least 1500, but fewer than 1527 consecutive nucleotides of SEQ ID NO: 7 or SEQ ID NO: 8. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-1524 of SEQ ID NO: 7 or SEQ ID NO: 8. In some embodiments, a polynucleotide of the present disclosure comprises the sequence of nucleic acids 1-1524 of SEQ ID NO: 7 or SEQ ID NO: 8.

In some embodiments, a polynucleotide of the present disclosure comprises the coding sequence of a human ALOX12B gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 9 or SEQ ID NO: 10. In some embodiments, a polynucleotide of the present disclosure comprises the sequence of SEQ ID NO: 9 or SEQ ID NO: 10.

In some embodiments, a polynucleotide of the present disclosure comprises a 5′ truncation, a 3′ truncation, or a fragment of the sequence of SEQ ID NO: 9 or SEQ ID NO: 10. In some embodiments, the 5′ truncation, 3′ truncation, or fragment of the sequence of SEQ ID NO: 9 or SEQ ID NO: 10 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, at least 1500, at least 1750, at least 2000, but fewer than 2106 consecutive nucleotides of SEQ ID NO: 9 or SEQ ID NO: 10. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-2103 of SEQ ID NO: 9 or SEQ ID NO: 10. In some embodiments, a polynucleotide of the present disclosure comprises the sequence of nucleic acids 1-2106 of SEQ ID NO: 9 or SEQ ID NO: 10.

In some embodiments, a polynucleotide of the present disclosure comprises the coding sequence of a human ALOXE3 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NOS: 11-14. In some embodiments, a polynucleotide of the present disclosure comprises a sequence selected from SEQ ID NOS:11-14.

In some embodiments, a polynucleotide of the present disclosure comprises a 5′ truncation, a 3′ truncation, or a fragment of the sequence of SEQ ID NO: 11 or SEQ ID NO: 12. In some embodiments, the 5′ truncation, 3′ truncation, or fragment of the sequence of SEQ ID NO: 11 or SEQ ID NO: 12 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, at least 1500, at least 1750, at least 2000, but fewer than 2136 consecutive nucleotides of SEQ ID NO: 11 or SEQ ID NO: 12. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-2133 of SEQ ID NO: 11 or SEQ ID NO: 12. In some embodiments, a polynucleotide of the present disclosure comprises the sequence of nucleic acids 1-2133 of SEQ ID NO: 11 or SEQ ID NO: 12.

In some embodiments, a polynucleotide of the present disclosure comprises a 5′ truncation, a 3′ truncation, or a fragment of the sequence of SEQ ID NO: 13 or SEQ ID NO: 14. In some embodiments, the 5′ truncation, 3′ truncation, or fragment of the sequence of SEQ ID NO: 13 or SEQ ID NO: 14 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, at least 1500, at least 1750, at least 2000, at least 2250, at least 2500, but fewer than 2532 consecutive nucleotides of SEQ ID NO: 13 or SEQ ID NO: 14. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-2529 of SEQ ID NO: 13 or SEQ ID NO: 14. In some embodiments, a polynucleotide of the present disclosure comprises the sequence of nucleic acids 1-2529 of SEQ ID NO: 13 or SEQ ID NO: 14.

In some embodiments, a polynucleotide of the present disclosure comprises the coding sequence of a human AP1S1 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 15 or SEQ ID NO: 16. In some embodiments, a polynucleotide of the present disclosure comprises the sequence of SEQ ID NO: 15 or SEQ ID NO: 16.

In some embodiments, a polynucleotide of the present disclosure comprises a 5′ truncation, a 3′ truncation, or a fragment of the sequence of SEQ ID NO: 15 or SEQ ID NO: 16. In some embodiments, the 5′ truncation, 3′ truncation, or fragment of the sequence of SEQ ID NO: 15 or SEQ ID NO: 16 is a polynucleotide that has at least 25, at least 50, at least or at least 350, at least 400, at least 450, but fewer than 477 consecutive nucleotides of SEQ ID NO: 15 or SEQ ID NO: 16. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-474 of SEQ ID NO: 15 or SEQ ID NO: 16. In some embodiments, a polynucleotide of the present disclosure comprises the sequence of nucleic acids 1-474 of SEQ ID NO: 15 or SEQ ID NO: 16.

In some embodiments, a polynucleotide of the present disclosure comprises the coding sequence of a human ARSE gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 17 or SEQ ID NO: 18. In some embodiments, a polynucleotide of the present disclosure comprises the sequence of SEQ ID NO: 17 or SEQ ID NO: 18.

In some embodiments, a polynucleotide of the present disclosure comprises a 5′ truncation, a 3′ truncation, or a fragment of the sequence of SEQ ID NO: 17 or SEQ ID NO: 18. In some embodiments, the 5′ truncation, 3′ truncation, or fragment of the sequence of SEQ ID NO: 17 or SEQ ID NO: 18 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, at least 1500, at least 1750, but fewer than 1770 consecutive nucleotides of SEQ ID NO: 17 or SEQ ID NO: 18. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-1767 of SEQ ID NO: 17 or SEQ ID NO: 18. In some embodiments, a polynucleotide of the present disclosure comprises the sequence of nucleic acids 1-1767 of SEQ ID NO: 17 or SEQ ID NO: 18.

In some embodiments, a polynucleotide of the present disclosure comprises the coding sequence of a human CASP14 gene (or a codon-optimized variant thereof). In some embodiments, a polynucleotide of the present disclosure comprises a sequence having at least 90%, at least 91%, at least 92%, at least 93%, at least 94%, at least 95%, at least 96%, at least 97%, at least 98%, at least 99%, or 100% sequence identity to the sequence of SEQ ID NO: 19 or SEQ ID NO: 20. In some embodiments, a polynucleotide of the present disclosure comprises the sequence of SEQ ID NO: 19 or SEQ ID NO: 20.

In some embodiments, a polynucleotide of the present disclosure comprises a 5′ truncation, a 3′ truncation, or a fragment of the sequence of SEQ ID NO: 19 or SEQ ID NO: 20. In some embodiments, the 5′ truncation, 3′ truncation, or fragment of the sequence of SEQ ID NO: 19 or SEQ ID NO: 20 is a polynucleotide that has at least 25, at least 50, at least 75, at least 100, at least 125, at least 150, at least 175, at least 200, at least 250, at least 300, or at least 350, at least 400, at least 450, at least 500, but fewer than 729 consecutive nucleotides of SEQ ID NO: 19 or SEQ ID NO: 20. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-726 of SEQ ID NO: 19 or SEQ ID NO: 20. In some embodiments, a polynucleotide of the present disclosure comprises the sequence of nucleic acids 1-726 of SEQ ID NO: 19 or SEQ ID NO: 20.

In some embodiments, a polynucleotide of the present disclosure comprises the coding sequence of a human CDSN gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 21 or SEQ ID NO: 22. In some embodiments, a polynucleotide of the present disclosure comprises the sequence of SEQ ID NO: 21 or SEQ ID NO: 22.

In some embodiments, a polynucleotide of the present disclosure comprises a 5′ truncation, a 3′ truncation, or a fragment of the sequence of SEQ ID NO: 21 or SEQ ID NO: 22. In some embodiments, the 5′ truncation, 3′ truncation, or fragment of the sequence of SEQ ID NO: 21 or SEQ ID NO: 22 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, at least 1500, but fewer than 1590 consecutive nucleotides of SEQ ID NO: 21 or SEQ ID NO: 22. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-1587 of SEQ ID NO: 21 or SEQ ID NO: 22. In some embodiments, a polynucleotide of the present disclosure comprises the sequence of nucleic acids 1-1587 of SEQ ID NO: 21 or SEQ ID NO: 22.

In some embodiments, a polynucleotide of the present disclosure comprises the coding sequence of a human CERS3 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 23 or SEQ ID NO: 24. In some embodiments, a polynucleotide of the present disclosure comprises the sequence of SEQ ID NO: 23 or SEQ ID NO: 24.

In some embodiments, a polynucleotide of the present disclosure comprises a 5′ truncation, a 3′ truncation, or a fragment of the sequence of SEQ ID NO: 23 or SEQ ID NO: 24. In some embodiments, the 5′ truncation, 3′ truncation, or fragment of the sequence of SEQ ID NO: 23 or SEQ ID NO: 24 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, but fewer than 1152 consecutive nucleotides of SEQ ID NO: 23 or SEQ ID NO: 24. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-1149 of SEQ ID NO: 23 or SEQ ID NO: 24. In some embodiments, a polynucleotide of the present disclosure comprises the sequence of nucleic acids 1-1149 of SEQ ID NO: 23 or SEQ ID NO: 24.

In some embodiments, a polynucleotide of the present disclosure comprises the coding sequence of a human CHST8 gene (or a codon-optimized variant thereof). In some embodiments, a polynucleotide of the present disclosure comprises a sequence having at least 97%, at least 98%, at least 99%, or 100% sequence identity to the sequence of SEQ ID NO: 25 or SEQ ID NO: 26. In some embodiments, a polynucleotide of the present disclosure comprises the sequence of SEQ ID NO: 25 or SEQ ID NO: 26.

In some embodiments, a polynucleotide of the present disclosure comprises a 5′ truncation, a 3′ truncation, or a fragment of the sequence of SEQ ID NO: 25 or SEQ ID NO: 26. In some embodiments, the 5′ truncation, 3′ truncation, or fragment of the sequence of SEQ ID NO: 25 or SEQ ID NO: 26 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, but fewer than 1275 consecutive nucleotides of SEQ ID NO: 25 or SEQ ID NO: 26. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-1272 of SEQ ID NO: 25 or SEQ ID NO: 26. In some embodiments, a polynucleotide of the present disclosure comprises the sequence of nucleic acids 1-272 of SEQ ID NO: 25 or SEQ ID NO: 26.

In some embodiments, a polynucleotide of the present disclosure comprises the coding sequence of a human CLDN1 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 27 or SEQ ID NO: 28. In some embodiments, a polynucleotide of the present disclosure comprises the sequence of SEQ ID NO: 27 or SEQ ID NO: 28.

In some embodiments, a polynucleotide of the present disclosure comprises a 5′ truncation, a 3′ truncation, or a fragment of the sequence of SEQ ID NO: 27 or SEQ ID NO: 28. In some embodiments, the 5′ truncation, 3′ truncation, or fragment of the sequence of SEQ ID NO: 27 or SEQ ID NO: 28 is a polynucleotide that has at least 25, at least 50, at least 75, at least 100, at least 125, at least 150, at least 175, at least 200, at least 250, at least 300, or at least 350, at least 400, at least 450, at least 500, but fewer than 636 consecutive nucleotides of SEQ ID NO: 27 or SEQ ID NO: 28. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-633 of SEQ ID NO: 27 or SEQ ID NO: 28. In some embodiments, a polynucleotide of the present disclosure comprises the sequence of nucleic acids 1-633 of SEQ ID NO: 27 or SEQ ID NO: 28.

In some embodiments, a polynucleotide of the present disclosure comprises the coding sequence of a human CSTA gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 29 or SEQ ID NO: 30. In some embodiments, a polynucleotide of the present disclosure comprises the sequence of SEQ ID NO: 29 or SEQ ID NO: 30.

In some embodiments, a polynucleotide of the present disclosure comprises a 5′ truncation, a 3′ truncation, or a fragment of the sequence of SEQ ID NO: 29 or SEQ ID NO: 30. In some embodiments, the 5′ truncation, 3′ truncation, or fragment of the sequence of SEQ ID NO: 29 or SEQ ID NO: 30 is a polynucleotide that has at least 25, at least 50, at least 75, at least 100, at least 125, at least 150, at least 175, at least 200, at least 250, but fewer than 297 consecutive nucleotides of SEQ ID NO: 29 or SEQ ID NO: 30. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-294 of SEQ ID NO: 29 or SEQ ID NO: 30. In some embodiments, a polynucleotide of the present disclosure comprises the sequence of nucleic acids 1-294 of SEQ ID NO: 29 or SEQ ID NO: 30.

In some embodiments, a polynucleotide of the present disclosure comprises the coding sequence of a human CYP4F22 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 31 or SEQ ID NO: 32. In some embodiments, a polynucleotide of the present disclosure comprises the sequence of SEQ ID NO: 31 or SEQ ID NO: 32.

In some embodiments, a polynucleotide of the present disclosure comprises a 5′ truncation, a 3′ truncation, or a fragment of the sequence of SEQ ID NO: 31 or SEQ ID NO: 32. In some embodiments, the 5′ truncation, 3′ truncation, or fragment of the sequence of SEQ ID NO: 31 or SEQ ID NO: 32 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, at least 1500, but fewer than 1596 consecutive nucleotides of SEQ ID NO: 31 or SEQ ID NO: 32. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-1593 of SEQ ID NO: 31 or SEQ ID NO: 32. In some embodiments, a polynucleotide of the present disclosure comprises the sequence of nucleic acids 1-1593 of SEQ ID NO: 31 or SEQ ID NO: 32.

In some embodiments, a polynucleotide of the present disclosure comprises the coding sequence of a human EBP gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 33 or SEQ ID NO: 34. In some embodiments, a polynucleotide of the present disclosure comprises the sequence of SEQ ID NO: 33 or SEQ ID NO: 34.

In some embodiments, a polynucleotide of the present disclosure comprises a 5′ truncation, a 3′ truncation, or a fragment of the sequence of SEQ ID NO: 33 or SEQ ID NO: 34. In some embodiments, the 5′ truncation, 3′ truncation, or fragment of the sequence of SEQ ID NO: 33 or SEQ ID NO: 34 is a polynucleotide that has at least 25, at least 50, at least 75, at least 100, at least 125, at least 150, at least 175, at least 200, at least 250, at least 300, or at least 350, at least 400, at least 450, at least 500, but fewer than 693 consecutive nucleotides of SEQ ID NO: 33 or SEQ ID NO: 34. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-690 of SEQ ID NO: 33 or SEQ ID NO: 34. In some embodiments, a polynucleotide of the present disclosure comprises the sequence of nucleic acids 1-690 of SEQ ID NO: 33 or SEQ ID NO: 34.

In some embodiments, a polynucleotide of the present disclosure comprises the coding sequence of a human ELOVL4 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 35 or SEQ ID NO: 36. In some embodiments, a polynucleotide of the present disclosure comprises the sequence of SEQ ID NO: 35 or SEQ ID NO: 36.

In some embodiments, a polynucleotide of the present disclosure comprises a 5′ truncation, a 3′ truncation, or a fragment of the sequence of SEQ ID NO: 35 or SEQ ID NO: 36. In some embodiments, the 5′ truncation, 3′ truncation, or fragment of the sequence of SEQ ID NO: 35 or SEQ ID NO: 36 is a polynucleotide that has at least 25, at least 50, at least 75, at least 100, at least 125, at least 150, at least 175, at least 200, at least 250, at least 300, or at least 350, at least 400, at least 450, at least 500, at least 750, but fewer than 945 consecutive nucleotides of SEQ ID NO: 35 or SEQ ID NO: 36. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-942 of SEQ ID NO: 35 or SEQ ID NO: 36. In some embodiments, a polynucleotide of the present disclosure comprises the sequence of nucleic acids 1-942 of SEQ ID NO: 35 or SEQ ID NO: 36.

In some embodiments, a polynucleotide of the present disclosure comprises the coding sequence of a human FLG gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 37 or SEQ ID NO: 38. In some embodiments, a polynucleotide of the present disclosure comprises the sequence of SEQ ID NO: 37 or SEQ ID NO: 38.

In some embodiments, a polynucleotide of the present disclosure comprises a 5′ truncation, a 3′ truncation, or a fragment of the sequence of SEQ ID NO: 37 or SEQ ID NO: 38. In some embodiments, the 5′ truncation, 3′ truncation, or fragment of the sequence of SEQ ID NO: 37 or SEQ ID NO: 38 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, at least 1500, at least 1750, at least 2000, at least 3000, at least 4000, at least 5000, at least 6000, at least 7000, at least 8000, at least 9000, at least 10000, at least 11000, at least 12000, but fewer than 12186 consecutive nucleotides of SEQ ID NO: 37 or SEQ ID NO: 38. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-12183 of SEQ ID NO: 37 or SEQ ID NO: 38. In some embodiments, a polynucleotide of the present disclosure comprises the sequence of nucleic acids 1-12183 of SEQ ID NO: 37 or SEQ ID NO: 38.

In some embodiments, a polynucleotide of the present disclosure comprises the coding sequence of a human FLG2 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 39. In some embodiments, a polynucleotide of the present disclosure comprises the sequence of SEQ ID NO: 39.

In some embodiments, a polynucleotide of the present disclosure comprises a 5′ truncation, a 3′ truncation, or a fragment of the sequence of SEQ ID NO: 39. In some embodiments, the 5′ truncation, 3′ truncation, or fragment of the sequence of SEQ ID NO: 39 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, at least 1500, at least 1750, at least 2000, at least 3000, at least 4000, at least 5000, at least 6000, at least 7000, but fewer than 7176 consecutive nucleotides of SEQ ID NO: 39. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-7173 of SEQ ID NO: 39. In some embodiments, a polynucleotide of the present disclosure comprises the sequence of nucleic acids 1-7173 of SEQ ID NO: 39.

In some embodiments, a polynucleotide of the present disclosure comprises the coding sequence of a human GJB2 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 40 or SEQ ID NO: 41. In some embodiments, a polynucleotide of the present disclosure comprises the sequence of SEQ ID NO: 40 or SEQ ID NO: 41.

In some embodiments, a polynucleotide of the present disclosure comprises a 5′ truncation, a 3′ truncation, or a fragment of the sequence of SEQ ID NO: 40 or SEQ ID NO: 41. In some embodiments, the 5′ truncation, 3′ truncation, or fragment of the sequence of SEQ ID NO: 40 or SEQ ID NO: 41 is a polynucleotide that has at least 25, at least 50, at least 75, at least 100, at least 125, at least 150, at least 175, at least 200, at least 250, at least 300, or at least 350, at least 400, at least 450, at least 500, but fewer than 681 consecutive nucleotides of SEQ ID NO: 40 or SEQ ID NO: 41. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-678 of SEQ ID NO: 40 or SEQ ID NO: 41. In some embodiments, a polynucleotide of the present disclosure comprises the sequence of nucleic acids 1-678 of SEQ ID NO: 40 or SEQ ID NO: 41.

In some embodiments, a polynucleotide of the present disclosure comprises the coding sequence of a human GJB3 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 42 or SEQ ID NO: 43. In some embodiments, a polynucleotide of the present disclosure comprises the sequence of SEQ ID NO: 42 or SEQ ID NO: 43.

In some embodiments, a polynucleotide of the present disclosure comprises a 5′ truncation, a 3′ truncation, or a fragment of the sequence of SEQ ID NO: 42 or SEQ ID NO: 43. In some embodiments, the 5′ truncation, 3′ truncation, or fragment of the sequence of SEQ ID NO: 42 or SEQ ID NO: 43 is a polynucleotide that has at least 25, at least 50, at least 75, at least 100, at least 125, at least 150, at least 175, at least 200, at least 250, at least 300, or at least 350, at least 400, at least 450, at least 500, at least 750, but fewer than 813 consecutive nucleotides of SEQ ID NO: 42 or SEQ ID NO: 43. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-810 of SEQ ID NO: 42 or SEQ ID NO: 43 In some embodiments, a polynucleotide of the present disclosure comprises the sequence of nucleic acids 1-810 of SEQ ID NO: 42 or SEQ ID NO: 43.

In some embodiments, a polynucleotide of the present disclosure comprises the coding sequence of a human GJB4 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 44 or SEQ ID NO: 45. In some embodiments, a polynucleotide of the present disclosure comprises the sequence of SEQ ID NO: 44 or SEQ ID NO: 45.

In some embodiments, a polynucleotide of the present disclosure comprises a 5′ truncation, a 3′ truncation, or a fragment of the sequence of SEQ ID NO: 44 or SEQ ID NO: 45. In some embodiments, the 5′ truncation, 3′ truncation, or fragment of the sequence of SEQ ID NO: 44 or SEQ ID NO: 45 is a polynucleotide that has at least 25, at least 50, at least 75, at least 100, at least 125, at least 150, at least 175, at least 200, at least 250, at least 300, or at least 350, at least 400, at least 450, at least 500, at least 750, but fewer than 801 consecutive nucleotides of SEQ ID NO: 44 or SEQ ID NO: 45. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-798 of SEQ ID NO: 44 or SEQ ID NO: 45. In some embodiments, a polynucleotide of the present disclosure comprises the sequence of nucleic acids 1-798 of SEQ ID NO: 44 or SEQ ID NO: 45.

In some embodiments, a polynucleotide of the present disclosure comprises the coding sequence of a human GJB6 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 46 or SEQ ID NO: 47. In some embodiments, a polynucleotide of the present disclosure comprises the sequence of SEQ ID NO: 46 or SEQ ID NO: 47.

In some embodiments, a polynucleotide of the present disclosure comprises a 5′ truncation, a 3′ truncation, or a fragment of the sequence of SEQ ID NO: 46 or SEQ ID NO: 47. In some embodiments, the 5′ truncation, 3′ truncation, or fragment of the sequence of SEQ ID NO: 46 or SEQ ID NO: 47 is a polynucleotide that has at least 25, at least 50, at least 75, at least 100, at least 125, at least 150, at least 175, at least 200, at least 250, at least 300, or at least 350, at least 400, at least 450, at least 500, at least 750, but fewer than 786 consecutive nucleotides of SEQ ID NO: 46 or SEQ ID NO: 47. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-783 of SEQ ID NO: 46 or SEQ ID NO: 47. In some embodiments, a polynucleotide of the present disclosure comprises the sequence of nucleic acids 1-783 of SEQ ID NO: 46 or SEQ ID NO: 47.

In some embodiments, a polynucleotide of the present disclosure comprises the coding sequence of a human KDSR gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 48 or SEQ ID NO: 49. In some embodiments, a polynucleotide of the present disclosure comprises the sequence of SEQ ID NO: 48 or SEQ ID NO: 49.

In some embodiments, a polynucleotide of the present disclosure comprises a 5′ truncation, a 3′ truncation, or a fragment of the sequence of SEQ ID NO: 48 or SEQ ID NO: 49. In some embodiments, the 5′ truncation, 3′ truncation, or fragment of the sequence of SEQ ID NO: 48 or SEQ ID NO: 49 is a polynucleotide that has at least 25, at least 50, at least 75, at least 100, at least 125, at least 150, at least 175, at least 200, at least 250, at least 300, or at least 350, at least 400, at least 450, at least 500, at least 750, but fewer than 999 consecutive nucleotides of SEQ ID NO: 48 or SEQ ID NO: 49. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-996 of SEQ ID NO: 48 or SEQ ID NO: 49. In some embodiments, a polynucleotide of the present disclosure comprises the sequence of nucleic acids 1-996 of SEQ ID NO: 48 or SEQ ID NO: 49.

In some embodiments, a polynucleotide of the present disclosure comprises the coding sequence of a human KRT1 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 50 or SEQ ID NO: 51. In some embodiments, a polynucleotide of the present disclosure comprises the sequence of SEQ ID NO: 50 or SEQ ID NO: 51.

In some embodiments, a polynucleotide of the present disclosure comprises a 5′ truncation, a 3′ truncation, or a fragment of the sequence of SEQ ID NO: 50 or SEQ ID NO: 51. In some embodiments, the 5′ truncation, 3′ truncation, or fragment of the sequence of SEQ ID NO: 50 or SEQ ID NO: 51 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, at least 1500, at least 1750, but fewer than 1935 consecutive nucleotides of SEQ ID NO: 50 or SEQ ID NO: 51. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-1932 of SEQ ID NO: 50 or SEQ ID NO: 51. In some embodiments, a polynucleotide of the present disclosure comprises the sequence of nucleic acids 1-1932 of SEQ ID NO: 50 or SEQ ID NO: 51.

In some embodiments, a polynucleotide of the present disclosure comprises the coding sequence of a human KRT2 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 52 or SEQ ID NO: 53. In some embodiments, a polynucleotide of the present disclosure comprises the sequence of SEQ ID NO: 52 or SEQ ID NO: 53.

In some embodiments, a polynucleotide of the present disclosure comprises a 5′ truncation, a 3′ truncation, or a fragment of the sequence of SEQ ID NO: 52 or SEQ ID NO: 53. In some embodiments, the 5′ truncation, 3′ truncation, or fragment of the sequence of SEQ ID NO: 52 or SEQ ID NO: 53 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, at least 1500, at least 1750, but fewer than 1920 consecutive nucleotides of SEQ ID NO: 52 or SEQ ID NO: 53. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-1917 of SEQ ID NO: 52 or SEQ ID NO: 53. In some embodiments, a polynucleotide of the present disclosure comprises the sequence of nucleic acids 1-1917 of SEQ ID NO: 52 or SEQ ID NO: 53.

In some embodiments, a polynucleotide of the present disclosure comprises the coding sequence of a human KRT9 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 97%, at least 98%, at least 99%, or 100% sequence identity to the sequence of SEQ ID NO: 54 or SEQ ID NO: 55. In some embodiments, a polynucleotide of the present disclosure comprises the sequence of SEQ ID NO: 54 or SEQ ID NO: 55.

In some embodiments, a polynucleotide of the present disclosure comprises a 5′ truncation, a 3′ truncation, or a fragment of the sequence of SEQ ID NO: 54 or SEQ ID NO: 55. In some embodiments, the 5′ truncation, 3′ truncation, or fragment of the sequence of SEQ ID NO: 54 or SEQ ID NO: 55 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, at least 1500, at least 1750, but fewer than 1872 consecutive nucleotides of SEQ ID NO: 54 or SEQ ID NO: 55. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-1869 of SEQ ID NO: 54 or SEQ ID NO: 55. In some embodiments, a polynucleotide of the present disclosure comprises the sequence of nucleic acids 1-1869 of SEQ ID NO: 54 or SEQ ID NO: 55.

In some embodiments, a polynucleotide of the present disclosure comprises the coding sequence of a human KRT10 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 56 or SEQ ID NO: 57. In some embodiments, a polynucleotide of the present disclosure comprises the sequence of SEQ ID NO: 56 or SEQ ID NO: 57.

In some embodiments, a polynucleotide of the present disclosure comprises a 5′ truncation, a 3′ truncation, or a fragment of the sequence of SEQ ID NO: 56 or SEQ ID NO: 57. In some embodiments, the 5′ truncation, 3′ truncation, or fragment of the sequence of SEQ ID NO: 56 or SEQ ID NO: 57 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, at least 1500, at least 1750, but fewer than 1755 consecutive nucleotides of SEQ ID NO: 56 or SEQ ID NO: 57. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-1752 of SEQ ID NO: 56 or SEQ ID NO: 57. In some embodiments, a polynucleotide of the present disclosure comprises the sequence of nucleic acids 1-1752 of SEQ ID NO: 56 or SEQ ID NO: 57.

In some embodiments, a polynucleotide of the present disclosure comprises the coding sequence of a human LIPN gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 58 or SEQ ID NO: 59. In some embodiments, a polynucleotide of the present disclosure comprises the sequence of SEQ ID NO: 58 or SEQ ID NO: 59.

In some embodiments, a polynucleotide of the present disclosure comprises a 5′ truncation, a 3′ truncation, or a fragment of the sequence of SEQ ID NO: 58 or SEQ ID NO: 59. In some embodiments, the 5′ truncation, 3′ truncation, or fragment of the sequence of SEQ ID NO: 58 or SEQ ID NO: 59 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, but fewer than 1197 consecutive nucleotides of SEQ ID NO: 58 or SEQ ID NO: 59. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-1194 of SEQ ID NO: 58 or SEQ ID NO: 59. In some embodiments, a polynucleotide of the present disclosure comprises the sequence of nucleic acids 1-1194 of SEQ ID NO: 58 or SEQ ID NO: 59.

In some embodiments, a polynucleotide of the present disclosure comprises the coding sequence of a human LOR gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 97%, at least 98%, at least 99%, or 100% sequence identity to the sequence of SEQ ID NO: 60 or SEQ ID NO: 61. In some embodiments, a polynucleotide of the present disclosure comprises the sequence of SEQ ID NO: 60 or SEQ ID NO: 61.

In some embodiments, a polynucleotide of the present disclosure comprises a 5′ truncation, a 3′ truncation, or a fragment of the sequence of SEQ ID NO: 60 or SEQ ID NO: 61. In some embodiments, the 5′ truncation, 3′ truncation, or fragment of the sequence of SEQ ID NO: 60 or SEQ ID NO: 61 is a polynucleotide that has at least 25, at least 50, at least 75, at least 100, at least 125, at least 150, at least 175, at least 200, at least 250, at least 300, or at least 350, at least 400, at least 450, at least 500, at least 750, but fewer than 939 consecutive nucleotides of SEQ ID NO: 60 or SEQ ID NO: 61. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-936 of SEQ ID NO: 60 or SEQ ID NO: 61. In some embodiments, a polynucleotide of the present disclosure comprises the sequence of nucleic acids 1-936 of SEQ ID NO: 60 or SEQ ID NO: 61.

In some embodiments, a polynucleotide of the present disclosure comprises the coding sequence of a human MBTPS2 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 62 or SEQ ID NO: 63. In some embodiments, a polynucleotide of the present disclosure comprises the sequence of SEQ ID NO: 62 or SEQ ID NO: 63.

In some embodiments, a polynucleotide of the present disclosure comprises a 5′ truncation, a 3′ truncation, or a fragment of the sequence of SEQ ID NO: 62 or SEQ ID NO: 63. In some embodiments, the 5′ truncation, 3′ truncation, or fragment of the sequence of SEQ ID NO: 62 or SEQ ID NO: 63 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, at least 1500, but fewer than 1560 consecutive nucleotides of SEQ ID NO: 62 or SEQ ID NO: 63. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-1557 of SEQ ID NO: 62 or SEQ ID NO: 63. In some embodiments, a polynucleotide of the present disclosure comprises the sequence of nucleic acids 1-1557 of SEQ ID NO: 62 or SEQ ID NO: 63.

In some embodiments, a polynucleotide of the present disclosure comprises the coding sequence of a human NIPAL4 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NOS: 64-67. In some embodiments, a polynucleotide of the present disclosure comprises a sequence selected from SEQ ID NOS: 64-67. In some embodiments, a polynucleotide of the present disclosure comprises the sequence of SEQ ID NO: 64 or SEQ ID NO: 65.

In some embodiments, a polynucleotide of the present disclosure comprises a 5′ truncation, a 3′ truncation, or a fragment of the sequence of SEQ ID NO: 64 or SEQ ID NO: 65. In some embodiments, the 5′ truncation, 3′ truncation, or fragment of the sequence of SEQ ID NO: 64 or SEQ ID NO: 65 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, but fewer than 1401 consecutive nucleotides of SEQ ID NO: 64 or SEQ ID NO: 65. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-1398 of SEQ ID NO: 64 or SEQ ID NO: 65. In some embodiments, a polynucleotide of the present disclosure comprises the sequence of nucleic acids 1-1398 of SEQ ID NO: 64 or SEQ ID NO: 65.

In some embodiments, a polynucleotide of the present disclosure comprises a 5′ truncation, a 3′ truncation, or a fragment of the sequence of SEQ ID NO: 66 or SEQ ID NO: 67. In some embodiments, the 5′ truncation, 3′ truncation, or fragment of the sequence of SEQ ID NO: 66 or SEQ ID NO: 67 is a polynucleotide that has at least 25, at least 50, at least or at least 350, at least 400, at least 450, at least 500, at least 750, at least 1000, at least 1250, but fewer than 1344 consecutive nucleotides of SEQ ID NO: 66 or SEQ ID NO: 67. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-1341 of SEQ ID NO: 66 or SEQ ID NO: 67. In some embodiments, a polynucleotide of the present disclosure comprises the sequence of nucleic acids 1-1341 of SEQ ID NO: 66 or SEQ ID NO: 67.

In some embodiments, a polynucleotide of the present disclosure comprises the coding sequence of a human NSDHL gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 68 or SEQ ID NO: 69. In some embodiments, a polynucleotide of the present disclosure comprises the sequence of SEQ ID NO: 68 or SEQ ID NO: 69.

In some embodiments, a polynucleotide of the present disclosure comprises a 5′ truncation, a 3′ truncation, or a fragment of the sequence of SEQ ID NO: 68 or SEQ ID NO: 69. In some embodiments, the 5′ truncation, 3′ truncation, or fragment of the sequence of SEQ ID NO: 68 or SEQ ID NO: 69 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, but fewer than 1122 consecutive nucleotides of SEQ ID NO: 68 or SEQ ID NO: 69. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-1119 of SEQ ID NO: 68 or SEQ ID NO: 69. In some embodiments, a polynucleotide of the present disclosure comprises the sequence of nucleic acids 1-1119 of SEQ ID NO: 68 or SEQ ID NO: 69.

In some embodiments, a polynucleotide of the present disclosure comprises the coding sequence of a human PEX7 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 70 or SEQ ID NO: 71. In some embodiments, a polynucleotide of the present disclosure comprises the sequence of SEQ ID NO: 70 or SEQ ID NO: 71.

In some embodiments, a polynucleotide of the present disclosure comprises a 5′ truncation, a 3′ truncation, or a fragment of the sequence of SEQ ID NO: 70 or SEQ ID NO: 71. In some embodiments, the 5′ truncation, 3′ truncation, or fragment of the sequence of SEQ ID NO: 70 or SEQ ID NO: 71 is a polynucleotide that has at least 25, at least 50, at least 75, at least 100, at least 125, at least 150, at least 175, at least 200, at least 250, at least 300, or at least 350, at least 400, at least 450, at least 500, at least 750, but fewer than 972 consecutive nucleotides of SEQ ID NO: 70 or SEQ ID NO: 71. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-969 of SEQ ID NO: 70 or SEQ ID NO: 71. In some embodiments, a polynucleotide of the present disclosure comprises the sequence of nucleic acids 1-969 of SEQ ID NO: 70 or SEQ ID NO: 71.

In some embodiments, a polynucleotide of the present disclosure comprises the coding sequence of a human PHGDH gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 72 or SEQ ID NO: 73. In some embodiments, a polynucleotide of the present disclosure comprises the sequence of SEQ ID NO: 72 or SEQ ID NO: 73.

In some embodiments, a polynucleotide of the present disclosure comprises a 5′ truncation, a 3′ truncation, or a fragment of the sequence of SEQ ID NO: 72 or SEQ ID NO: 73. In some embodiments, the 5′ truncation, 3′ truncation, or fragment of the sequence of SEQ ID NO: 72 or SEQ ID NO: 73 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, at least 1500, but fewer than 1602 consecutive nucleotides of SEQ ID NO: 72 or SEQ ID NO: 73. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-1599 of SEQ ID NO: 72 or SEQ ID NO: 73. In some embodiments, a polynucleotide of the present disclosure comprises the sequence of nucleic acids 1-1599 of SEQ ID NO: 72 or SEQ ID NO: 73.

In some embodiments, a polynucleotide of the present disclosure comprises the coding sequence of a human PHYH gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 74 or SEQ ID NO: 75. In some embodiments, a polynucleotide of the present disclosure comprises the sequence of SEQ ID NO: 74 or SEQ ID NO: 75.

In some embodiments, a polynucleotide of the present disclosure comprises a 5′ truncation, a 3′ truncation, or a fragment of the sequence of SEQ ID NO: 74 or SEQ ID NO: 75. In some embodiments, the 5′ truncation, 3′ truncation, or fragment of the sequence of SEQ ID NO: 74 or SEQ ID NO: 75 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, but fewer than 1017 consecutive nucleotides of SEQ ID NO: 74 or SEQ ID NO: 75. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-1014 of SEQ ID NO: 74 or SEQ ID NO: 75. In some embodiments, a polynucleotide of the present disclosure comprises the sequence of nucleic acids 1-1014 of SEQ ID NO: 74 or SEQ ID NO: 75.

In some embodiments, a polynucleotide of the present disclosure comprises the coding sequence of a human PNPLA1 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence selected from SEQ ID NOS: 76-81. In some embodiments, a polynucleotide of the present disclosure comprises a sequence selected from SEQ ID NOS: 76-81.

In some embodiments, a polynucleotide of the present disclosure comprises a 5′ truncation, a 3′ truncation, or a fragment of the sequence of SEQ ID NO: 76 or SEQ ID NO: 77. In some embodiments, the 5′ truncation, 3′ truncation, or fragment of the sequence of SEQ ID NO: 76 or SEQ ID NO: 77 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, at least 1500, but fewer than 1599 consecutive nucleotides of SEQ ID NO: 76 or SEQ ID NO: 77. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-1596 of SEQ ID NO: 76 or SEQ ID NO: 77. In some embodiments, a polynucleotide of the present disclosure comprises the sequence of nucleic acids 1-1596 of SEQ ID NO: 76 or SEQ ID NO: 77.

In some embodiments, a polynucleotide of the present disclosure comprises a 5′ truncation, a 3′ truncation, or a fragment of the sequence of SEQ ID NO: 78 or SEQ ID NO: 79. In some embodiments, the 5′ truncation, 3′ truncation, or fragment of the sequence of SEQ ID NO: 78 or SEQ ID NO: 79 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, but fewer than 1314 consecutive nucleotides of SEQ ID NO: 78 or SEQ ID NO: 79. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-1311 of SEQ ID NO: 78 or SEQ ID NO: 79. In some embodiments, a polynucleotide of the present disclosure comprises the sequence of nucleic acids 1-1311 of SEQ ID NO: 78 or SEQ ID NO: 79.

In some embodiments, a polynucleotide of the present disclosure comprises a 5′ truncation, a 3′ truncation, or a fragment of the sequence of SEQ ID NO: 80 or SEQ ID NO: 81. In some embodiments, the 5′ truncation, 3′ truncation, or fragment of the sequence of SEQ ID NO: 80 or SEQ ID NO: 81 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, but fewer than 1341 consecutive nucleotides of SEQ ID NO: 80 or SEQ ID NO: 81. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-1338 of SEQ ID NO: 80 or SEQ ID NO: 81. In some embodiments, a polynucleotide of the present disclosure comprises the sequence of nucleic acids 1-1338 of SEQ ID NO: 80 or SEQ ID NO: 81.

In some embodiments, a polynucleotide of the present disclosure comprises the coding sequence of a human POMP gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 82 or SEQ ID NO: 83. In some embodiments, a polynucleotide of the present disclosure comprises the sequence of SEQ ID NO: 82 or SEQ ID NO: 83.

In some embodiments, a polynucleotide of the present disclosure comprises a 5′ truncation, a 3′ truncation, or a fragment of the sequence of SEQ ID NO: 82 or SEQ ID NO: 83. In some embodiments, the 5′ truncation, 3′ truncation, or fragment of the sequence of SEQ ID NO: 82 or SEQ ID NO: 83 is a polynucleotide that has at least 25, at least 50, at least 75, at least 100, at least 125, at least 150, at least 175, at least 200, at least 250, at least 300, or at least 350, at least 400, but fewer than 426 consecutive nucleotides of SEQ ID NO: 82 or SEQ ID NO: 83. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-423 of SEQ ID NO: 82 or SEQ ID NO: 83. In some embodiments, a polynucleotide of the present disclosure comprises the sequence of nucleic acids 1-423 of SEQ ID NO: 82 or SEQ ID NO: 83.

In some embodiments, a polynucleotide of the present disclosure comprises the coding sequence of a human PSAT1 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 84 or SEQ ID NO: 85. In some embodiments, a polynucleotide of the present disclosure comprises the sequence of SEQ ID NO: 84 or SEQ ID NO: 85.

In some embodiments, a polynucleotide of the present disclosure comprises a 5′ truncation, a 3′ truncation, or a fragment of the sequence of SEQ ID NO: 84 or SEQ ID NO: 85. In some embodiments, the 5′ truncation, 3′ truncation, or fragment of the sequence of SEQ ID NO: 84 or SEQ ID NO: 85 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, but fewer than 1113 consecutive nucleotides of SEQ ID NO: 84 or SEQ ID NO: 85. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-1110 of SEQ ID NO: 84 or SEQ ID NO: 85. In some embodiments, a polynucleotide of the present disclosure comprises the sequence of nucleic acids 1-1110 of SEQ ID NO: 84 or SEQ ID NO: 85.

In some embodiments, a polynucleotide of the present disclosure comprises the coding sequence of a human SDR9C7 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 86 or SEQ ID NO: 87. In some embodiments, a polynucleotide of the present disclosure comprises the sequence of SEQ ID NO: 86 or SEQ ID NO: 87.

In some embodiments, a polynucleotide of the present disclosure comprises a 5′ truncation, a 3′ truncation, or a fragment of the sequence of SEQ ID NO: 86 or SEQ ID NO: 87. In some embodiments, the 5′ truncation, 3′ truncation, or fragment of the sequence of SEQ ID NO: 86 or SEQ ID NO: 87 is a polynucleotide that has at least 25, at least 50, at least 75, at least 100, at least 125, at least 150, at least 175, at least 200, at least 250, at least 300, or at least 350, at least 400, at least 450, at least 500, at least 750, but fewer than 942 consecutive nucleotides of SEQ ID NO: 86 or SEQ ID NO: 87. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-939 of SEQ ID NO: 86 or SEQ ID NO: 87. In some embodiments, a polynucleotide of the present disclosure comprises the sequence of nucleic acids 1-939 of SEQ ID NO: 86 or SEQ ID NO: 87.

In some embodiments, a polynucleotide of the present disclosure comprises the coding sequence of a human SERPINB8 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 88 or SEQ ID NO: 89. In some embodiments, a polynucleotide of the present disclosure comprises the sequence of SEQ ID NO: 88 or SEQ ID NO: 89.

In some embodiments, a polynucleotide of the present disclosure comprises a 5′ truncation, a 3′ truncation, or a fragment of the sequence of SEQ ID NO: 88 or SEQ ID NO: 89. In some embodiments, the 5′ truncation, 3′ truncation, or fragment of the sequence of SEQ ID NO: 88 or SEQ ID NO: 89 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, but fewer than 1125 consecutive nucleotides of SEQ ID NO: 88 or SEQ ID NO: 89. In some embodiments, a polynucleotide of the present disclosure comprises a sequence having at least 97%, at least 98%, at least 99%, or 100% sequence identity to the sequence of nucleic acids 1-1122 of SEQ ID NO: 88 or SEQ ID NO: 89. In some embodiments, a polynucleotide of the present disclosure comprises the sequence of nucleic acids 1-1125 of SEQ ID NO: 88 or SEQ ID NO: 89.

In some embodiments, a polynucleotide of the present disclosure comprises the coding sequence of a human SLC27A4 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 90 or SEQ ID NO: 91. In some embodiments, a polynucleotide of the present disclosure comprises the sequence of SEQ ID NO: 90 or SEQ ID NO: 91.

In some embodiments, a polynucleotide of the present disclosure comprises a 5′ truncation, a 3′ truncation, or a fragment of the sequence of SEQ ID NO: 90 or SEQ ID NO: 91. In some embodiments, the 5′ truncation, 3′ truncation, or fragment of the sequence of SEQ ID NO: 90 or SEQ ID NO: 91 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, at least 1500, at least 1750, but fewer than 1932 consecutive nucleotides of SEQ ID NO: 90 or SEQ ID NO: 91. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-1929 of SEQ ID NO: 90 or SEQ ID NO: 91. In some embodiments, a polynucleotide of the present disclosure comprises the sequence of nucleic acids 1-1929 of SEQ ID NO: 90 or SEQ ID NO: 91.

In some embodiments, a polynucleotide of the present disclosure comprises the coding sequence of a human SNAP29 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 92 or SEQ ID NO: 93. In some embodiments, a polynucleotide of the present disclosure comprises the sequence of SEQ ID NO: 92 or SEQ ID NO: 93.

In some embodiments, a polynucleotide of the present disclosure comprises a 5′ truncation, a 3′ truncation, or a fragment of the sequence of SEQ ID NO: 92 or SEQ ID NO: 93. In some embodiments, the 5′ truncation, 3′ truncation, or fragment of the sequence of SEQ ID NO: 92 or SEQ ID NO: 93 is a polynucleotide that has at least 25, at least 50, at least 75, at least 100, at least 125, at least 150, at least 175, at least 200, at least 250, at least 300, or at least 350, at least 400, at least 450, at least 500, at least 750, but fewer than 777 consecutive nucleotides of SEQ ID NO: 92 or SEQ ID NO: 93. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-774 of SEQ ID NO: 92 or SEQ ID NO: 93. In some embodiments, a polynucleotide of the present disclosure comprises the sequence of nucleic acids 1-774 of SEQ ID NO: 92 or SEQ ID NO: 93.

In some embodiments, a polynucleotide of the present disclosure comprises the coding sequence of a human ST14 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 94 or SEQ ID NO: 95. In some embodiments, a polynucleotide of the present disclosure comprises the sequence of SEQ ID NO: 94 or SEQ ID NO: 95.

In some embodiments, a polynucleotide of the present disclosure comprises a 5′ truncation, a 3′ truncation, or a fragment of the sequence of SEQ ID NO: 94 or SEQ ID NO: 95. In some embodiments, the 5′ truncation, 3′ truncation, or fragment of the sequence of SEQ ID NO: 94 or SEQ ID NO: 95 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, at least 1500, at least 1750, at least 2000, at least 2500, but fewer than 2568 consecutive nucleotides of SEQ ID NO: 94 or SEQ ID NO: 95. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-2565 of SEQ ID NO: 94 or SEQ ID NO: 95. In some embodiments, a polynucleotide of the present disclosure comprises the sequence of nucleic acids 1-2565 of SEQ ID NO: 94 or SEQ ID NO: 95.

In some embodiments, a polynucleotide of the present disclosure comprises the coding sequence of a human STS gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 96 or SEQ ID NO: 97. In some embodiments, a polynucleotide of the present disclosure comprises the sequence of SEQ ID NO: 96 or SEQ ID NO: 97.

In some embodiments, a polynucleotide of the present disclosure comprises a 5′ truncation, a 3′ truncation, or a fragment of the sequence of SEQ ID NO: 96 or SEQ ID NO: 97. In some embodiments, the 5′ truncation, 3′ truncation, or fragment of the sequence of SEQ ID NO: 96 or SEQ ID NO: 97 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, at least 1500, but fewer than 1752 consecutive nucleotides of SEQ ID NO: 96 or SEQ ID NO: 97. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-1749 of SEQ ID NO: 96 or SEQ ID NO: 97. In some embodiments, a polynucleotide of the present disclosure comprises the sequence of nucleic acids 1-1749 of SEQ ID NO: 96 or SEQ ID NO: 97.

In some embodiments, a polynucleotide of the present disclosure comprises the coding sequence of a human SULT2B1 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 153 or SEQ ID NO: 154. In some embodiments, a polynucleotide of the present disclosure comprises the sequence of SEQ ID NO: 153 or SEQ ID NO: 154.

In some embodiments, a polynucleotide of the present disclosure comprises a 5′ truncation, a 3′ truncation, or a fragment of the sequence of SEQ ID NO: 153 or SEQ ID NO: 154. In some embodiments, the 5′ truncation, 3′ truncation, or fragment of the sequence of SEQ ID NO: 153 or SEQ ID NO: 154 is a polynucleotide that has at least 25, at least 50, at least 75, at least 100, at least 125, at least 150, at least 175, at least 200, at least 250, at least 300, or at least 350, at least 400, at least 450, at least 500, at least 600, at least 700, at least 800, at least 900, at least 1000, at least 1050, but fewer than 1098 consecutive nucleotides of SEQ ID NO: 153 or SEQ ID NO: 154. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-1095 of SEQ ID NO: 153 or SEQ ID NO: 154. In some embodiments, a polynucleotide of the present disclosure comprises the sequence of nucleic acids 1-1095 of SEQ ID NO: 153 or SEQ ID NO: 154.

In some embodiments, a polynucleotide of the present disclosure comprises the coding sequence of a human VPS33B gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 98 or SEQ ID NO: 99. In some embodiments, a polynucleotide of the present disclosure comprises the sequence of SEQ ID NO: 98 or SEQ ID NO: 99.

In some embodiments, a polynucleotide of the present disclosure comprises a 5′ truncation, a 3′ truncation, or a fragment of the sequence of SEQ ID NO: 98 or SEQ ID NO: 99. In some embodiments, the 5′ truncation, 3′ truncation, or fragment of the sequence of SEQ ID NO: 98 or SEQ ID NO: 99 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, at least 1500, at least 1750, but fewer than 1854 consecutive nucleotides of SEQ ID NO: 98 or SEQ ID NO: 99. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-1851 of SEQ ID NO: 98 or SEQ ID NO: 99. In some embodiments, a polynucleotide of the present disclosure comprises the sequence of nucleic acids 1-1851 of SEQ ID NO: 98 or SEQ ID NO: 99.

In some embodiments, a polynucleotide of the present disclosure comprises the coding sequence of a human ZMPSTE24 gene (or a codon-optimized variant thereof). In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 100 or SEQ ID NO: 101. In some embodiments, a polynucleotide of the present disclosure comprises the sequence of SEQ ID NO: 100 or SEQ ID NO: 101.

In some embodiments, a polynucleotide of the present disclosure comprises a 5′ truncation, a 3′ truncation, or a fragment of the sequence of SEQ ID NO: 100 or SEQ ID NO: 101. In some embodiments, the 5′ truncation, 3′ truncation, or fragment of the sequence of SEQ ID NO: 100 or SEQ ID NO: 101 is a polynucleotide that has at least 25, at least 50, at least 75, at least 100, at least 125, at least 150, at least 175, at least 200, at least 250, at least 300, or at least 350, at least 400, at least 450, at least 500, at least 750, at least 1000, at least 1250, but fewer than 1428 consecutive nucleotides of SEQ ID NO: 100 or SEQ ID NO: 101. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of nucleic acids 1-1425 of SEQ ID NO: 100 or SEQ ID NO: 101. In some embodiments, a polynucleotide of the present disclosure comprises the sequence of nucleic acids 1-1425 of SEQ ID NO: 100 or SEQ ID NO: 101.

In some embodiments, expression of an ichthyosis-associated gene (e.g., as described above) in one or more cells of a subject in need thereof (e.g., a subject harboring one or more pathogenic variants and/or loss-of-function mutations in one or both copies of the corresponding endogenous gene) is beneficial for providing prophylactic, palliative, and/or therapeutic relief of one or more signs or symptoms of an autosomal dominant, autosomal semi-dominant, autosomal recessive, X-linked dominant, and/or X-linked recessive form of congenital ichthyosis.

In some embodiments, expression of an ichthyosis-associated gene (e.g., ABCA12, ABHD5, ALDH3A2, ALOX12B, ALOXE3, AP1S1, ARSE, CASP14, CDSN, CERS3, CHST8, CLDN1, CSTA, CYP4F22, ELOVL4, KDSR, LIPN, MBTPS2, NIPAL4, PEX7, PHGDH, PHYH, PNPLA1, POMP, PSAT1, SDR9C7, SERPINB8, SLC27A4, SNAP29, ST14, STS, SULT2B1, VPS33B, ZMPSTE24) in one or more cells of a subject in need thereof (e.g., a subject harboring one or more pathogenic variants and/or loss-of-function mutations in one or both copies of the corresponding endogenous gene) is beneficial for providing prophylactic, palliative, and/or therapeutic relief of one or more signs or symptoms of an autosomal recessive and/or X-linked recessive form of congenital ichthyosis.

In some embodiments, expression of an ichthyosis-associated gene (e.g., ABCA12, ABHD5, ALDH3A2, ALOX12B, ALOXE3, AP1S1, CASP14, CDSN, CERS3, CHST8, CLDN1, CSTA, CYP4F22, ELOVL4, KDSR, LIPN, NIPAL4, PEX7, PHGDH, PHYH, PNPLA1, POMP, PSAT1, SDR9C7, SERPINB8, SLC27A4, SNAP29, ST14, SULT2B1, VPS33B, ZMPSTE24) in one or more cells of a subject in need thereof (e.g., a subject harboring one or more pathogenic variants and/or loss-of-function mutations in one or both copies of the corresponding endogenous gene) is beneficial for providing prophylactic, palliative, and/or therapeutic relief of one or more signs or symptoms of an autosomal recessive form of congenital ichthyosis.

In some embodiments, expression of an ichthyosis-associated gene (e.g., ARSE, MBTPS2, STS) in one or more cells of a subject in need thereof (e.g., a subject harboring one or more pathogenic variants and/or loss-of-function mutations in one or both copies of the corresponding endogenous gene) is beneficial for providing prophylactic, palliative, and/or therapeutic relief of one or more signs or symptoms of an X-linked recessive form of congenital ichthyosis.

A polynucleotide of the present disclosure (e.g., comprising the coding sequence of an ichthyosis-associated gene (i.e., encoding an ichthyosis-associated polypeptide)) may further encode additional coding and non-coding sequences. Examples of additional coding and non-coding sequences may include, but are not limited to, sequences encoding additional polypeptide tags (e.g., encoded in-frame with the ichthyosis-associated polypeptide in order to produce a fusion protein), introns (e.g., native, modified, or heterologous introns), 5′ and/or 3′ UTRs (e.g., native, modified, or heterologous 5′ and/or 3′ UTRs), and the like. Examples of suitable polypeptide tags may include, but are not limited, to any combination of purification tags, such as his-tags, flag-tags, maltose binding protein and glutathione-S-transferase tags, detection tags, such as tags that may be detected photometrically (e.g., green fluorescent protein, red fluorescent protein, etc.) and tags that have a detectable enzymatic activity (e.g., alkaline phosphatase, etc.), tags containing secretory sequences, signal sequences, leader sequences, and/or stabilizing sequences, protease cleavage sites (e.g., furin cleavage sites, TEV cleavage sites, Thrombin cleavage sites, etc.), and the like. In some embodiments, the 5′ and/or 3′UTRs increase the stability, localization, and/or translational efficiency of the polynucleotides. In some embodiments, the 5′ and/or 3′UTRs improve the level and/or duration of protein expression. In some embodiments, the 5′ and/or 3′UTRs include elements (e.g., one or more miRNA binding sites, etc.) that may block or reduce off-target expression (e.g., inhibiting expression in specific cell types (e.g., neuronal cells), at specific times in the cell cycle, at specific developmental stages, etc.). In some embodiments, the 5′ and/or 3′UTRs include elements (e.g., one or more miRNA binding sites, etc.) that may enhance effector protein expression in specific cell types (such as human keratinocytes and/or fibroblasts).

In some embodiments, a polynucleotide of the present disclosure is operably linked to one or more (e.g., one or more, two or more, three or more, four or more, five or more, ten or more, etc.) regulatory sequences. The term “regulatory sequence” may include enhancers, insulators, promoters, and other expression control elements (e.g., polyadenylation signals). Any suitable enhancer(s) known in the art may be used, including, for example, enhancer sequences from mammalian genes (such as globin, elastase, albumin, α-fetoprotein, insulin and the like), enhancer sequences from a eukaryotic cell virus (such as SV40 enhancer on the late side of the replication origin (bp 100-270), the cytomegalovirus early promoter enhancer, the polyoma enhancer on the late side of the replication origin, adenovirus enhancers, and the like), and any combinations thereof. Any suitable insulator(s) known in the art may be used, including, for example, herpes simplex virus (HSV) chromatin boundary (CTRL/CTCF-binding/insulator) elements CTRL1 and/or CTRL2, chicken hypersensitive site 4 insulator (cHS4), human HNRPA2B1-CBX3 ubiquitous chromatin opening element (UCOE), the scaffold/matrix attachment region (S/MAR) from the human interferon beta gene (IFNB1), and any combinations thereof. Any suitable promoter (e.g., suitable for transcription in mammalian host cells) known in the art may be used, including, for example, promoters obtained from the genomes of viruses (such as polyoma virus, fowlpox virus, adenovirus (such as Adenovirus 2), bovine papilloma virus, avian sarcoma virus, cytomegalovirus, a retrovirus, hepatitis-B virus, Simian Virus 40 (SV40), and the like), promoters from heterologous mammalian genes (such as the actin promoter (e.g., the (3-actin promoter), a ubiquitin promoter (e.g., a ubiquitin C (UbC) promoter), a phosphoglycerate kinase (PGK) promoter, an immunoglobulin promoter, from heat-shock protein promoters, and the like), promoters from native and/or homologous mammalian genes, synthetic promoters (such as the CAGG promoter), and any combinations thereof, provided such promoters are compatible with the host cells. Regulatory sequences may include those which direct constitutive expression of a nucleic acid, as well as tissue-specific regulatory and/or inducible or repressible sequences.

In some embodiments, a polynucleotide of the present disclosure is operably linked to one or more heterologous promoters. In some embodiments, the one or more heterologous promoters are one or more of constitutive promoters, tissue-specific promoters, temporal promoters, spatial promoters, inducible promoters, and repressible promoters. In some embodiments, the one or more heterologous promoters are one or more of the human cytomegalovirus (HCMV) immediate early promoter, the human elongation factor-1 (EF1) promoter, the human (3-actin promoter, the human UbC promoter, the human PGK promoter, the synthetic CAGG promoter, and any combinations thereof. In some embodiments, a polynucleotide of the present disclosure is operably linked to an HCMV promoter.

In some embodiments, a recombinant nucleic acid of the present disclosure does not comprise a polynucleotide comprising the coding sequence of an ichthyosis-associated gene (i.e., encoding an ichthyosis-associated polypeptide) does not comprise the coding sequence of a transglutaminase gene (i.e., does not encode a transglutaminase polypeptide), such as a TGM1 gene or a TGM5 gene. In some embodiments, a recombinant nucleic acid of the present disclosure does not comprise a polynucleotide comprising the coding sequence of a human transglutaminase gene (i.e., does not encode a human transglutaminase polypeptide), such as a human TGM1 gene or a human TGM5 gene. In some embodiments, a recombinant nucleic acid of the present disclosure does not comprise a polynucleotide comprising the coding sequence of a filaggrin gene or filaggrin 2 gene (i.e., does not encode a filaggrin or filaggrin 2 polypeptide), such as a human FLG gene or a human FLG2 gene. In some embodiments, a recombinant nucleic acid of the present disclosure does not comprise a polynucleotide comprising the coding sequence of a keratin gene (i.e., does not encode a keratin polypeptide), such as a KRT1, KRT2, KRT9, KRT10, and/or KRT17 gene. In some embodiments, a recombinant nucleic acid of the present disclosure does not comprise a polynucleotide comprising the coding sequence of a human keratin gene (i.e., does not encode a human keratin polypeptide), such as a human KRT1 gene, a human KRT2 gene, a human KRT9 gene, a human KRT10 gene, and/or a human KRT17 gene.

In some embodiments, a recombinant nucleic acid of the present disclosure does not comprise a polynucleotide comprising the coding sequence of (e.g., a transgene encoding) a Collagen alpha-1 (VII) chain polypeptide (COLT). In some embodiments, a recombinant nucleic acid of the present disclosure does not comprise a polynucleotide comprising the coding sequence of (e.g., a transgene encoding) a Lysyl hydroxylase 3 polypeptide (LH3). In some embodiments, a recombinant nucleic acid of the present disclosure does not comprise a polynucleotide comparing the coding sequence of (e.g., a transgene encoding) a Keratin type I cytoskeletal 17 polypeptide (KRT17). In some embodiments, a recombinant nucleic acid of the present disclosure does not comprise a polynucleotide comprising the coding sequence of (e.g., a transgene encoding) a transglutaminase (TGM) polypeptide (e.g., a human transglutaminase polypeptide such as a human TGM1 polypeptide and/or a human TGM5 polypeptide). In some embodiments, a recombinant nucleic acid of the present disclosure does not comprise a polynucleotide comprising the coding sequence of (e.g., a transgene encoding) a cosmetic protein (e.g., collagen proteins, fibronectins, elastins, lumicans, vitronectins/vitronectin receptors, laminins, neuromodulators, fibrillins, additional dermal extracellular matrix proteins, etc.). In some embodiments, a recombinant nucleic acid of the present disclosure does not comprise a polynucleotide comprising the coding sequence of (e.g., a transgene encoding) an antibody (e.g., a full-length antibody, an antibody fragments, etc.). In some embodiments, a recombinant nucleic acid of the present disclosure does not comprise a polynucleotide comprising the coding sequence of (e.g., a transgene encoding) a serine protease inhibitor kazal-type (SPINK) polypeptide (e.g., a human SPINK polypeptide such as a human SPINK5 polypeptide). In some embodiments, a recombinant nucleic acid of the present disclosure does not comprise a polynucleotide comprising the coding sequence of (e.g., a transgene encoding) a laminin polypeptide (e.g., a human laminin polypeptide such as a human LAMA3, LAMB3, and/or LAMC2 polypeptide). In some embodiments, a recombinant nucleic acid of the present disclosure does not comprise a polynucleotide comprising the coding sequence of (e.g., a transgene encoding) a cystic fibrosis transmembrane conductance regulator (CFTR) polypeptide (e.g., a human CFTR polypeptide). In some embodiments, a recombinant nucleic acid of the present disclosure does not comprise a polynucleotide comprising the coding sequence of (e.g., a transgene encoding) a Collagen alpha-1 (VII) chain polypeptide, a Lysyl hydroxylase 3 polypeptide, a Keratin type I cytoskeletal 17 polypeptide, and/or any chimeric polypeptides thereof. In some embodiments, a recombinant nucleic acid of the present disclosure does not comprise a polynucleotide comprising the coding sequence of (e.g., a transgene encoding) a Collagen alpha-1 (VII) chain polypeptide, a Lysyl hydroxylase 3 polypeptide, a Keratin type I cytoskeletal 17 polypeptide, a transglutaminase (TGM) polypeptide, a filaggrin polypeptide, a SPINK polypeptide, a CFTR polypeptide, a cosmetic protein, an antibody, and/or any chimeric polypeptides thereof.

Ichthyosis-Associated Polypeptides

In some embodiments, the present disclosure relates to one or more polynucleotides encoding a full-length ichthyosis-associated polypeptide, or any portions thereof (e.g., functional fragments). Ichthyosis-associated polypeptides may be encoded by any of the ichthyosis-associated genes described herein. Any suitable ichthyosis-associated polypeptide known in the art may be encoded by a polynucleotide of the present disclosure, including, for example, an ATP-binding cassette sub-family A member 12 polypeptide (such as a human ATP-binding cassette sub-family A member 12 polypeptide, e.g., as disclosed by UniProt accession number: Q86UK0), a 1-acylglycerol-3-phosphate O-acyltransferase ABHD5 polypeptide (such as a human 1-acylglycerol-3-phosphate O-acyltransferase ABHD5 polypeptide, e.g., as disclosed by UniProt accession number: Q8WTS1), an Aldehyde dehydrogenase family 3 member A2 polypeptide (such as a human Aldehyde dehydrogenase family 3 member A2 polypeptide, e.g., as disclosed by UniProt accession number: P51648), an Arachidonate 12-lipoxygenase 12R-type polypeptide (such as a human Arachidonate 12-lipoxygenase 12R-type polypeptide, e.g., as disclosed by UniProt accession number: 075342), a Hydroperoxide isomerase ALOXE3 polypeptide (such as a human Hydroperoxide isomerase ALOXE3 polypeptide, e.g., as disclosed by UniProt accession number: Q9BYJ1), an AP-1 complex subunit sigma-1A polypeptide (such as a human AP-1 complex subunit sigma-1A polypeptide, e.g., as disclosed by UniProt accession number: P61966), an Arylsulfatase E polypeptide (such as a human Arylsulfatase E polypeptide, e.g., as disclosed by UniProt accession number: P51690), a Caspase-14 polypeptide (such as a human Caspase-14 polypeptide, e.g., as disclosed by UniProt accession number: P31944), a Corneodesmosin polypeptide (such as a human Corneodesmosin polypeptide, e.g., as disclosed by UniProt accession number: Q15517), a Ceramide synthase 3 polypeptide (such as a human Ceramide synthase 3 polypeptide, e.g., as disclosed by UniProt accession number: Q8IU89), a Carbohydrate sulfotransferase 8 polypeptide (such as a human Carbohydrate sulfotransferase 8 polypeptide, e.g., as disclosed by UniProt accession number: Q9H2A9), a Claudin-1 polypeptide (such as a human Claudin-1 polypeptide, e.g., as disclosed by UniProt accession number: 095832), a Cystatin-A polypeptide (such as a human Cystatin-A polypeptide, e.g., as disclosed by UniProt accession number: P01040), a Cytochrome P450 4F22 polypeptide (such as a human Cytochrome P450 4F22 polypeptide, e.g., as disclosed by UniProt accession number: Q6NT55), a 3-beta-hydroxysteroid-Delta(8),Delta(7)-isomerase polypeptide (such as a human 3-beta-hydroxysteroid-Delta(8),Delta(7)-isomerase polypeptide, e.g., as disclosed by UniProt accession number: Q15125), an Elongation of very long chain fatty acids protein 4 polypeptide (such as a human Elongation of very long chain fatty acids protein 4 polypeptide, e.g., as disclosed by UniProt accession number: Q9GZR5), a Filaggrin polypeptide (such as a human Filaggrin polypeptide, e.g., as disclosed by UniProt accession number: P20930), a Filaggrin-2 polypeptide (such as a human Filaggrin-2 polypeptide, e.g., as disclosed by UniProt accession number: Q5D862), a Gap junction beta-2 polypeptide (such as a human Gap junction beta-2 polypeptide, e.g., as disclosed by UniProt accession number: P29033), a Gap junction beta-3 polypeptide (such as a human Gap junction beta-3 polypeptide, e.g., as disclosed by UniProt accession number: 075712), a Gap junction beta-4 polypeptide (such as a human Gap junction beta-4 polypeptide, e.g., as disclosed by UniProt accession number: Q9NTQ9), a Gap junction beta-6 polypeptide (such as a human Gap junction beta-6 polypeptide, e.g., as disclosed by UniProt accession number: 095452), a 3-ketodihydrosphingosine reductase polypeptide (such as a human 3-ketodihydrosphingosine reductase polypeptide, e.g., as disclosed by UniProt accession number: Q06136), a Keratin, type II cytoskeletal 1 polypeptide (such as a human Keratin, type II cytoskeletal 1 polypeptide, e.g., as disclosed by UniProt accession number: P04264), a Keratin, type II cytoskeletal 2 epidermal polypeptide (such as a human Keratin, type II cytoskeletal 2 epidermal polypeptide, e.g., as disclosed by UniProt accession number: P35908), a Keratin, type I cytoskeletal 9 polypeptide (such as a human Keratin, type I cytoskeletal 9 polypeptide, e.g., as disclosed by UniProt accession number: P35527), a Keratin, type I cytoskeletal 10 polypeptide (such as a human Keratin, type I cytoskeletal 10 polypeptide, e.g., as disclosed by UniProt accession number: P13645), a Lipase member N polypeptide (such as a human Lipase member N polypeptide, e.g., as disclosed by UniProt accession number: Q5VXI9), a Loricrin polypeptide (such as a human Loricrin polypeptide, e.g., as disclosed by UniProt accession number: P23490), a Membrane-bound transcription factor site-2 protease polypeptide (such as a human Membrane-bound transcription factor site-2 protease polypeptide, e.g., as disclosed by UniProt accession number: 043462), a Magnesium transporter NIPA4 polypeptide (such as a human Magnesium transporter NIPA4 polypeptide, e.g., as disclosed by UniProt accession number: Q0D2K0), a Sterol-4-alpha-carboxylate 3-dehydrogenase, decarboxylating polypeptide (such as a human Sterol-4-alpha-carboxylate 3-dehydrogenase, decarboxylating polypeptide, e.g., as disclosed by UniProt accession number: Q15738), a Peroxisomal targeting signal 2 receptor polypeptide (such as a human Peroxisomal targeting signal 2 receptor polypeptide, e.g., as disclosed by UniProt accession number: 000628), a D-3-phosphoglycerate dehydrogenase polypeptide (such as a human Peroxisomal targeting signal 2 receptor polypeptide, e.g., as disclosed by UniProt accession number: 043175), a Phytanoyl-CoA dioxygenase, peroxisomal polypeptide (such as a human Phytanoyl-CoA dioxygenase, peroxisomal polypeptide, e.g., as disclosed by UniProt accession number: 014832), a Patatin-like phospholipase domain-containing protein 1 polypeptide (such as a human Patatin-like phospholipase domain-containing protein 1 polypeptide, e.g., as disclosed by UniProt accession number: Q8N8W4), a Proteasome maturation protein polypeptide (such as a human Proteasome maturation protein polypeptide, e.g., as disclosed by UniProt accession number: Q9Y244), a Phosphoserine aminotransferase polypeptide (such as a human Phosphoserine aminotransferase polypeptide, e.g., as disclosed by UniProt accession number: Q9Y617), a Short-chain dehydrogenase/reductase family 9C member 7 polypeptide (such as a human Short-chain dehydrogenase/reductase family 9C member 7 polypeptide, e.g., as disclosed by UniProt accession number: Q8NEX9), a Serpin B8 polypeptide (such as a human Serpin B8 polypeptide, e.g., as disclosed by UniProt accession number: P50452), a Long-chain fatty acid transport protein 4 polypeptide (such as a human Long-chain fatty acid transport protein 4 polypeptide, e.g., as disclosed by UniProt accession number: Q6P1M0), a Synaptosomal-associated protein 29 polypeptide (such as a human Synaptosomal-associated protein 29 polypeptide, e.g., as disclosed by UniProt accession number: 095721), a Suppressor of tumorigenicity 14 protein polypeptide (such as a human Suppressor of tumorigenicity 14 protein polypeptide, e.g., as disclosed by UniProt accession number: Q9Y5Y6), a Steryl-sulfatase polypeptide (such as a human Steryl-sulfatase polypeptide, e.g., as disclosed by UniProt accession number: P08842), a Sulfotransferase 2B1 polypeptide (such as a human Sulfotransferase 2B1 polypeptide, e.g., as disclosed by UniProt accession number: 000204), a Vacuolar protein sorting-associated protein 33B polypeptide (such as a human Vacuolar protein sorting-associated protein 33B polypeptide, e.g., as disclosed by UniProt accession number: Q9H267), a CAAX prenyl protease 1 homolog polypeptide (such as a human CAAX prenyl protease 1 homolog polypeptide, e.g., as disclosed by UniProt accession number: 075844), etc. Methods of identifying ichthyosis-associated polypeptide homologs/orthologs from additional species are known to one of ordinary skill in the art, including, for example, using an amino acid sequence alignment program such as the BLAST® blastp suite or OrthoDB. In some embodiments, an ichthyosis-associated polypeptide of the preset disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of any of the ichthyosis-associated polypeptides described herein or known in the art.

In some embodiments, a polynucleotide of the present disclosure encodes a human ATP-binding cassette sub-family A member 12 polypeptide. In some embodiments, the polynucleotide comprises the coding sequence of an ABCA12 gene as described herein. In some embodiments, a polynucleotide encoding an ATP-binding cassette sub-family A member 12 polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 102 or SEQ ID NO: 103. In some embodiments, a polynucleotide encoding an ATP-binding cassette sub-family A member 12 polypeptide is a polynucleotide that encodes a polypeptide comprising the amino acid sequence of SEQ ID NO: 102 or SEQ ID NO: 103.

In some embodiments, a polynucleotide encoding an ATP-binding cassette sub-family A member 12 polypeptide is a polynucleotide that encodes an N-terminal truncation, a C-terminal truncation, or a fragment of the amino acid sequence of SEQ ID NO: 102. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, at least 500, at least 600, at least 700, at least 800, at least 900, at least 1000, at least 1250, at least 1500, at least 1750, at least 2000, at least 2250, at least 2500, but fewer than 2595 consecutive amino acids of SEQ ID NO: 102.

In some embodiments, a polynucleotide encoding an ATP-binding cassette sub-family A member 12 polypeptide is a polynucleotide that encodes an N-terminal truncation, a C-terminal truncation, or a fragment of the amino acid sequence of SEQ ID NO: 103. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, at least 500, at least 600, at least 700, at least 800, at least 900, at least 1000, at least 1250, at least 1500, at least 1750, at least 2000, but fewer than 2277 consecutive amino acids of SEQ ID NO: 103.

In some embodiments, a polynucleotide of the present disclosure encodes a human 1-acylglycerol-3-phosphate O-acyltransferase ABHD5 polypeptide. In some embodiments, the polynucleotide comprises the coding sequence of an ABHD5 gene as described herein. In some embodiments, a polynucleotide encoding a 1-acylglycerol-3-phosphate O-acyltransferase ABHD5 polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 104. In some embodiments, a polynucleotide encoding a 1-acylglycerol-3-phosphate O-acyltransferase ABHD5 polypeptide is a polynucleotide that encodes a polypeptide comprising the amino acid sequence of SEQ ID NO: 104.

In some embodiments, a polynucleotide encoding a 1-acylglycerol-3-phosphate O-acyltransferase ABHD5 polypeptide is a polynucleotide that encodes an N-terminal truncation, a C-terminal truncation, or a fragment of the amino acid sequence of SEQ ID NO: 104. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, but fewer than 349 consecutive amino acids of SEQ ID NO: 104.

In some embodiments, a polynucleotide of the present disclosure encodes a human Aldehyde dehydrogenase family 3 member A2 polypeptide. In some embodiments, the polynucleotide comprises the coding sequence of an ALDH3A2 gene as described herein. In some embodiments, a polynucleotide encoding an Aldehyde dehydrogenase family 3 member A2 polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 105. In some embodiments, a polynucleotide encoding an Aldehyde dehydrogenase family 3 member A2 polypeptide is a polynucleotide that encodes a polypeptide comprising the amino acid sequence of SEQ ID NO: 105.

In some embodiments, a polynucleotide encoding an Aldehyde dehydrogenase family 3 member A2 polypeptide is a polynucleotide that encodes an N-terminal truncation, a C-terminal truncation, or a fragment of the amino acid sequence of SEQ ID NO: 105. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, at least 500, but fewer than 508 consecutive amino acids of SEQ ID NO: 105.

In some embodiments, a polynucleotide of the present disclosure encodes a human Arachidonate 12-lipoxygenase 12R-type polypeptide. In some embodiments, the polynucleotide comprises the coding sequence of an ALOX12B gene as described herein. In some embodiments, a polynucleotide encoding an Arachidonate 12-lipoxygenase 12R-type polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 106. In some embodiments, a polynucleotide encoding an Arachidonate 12-lipoxygenase 12R-type polypeptide is a polynucleotide that encodes a polypeptide comprising the amino acid sequence of SEQ ID NO: 106.

In some embodiments, a polynucleotide encoding an Arachidonate 12-lipoxygenase 12R-type polypeptide is a polynucleotide that encodes an N-terminal truncation, a C-terminal truncation, or a fragment of the amino acid sequence of SEQ ID NO: 106. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, at least 500, at least 600, at least 700, but fewer than 701 consecutive amino acids of SEQ ID NO: 106.

In some embodiments, a polynucleotide of the present disclosure encodes a human Hydroperoxide isomerase ALOXE3 polypeptide. In some embodiments, the polynucleotide comprises the coding sequence of an ALOXE3 gene as described herein. In some embodiments, a polynucleotide encoding a Hydroperoxide isomerase ALOXE3 polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 107 or SEQ ID NO: 108. In some embodiments, a polynucleotide encoding a Hydroperoxide isomerase ALOXE3 polypeptide is a polynucleotide that encodes a polypeptide comprising the amino acid sequence of SEQ ID NO: 107 or SEQ ID NO: 108.

In some embodiments, a polynucleotide encoding a Hydroperoxide isomerase ALOXE3 polypeptide is a polynucleotide that encodes an N-terminal truncation, a C-terminal truncation, or a fragment of the amino acid sequence of SEQ ID NO: 107. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, at least 500, at least 600, at least 700, but fewer than 711 consecutive amino acids of SEQ ID NO: 107.

In some embodiments, a polynucleotide encoding a Hydroperoxide isomerase ALOXE3 polypeptide is a polynucleotide that encodes an N-terminal truncation, a C-terminal truncation, or a fragment of the amino acid sequence of SEQ ID NO: 108. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, at least 500, at least 600, at least 700, at least 800, but fewer than 843 consecutive amino acids of SEQ ID NO: 108.

In some embodiments, a polynucleotide of the present disclosure encodes a human AP-1 complex subunit sigma-1A polypeptide. In some embodiments, the polynucleotide comprises the coding sequence of an AP1S1 gene as described herein. In some embodiments, a polynucleotide encoding an AP-1 complex subunit sigma-1A polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 109. In some embodiments, a polynucleotide encoding an AP-1 complex subunit sigma-1A polypeptide is a polynucleotide that encodes a polypeptide comprising the amino acid sequence of SEQ ID NO: 109.

In some embodiments, a polynucleotide encoding an AP-1 complex subunit sigma-1A polypeptide is a polynucleotide that encodes an N-terminal truncation, a C-terminal truncation, or a fragment of the amino acid sequence of SEQ ID NO: 109. N-terminal truncations, C-terminal truncations, or fragments may comprise at least 10, at least 12, at least 14, at least 16, at least 18, at least 20, at least 30, at least 40, at least 50, at least 75, at least 100, but fewer than 158 consecutive amino acids of SEQ ID NO: 109.

In some embodiments, a polynucleotide of the present disclosure encodes a human Arylsulfatase E polypeptide. In some embodiments, the polynucleotide comprises the coding sequence of an ARSE gene as described herein. In some embodiments, a polynucleotide encoding an Arylsulfatase E polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 110. In some embodiments, a polynucleotide encoding an Arylsulfatase E polypeptide is a polynucleotide that encodes a polypeptide comprising the amino acid sequence of SEQ ID NO: 110.

In some embodiments, a polynucleotide encoding an Arylsulfatase E polypeptide is a polynucleotide that encodes an N-terminal truncation, a C-terminal truncation, or a fragment of the amino acid sequence of SEQ ID NO: 110. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, at least 500, but fewer than 589 consecutive amino acids of SEQ ID NO: 110.

In some embodiments, a polynucleotide of the present disclosure encodes a human Caspase-14 polypeptide. In some embodiments, the polynucleotide comprises the coding sequence of a CASP14 gene as described herein. In some embodiments, a polynucleotide encoding a Caspase-14 polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 111. In some embodiments, a polynucleotide encoding a Caspase-14 polypeptide is a polynucleotide that encodes a polypeptide comprising the amino acid sequence of SEQ ID NO: 111.

In some embodiments, a polynucleotide encoding a Caspase-14 polypeptide is a polynucleotide that encodes an N-terminal truncation, a C-terminal truncation, or a fragment of the amino acid sequence of SEQ ID NO: 111. N-terminal truncations, C-terminal truncations, or fragments may comprise at least 10, at least 12, at least 14, at least 16, at least 18, at least 20, at least 30, at least 40, at least 50, at least 75, at least 100, at least 200, but fewer than 242 consecutive amino acids of SEQ ID NO: 111.

In some embodiments, a polynucleotide of the present disclosure encodes a human Corneodesmosin polypeptide. In some embodiments, the polynucleotide comprises the coding sequence of a CDSN gene as described herein. In some embodiments, a polynucleotide encoding a Corneodesmosin polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 112. In some embodiments, a polynucleotide encoding a Corneodesmosin polypeptide is a polynucleotide that encodes a polypeptide comprising the amino acid sequence of SEQ ID NO: 112.

In some embodiments, a polynucleotide encoding a Corneodesmosin polypeptide is a polynucleotide that encodes an N-terminal truncation, a C-terminal truncation, or a fragment of the amino acid sequence of SEQ ID NO: 112. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, at least 500, but fewer than 529 consecutive amino acids of SEQ ID NO: 112.

In some embodiments, a polynucleotide of the present disclosure encodes a human Ceramide synthase 3 polypeptide. In some embodiments, the polynucleotide comprises the coding sequence of a CERS3 gene as described herein. In some embodiments, a polynucleotide encoding a Ceramide synthase 3 polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 113. In some embodiments, a polynucleotide encoding a Ceramide synthase 3 polypeptide is a polynucleotide that encodes a polypeptide comprising the amino acid sequence of SEQ ID NO: 113.

In some embodiments, a polynucleotide encoding a Ceramide synthase 3 polypeptide is a polynucleotide that encodes an N-terminal truncation, a C-terminal truncation, or a fragment of the amino acid sequence of SEQ ID NO: 113. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, but fewer than 383 consecutive amino acids of SEQ ID NO: 113.

In some embodiments, a polynucleotide of the present disclosure encodes a human Carbohydrate sulfotransferase 8 polypeptide. In some embodiments, the polynucleotide comprises the coding sequence of a CHST8 gene as described herein. In some embodiments, a polynucleotide encoding a Carbohydrate sulfotransferase 8 polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 114. In some embodiments, a polynucleotide encoding a Carbohydrate sulfotransferase 8 polypeptide is a polynucleotide that encodes a polypeptide comprising the amino acid sequence of SEQ ID NO: 114.

In some embodiments, a polynucleotide encoding a Carbohydrate sulfotransferase 8 polypeptide is a polynucleotide that encodes an N-terminal truncation, a C-terminal truncation, or a fragment of the amino acid sequence of SEQ ID NO: 114. N-terminal truncations, C-terminal truncations, or fragments may comprise at least 10, at least 12, at least 14, at least 16, at least 18, at least 20, at least 30, at least 40, at least 50, at least 75, at least 100, at least 150, at least 200, at least 250, at least 300, at least 350, at least 400, but fewer than 424 consecutive amino acids of SEQ ID NO: 114.

In some embodiments, a polynucleotide of the present disclosure encodes a human Claudin-1 polypeptide. In some embodiments, the polynucleotide comprises the coding sequence of a CLDN1 gene as described herein. In some embodiments, a polynucleotide encoding a Claudin-1 polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 115. In some embodiments, a polynucleotide encoding a Claudin-1 polypeptide is a polynucleotide that encodes a polypeptide comprising the amino acid sequence of SEQ ID NO: 115.

In some embodiments, a polynucleotide encoding a Claudin-1 polypeptide is a polynucleotide that encodes an N-terminal truncation, a C-terminal truncation, or a fragment of the amino acid sequence of SEQ ID NO: 115. N-terminal truncations, C-terminal truncations, or fragments may comprise at least 10, at least 12, at least 14, at least 16, at least 18, at least 20, at least 30, at least 40, at least 50, at least 75, at least 100, at least 200, but fewer than 211 consecutive amino acids of SEQ ID NO: 115.

In some embodiments, a polynucleotide of the present disclosure encodes a human Cystatin-A polypeptide. In some embodiments, the polynucleotide comprises the coding sequence of a CSTA gene as described herein. In some embodiments, a polynucleotide encoding a Cystatin-A polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 116. In some embodiments, a polynucleotide encoding a Cystatin-A polypeptide is a polynucleotide that encodes a polypeptide comprising the amino acid sequence of SEQ ID NO: 116.

In some embodiments, a polynucleotide encoding a Cystatin-A polypeptide is a polynucleotide that encodes an N-terminal truncation, a C-terminal truncation, or a fragment of the amino acid sequence of SEQ ID NO: 116. N-terminal truncations, C-terminal truncations, or fragments may comprise at least 10, at least 12, at least 14, at least 16, at least 18, at least 20, at least 30, at least 40, at least 50, at least 75, but fewer than 98 consecutive amino acids of SEQ ID NO: 116.

In some embodiments, a polynucleotide of the present disclosure encodes a human Cytochrome P450 4F22 polypeptide. In some embodiments, the polynucleotide comprises the coding sequence of a CYP4F22 gene as described herein. In some embodiments, a polynucleotide encoding a Cytochrome P450 4F22 polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 117. In some embodiments, a polynucleotide encoding a Cytochrome P450 4F22 polypeptide is a polynucleotide that encodes a polypeptide comprising the amino acid sequence of SEQ ID NO: 117.

In some embodiments, a polynucleotide encoding a Cytochrome P450 4F22 polypeptide is a polynucleotide that encodes an N-terminal truncation, a C-terminal truncation, or a fragment of the amino acid sequence of SEQ ID NO: 117. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, at least 500, but fewer than 531 consecutive amino acids of SEQ ID NO: 117.

In some embodiments, a polynucleotide of the present disclosure encodes a human 3-beta-hydroxysteroid-Delta(8),Delta(7)-isomerase polypeptide. In some embodiments, the polynucleotide comprises the coding sequence of an EBP gene as described herein. In some embodiments, a polynucleotide encoding a 3-beta-hydroxysteroid-Delta(8),Delta(7)-isomerase polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 118. In some embodiments, a polynucleotide encoding a 3-beta-hydroxysteroid-Delta(8),Delta(7)-isomerase polypeptide is a polynucleotide that encodes a polypeptide comprising the amino acid sequence of SEQ ID NO: 118.

In some embodiments, a polynucleotide encoding a 3-beta-hydroxysteroid-Delta(8),Delta(7)-isomerase polypeptide is a polynucleotide that encodes an N-terminal truncation, a C-terminal truncation, or a fragment of the amino acid sequence of SEQ ID NO: 118. N-terminal truncations, C-terminal truncations, or fragments may comprise at least 10, at least 12, at least 14, at least 16, at least 18, at least 20, at least 30, at least 40, at least 50, at least 75, at least 100, at least 125, at least 150, at least 175, at least 200, at least 225, but fewer than 230 consecutive amino acids of SEQ ID NO: 118.

In some embodiments, a polynucleotide of the present disclosure encodes a human Elongation of very long chain fatty acids protein 4 polypeptide. In some embodiments, the polynucleotide comprises the coding sequence of an ELOVL4 gene as described herein. In some embodiments, a polynucleotide encoding an Elongation of very long chain fatty acids protein 4 polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 119. In some embodiments, a polynucleotide encoding an Elongation of very long chain fatty acids protein 4 polypeptide is a polynucleotide that encodes a polypeptide comprising the amino acid sequence of SEQ ID NO: 119.

In some embodiments, a polynucleotide encoding an Elongation of very long chain fatty acids protein 4 polypeptide is a polynucleotide that encodes an N-terminal truncation, a C-terminal truncation, or a fragment of the amino acid sequence of SEQ ID NO: 119. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, but fewer than 314 consecutive amino acids of SEQ ID NO: 119.

In some embodiments, a polynucleotide of the present disclosure encodes a human Filaggrin polypeptide. In some embodiments, the polynucleotide comprises the coding sequence of a FLG gene as described herein. In some embodiments, a polynucleotide encoding a Filaggrin polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 120. In some embodiments, a polynucleotide encoding a Filaggrin polypeptide is a polynucleotide that encodes a polypeptide comprising the amino acid sequence of SEQ ID NO: 120.

In some embodiments, a polynucleotide encoding a Filaggrin polypeptide is a polynucleotide that encodes an N-terminal truncation, a C-terminal truncation, or a fragment of the amino acid sequence of SEQ ID NO: 120. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, at least 500, at least 1000, at least 1500, at least 2000, at least 2500, at least 3000, at least 3500, at least 4000, but fewer than 4061 consecutive amino acids of SEQ ID NO: 120.

In some embodiments, a polynucleotide of the present disclosure encodes a human Filaggrin 2 polypeptide. In some embodiments, the polynucleotide comprises the coding sequence of a FLG2 gene as described herein. In some embodiments, a polynucleotide encoding a Filaggrin 2 polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 121. In some embodiments, a polynucleotide encoding a Filaggrin 2 polypeptide is a polynucleotide that encodes a polypeptide comprising the amino acid sequence of SEQ ID NO: 121.

In some embodiments, a polynucleotide encoding a Filaggrin 2 polypeptide is a polynucleotide that encodes an N-terminal truncation, a C-terminal truncation, or a fragment of the amino acid sequence of SEQ ID NO: 121. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, at least 500, at least 750, at least 1000, at least 1250, at least 1500, at least 1750, at least 2000, at least 2250, but fewer than 2391 consecutive amino acids of SEQ ID NO: 121.

In some embodiments, a polynucleotide of the present disclosure encodes a human Gap junction beta-2 polypeptide. In some embodiments, the polynucleotide comprises the coding sequence of a GJB2 gene as described herein. In some embodiments, a polynucleotide encoding a Gap junction beta-2 polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 122. In some embodiments, a polynucleotide encoding a Gap junction beta-2 polypeptide is a polynucleotide that encodes a polypeptide comprising the amino acid sequence of SEQ ID NO: 122.

In some embodiments, a polynucleotide encoding a Gap junction beta-2 polypeptide is a polynucleotide that encodes an N-terminal truncation, a C-terminal truncation, or a fragment of the amino acid sequence of SEQ ID NO: 122. N-terminal truncations, C-terminal truncations, or fragments may comprise at least 10, at least 12, at least 14, at least 16, at least 18, at least 20, at least 30, at least 40, at least 50, at least 75, at least 100, at least 125, at least 150, at least 175, at least 200, but fewer than 226 consecutive amino acids of SEQ ID NO: 122.

In some embodiments, a polynucleotide of the present disclosure encodes a human Gap junction beta-3 polypeptide. In some embodiments, the polynucleotide comprises the coding sequence of a GJB3 gene as described herein. In some embodiments, a polynucleotide encoding a Gap junction beta-3 polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 123. In some embodiments, a polynucleotide encoding a Gap junction beta-3 polypeptide is a polynucleotide that encodes a polypeptide comprising the amino acid sequence of SEQ ID NO: 123.

In some embodiments, a polynucleotide encoding a Gap junction beta-3 polypeptide is a polynucleotide that encodes an N-terminal truncation, a C-terminal truncation, or a fragment of the amino acid sequence of SEQ ID NO: 123. N-terminal truncations, C-terminal truncations, or fragments may comprise at least 10, at least 12, at least 14, at least 16, at least 18, at least 20, at least 30, at least 40, at least 50, at least 75, at least 100, at least 150, at least 200, at least 250, but fewer than 270 consecutive amino acids of SEQ ID NO: 123.

In some embodiments, a polynucleotide of the present disclosure encodes a human Gap junction beta-4 polypeptide. In some embodiments, the polynucleotide comprises the coding sequence of a GJB4 gene as described herein. In some embodiments, a polynucleotide encoding a Gap junction beta-4 polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 124. In some embodiments, a polynucleotide encoding a Gap junction beta-4 polypeptide is a polynucleotide that encodes a polypeptide comprising the amino acid sequence of SEQ ID NO: 124.

In some embodiments, a polynucleotide encoding a Gap junction beta-4 polypeptide is a polynucleotide that encodes an N-terminal truncation, a C-terminal truncation, or a fragment of the amino acid sequence of SEQ ID NO: 124. N-terminal truncations, C-terminal truncations, or fragments may comprise at least 10, at least 12, at least 14, at least 16, at least 18, at least 20, at least 30, at least 40, at least 50, at least 75, at least 100, at least 150, at least 200, at least 250, but fewer than 266 consecutive amino acids of SEQ ID NO: 124.

In some embodiments, a polynucleotide of the present disclosure encodes a human Gap junction beta-6 polypeptide. In some embodiments, the polynucleotide comprises the coding sequence of a GJB6 gene as described herein. In some embodiments, a polynucleotide encoding a Gap junction beta-6 polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 125. In some embodiments, a polynucleotide encoding a Gap junction beta-6 polypeptide is a polynucleotide that encodes a polypeptide comprising the amino acid sequence of SEQ ID NO: 125.

In some embodiments, a polynucleotide encoding a Gap junction beta-6 polypeptide is a polynucleotide that encodes an N-terminal truncation, a C-terminal truncation, or a fragment of the amino acid sequence of SEQ ID NO: 125. N-terminal truncations, C-terminal truncations, or fragments may comprise at least 10, at least 12, at least 14, at least 16, at least 18, at least 20, at least 30, at least 40, at least 50, at least 75, at least 100, at least 150, at least 200, at least 250, but fewer than 261 consecutive amino acids of SEQ ID NO: 125.

In some embodiments, a polynucleotide of the present disclosure encodes a human 3-ketodihydrosphingosine reductase polypeptide. In some embodiments, the polynucleotide comprises the coding sequence of a KDSR gene as described herein. In some embodiments, a polynucleotide encoding a 3-ketodihydrosphingosine reductase polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 126. In some embodiments, a polynucleotide encoding a 3-ketodihydrosphingosine reductase polypeptide is a polynucleotide that encodes a polypeptide comprising the amino acid sequence of SEQ ID NO: 126.

In some embodiments, a polynucleotide encoding a 3-ketodihydrosphingosine reductase polypeptide is a polynucleotide that encodes an N-terminal truncation, a C-terminal truncation, or a fragment of the amino acid sequence of SEQ ID NO: 126. N-terminal truncations, C-terminal truncations, or fragments may comprise at least 10, at least 12, at least 14, at least 16, at least 18, at least 20, at least 30, at least 40, at least 50, at least 75, at least 100, at least 150, at least 200, at least 250, at least 300, but fewer than 332 consecutive amino acids of SEQ ID NO: 126.

In some embodiments, a polynucleotide of the present disclosure encodes a human Keratin, type II cytoskeletal 1 polypeptide. In some embodiments, the polynucleotide comprises the coding sequence of a KRT1 gene as described herein. In some embodiments, a polynucleotide encoding a Keratin, type II cytoskeletal 1 polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 127. In some embodiments, a polynucleotide encoding a Keratin, type II cytoskeletal 1 polypeptide is a polynucleotide that encodes a polypeptide comprising the amino acid sequence of SEQ ID NO: 127.

In some embodiments, a polynucleotide encoding a Keratin, type II cytoskeletal 1 polypeptide is a polynucleotide that encodes an N-terminal truncation, a C-terminal truncation, or a fragment of the amino acid sequence of SEQ ID NO: 127. N-terminal truncations, C-terminal truncations, or fragments may comprise at least 10, at least 12, at least 14, at least 16, at least 18, at least 20, at least 30, at least 40, at least 50, at least 75, at least 100, at least 150, at least 200, at least 250, at least about 300, at least about 400, at least about 500, at least about 600, but fewer than 644 consecutive amino acids of SEQ ID NO: 127.

In some embodiments, a polynucleotide of the present disclosure encodes a human Keratin, type II cytoskeletal 2 epidermal polypeptide. In some embodiments, the polynucleotide comprises the coding sequence of a KRT2 gene as described herein. In some embodiments, a polynucleotide encoding a Keratin, type II cytoskeletal 2 epidermal polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 128. In some embodiments, a polynucleotide encoding a Keratin, type II cytoskeletal 2 epidermal polypeptide is a polynucleotide that encodes a polypeptide comprising the amino acid sequence of SEQ ID NO: 128.

In some embodiments, a polynucleotide encoding a Keratin, type II cytoskeletal 2 epidermal polypeptide is a polynucleotide that encodes an N-terminal truncation, a C-terminal truncation, or a fragment of the amino acid sequence of SEQ ID NO: 128. N-terminal truncations, C-terminal truncations, or fragments may comprise at least 10, at least 12, at least 14, at least 16, at least 18, at least 20, at least 30, at least 40, at least 50, at least 75, at least 100, at least 150, at least 200, at least 250, at least 300, at least 400, at least 500, at least 600, but fewer than 639 consecutive amino acids of SEQ ID NO: 128.

In some embodiments, a polynucleotide of the present disclosure encodes a human Keratin, type I cytoskeletal 9 polypeptide. In some embodiments, the polynucleotide comprises the coding sequence of a KRT9 gene as described herein. In some embodiments, a polynucleotide encoding a Keratin, type I cytoskeletal 9 polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 129. In some embodiments, a polynucleotide encoding a Keratin, type I cytoskeletal 9 polypeptide is a polynucleotide that encodes a polypeptide comprising the amino acid sequence of SEQ ID NO: 129.

In some embodiments, a polynucleotide encoding a Keratin, type I cytoskeletal 9 polypeptide is a polynucleotide that encodes an N-terminal truncation, a C-terminal truncation, or a fragment of the amino acid sequence of SEQ ID NO: 129. N-terminal truncations, C-terminal truncations, or fragments may comprise at least 10, at least 12, at least 14, at least 16, at least 18, at least 20, at least 30, at least 40, at least 50, at least 75, at least 100, at least 150, at least 200, at least 250, at least 300, at least 400, at least 500, at least 600, but fewer than 623 consecutive amino acids of SEQ ID NO: 129.

In some embodiments, a polynucleotide of the present disclosure encodes a human Keratin, type I cytoskeletal 10 polypeptide. In some embodiments, the polynucleotide comprises the coding sequence of a KRT10 gene as described herein. In some embodiments, a polynucleotide encoding a Keratin, type I cytoskeletal 10 polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 130. In some embodiments, a polynucleotide encoding a Keratin, type I cytoskeletal 10 polypeptide is a polynucleotide that encodes a polypeptide comprising the amino acid sequence of SEQ ID NO: 130.

In some embodiments, a polynucleotide encoding a Keratin, type I cytoskeletal 10 polypeptide is a polynucleotide that encodes an N-terminal truncation, a C-terminal truncation, or a fragment of the amino acid sequence of SEQ ID NO: 130. N-terminal truncations, C-terminal truncations, or fragments may comprise at least 10, at least 12, at least 14, at least 16, at least 18, at least 20, at least 30, at least 40, at least 50, at least 75, at least 100, at least 150, at least 200, at least 250, at least 300, at least 400, at least 500, but fewer than 584 consecutive amino acids of SEQ ID NO: 130.

In some embodiments, a polynucleotide of the present disclosure encodes a human Lipase member N polypeptide. In some embodiments, the polynucleotide comprises the coding sequence of a LIPN gene as described herein. In some embodiments, a polynucleotide encoding a Lipase member N polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 131. In some embodiments, a polynucleotide encoding a Lipase member N polypeptide is a polynucleotide that encodes a polypeptide comprising the amino acid sequence of SEQ ID NO: 131.

In some embodiments, a polynucleotide encoding a Lipase member N polypeptide is a polynucleotide that encodes an N-terminal truncation, a C-terminal truncation, or a fragment of the amino acid sequence of SEQ ID NO: 131. N-terminal truncations, C-terminal truncations, or fragments may comprise at least 10, at least 12, at least 14, at least 16, at least 18, at least 20, at least 30, at least 40, at least 50, at least 75, at least 100, at least 150, at least 200, at least 250, at least 300, at least 350, but fewer than 398 consecutive amino acids of SEQ ID NO: 131.

In some embodiments, a polynucleotide of the present disclosure encodes a human Loricrin polypeptide. In some embodiments, the polynucleotide comprises the coding sequence of a LOR gene as described herein. In some embodiments, a polynucleotide encoding a Loricrin polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 132. In some embodiments, a polynucleotide encoding a Loricrin polypeptide is a polynucleotide that encodes a polypeptide comprising the amino acid sequence of SEQ ID NO: 132.

In some embodiments, a polynucleotide encoding a Loricrin polypeptide is a polynucleotide that encodes an N-terminal truncation, a C-terminal truncation, or a fragment of the amino acid sequence of SEQ ID NO: 132. N-terminal truncations, C-terminal truncations, or fragments may comprise at least 10, at least 12, at least 14, at least 16, at least 18, at least 20, at least 30, at least 40, at least 50, at least 75, at least 100, at least 150, at least 200, at least 250, at least 300, but fewer than 312 consecutive amino acids of SEQ ID NO: 132.

In some embodiments, a polynucleotide of the present disclosure encodes a human Membrane-bound transcription factor site-2 protease polypeptide. In some embodiments, the polynucleotide comprises the coding sequence of a MBTPS2 gene as described herein. In some embodiments, a polynucleotide encoding a Membrane-bound transcription factor site-2 protease polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 133. In some embodiments, a polynucleotide encoding a Membrane-bound transcription factor site-2 protease polypeptide is a polynucleotide that encodes a polypeptide comprising the amino acid sequence of SEQ ID NO: 133.

In some embodiments, a polynucleotide encoding a Membrane-bound transcription factor site-2 protease polypeptide is a polynucleotide that encodes an N-terminal truncation, a C-terminal truncation, or a fragment of the amino acid sequence of SEQ ID NO: 133. N-terminal truncations, C-terminal truncations, or fragments may comprise at least 10, at least 12, at least 14, at least 16, at least 18, at least 20, at least 30, at least 40, at least 50, at least 75, at least 100, at least 150, at least 200, at least 250, at least 300, at least 350, at least 400, at least 450, at least 500, but fewer than 519 consecutive amino acids of SEQ ID NO: 133.

In some embodiments, a polynucleotide of the present disclosure encodes a human Magnesium transporter NIPA4 polypeptide. In some embodiments, the polynucleotide comprises the coding sequence of an NIPAL4 gene as described herein. In some embodiments, a polynucleotide encoding a Magnesium transporter NIPA4 polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 134 or SEQ ID NO: 135. In some embodiments, a polynucleotide encoding a Magnesium transporter NIPA4 polypeptide is a polynucleotide that encodes a polypeptide comprising the amino acid sequence of SEQ ID NO: 134 or SEQ ID NO: 135.

In some embodiments, a polynucleotide encoding a Magnesium transporter NIPA4 polypeptide is a polynucleotide that encodes an N-terminal truncation, a C-terminal truncation, or a fragment of the amino acid sequence of SEQ ID NO: 134. N-terminal truncations, C-terminal truncations, or fragments may comprise at least 10, at least 12, at least 14, at least 16, at least 18, at least 20, at least 30, at least 40, at least 50, at least 75, at least 100, at least 150, at least 200, at least 250, at least 300, at least 350, at least 400, at least 450, but fewer than 466 consecutive amino acids of SEQ ID NO: 134.

In some embodiments, a polynucleotide encoding a Magnesium transporter NIPA4 polypeptide is a polynucleotide that encodes an N-terminal truncation, a C-terminal truncation, or a fragment of the amino acid sequence of SEQ ID NO: 135. N-terminal truncations, C-terminal truncations, or fragments may comprise at least 10, at least 12, at least 14, at least 16, at least 18, at least 20, at least 30, at least 40, at least 50, at least 75, at least 100, at least 150, at least 200, at least 250, at least 300, at least 350, at least 400, but fewer than 447 consecutive amino acids of SEQ ID NO: 135.

In some embodiments, a polynucleotide of the present disclosure encodes a human Sterol-4-alpha-carboxylate 3-dehydrogenase, decarboxylating polypeptide. In some embodiments, the polynucleotide comprises the coding sequence of an NSDHL gene as described herein. In some embodiments, a polynucleotide encoding a Sterol-4-alpha-carboxylate 3-dehydrogenase, decarboxylating polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 136. In some embodiments, a polynucleotide encoding a Sterol-4-alpha-carboxylate 3-dehydrogenase, decarboxylating polypeptide is a polynucleotide that encodes a polypeptide comprising the amino acid sequence of SEQ ID NO: 136.

In some embodiments, a polynucleotide encoding a Sterol-4-alpha-carboxylate 3-dehydrogenase, decarboxylating polypeptide is a polynucleotide that encodes an N-terminal truncation, a C-terminal truncation, or a fragment of the amino acid sequence of SEQ ID NO: 136. N-terminal truncations, C-terminal truncations, or fragments may comprise at least 10, at least 12, at least 14, at least 16, at least 18, at least 20, at least 30, at least 40, at least 50, at least 75, at least 100, at least 150, at least 200, at least 250, at least 300, at least 350, but fewer than 373 consecutive amino acids of SEQ ID NO: 136.

In some embodiments, a polynucleotide of the present disclosure encodes a human Peroxisomal targeting signal 2 receptor polypeptide. In some embodiments, the polynucleotide comprises the coding sequence of a PEX7 gene as described herein. In some embodiments, a polynucleotide encoding a Peroxisomal targeting signal 2 receptor polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 137. In some embodiments, a polynucleotide encoding a Peroxisomal targeting signal 2 receptor polypeptide is a polynucleotide that encodes a polypeptide comprising the amino acid sequence of SEQ ID NO: 137.

In some embodiments, a polynucleotide encoding a Peroxisomal targeting signal 2 receptor polypeptide is a polynucleotide that encodes an N-terminal truncation, a C-terminal truncation, or a fragment of the amino acid sequence of SEQ ID NO: 137. N-terminal truncations, C-terminal truncations, or fragments may comprise at least 10, at least 12, at least 14, at least 16, at least 18, at least 20, at least 30, at least 40, at least 50, at least 75, at least 100, at least 150, at least 200, at least 250, at least 300, but fewer than 323 consecutive amino acids of SEQ ID NO: 137.

In some embodiments, a polynucleotide of the present disclosure encodes a human D-3-phosphoglycerate dehydrogenase polypeptide. In some embodiments, the polynucleotide comprises the coding sequence of a PHGDH gene as described herein. In some embodiments, a polynucleotide encoding a D-3-phosphoglycerate dehydrogenase polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 138. In some embodiments, a polynucleotide encoding a D-3-phosphoglycerate dehydrogenase polypeptide is a polynucleotide that encodes a polypeptide comprising the amino acid sequence of SEQ ID NO: 138.

In some embodiments, a polynucleotide encoding a D-3-phosphoglycerate dehydrogenase polypeptide is a polynucleotide that encodes an N-terminal truncation, a C-terminal truncation, or a fragment of the amino acid sequence of SEQ ID NO: 138. N-terminal truncations, C-terminal truncations, or fragments may comprise at least 10, at least 12, at least 14, at least 16, at least 18, at least 20, at least 30, at least 40, at least 50, at least 75, at least 100, at least 150, at least 200, at least 250, at least 300, at least 350, at least 400, at least 450, at least 500, but fewer than 533 consecutive amino acids of SEQ ID NO: 138.

In some embodiments, a polynucleotide of the present disclosure encodes a human Phytanoyl-CoA dioxygenase, peroxisomal polypeptide. In some embodiments, the polynucleotide comprises the coding sequence of a PHYH gene as described herein. In some embodiments, a polynucleotide encoding a Phytanoyl-CoA dioxygenase, peroxisomal polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 139. In some embodiments, a polynucleotide encoding a Phytanoyl-CoA dioxygenase, peroxisomal polypeptide is a polynucleotide that encodes a polypeptide comprising the amino acid sequence of SEQ ID NO: 139.

In some embodiments, a polynucleotide encoding a Phytanoyl-CoA dioxygenase, peroxisomal polypeptide is a polynucleotide that encodes an N-terminal truncation, a C-terminal truncation, or a fragment of the amino acid sequence of SEQ ID NO: 139. N-terminal truncations, C-terminal truncations, or fragments may comprise at least 10, at least 12, at least 14, at least 16, at least 18, at least 20, at least 30, at least 40, at least 50, at least 75, at least 100, at least 150, at least 200, at least 250, at least 300, but fewer than 338 consecutive amino acids of SEQ ID NO: 139.

In some embodiments, a polynucleotide of the present disclosure encodes a human Patatin-like phospholipase domain-containing protein 1 polypeptide. In some embodiments, the polynucleotide comprises the coding sequence of a PNPLA1 gene as described herein. In some embodiments, a polynucleotide encoding a Patatin-like phospholipase domain-containing protein 1 polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a sequence selected from SEQ ID NOS: 140-142. In some embodiments, a polynucleotide encoding a Patatin-like phospholipase domain-containing protein 1 polypeptide is a polynucleotide that encodes a polypeptide comprising an amino acid sequence selected from SEQ ID NOS: 140-142.

In some embodiments, a polynucleotide encoding a Patatin-like phospholipase domain-containing protein 1 polypeptide is a polynucleotide that encodes an N-terminal truncation, a C-terminal truncation, or a fragment of the amino acid sequence of SEQ ID NO: 140. N-terminal truncations, C-terminal truncations, or fragments may comprise at least 10, at least 12, at least 14, at least 16, at least 18, at least 20, at least 30, at least 40, at least 50, at least 75, at least 100, at least 150, at least 200, at least 250, at least 300, at least 350, at least 400, at least 450, at least 500, but fewer than 532 consecutive amino acids of SEQ ID NO: 140.

In some embodiments, a polynucleotide encoding a Patatin-like phospholipase domain-containing protein 1 polypeptide is a polynucleotide that encodes an N-terminal truncation, a C-terminal truncation, or a fragment of the amino acid sequence of SEQ ID NO: 141. N-terminal truncations, C-terminal truncations, or fragments may comprise at least 10, at least 12, at least 14, at least 16, at least 18, at least 20, at least 30, at least 40, at least 50, at least 75, at least 100, at least 150, at least 200, at least 250, at least 300, at least 350, at least 400, but fewer than 437 consecutive amino acids of SEQ ID NO: 141.

In some embodiments, a polynucleotide encoding a Patatin-like phospholipase domain-containing protein 1 polypeptide is a polynucleotide that encodes an N-terminal truncation, a C-terminal truncation, or a fragment of the amino acid sequence of SEQ ID NO: 142. N-terminal truncations, C-terminal truncations, or fragments may comprise at least 10, at least 12, at least 14, at least 16, at least 18, at least 20, at least 30, at least 40, at least 50, at least 75, at least 100, at least 150, at least 200, at least 250, at least 300, at least 350, at least 400, but fewer than 446 consecutive amino acids of SEQ ID NO: 142.

In some embodiments, a polynucleotide of the present disclosure encodes a human Proteasome maturation protein polypeptide. In some embodiments, the polynucleotide comprises the coding sequence of a POMP gene as described herein. In some embodiments, a polynucleotide encoding a Proteasome maturation protein polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 143. In some embodiments, a polynucleotide encoding a Proteasome maturation protein polypeptide is a polynucleotide that encodes a polypeptide comprising the amino acid sequence of SEQ ID NO: 143.

In some embodiments, a polynucleotide encoding a Proteasome maturation protein polypeptide is a polynucleotide that encodes an N-terminal truncation, a C-terminal truncation, or a fragment of the amino acid sequence of SEQ ID NO: 143. N-terminal truncations, C-terminal truncations, or fragments may comprise at least 10, at least 12, at least 14, at least 16, at least 18, at least 20, at least 30, at least 40, at least 50, at least 75, at least 100, at least 125, but fewer than 141 consecutive amino acids of SEQ ID NO: 143.

In some embodiments, a polynucleotide of the present disclosure encodes a human Phosphoserine aminotransferase polypeptide. In some embodiments, the polynucleotide comprises the coding sequence of a PSAT1 gene as described herein. In some embodiments, a polynucleotide encoding a Phosphoserine aminotransferase polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 144. In some embodiments, a polynucleotide encoding a Phosphoserine aminotransferase polypeptide is a polynucleotide that encodes a polypeptide comprising the amino acid sequence of SEQ ID NO: 144.

In some embodiments, a polynucleotide encoding a Phosphoserine aminotransferase polypeptide is a polynucleotide that encodes an N-terminal truncation, a C-terminal truncation, or a fragment of the amino acid sequence of SEQ ID NO: 144. N-terminal truncations, C-terminal truncations, or fragments may comprise at least 10, at least 12, at least 14, at least 16, at least 18, at least 20, at least 30, at least 40, at least 50, at least 75, at least 100, at least 150, at least 200, at least 250, at least 300, at least 350, but fewer than 370 consecutive amino acids of SEQ ID NO: 144.

In some embodiments, a polynucleotide of the present disclosure encodes a human Short-chain dehydrogenase/reductase family 9C member 7 polypeptide. In some embodiments, the polynucleotide comprises the coding sequence of a SDR9C7 gene as described herein. In some embodiments, a polynucleotide encoding a Short-chain dehydrogenase/reductase family 9C member 7 polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 145. In some embodiments, a polynucleotide encoding a Short-chain dehydrogenase/reductase family 9C member 7 polypeptide is a polynucleotide that encodes a polypeptide comprising the amino acid sequence of SEQ ID NO: 145.

In some embodiments, a polynucleotide encoding a Short-chain dehydrogenase/reductase family 9C member 7 polypeptide is a polynucleotide that encodes an N-terminal truncation, a C-terminal truncation, or a fragment of the amino acid sequence of SEQ ID NO: 145. N-terminal truncations, C-terminal truncations, or fragments may comprise at least 10, at least 12, at least 14, at least 16, at least 18, at least 20, at least 30, at least 40, at least 50, at least 75, at least 100, at least 150, at least 200, at least 250, at least 300, but fewer than 313 consecutive amino acids of SEQ ID NO: 145.

In some embodiments, a polynucleotide of the present disclosure encodes a human Serpin B8 polypeptide. In some embodiments, the polynucleotide comprises the coding sequence of a SERPINB8 gene as described herein. In some embodiments, a polynucleotide encoding a Serpin B8 polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 146. In some embodiments, a polynucleotide encoding a Serpin B8 polypeptide is a polynucleotide that encodes a polypeptide comprising the amino acid sequence of SEQ ID NO: 146.

In some embodiments, a polynucleotide encoding a Serpin B8 polypeptide is a polynucleotide that encodes an N-terminal truncation, a C-terminal truncation, or a fragment of the amino acid sequence of SEQ ID NO: 146. N-terminal truncations, C-terminal truncations, or fragments may comprise at least 10, at least 12, at least 14, at least 16, at least 18, at least 20, at least 30, at least 40, at least 50, at least 75, at least 100, at least 150, at least 200, at least 250, at least 300, at least 350, but fewer than 374 consecutive amino acids of SEQ ID NO: 146.

In some embodiments, a polynucleotide of the present disclosure encodes a human Long-chain fatty acid transport protein 4 polypeptide. In some embodiments, the polynucleotide comprises the coding sequence of a SLC27A4 gene as described herein. In some embodiments, a polynucleotide encoding a Long-chain fatty acid transport protein 4 polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 147. In some embodiments, a polynucleotide encoding a Long-chain fatty acid transport protein 4 polypeptide is a polynucleotide that encodes a polypeptide comprising the amino acid sequence of SEQ ID NO: 147.

In some embodiments, a polynucleotide encoding a Long-chain fatty acid transport protein 4 polypeptide is a polynucleotide that encodes an N-terminal truncation, a C-terminal truncation, or a fragment of the amino acid sequence of SEQ ID NO: 147. N-terminal truncations, C-terminal truncations, or fragments may comprise at least 10, at least 12, at least 14, at least 16, at least 18, at least 20, at least 30, at least 40, at least 50, at least 75, at least 100, at least 150, at least 200, at least 250, at least 300, at least 350, at least 400, at least 450, at least 500, at least 550, at least 600, but fewer than 643 consecutive amino acids of SEQ ID NO: 147.

In some embodiments, a polynucleotide of the present disclosure encodes a human Synaptosomal-associated protein 29 polypeptide. In some embodiments, the polynucleotide comprises the coding sequence of a SNAP29 gene as described herein. In some embodiments, a polynucleotide encoding a Synaptosomal-associated protein 29 polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 148. In some embodiments, a polynucleotide encoding a Synaptosomal-associated protein 29 polypeptide is a polynucleotide that encodes a polypeptide comprising the amino acid sequence of SEQ ID NO: 148.

In some embodiments, a polynucleotide encoding a Synaptosomal-associated protein 29 polypeptide is a polynucleotide that encodes an N-terminal truncation, a C-terminal truncation, or a fragment of the amino acid sequence of SEQ ID NO: 148. N-terminal truncations, C-terminal truncations, or fragments may comprise at least 10, at least 12, at least 14, at least 16, at least 18, at least 20, at least 30, at least 40, at least 50, at least 75, at least 100, at least 125, at least 150, at least 175, at least 200, at least 225, at least 250, but fewer than 258 consecutive amino acids of SEQ ID NO: 148.

In some embodiments, a polynucleotide of the present disclosure encodes a human Suppressor of tumorigenicity 14 polypeptide. In some embodiments, the polynucleotide comprises the coding sequence of a ST14 gene as described herein. In some embodiments, a polynucleotide encoding a Suppressor of tumorigenicity 14 polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 149. In some embodiments, a polynucleotide encoding a Suppressor of tumorigenicity 14 polypeptide is a polynucleotide that encodes a polypeptide comprising the amino acid sequence of SEQ ID NO: 149.

In some embodiments, a polynucleotide encoding a Suppressor of tumorigenicity 14 polypeptide is a polynucleotide that encodes an N-terminal truncation, a C-terminal truncation, or a fragment of the amino acid sequence of SEQ ID NO: 149. N-terminal truncations, C-terminal truncations, or fragments may comprise at least 10, at least 12, at least 14, at least 16, at least 18, at least 20, at least 30, at least 40, at least 50, at least 75, at least 100, at least 150, at least 200, at least 250, at least 300, at least 350, at least 400, at least 450, at least 500, at least 550, at least 600, at least 650, at least 700, at least 750, at least 800, at least 850, but fewer than 855 consecutive amino acids of SEQ ID NO: 149.

In some embodiments, a polynucleotide of the present disclosure encodes a human Steryl-sulfatase polypeptide. In some embodiments, the polynucleotide comprises the coding sequence of a STS gene as described herein. In some embodiments, a polynucleotide encoding a Steryl-sulfatase polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 150. In some embodiments, a polynucleotide encoding a Steryl-sulfatase polypeptide is a polynucleotide that encodes a polypeptide comprising the amino acid sequence of SEQ ID NO: 150.

In some embodiments, a polynucleotide encoding a Steryl-sulfatase polypeptide is a polynucleotide that encodes an N-terminal truncation, a C-terminal truncation, or a fragment of the amino acid sequence of SEQ ID NO: 150. N-terminal truncations, C-terminal truncations, or fragments may comprise at least 10, at least 12, at least 14, at least 16, at least 18, at least 20, at least 30, at least 40, at least 50, at least 75, at least 100, at least 150, at least 200, at least 250, at least 300, at least 350, at least 400, at least 450, at least 500, at least 550, but fewer than 583 consecutive amino acids of SEQ ID NO: 150.

In some embodiments, a polynucleotide of the present disclosure encodes a human Sulfotransferase 2B1 polypeptide. In some embodiments, the polynucleotide comprises the coding sequence of a SULT2B1 gene as described herein. In some embodiments, a polynucleotide encoding a Sulfotransferase 2B1 polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 155. In some embodiments, a polynucleotide encoding a Sulfotransferase 2B1 polypeptide is a polynucleotide that encodes a polypeptide comprising the amino acid sequence of SEQ ID NO: 155.

In some embodiments, a polynucleotide encoding a Sulfotransferase 2B1 polypeptide is a polynucleotide that encodes an N-terminal truncation, a C-terminal truncation, or a fragment of the amino acid sequence of SEQ ID NO: 155. N-terminal truncations, C-terminal truncations, or fragments may comprise at least 10, at least 12, at least 14, at least 16, at least 18, at least 20, at least 30, at least 40, at least 50, at least 75, at least 100, at least 150, at least 200, at least 250, at least 300, at least 350, but fewer than 365 consecutive amino acids of SEQ ID NO: 155.

In some embodiments, a polynucleotide of the present disclosure encodes a human Vacuolar protein sorting-associated protein 33B polypeptide. In some embodiments, the polynucleotide comprises the coding sequence of a VPS33B gene as described herein. In some embodiments, a polynucleotide encoding a Vacuolar protein sorting-associated protein 33B polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 151. In some embodiments, a polynucleotide encoding a Vacuolar protein sorting-associated protein 33B polypeptide is a polynucleotide that encodes a polypeptide comprising the amino acid sequence of SEQ ID NO: 151.

In some embodiments, a polynucleotide encoding a Vacuolar protein sorting-associated protein 33B polypeptide is a polynucleotide that encodes an N-terminal truncation, a C-terminal truncation, or a fragment of the amino acid sequence of SEQ ID NO: 151. N-terminal truncations, C-terminal truncations, or fragments may comprise at least 10, at least 12, at least 14, at least 16, at least 18, at least 20, at least 30, at least 40, at least 50, at least 75, at least 100, at least 150, at least 200, at least 250, at least 300, at least 350, at least 400, at least 450, at least 500, at least 550, at least 600, but fewer than 617 consecutive amino acids of SEQ ID NO: 151.

In some embodiments, a polynucleotide of the present disclosure encodes a human CAAX prenyl protease 1 homolog polypeptide. In some embodiments, the polynucleotide comprises the coding sequence of a ZMPSTE24 gene as described herein. In some embodiments, a polynucleotide encoding a CAAX prenyl protease 1 homolog polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 152. In some embodiments, a polynucleotide encoding a CAAX prenyl protease 1 homolog polypeptide is a polynucleotide that encodes a polypeptide comprising the amino acid sequence of SEQ ID NO: 152.

In some embodiments, a polynucleotide encoding a CAAX prenyl protease 1 homolog polypeptide is a polynucleotide that encodes an N-terminal truncation, a C-terminal truncation, or a fragment of the amino acid sequence of SEQ ID NO: 152. N-terminal truncations, C-terminal truncations, or fragments may comprise at least 10, at least 12, at least 14, at least 16, at least 18, at least 20, at least 30, at least 40, at least 50, at least 75, at least 100, at least 150, at least 200, at least 250, at least 300, at least 350, at least 400, at least 450, but fewer than 475 consecutive amino acids of SEQ ID NO: 152.

In some embodiments, a polynucleotide of the present disclosure encoding an ichthyosis-associated polypeptide (e.g., a human ichthyosis-associated polypeptide) expresses the ichthyosis-associated polypeptide when the polynucleotide is delivered into one or more target cells of a subject (e.g., one or more cells of the epidermis of the subject). In some embodiments, expression of the ichthyosis-associated polypeptide (e.g., a human ichthyosis-associated polypeptide) enhances, increases, augments, and/or supplements the levels, function, and/or activity of the ichthyosis-associated polypeptide in one or more target cells of a subject (e.g., as compared to prior to expression of the ichthyosis-associated polypeptide). In some embodiments, expression of the ichthyosis-associated polypeptide (e.g., a human ichthyosis-associated polypeptide) provides prophylactic, palliative, or therapeutic relief to one or more signs or symptoms of a congenital ichthyosis in the subject (e.g., as compared to prior to expression of the ichthyosis-associated polypeptide).

Recombinant Nucleic Acids

In some embodiments, the present disclosure relates to recombinant nucleic acids comprising any one or more of the polynucleotides described herein. In some embodiments, the recombinant nucleic acid is a vector (e.g., an expression vector, a display vector, etc.). In some embodiments, the vector is a DNA vector or an RNA vector. Generally, vectors suitable to maintain, propagate, and/or express polynucleotides to produce one or more polypeptides in a subject may be used. Examples of suitable vectors may include, for example, plasmids, cosmids, episomes, transposons, and viral vectors (e.g., adenoviral vectors, adeno-associated viral vectors, vaccinia viral vectors, Sindbis-viral vectors, measles vectors, herpes viral vectors, lentiviral vectors, retroviral vectors, etc.). In some embodiments, the vector is a herpes viral vector. In some embodiments, the vector is capable of autonomous replication in a host cell. In some embodiments, the vector is incapable of autonomous replication in a host cell. In some embodiments, the vector can integrate into a host DNA. In some embodiments, the vector cannot integrate into a host DNA (e.g., is episomal). Methods of making vectors containing one or more polynucleotides of interest are well known to one of ordinary skill in the art, including, for example, by chemical synthesis or by artificial manipulation of isolated segments of nucleic acids (e.g., by genetic engineering techniques).

In some embodiments, a recombinant nucleic acid of the present disclosure is a herpes simplex virus (HSV) amplicon. Herpes virus amplicons, including the structural features and methods of making the same, are generally known to one of ordinary skill in the art (see e.g., de Silva S. and Bowers W. “Herpes Virus Amplicon Vectors”. Viruses 2009, 1, 594-629). In some embodiments, the herpes simplex virus amplicon is an HSV-1 amplicon. In some embodiments, the herpes simplex virus amplicon is an HSV-1 hybrid amplicon. Examples of HSV-1 hybrid amplicons may include, but are not limited to, HSV/AAV hybrid amplicons, HSV/EBV hybrid amplicons, HSV/EBV/RV hybrid amplicons, and/or HSV/Sleeping Beauty hybrid amplicons. In some embodiments, the amplicon is an HSV/AAV hybrid amplicon. In some embodiments, the amplicon is an HSV/Sleeping Beauty hybrid amplicon.

In some embodiments, a recombinant nucleic acid of the present disclosure is a recombinant herpes virus genome. The recombinant herpes virus genome may be a recombinant genome from any member of the Herpesviridae family of DNA viruses known in the art, including, for example, a recombinant herpes simplex virus genome, a recombinant varicella zoster virus genome, a recombinant human cytomegalovirus genome, a recombinant herpesvirus 6A genome, a recombinant herpesvirus 6B genome, a recombinant herpesvirus 7 genome, a recombinant Kaposi's sarcoma-associated herpesvirus genome, and any combinations or any derivatives thereof. In some embodiments, the recombinant herpes virus genome comprises one or more (e.g., one or more, two or more, three or more, four or more, five or more, six or more, seven or more, eight or more, nine or more, ten or more, etc.) inactivating mutations. In some embodiments, the one or more inactivating mutations are in one or more (e.g., one or more, two or more, three or more, four or more, five or more, six or more, seven or more, eight or more, nine or more, ten or more, etc.) herpes virus genes. In some embodiments, the recombinant herpes virus genome is attenuated (e.g., as compared to a corresponding, wild-type herpes virus genome). In some embodiments, the recombinant herpes virus genome is replication competent. In some embodiments, the recombinant herpes virus genome is replication defective.

In some embodiments, the recombinant nucleic acid is a recombinant herpes simplex virus (HSV) genome. In some embodiments, the recombinant herpes simplex virus genome is a recombinant herpes simplex virus type 1 (HSV-1) genome, a recombinant herpes simplex virus type 2 (HSV-2) genome, or any derivatives thereof. In some embodiments, the recombinant herpes simplex virus genome is a recombinant HSV-1 genome. In some embodiments, the recombinant HSV-1 genome may be from any HSV-1 strain known in the art, including, for example, strains 17, Ty25, R62, S25, Ku86, S23, R11, Ty148, Ku47, H166_{syn}, 1319-2005, F-13, M-12, 90237, F-17, KOS, 3083-2008, F12g, L2, CD38, H193, M-15, India 2011, 0116209, F-11I, 66-207, 2762, 369-2007, 3355, MacIntyre, McKrae, 7862, 7-hse, HF10, 1394, 2005, 270-2007, OD4, SC16, M-19, 4J1037, 5J1060, J1060, KOS79, 132-1988, 160-1982, H166, 2158-2007, RE, 78326, F18g, F11, 172-2010, H129, F, E4, CJ994, F14g, E03, E22, E10, E06, E11, E25, E23, E35, E15, E07, E12, E14, E08, E19, E13, ATCC 2011, etc. (see e.g., Bowen et al. J Virol. 2019 Apr. 3; 93(8)). In some embodiments, the recombinant HSV-1 genome is from the KOS strain. In some embodiments, the recombinant HSV-1 genome is not from the McKrae strain. In some embodiments, the recombinant herpes simplex virus genome is attenuated. In some embodiments, the recombinant herpes simplex virus genome is replication competent. In some embodiments, the recombinant herpes simplex virus genome is replication defective. In some embodiments, the recombinant herpes simplex virus genome comprises one or more (e.g., one or more, two or more, three or more, four or more, five or more, six or more, seven or more, eight or more, nine or more, ten or more, etc.) inactivating mutations. In some embodiments, the one or more inactivating mutations are in one or more (e.g., one or more, two or more, three or more, four or more, five or more, six or more, seven or more, eight or more, nine or more, ten or more, etc.) herpes simplex virus genes. As used herein, an “inactivating mutation” may refer to any mutation that results in a gene or regulon product (RNA or protein) having reduced, undetectable, or eliminated quantity and/or function (e.g., as compared to a corresponding sequence lacking the inactivating mutation). Examples of inactivating mutations may include, but are not limited to, deletions, insertions, point mutations, and rearrangements in transcriptional control sequences (promoters, enhancers, insulators, etc.) and/or coding sequences of a given gene or regulon. Any suitable method of measuring the quantity of a gene or regulon product known in the art may be used, including, for example, qPCR, Northern blots, RNAseq, western blots, ELISAs, etc.

In some embodiments, the recombinant herpes simplex virus genome comprises an inactivating mutation in at least one, at least two, at least three, at least four, at least five, at least six, at least seven, or all eight of the Infected Cell Protein (or Infected Cell Polypeptide) (ICP) 0, ICP4, ICP22, ICP27, ICP47, thymidine kinase (tk), Long Unique Region (UL) 41 and/or UL55 herpes simplex virus genes. In some embodiments, the recombinant herpes simplex virus genome does not comprise an inactivating mutation in the ICP34.5 (one or both copies) and/or ICP47 herpes simplex virus genes (e.g., to avoid production of an immune-stimulating virus). In some embodiments, the recombinant herpes simplex virus genome does not comprise an inactivating mutation in the ICP34.5 (one or both copies) herpes simplex virus gene. In some embodiments, the recombinant herpes simplex virus genome does not comprise an inactivating mutation in the ICP47 herpes simplex virus gene. In some embodiments, the recombinant herpes simplex virus genome does not comprise an inactivating mutation in the ICP34.5 (one or both copies) and ICP47 herpes simplex virus genes. In some embodiments, the recombinant herpes simplex virus genome is not oncolytic.

In some embodiments, the recombinant herpes simplex virus genome comprises an inactivating mutation in the ICP0 gene (one or both copies). In some embodiments, the recombinant herpes simplex virus genome comprises an inactivating mutation in the ICP0 gene (one or both copies), and further comprises an inactivating mutation in the ICP4 (one or both copies), ICP22, ICP27, ICP47, UL41, and/or UL55 genes. In some embodiments, the recombinant herpes simplex virus genome comprises an inactivating mutation in the ICP0 gene (one or both copies), and an inactivating mutation in the ICP4 gene (one or both copies). In some embodiments, the recombinant herpes simplex virus genome comprises an inactivating mutation in the ICP0 gene (one or both copies), and an inactivating mutation in the ICP22 gene. In some embodiments, the recombinant herpes simplex virus genome comprises an inactivating mutation in the ICP0 gene (one or both copies), and an inactivating mutation in the UL41 gene. In some embodiments, the recombinant herpes simplex virus genome comprises an inactivating mutation in the ICP0 gene (one or both copies), an inactivating mutation in the ICP4 gene (one or both copies), and an inactivating mutation in the ICP22 gene. In some embodiments, the recombinant herpes simplex virus genome comprises an inactivating mutation in the ICP0 gene (one or both copies), an inactivating mutation in the ICP4 gene (one or both copies), and an inactivating mutation in the UL41 gene. In some embodiments, the recombinant herpes simplex virus genome comprises an inactivating mutation in the ICP0 gene (one or both copies), an inactivating mutation in the ICP22 gene, and an inactivating mutation in the UL41 gene. In some embodiments, the recombinant herpes simplex virus genome comprises an inactivating mutation in the ICP0 gene (one or both copies), an inactivating mutation in the ICP4 gene (one or both copies), an inactivating mutation in the ICP22 gene, and an inactivating mutation in the UL41 gene. In some embodiments, the inactivating mutation is a deletion of the coding sequence of the ICP0 (one or both copies), ICP4 (one or both copies), ICP22, and/or UL41 genes. In some embodiments, the recombinant herpes simplex virus genome further comprises an inactivating mutation in the ICP27, ICP47, and/or UL55 genes.

In some embodiments, the recombinant herpes simplex virus genome comprises an inactivating mutation in the ICP4 gene (one or both copies). In some embodiments, the recombinant herpes simplex virus genome comprises an inactivating mutation in the ICP4 gene (one or both copies), and further comprises an inactivating mutation in the ICP0 (one or both copies), ICP22, ICP27, ICP47, UL41, and/or UL55 genes. In some embodiments, the recombinant herpes simplex virus genome comprises an inactivating mutation in the ICP4 gene (one or both copies), and an inactivating mutation in the ICP22 gene. In some embodiments, the recombinant herpes simplex virus genome comprises an inactivating mutation in the ICP4 gene (one or both copies), and an inactivating mutation in the UL41 gene. In some embodiments, the recombinant herpes simplex virus genome comprises an inactivating mutation in the ICP4 gene (one or both copies), an inactivating mutation in the ICP22 gene, and an inactivating mutation in the UL41 gene. In some embodiments, the inactivating mutation is a deletion of the coding sequence of the ICP4 (one or both copies), ICP22, and/or UL41 genes. In some embodiments, the recombinant herpes simplex virus genome further comprises an inactivating mutation in the ICP0 (one or both copies), ICP27, ICP47, and/or UL55 genes.

In some embodiments, the recombinant herpes simplex virus genome comprises an inactivating mutation in the ICP22 gene. In some embodiments, the recombinant herpes simplex virus genome comprises an inactivating mutation in the ICP22 gene, and further comprises an inactivating mutation in the ICP0 (one or both copies), ICP4 (one or both copies), ICP27, ICP47, UL41, and/or UL55 genes. In some embodiments, the recombinant herpes simplex virus genome comprises an inactivating mutation in the ICP22 gene, and an inactivating mutation UL41 gene. In some embodiments, the inactivating mutation is a deletion of the coding sequence of the ICP22 and/or UL41 genes. In some embodiments, the recombinant herpes simplex virus genome further comprises an inactivating mutation in the ICP0 (one or both copies), ICP4 (one or both copies), ICP27, ICP47, and/or UL55 genes.

In some embodiments, the recombinant herpes simplex virus genome comprises an inactivating mutation in the ICP27 gene. In some embodiments, the recombinant herpes simplex virus genome comprises an inactivating mutation in the ICP27 gene, and further comprises an inactivating mutation in the ICP0 (one or both copies), ICP4 (one or both copies), ICP22, ICP47, UL41, and/or UL55 genes. In some embodiments, the inactivating mutation is a deletion of the coding sequence of the ICP27 gene.

In some embodiments, the recombinant herpes simplex virus genome comprises an inactivating mutation in the ICP47 gene. In some embodiments, the recombinant herpes simplex virus genome comprises an inactivating mutation in the ICP47 gene, and further comprises an inactivating mutation in the ICP0 (one or both copies), ICP4 (one or both copies), ICP22, ICP27, UL41, and/or UL55 genes. In some embodiments, the inactivating mutation is a deletion of the coding sequence of the ICP47 gene.

In some embodiments, the recombinant herpes simplex virus genome comprises an inactivating mutation in the UL41 gene. In some embodiments, the recombinant herpes simplex virus genome comprises an inactivating mutation in the UL41 gene, and further comprises an inactivating mutation in the ICP0 (one or both copies), ICP4 (one or both copies), ICP22, ICP27, ICP47, and/or UL55 genes. In some embodiments, the inactivating mutation is a deletion of the coding sequence of the UL41 gene.

In some embodiments, the recombinant herpes simplex virus genome comprises an inactivating mutation in the UL55 gene. In some embodiments, the recombinant herpes simplex virus genome comprises an inactivating mutation in the UL55 gene, and further comprises an inactivating mutation in the ICP0 (one or both copies), ICP4 (one or both copies), ICP22, ICP27, ICP47, and/or UL41 genes. In some embodiments, the inactivating mutation is a deletion of the coding sequence of the UL55 gene.

In some embodiments, the recombinant herpes simplex virus genome comprises an inactivating mutation in (e.g., a deletion of) the internal repeat (Joint) region comprising the internal repeat long (IRL) and internal repeat short (IRs) regions. In some embodiments, inactivation (e.g., deletion) of the Joint region eliminates one copy each of the ICP4 and ICP0 genes. In some embodiments, inactivation (e.g., deletion) of the Joint region further inactivates (e.g., deletes) the promoter for the ICP22 and ICP47 genes. If desired, expression of one or both of these genes can be restored by insertion of an immediate early promoter into the recombinant herpes simplex virus genome (see e.g., Hill et al. (1995). Nature 375(6530): 411-415; Goldsmith et al. (1998). J Exp Med 187(3): 341-348). Without wishing to be bound by theory, it is believed that inactivating (e.g., deleting) the Joint region may contribute to the stability of the recombinant herpes simplex virus genome and/or allow for the recombinant herpes simplex virus genome to accommodate more and/or larger transgenes.

In some embodiments, the recombinant herpes simplex virus genome comprises an inactivating mutation in the ICP4 (one or both copies), ICP22, and ICP27 genes. In some embodiments, the recombinant herpes simplex virus genome comprises an inactivating mutation in the ICP4 (one or both copies), ICP27, and UL55 genes. In some embodiments, the recombinant herpes simplex virus genome comprises an inactivating mutation in the ICP4 (one or both copies), ICP22, ICP27, ICP47, and UL55 genes. In some embodiments, the inactivating mutation in the ICP4 (one or both copies), ICP27, and/or UL55 genes is a deletion of the coding sequence of the ICP4 (one or both copies), ICP27, and/or UL55 genes. In some embodiments, the inactivating mutation in the ICP22 and ICP47 genes is a deletion in the promoter region of the ICP22 and ICP47 genes (e.g., the ICP22 and ICP47 coding sequences are intact but are not transcriptionally active). In some embodiments, the recombinant herpes simplex virus genome comprises a deletion in the coding sequence of the ICP4 (one or both copies), ICP27, and UL55 genes, and a deletion in the promoter region of the ICP22 and ICP47 genes. In some embodiments, the recombinant herpes simplex virus genome further comprises an inactivating mutation in the ICP0 (one or both copies) and/or UL41 genes.

In some embodiments, the recombinant herpes simplex virus genome comprises an inactivating mutation in the ICP0 (one or both copies) and ICP4 (one or both copies) genes. In some embodiments, the recombinant herpes simplex virus genome comprises an inactivating mutation in the ICP0 (one or both copies), ICP4 (one or both copies), and ICP22 genes. In some embodiments, the recombinant herpes simplex virus genome comprises an inactivating mutation in the ICP0 (one or both copies), ICP4 (one or both copies), ICP22, and ICP27 genes. In some embodiments, the recombinant herpes simplex virus genome comprises an inactivating mutation in the ICP0 (one or both copies), ICP4 (one or both copies), ICP22, ICP27 and UL55 genes. In some embodiments, the inactivating mutation in the ICP0 (one or both copies), ICP4 (one or both copies), ICP22, ICP27 and/or UL55 genes comprises a deletion of the coding sequence of the ICP0 (one or both copies), ICP4 (one or both copies), ICP22, ICP27 and/or UL55 genes. In some embodiments, the recombinant herpes simplex virus genome further comprises an inactivating mutation in the ICP47 and/or the UL41 genes.

In some embodiments, a recombinant herpes simplex virus genome comprises one or more polynucleotides of the present disclosure within one, two, three, four, five, six, seven or more viral gene loci. Examples of suitable viral loci may include, without limitation, the ICP0 (one or both copies), ICP4 (one or both copies), ICP22, ICP27, ICP47, tk, UL41 and/or UL55 herpes simplex viral gene loci. In some embodiments, a recombinant herpes simplex virus genome comprises one or more polynucleotides of the present disclosure within one or both of the viral ICP4 gene loci (e.g., a recombinant virus comprising a polynucleotide encoding an ichthyosis-associated polypeptide in one or both of the ICP4 loci). In some embodiments, a recombinant herpes simplex virus genome comprises one or more polynucleotides of the present disclosure within the viral ICP22 gene locus (e.g., a recombinant virus carrying a polynucleotide encoding an ichthyosis-associated polypeptide in the ICP22 locus). In some embodiments, a recombinant herpes simplex virus genome comprises one or more polynucleotides of the present disclosure within the viral UL41 gene locus (e.g., a recombinant virus carrying a polynucleotide encoding an ichthyosis-associated polypeptide in the UL41 locus). In some embodiments, a recombinant herpes simplex virus genome comprises one or more polynucleotides of the present disclosure within the viral ICP27 gene locus (e.g., a recombinant virus carrying a polynucleotide encoding an ichthyosis-associated polypeptide in the ICP27 locus). In some embodiments, a recombinant herpes simplex virus genome comprises one or more polynucleotides of the present disclosure within the viral ICP47 gene locus (e.g., a recombinant virus carrying a polynucleotide encoding an ichthyosis-associated polypeptide in the ICP47 locus).

In some embodiments, a recombinant herpes simplex virus genome comprises one or more polynucleotides of the present disclosure within one or both of the viral ICP4 gene loci, and one or more polynucleotides of the present disclosure within the viral ICP22 gene locus (e.g., a recombinant virus comprising a polynucleotide encoding a first ichthyosis-associated polypeptide in one or both of the ICP4 loci, and a polynucleotide encoding a second ichthyosis-associated polypeptide in the ICP22 locus; etc.). In some embodiments, the first and second ichthyosis-associated polypeptides are the same. In some embodiments, the first and second ichthyosis-associated polypeptides are different. In some embodiments, a recombinant herpes simplex virus genome comprises one or more polynucleotides of the present disclosure within one or both of the viral ICP4 gene loci, and one or more polynucleotides of the present disclosure within the viral UL41 gene locus (e.g., a recombinant virus comprising a polynucleotide encoding a first ichthyosis-associated polypeptide in one or both of the ICP4 loci, and a polynucleotide encoding a second ichthyosis-associated polypeptide in the UL41 locus etc.). In some embodiments, the first and second ichthyosis-associated polypeptides are the same. In some embodiments, the first and second ichthyosis-associated polypeptides are different. In some embodiments, a recombinant herpes simplex virus genome comprises one or more polynucleotides of the present disclosure within the viral UL41 gene locus, and one or more polynucleotides of the present disclosure within the viral ICP22 gene locus (e.g., a recombinant virus comprising a polynucleotide encoding a first ichthyosis-associated polypeptide in the UL41 locus, and a polynucleotide encoding second ichthyosis-associated polypeptide in the ICP22 locus; etc.). In some embodiments, the first and second ichthyosis-associated polypeptides are the same. In some embodiments, the first and second ichthyosis-associated polypeptides are different. In some embodiments, a recombinant herpes simplex virus genome comprises one or more polynucleotides of the present disclosure within one or both of the viral ICP4 gene loci, one or more polynucleotides of the present disclosure within the viral ICP22 gene locus, and one or more polynucleotides of the present disclosure within the viral UL41 gene locus (e.g., a recombinant virus comprising a polynucleotide encoding a first ichthyosis-associated polypeptide in one or both of the ICP4 loci, a polynucleotide encoding a second ichthyosis-associated polypeptide in the ICP22 locus, and a polynucleotide encoding a third ichthyosis-associated polypeptide in the UL41 locus; etc.). In some embodiments, the first, second, and/or third ichthyosis-associated polypeptides are the same. In some embodiments, the first, second, and/or third ichthyosis-associated polypeptides are different.

In some embodiments, the recombinant herpes virus genome (e.g., a recombinant herpes simplex virus genome) has been engineered to decrease or eliminate expression of one or more herpes virus genes (e.g., one or more toxic herpes virus genes), such as one or both copies of the HSV ICP0 gene, one or both copies of the HSV ICP4 gene, the HSV ICP22 gene, the HSV UL41 gene, the HSV ICP27 gene, etc. In some embodiments, the recombinant herpes virus genome (e.g., recombinant herpes simplex virus genome) has been engineered to reduce cytotoxicity of the recombinant genome (e.g., when introduced into a target cell) as compared to a corresponding wild-type herpes virus genome (e.g., a wild-type herpes simplex virus genome). In some embodiments, the target cell is a human cell. In some embodiments, the target cell is a cell of the epidermis and/or dermis (e.g., a cell of the human epidermis and/or dermis). In some embodiments, the target cell is a keratinocyte or fibroblast (e.g., a human keratinocyte or human fibroblast). In some embodiments, the target cell is a cell of the mucosa. In some embodiments, cytotoxicity (e.g., in human keratinocytes and/or fibroblast cells) of the recombinant herpes virus genome (e.g., a recombinant herpes simplex virus genome) is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% as compared to a corresponding wild-type herpes virus genome (e.g., measuring the relative cytotoxicity of a recombinant ΔICP4 (one or both copies) herpes simplex virus genome vs. a wild-type herpes simplex virus genome in human keratinocytes or fibroblasts (primary cells or cell lines); measuring the relative cytotoxicity of a recombinant ΔICP4 (one or both copies)/ΔICP22 herpes simplex virus genome vs. a wild-type herpes simplex virus genome in human keratinocytes or fibroblasts (primary cells or cell lines); etc.). In some embodiments, cytotoxicity (e.g., in human keratinocytes and/or fibroblast cells) of the recombinant herpes virus genome (e.g., a recombinant herpes simplex virus genome) is reduced by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, at least about 100-fold, at least about 250-fold, at least about 500-fold, at least about 750-fold, at least about 1000-fold, or more as compared to a corresponding wild-type herpes virus genome (e.g., measuring the relative cytotoxicity of a recombinant ΔICP4 (one or both copies) herpes simplex virus genome vs. a wild-type herpes simplex virus genome in human keratinocytes or fibroblasts (primary cells or cell lines); measuring the relative cytotoxicity of a recombinant ΔICP4 (one or both copies)/ΔICP22 herpes simplex virus genome vs. a wild-type herpes simplex virus genome in human keratinocytes or fibroblasts (primary cells or cell lines); etc.). Methods of measuring cytotoxicity are known to one of ordinary skill in the art, including, for example, through the use of vital dyes (formazan dyes), protease biomarkers, an MTT assay (or an assay using related tetrazolium salts such as XTT, MTS, water-soluble tetrazolium salts, etc.), measuring ATP content, etc.

In some embodiments, the recombinant herpes virus genome (e.g., a recombinant herpes simplex virus genome) has been engineered to reduce its impact on host cell proliferation after exposure of a target cell to the recombinant genome, as compared to a corresponding wild-type herpes virus genome (e.g., a wild-type herpes simplex virus genome). In some embodiments, the target cell is a human cell. In some embodiments, the target cell is a cell of the epidermis and/or dermis (e.g., a cell of the human epidermis and/or dermis). In some embodiments, the target cell is a keratinocyte or fibroblast (e.g., a human keratinocyte or human fibroblast. In some embodiments, host cell proliferation (e.g., of human keratinocytes and/or fibroblasts) after exposure to the recombinant genome is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% faster as compared to host cell proliferation after exposure to a corresponding wild-type herpes virus genome (e.g., measuring the relative cellular proliferation after exposure to a recombinant ΔICP4 (one or both copies) herpes simplex virus genome vs. cellular proliferation after exposure to a wild-type herpes simplex virus genome in human keratinocytes or fibroblasts (primary cells or cell lines); measuring the relative cellular proliferation after exposure to a recombinant ΔICP4 (one or both copies)/ΔICP22 herpes simplex virus genome vs. cellular proliferation after exposure to a wild-type herpes simplex virus genome in human keratinocytes or fibroblasts (primary cells or cell lines); etc.). In some embodiments, host cell proliferation (e.g., of human keratinocytes and/or fibroblasts) after exposure to the recombinant genome is at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, at least about 100-fold, at least about 250-fold, at least about 500-fold, at least about 750-fold, or at least about 1000-fold faster as compared to host cell proliferation after exposure to a corresponding wild-type herpes virus genome (e.g., measuring the relative cellular proliferation after exposure to a recombinant ΔICP4 (one or both copies) herpes simplex virus genome vs. cellular proliferation after exposure to a wild-type herpes simplex virus genome in human keratinocytes or fibroblasts (primary cells or cell lines); measuring the relative cellular proliferation after exposure to a recombinant ΔICP4 (one or both copies)/ΔICP22 herpes simplex virus genome vs. cellular proliferation after exposure to a wild-type herpes simplex virus genome in human keratinocytes or fibroblasts (primary cells or cell lines); etc.). Methods of measuring cellular proliferation are known to one of ordinary skill in the art, including, for example, through the use of a Ki67 cell proliferation assay, a BrdU cell proliferation assay, etc.

A vector (e.g., herpes viral vector) may include one or more polynucleotides of the present disclosure in a form suitable for expression of the polynucleotide in a host cell. Vectors may include one or more regulatory sequences operatively linked to the polynucleotide to be expressed (e.g., as described above).

In some embodiments, a recombinant nucleic acid (e.g., a recombinant herpes virus genome, such as a recombinant herpes simplex virus genome) of the present disclosure comprises one or more of the polynucleotides described herein inserted in any orientation in the recombinant nucleic acid. If the recombinant nucleic acid comprises two or more polynucleotides described herein (e.g., two or more, three or more, etc.), the polynucleotides may be inserted in the same orientation or opposite orientations to one another. Without wishing to be bound be theory, incorporating two polynucleotides (e.g., two transgenes) into a recombinant nucleic acid (e.g., a vector) in an antisense orientation may help to avoid read-through and ensure proper expression of each polynucleotide.

In some embodiments, the present disclosure relates to one or more heterologous polynucleotides (e.g., a bacterial artificial chromosome (BAC)) comprising any of the recombinant nucleic acids described herein.

### IV. Viruses

Certain aspects of the present disclosure relate to viruses comprising any of the polynucleotides and/or recombinant nucleic acids described herein. In some embodiments, the virus is capable of infecting one or more target cells of a subject (e.g., a human). In some embodiments, the virus is suitable for delivering the polynucleotides and/or recombinant nucleic acids into one or more target cells of a subject (e.g., a human). In some embodiments, the present disclosure relates to one or more viral particles comprising any of the polynucleotides and/or recombinant nucleic acids described herein. In some embodiments, the one or more target cells are one or more human cells. In some embodiments, the one or more target cells are one or more cells of the skin (e.g., one or more cells of the epidermis, dermis, and/or subcutis). In some embodiments, the one or more target cells are cells of the epidermis and/or dermis (e.g., cells of the human epidermis and/or dermis). In some embodiments, the one or more target cells are selected from keratinocytes, melanocytes, Langerhans cells, Merkel cells, mast cells, fibroblasts, and/or adipocytes. In some embodiments, the one or more target cells are keratinocytes. In some embodiments, the one or more target cells reside in the stratum corneum, stratum granulosum, stratum spinulosum, stratum basale, and/or basement membrane. In some embodiments, the one or more target cells are one or more epidermal cells. In some embodiments, the one or more target cells are one or more dermal cells.

Any suitable virus known in the art may be used, including, for example, adenovirus, adeno-associated virus, retrovirus, lentivirus, sendai virus, papillomavirus, herpes virus (e.g., a herpes simplex virus), vaccinia virus, and/or any hybrid or derivative viruses thereof. In some embodiments, the virus is attenuated. In some embodiments, the virus is replication defective. In some embodiments, the virus is replication competent. In some embodiments, the virus has been modified to alter its tissue tropism relative to the tissue tropism of a corresponding unmodified, wild-type virus. In some embodiments, the virus has reduced cytotoxicity as compared to a corresponding wild-type virus. Methods of producing a virus comprising recombinant nucleic acids are well known to one of ordinary skill in the art.

In some embodiments, the virus is a member of the Herpesviridae family of DNA viruses, including, for example, a herpes simplex virus, a varicella zoster virus, a human cytomegalovirus, a herpesvirus 6A, a herpesvirus 6B, a herpesvirus 7, and a Kaposi's sarcoma-associated herpesvirus, etc. In some embodiments, the herpes virus is attenuated. In some embodiments, the herpes virus is replication defective. In some embodiments, the herpes virus is replication competent. In some embodiments, the herpes virus has reduced cytotoxicity as compared to a corresponding wild-type herpes virus. In some embodiments, the herpes virus is not oncolytic.

In some embodiments, the herpes virus is a herpes simplex virus. Herpes simplex viruses comprising recombinant nucleic acids may be produced by a process disclosed, for example, in WO2015/009952 and/or WO2017/176336. In some embodiments, the herpes simplex virus is attenuated. In some embodiments, the herpes simplex virus is replication competent. In some embodiments, the herpes simplex virus is replication defective. In some embodiments, the herpes simplex virus is a herpes simplex virus type 1 (HSV-1), a herpes simplex virus type 2 (HSV-2), or any derivatives thereof. In some embodiments, the herpes simplex virus is a herpes simplex virus type 1 (HSV-1). In some embodiments, the HSV-1 is replication defective. In some embodiments, the hsv-1 is replication competent. In some embodiments, the HSV-1 is attenuated. In some embodiments, the herpes simplex virus (e.g., the HSV-1) has reduced cytotoxicity as compared to a corresponding wild-type herpes simplex virus (e.g., a wild-type HSV-1). In some embodiments, the herpes simplex virus (e.g., the HSV-1) is not oncolytic.

In some embodiments, the herpes simplex virus has been modified to alter its tissue tropism relative to the tissue tropism of an unmodified, wild-type herpes simplex virus. In some embodiments, the herpes simplex virus comprises a modified envelope. In some embodiments, the modified envelope comprises one or more (e.g., one or more, two or more, three or more, four or more, etc.) mutant herpes simplex virus glycoproteins. Examples of herpes simplex virus glycoproteins may include, but are not limited to, the glycoproteins gB, gC, gD, gH, and gL. In some embodiments, the modified envelope alters the herpes simplex virus tissue tropism relative to a wild-type herpes simplex virus.

In some embodiments, the transduction efficiency (in vitro and/or in vivo) of a virus of the present disclosure (e.g., a herpes virus such as a herpes simplex virus) for one or more target cells (e.g., one or more human keratinocytes and/or fibroblasts) is at least about 25%. For example, the transduction efficiency of the virus for one or more target cells may be at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, at least about 99.5%, or more. In some embodiments, the virus is a herpes simplex virus and the transduction efficiency of the virus for one or more target cells (e.g., one or more human keratinocytes and/or fibroblasts) is about 85% to about 100%. In some embodiments, the virus is a herpes simplex virus and the transduction efficiency of the virus for one or more target cells (e.g., one or more human keratinocytes and/or fibroblasts) is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100%. Methods of measuring viral transduction efficiency in vitro or in vivo are well known to one of ordinary skill in the art, including, for example, qPCR analysis, deep sequencing, western blotting, fluorometric analysis (such as fluorescent in situ hybridization (FISH), fluorescent reporter gene expression, immunofluorescence, FACS), etc.

### V. Pharmaceutical Compositions and Formulations

Certain aspects of the present disclosure relate to pharmaceutical compositions and/or formulations comprising any of the recombinant nucleic acids (e.g., recombinant herpes virus genomes) and/or viruses (e.g., herpes viruses comprising a recombinant genomes) described herein (such as a herpes simplex virus comprising a recombinant herpes simplex virus genome), and a pharmaceutically acceptable excipient or carrier.

In some embodiments, the pharmaceutical composition or formulation comprises any one or more of the viruses (e.g., herpes viruses) described herein. In some embodiments, the pharmaceutical composition or formulation comprises from about 10⁴ to about 10¹² plaque forming units (PFU)/mL of the virus. For example, the pharmaceutical composition or formulation may comprise from about 10⁴ to about 10¹², about 10⁵ to about 10¹², about 10⁶ to about 10¹², about 10⁷ to about 10¹², about 10⁸ to about 10¹², about 10⁹ to about 10¹², about 10¹⁰ to about 10¹², about 10¹¹ to about 10¹², about 10⁴ to about 10¹¹, about 10⁵ to about 10¹¹, about 10⁶ to about 10¹¹, about 10⁷ to about 10¹¹, about 10⁸ to about 10¹¹, about 10⁹ to about 10¹¹, about 10¹⁰ to about 10¹¹, about 10⁴ to about 10¹⁰, about 10⁵ to about 10¹⁰, about 10⁶ to about 10¹⁰, about 10⁷ to about 10¹⁰, about 10⁸ to about 10¹⁰, about 10⁹ to about 10¹⁰, about 10⁴ to about 10⁹, about 10⁵ to about 10⁹, about 10⁶ to about 10⁹, about 10⁷ to about 10⁹, about 10⁸ to about 10⁹, about 10⁴ to about 10⁸, about 10⁵ to about 10⁸, about 10⁶ to about 108, about 10⁷ to about 10⁸, about 10⁴ to about 10⁷, about 10⁵ to about 10⁷, about 10⁶ to about 10⁷, about 10⁴ to about 10⁶, about 10⁵ to about 10⁶, or about 10⁴ to about 10⁵ PFU/mL of the virus. In some embodiments, the pharmaceutical composition or formulation comprises about 10⁴, about 10⁵, about 10⁶, about 10⁷, about 10⁸, about 10⁹, about 10¹⁰, about 10¹¹, or about 10¹² PFU/mL of the virus.

Pharmaceutical compositions and formulations can be prepared by mixing the active ingredient(s) (such as a recombinant nucleic acid and/or a virus) having the desired degree of purity with one or more pharmaceutically acceptable carriers or excipients. Pharmaceutically acceptable carriers or excipients are generally nontoxic to recipients at the dosages and concentrations employed, and may include, but are not limited to: buffers (such as phosphate, citrate, acetate, and other organic acids); antioxidants (such as ascorbic acid and methionine); preservatives (such as octadecyldimethylbenzyl ammonium chloride, benzalkonium chloride, benzethonium chloride, phenol, butyl or benzyl alcohol, alkyl parabens, catechol, resorcinol, cyclohexanol, 3-pentanol, and m-cresol); amino acids (such as glycine, glutamine, asparagine, histidine, arginine, or lysine); low molecular weight (less than about 10 residues) polypeptides; proteins (such as serum albumin, gelatin, or immunoglobulins); polyols (such as glycerol, e.g., formulations including 1%, 2%, 3%, 4%, 5%, 6%, 7%, 8%, 9%, 10%, 11%, 12%, 13%, 14%, 15%, etc. glycerol); hydrophilic polymers (such as polyvinylpyrrolidone); monosaccharides, disaccharides, and other carbohydrates (including glucose, mannose, or dextrins); chelating agents (such as EDTA); sugars (such as sucrose, mannitol, trehalose, or sorbitol); salt-forming counter-ions (such as sodium); metal complexes (such as Zn-protein complexes); and/or non-ionic surfactants (such as polyethylene glycol (PEG)). A thorough discussion of pharmaceutically acceptable carriers is available in REMINGTON'S PHARMACEUTICAL SCIENCES (Mack Pub. Co., N.J. 1991).

In some embodiments, the pharmaceutical composition or formulation comprises one or more lipid (e.g., cationic lipid) carriers. In some embodiments, the pharmaceutical composition or formulation comprises one or more nanoparticle carriers. Nanoparticles are submicron (less than about 1000 nm) sized drug delivery vehicles that can carry encapsulated drugs (such as synthetic small molecules, proteins, peptides, cells, viruses, and nucleic acid-based biotherapeutics) for rapid or controlled release. A variety of molecules (e.g., proteins, peptides, recombinant nucleic acids, etc.) can be efficiently encapsulated in nanoparticles using processes well known in the art. In some embodiments, a molecule “encapsulated” in a nanoparticle may refer to a molecule (such as a virus) that is contained within the nanoparticle or attached to and/or associated with the surface of the nanoparticle, or any combination thereof. Nanoparticles for use in the compositions or formulations described herein may be any type of biocompatible nanoparticle known in the art, including, for example, nanoparticles comprising poly(lactic acid), poly(glycolic acid), PLGA, PLA, PGA, and any combinations thereof (see e.g., Vauthier et al. Adv Drug Del Rev. (2003) 55: 519-48; US2007/0148074; US2007/0092575; US2006/0246139; U.S. Pat. Nos. 5,753,234; 7,081,483; and WO2006/052285).

In some embodiments, the pharmaceutically acceptable carrier or excipient may be adapted for or suitable for any administration route known in the art, including, for example, intravenous, intramuscular, subcutaneous, cutaneous, oral, nasal, intratracheal, sublingual, buccal, topical, transdermal, intradermal, intraperitoneal, intraorbital, subretinal, intravitreal, transmucosal, intraarticular, by implantation, by inhalation, intrathecal, intraventricular, and/or intranasal administration. In some embodiments, the pharmaceutical composition or formulation is adapted for or suitable for any administration route known in the art, including, for example, intravenous, intramuscular, subcutaneous, cutaneous, oral, nasal, intratracheal, sublingual, buccal, topical, transdermal, intradermal, intraperitoneal, intraorbital, intravitreal, subretinal, transmucosal, intraarticular, by implantation, by inhalation, intrathecal, intraventricular, and/or intranasal administration. In some embodiments, the pharmaceutically acceptable carrier or excipient is adapted for or suitable for topical, transdermal, subcutaneous, intradermal, and/or transmucosal administration. In some embodiments, the pharmaceutical composition or formulation is adapted for or suitable for topical, transdermal, subcutaneous, intradermal, and/or transmucosal administration. In some embodiments, the pharmaceutically acceptable carrier or excipient is adapted for or suitable for topical, transdermal, subcutaneous, and/or intradermal administration. In some embodiments, the pharmaceutical composition or formulation is adapted for or suitable for topical, transdermal, subcutaneous, and/or intradermal administration. In some embodiments, the pharmaceutically acceptable carrier or excipient is adapted for or suitable for topical, transdermal, and/or intradermal administration. In some embodiments, the pharmaceutical composition or formulation is adapted for or suitable for topical, transdermal, and/or intradermal administration. In some embodiments, the pharmaceutically acceptable carrier or excipient is adapted for or suitable for topical administration. In some embodiments, the pharmaceutical composition or formulation is adapted for or suitable for topical administration.

Examples of carriers or excipients adapted for or suitable for use in pharmaceutical compositions or formulations of the present disclosure may include, but are not limited to, ointments, oils, pastes, creams, aerosols, suspensions, emulsions, fatty ointments, gels (e.g., methylcellulose gels, such as carboxy methylcellulose, hydroxypropyl methylcellulose, etc.), powders, liquids, lotions, solutions, sprays, patches (e.g., transdermal patches or microneedle patches), adhesive strips, a microneedle or microneedle arrays, and inhalants. In some embodiments, the carrier or excipient (e.g., the pharmaceutically acceptable carrier or excipient) comprises one or more (e.g., one or more, two or more, three or more, four or more, five or more, etc.) of an ointment, oil, paste, cream, aerosol, suspension, emulsion, fatty ointment, gel, powder, liquid, lotion, solution, spray, patch, adhesive strip and an inhalant. In some embodiments, the carrier comprises a patch (e.g. a patch that adheres to the skin), such as a transdermal patch or a microneedle patch. In some embodiments, the carrier comprises a microneedle or microneedle array. Methods for making and using microneedle arrays suitable for composition delivery are generally known in the art (see e.g., Kim Y. et al. “Microneedles for drug and vaccine delivery”. Advanced Drug Delivery Reviews 2012, 64 (14): 1547-68).

In some embodiments, the pharmaceutical composition or formulation further comprises one or more additional components. Examples of additional components may include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); wetting agents (e.g., sodium lauryl sulphate, etc.); salt solutions; alcohols; polyethylene glycols; gelatin; lactose; amylase; magnesium stearate; talc; silicic acid; viscous paraffin; methylcellulose (e.g., carboxy methylcellulose, hydroxypropyl methylcellulose, etc.); polyvinylpyrrolidone; sweetenings; flavorings; perfuming agents; colorants; moisturizers; sunscreens; antibacterial agents; agents able to stabilize polynucleotides or prevent their degradation, and the like. In some embodiments, the pharmaceutical composition or formulation comprises a methylcellulose gel, such as hydroxypropyl methylcellulose, carboxy methylcellulose, etc., (e.g., at about 0.5%, at about 1%, at about 1.5%, at about 2%, at about 2.5%, at about 3%, at about 3.5%, at about 4%, at about 4.5%, at about 5%, at about 5.5%, at about 6%, at about 6.5%, at about 7%, at about 7.5%, at about 8%, at about 8.5%, at about 9%, at about 9.5%, at about 10%, at about 10.5%, at about 11%, at about 11.5%, at about 12%, etc.). In some embodiments, the pharmaceutical composition or formulation comprises a phosphate buffer. In some embodiments, the pharmaceutical composition or formulation comprises glycerol (e.g., at about 1%, about 2%, about 3%, about 4%, about 5%, about 6%, about 7%, about 8%, about 9%, about 10%, about 11%, about 12%, about 13%, about 14%, about 15%, etc.). In some embodiments, the pharmaceutical composition or formulation comprises a methylcellulose gel (e.g., hydroxypropyl methylcellulose, carboxy methylcellulose, etc.), a phosphate buffer, and/or glycerol.

Compositions and formulations (e.g., pharmaceutical compositions and formulations) to be used for in vivo administration are generally sterile. Sterility may be readily accomplished, e.g., by filtration through sterile filtration membranes.

In some embodiments, any of the recombinant nucleic acids, viruses, and/or pharmaceutical compositions or formulations described herein may be used to deliver one or more polynucleotides encoding an ichthyosis-associated polypeptide (e.g., a human Steryl-sulfatase polypeptide) into one or more cells of a subject (e.g., one or more Steryl-sulfatase deficient cells, one or more cells harboring an STS gene mutation, etc.). In some embodiments, any of the recombinant nucleic acids, viruses, and/or pharmaceutical compositions or formulations described herein may be used in a therapy. In some embodiments, any of the recombinant nucleic acids, viruses, and/or pharmaceutical compositions or formulations described herein may be used in the treatment of a disease, disorder, defect, or condition that would benefit from the expression of an ichthyosis-associated polypeptide (e.g., one or more forms of congenital ichthyosis; a disease, disorder, defect, or condition associated with an ichthyosis-associated polypeptide deficiency (such as X-linked ichthyosis); a disease, disorder, defect, or condition associated a ichthyosis-associated gene mutation, etc.). In some embodiments, any of the recombinant nucleic acids, viruses, and/or pharmaceutical compositions or formulations described herein may be used in the treatment of one or more forms of congenital ichthyosis (e.g., used in the treatment of one or more of harlequin ichthyosis (HI), autosomal recessive congenital ichthyosis (ARCI), lamellar ichthyosis (LI), congenital ichthyosiform erythroderma (CIE), Chanarin-Dorfman syndrome (CDS), Sjogren-Larsson syndrome (SLS), mental retardation, enteropathy, deafness, peripheral neuropathy, ichthyosis, and keratoderma (MEDNIK) syndrome, chondrodysplasia punctata 1 (CDPX1), chondrodysplasia punctata 2 (CDPX2), peeling skin syndrome (PSS), neonatal ichthyosis-sclerosing cholangitis (NISCH) syndrome, ichthyosis vulgaris, keratitis-ichthyosis-deafness syndrome (KID), palmoplantar keratoderma (PPK), palmoplantar keratoderma with sensorineural hearing loss (PPK/SNHL), epidermolytic palmoplantar keratoderma (EPPK), erythrokeratodermia variabilis (EKV), Clouston syndrome, progressive symmetric erythrokeratodermia, epidermolytic ichthyosis (EI), superficial epidermolytic ichthyosis (SEI), loricrin keratoderma, ichthyosis follicularis, alopecia, and photophobia (IFAP) syndrome, Olmsted syndrome, congenital hemidysplasia with ichthyosiform nevus and limb defects (CHILD) syndrome, Refsum disease, Neu-Laxova syndrome, keratosis linearis with ichthyosis congenita and sclerosing keratoderma (KLICK) syndrome, ichthyosis prematurity syndrome (IPS), cerebral dysgenesis, neuropathy, ichthyosis, and palmoplantar keratoderma (CEDNIK) syndrome, X-linked ichthyosis, arthrogryposis-renal dysfunction-cholestasis (ARC) syndrome, and/or restrictive dermopathy). In some embodiments, the congenital ichthyosis is not ARCI. In some embodiments, any of the recombinant nucleic acids, viruses, and/or pharmaceutical compositions or formulations described herein may be used in the treatment of X-linked ichthyosis.

In some embodiments, any of the recombinant nucleic acids, viruses, and/or pharmaceutical compositions or formulations described herein may be used in the preparation or manufacture of a medicament. In some embodiments, any of the recombinant nucleic acids, viruses, and/or pharmaceutical compositions or formulations described herein may be used in the preparation or manufacture of a medicament useful for delivering one or more polynucleotides encoding an ichthyosis-associated polypeptide (e.g., a human Steryl-sulfatase polypeptide) into one or more cells of a subject (e.g., one or more Steryl-sulfatase deficient cells, one or more cells harboring an STS gene mutation, etc.). In some embodiments, any of the recombinant nucleic acids, viruses, and/or pharmaceutical compositions or formulations described herein may be used in the preparation or manufacture of a medicament useful for the treatment of a disease, disorder, defect, or condition that would benefit from the expression of an ichthyosis-associated polypeptide (e.g., one or more forms of congenital ichthyosis; a disease, disorder, defect, or condition associated with an ichthyosis-associated polypeptide deficiency (such as X-linked ichthyosis); a disease, disorder, defect, or condition associated with a ichthyosis-associated gene mutation, etc.). In some embodiments, any of the recombinant nucleic acids, viruses, and/or pharmaceutical compositions or formulations described herein may be used in the preparation or manufacture of a medicament useful for the treatment of one or more forms of congenital ichthyosis (e.g., useful for the treatment of one or more of harlequin ichthyosis (HI), autosomal recessive congenital ichthyosis (ARCI), lamellar ichthyosis (LI), congenital ichthyosiform erythroderma (CIE), Chanarin-Dorfman syndrome (CDS), Sjogren-Larsson syndrome (SLS), mental retardation, enteropathy, deafness, peripheral neuropathy, ichthyosis, and keratoderma (MEDNIK) syndrome, chondrodysplasia punctata 1 (CDPX1), chondrodysplasia punctata 2 (CDPX2), peeling skin syndrome (PSS), neonatal ichthyosis-sclerosing cholangitis (NISCH) syndrome, ichthyosis vulgaris, keratitis-ichthyosis-deafness syndrome (KID), palmoplantar keratoderma (PPK), palmoplantar keratoderma with sensorineural hearing loss (PPK/SNHL), epidermolytic palmoplantar keratoderma (EPPK), erythrokeratodermia variabilis (EKV), Clouston syndrome, progressive symmetric erythrokeratodermia, epidermolytic ichthyosis (EI), superficial epidermolytic ichthyosis (SEI), loricrin keratoderma, ichthyosis follicularis, alopecia, and photophobia (IFAP) syndrome, Olmsted syndrome, congenital hemidysplasia with ichthyosiform nevus and limb defects (CHILD) syndrome, Refsum disease, Neu-Laxova syndrome, keratosis linearis with ichthyosis congenita and sclerosing keratoderma (KLICK) syndrome, ichthyosis prematurity syndrome (IPS), cerebral dysgenesis, neuropathy, ichthyosis, and palmoplantar keratoderma (CEDNIK) syndrome, X-linked ichthyosis, arthrogryposis-renal dysfunction-cholestasis (ARC) syndrome, and/or restrictive dermopathy). In some embodiments, the congenital ichthyosis is not ARCI. In some embodiments, any of the recombinant nucleic acids, viruses, and/or pharmaceutical compositions or formulations described herein may be used in the preparation or manufacture of a medicament useful for the treatment of X-linked ichthyosis.

### VI. Methods

Certain aspects of the present disclosure relate to enhancing, increasing, augmenting, and/or supplementing the levels of an ichthyosis-associated polypeptide (e.g., a human Steryl-sulfatase polypeptide) in one or more cells of a subject comprising administering to the subject an effective amount of any of the recombinant nucleic acids, viruses, medicaments, and/or pharmaceutical compositions or formulations described herein. In some embodiments, the subject is a human. In some embodiments, the subject suffers from one or more forms of congenital ichthyosis (e.g., one or more of harlequin ichthyosis (HI), autosomal recessive congenital ichthyosis (ARCI), lamellar ichthyosis (LI), congenital ichthyosiform erythroderma (CIE), Chanarin-Dorfman syndrome (CDS), Sjogren-Larsson syndrome (SLS), mental retardation, enteropathy, deafness, peripheral neuropathy, ichthyosis, and keratoderma (MEDNIK) syndrome, chondrodysplasia punctata 1 (CDPX1), chondrodysplasia punctata 2 (CDPX2), peeling skin syndrome (PSS), neonatal ichthyosis-sclerosing cholangitis (NISCH) syndrome, ichthyosis vulgaris, keratitis-ichthyosis-deafness syndrome (KID), palmoplantar keratoderma (PPK), palmoplantar keratoderma with sensorineural hearing loss (PPK/SNHL), epidermolytic palmoplantar keratoderma (EPPK), erythrokeratodermia variabilis (EKV), Clouston syndrome, progressive symmetric erythrokeratodermia, epidermolytic ichthyosis (EI), superficial epidermolytic ichthyosis (SEI), loricrin keratoderma, ichthyosis follicularis, alopecia, and photophobia (IFAP) syndrome, Olmsted syndrome, congenital hemidysplasia with ichthyosiform nevus and limb defects (CHILD) syndrome, Refsum disease, Neu-Laxova syndrome, keratosis linearis with ichthyosis congenita and sclerosing keratoderma (KLICK) syndrome, ichthyosis prematurity syndrome (IPS), cerebral dysgenesis, neuropathy, ichthyosis, and palmoplantar keratoderma (CEDNIK) syndrome, X-linked ichthyosis, arthrogryposis-renal dysfunction-cholestasis (ARC) syndrome, and/or restrictive dermopathy). In some embodiments, the congenital ichthyosis is not ARCI. In some embodiments, the subject's genome comprises a mutation (e.g., a loss-of-function mutation, a pathogenic variant) in an endogenous ichthyosis-associated gene (one or both copies), such as a loss-of-function mutation in the STS gene.

In some embodiments, administration of the recombinant nucleic acid, virus, medicament, and/or pharmaceutical composition or formulation to the subject increases ichthyosis-associated polypeptide levels (transcript or protein levels) by at least about 25% in one or more contacted or treated cells of the subject, as compared to the endogenous levels of the ichthyosis-associated polypeptide in one or more corresponding untreated cells of the subject. In some embodiments, administration of the recombinant nucleic acid, virus, medicament, and/or pharmaceutical composition or formulation to the subject increases ichthyosis-associated polypeptide levels (transcript or protein levels) by at least about 25%, at least about 30%, at least about 40%, at least about 50%, at least about 50%, at least about 60%, at least about 70%, at least about 80%, at least about 90%, at least about 95%, at least about 99%, or more in one or more contacted or treated cells of the subject, as compared to the endogenous levels of the ichthyosis-associated polypeptide in one or more corresponding untreated cells of the subject. In some embodiments, administration of the recombinant nucleic acid, virus, medicament, and/or pharmaceutical composition or formulation to the subject increases ichthyosis-associated polypeptide levels (transcript or protein levels) by at least about 2-fold in one or more contacted or treated cells of the subject, as compared to the endogenous levels of the ichthyosis-associated polypeptide in one or more corresponding untreated cells in the subject. For example, administration of the recombinant nucleic acid, virus, medicament, and/or pharmaceutical composition or formulation may increase ichthyosis-associated polypeptide levels (transcript or protein levels) by at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, at least about 100-fold, at least about 250-fold, at least about 500-fold, at least about 750-fold, at least about 1000-fold, or more in one or more contacted or treated cells of the subject, as compared to the endogenous levels of the ichthyosis-associated polypeptide in one or more corresponding untreated cells in the subject. In some embodiments, the one or more contacted or treated cells are one or more cells of the epidermis, dermis, and/or mucosa. In some embodiments, the one or more contacted or treated cells are one or more cells of the epidermis and/or dermis (e.g., a keratinocyte or fibroblast). Methods of measuring transcript or protein levels from a sample are well known to one of ordinary skill in the art, including, for example, by qPCR, western blot, mass spectrometry, etc.

Other aspects of the present disclosure relate to a method of providing prophylactic, palliative, or therapeutic relief to one or more signs or symptoms of congenital ichthyosis in a subject in need thereof comprising administering to the subject an effective amount of any of the recombinant nucleic acids, viruses, medicaments, and/or pharmaceutical compositions or formulations described herein. In some embodiments, the subject is a human. In some embodiments, the subject suffers from one or more of harlequin ichthyosis (HI), autosomal recessive congenital ichthyosis (ARCI), lamellar ichthyosis (LI), congenital ichthyosiform erythroderma (CIE), Chanarin-Dorfman syndrome (CDS), Sjogren-Larsson syndrome (SLS), mental retardation, enteropathy, deafness, peripheral neuropathy, ichthyosis, and keratoderma (MEDNIK) syndrome, chondrodysplasia punctata 1 (CDPX1), chondrodysplasia punctata 2 (CDPX2), peeling skin syndrome (PSS), neonatal ichthyosis-sclerosing cholangitis (NISCH) syndrome, ichthyosis vulgaris, keratitis-ichthyosis-deafness syndrome (KID), palmoplantar keratoderma (PPK), palmoplantar keratoderma with sensorineural hearing loss (PPK/SNHL), epidermolytic palmoplantar keratoderma (EPPK), erythrokeratodermia variabilis (EKV), Clouston syndrome, progressive symmetric erythrokeratodermia, epidermolytic ichthyosis (EI), superficial epidermolytic ichthyosis (SEI), loricrin keratoderma, ichthyosis follicularis, alopecia, and photophobia (IFAP) syndrome, Olmsted syndrome, congenital hemidysplasia with ichthyosiform nevus and limb defects (CHILD) syndrome, Refsum disease, Neu-Laxova syndrome, keratosis linearis with ichthyosis congenita and sclerosing keratoderma (KLICK) syndrome, ichthyosis prematurity syndrome (IPS), cerebral dysgenesis, neuropathy, ichthyosis, and palmoplantar keratoderma (CEDNIK) syndrome, X-linked ichthyosis, arthrogryposis-renal dysfunction-cholestasis (ARC) syndrome, and restrictive dermopathy. In some embodiments, the congenital ichthyosis is not ARCI. In some embodiments, the congenital ichthyosis is X-linked ichthyosis.

Other aspects of the present disclosure relate to a method of providing prophylactic, palliative, or therapeutic relief to one or more signs or symptoms of harlequin ichthyosis in a subject in need thereof comprising administering to the subject an effective amount of any of the recombinant nucleic acids, viruses, medicaments, and/or pharmaceutical compositions or formulations described herein. In some embodiments, the subject is a human. In some embodiments, the subject's genome comprises a mutation (e.g., a loss-of-function mutation, a pathogenic variant) in the ABCA12 gene (one or both copies). In some embodiments, the recombinant nucleic acid (e.g., a recombinant herpes virus genome) comprises one or more polynucleotides encoding an ATP-binding sub-family A member 12 polypeptide (ABCA12), e.g., a human ABCA12 polypeptide. Signs and/or symptoms of harlequin ichthyosis may include, but are not limited to, thick plate-like scales of the skin, ectropion, eclabium, severe restriction of the chest and abdomen due to tightness of the skin, difficulty breathing and/or eating, low body temperature, swelling of the mouth, dehydration, lack of hydration to the corneas, hypernatremia, etc.

Other aspects of the present disclosure relate to a method of providing prophylactic, palliative, or therapeutic relief to one or more signs or symptoms of Chanarin-Dorfman syndrome (CDS) in a subject in need thereof comprising administering to the subject an effective amount of any of the recombinant nucleic acids, viruses, medicaments, and/or pharmaceutical compositions or formulations described herein. In some embodiments, the subject is a human. In some embodiments, the subject's genome comprises a mutation (e.g., a loss-of-function mutation, a pathogenic variant) in the ABHD5 gene (one or both copies). In some embodiments, the recombinant nucleic acid (e.g., a recombinant herpes virus genome) comprises one or more polynucleotides encoding a 1-acylglycerol-3-phosphate O-acyltransferase ABHD5 polypeptide (ABHD5), e.g., a human ABHD5 polypeptide. Signs and/or symptoms of CDS may include, but are not limited to, redness, fine scaling, dark pigmentation, and severe itching of the skin, liver disease with lipid storage, progressive weakness of the proximal arms and legs, CK elevation in the blood, early fatigability, cataracts, ectropion, progressive hearing loss, cognitive impairment, short stature, growth retardation, steatorrhea, an enlarged spleen, orthopedic problems, kidney dysfunction, etc.

Other aspects of the present disclosure relate to a method of providing prophylactic, palliative, or therapeutic relief to one or more signs or symptoms of Sjogren-Larsson syndrome in a subject in need thereof comprising administering to the subject an effective amount of any of the recombinant nucleic acids, viruses, medicaments, and/or pharmaceutical compositions or formulations described herein. In some embodiments, the subject is a human. In some embodiments, the subject's genome comprises a mutation (e.g., a loss-of-function mutation, a pathogenic variant) in the ALDH3A2 gene (one or both copies). In some embodiments, the recombinant nucleic acid (e.g., a recombinant herpes virus genome) comprises one or more polynucleotides encoding a Aldehyde dehydrogenase family 3 member A2 polypeptide (ALDH3A2), e.g., a human ALDH3A2 polypeptide. Signs and/or symptoms of Sjogren-Larsson syndrome may include, but are not limited to, erythema, dry/rough/scaly skin with a brownish or yellowish tone, mild to severe itchiness, leukoencephalopathy, mild to profound intellectual disabilities, delayed speech and speech difficulties, seizures, delayed development of motor skills, abnormal muscle stiffness, tiny crystals/white dots formed in the light-sensitive tissues of the eye, etc.

Other aspects of the present disclosure relate to a method of providing prophylactic, palliative, or therapeutic relief to one or more signs or symptoms of autosomal recessive congenital ichthyosis (ARCI) in a subject in need thereof comprising administering to the subject an effective amount of any of the recombinant nucleic acids, viruses, medicaments, and/or pharmaceutical compositions or formulations described herein. In some embodiments, the subject is a human. In some embodiments, the subject's genome comprises a mutation (e.g., a loss-of-function mutation, a pathogenic variant) in one or more of the ALOX12B, ALOXE3, CASP14, CERS3, CYP4F22, LIPN, NIPAL4, PNPLA1, SDR9C7, SLC27A4, ST14, and/or SULT2B1 genes (one or both copies). In some embodiments, the recombinant nucleic acid (e.g., a recombinant herpes virus genome) comprises one or more polynucleotides encoding one or more (e.g., one or more, two or more, three or more, four our more, five or more, six or more, seven or more, eight or more, nine or more, 10 or more, 11 or more, or all 12) of a Arachidonate 12-lipoxygenase 12R-type polypeptide (ALOX12B), Hydroperoxide isomerase ALOXE3 polypeptide (ALOXE3), Caspase-14 polypeptide (CASP14), Ceramide synthase 3 polypeptide (CERS3), Cytochrome P450 4F22 polypeptide (CYP4F22), Lipase member N polypeptide (LIPN), Magnesium transporter NIPA4 polypeptide (NIPAL4), Patatin-like phospholipase domain-containing protein 1 polypeptide (PNPLA1), Short-chain dehydrogenase/reductase family 9C member 7 polypeptide (SDR9C7), Long-chain fatty acid transport protein 4 polypeptide (SLC27A4), Suppressor of tumorigenicity 14 protein polypeptide (ST14), and/or Sulfotransferase 2B1 polypeptide (SULT2B1), e.g., one or more of a human ALOX12B, ALOXE3, CASP14, CERS3, CYP4F22, LIPN, NIPAL4, PNPLA1, SDR9C7, SLC27A4, ST14, and/or SULT2B1 polypeptide. In some embodiments, the ARCI is not TGM1-deficient ARCI and/or TGM5-deficient ARCI. Signs and/or symptoms of ARCI may include, but are not limited to, an abnormal stratum corneum, incomplete thickening of the cornified cell envelope, defects in the intercellular lipid layers in the stratum corneum, generalized scaling with variable redness of the skin, formation of large plate-like scales, accelerated epidermal turnover, palmoplantar hyperkeratosis, defective barrier function, recurrent skin infections, exposure keratitis, hypohidrosis, heat intolerance, corneal perforation, rickets, nail abnormalities, dehydration, respiratory problems, ectropion, eclabium, hypoplasia of joint and nasal cartilage, scarring alopecia, etc.

Other aspects of the present disclosure relate to a method of providing prophylactic, palliative, or therapeutic relief to one or more signs or symptoms of erythrokeratodermia variabilis/mental retardation, enteropathy, deafness, peripheral neuropathy, ichthyosis, and keratoderma (MEDNIK) syndrome in a subject in need thereof comprising administering to the subject an effective amount of any of the recombinant nucleic acids, viruses, medicaments, and/or pharmaceutical compositions or formulations described herein. In some embodiments, the subject is a human. In some embodiments, the subject's genome comprises a mutation (e.g., a loss-of-function mutation, a pathogenic variant) in one or more of the AP1S1, GJB3, and/or GJB4 genes (one or both copies). In some embodiments, the recombinant nucleic acid (e.g., a recombinant herpes virus genome) comprises one or more polynucleotides encoding one or more (e.g., one or more, two or more, or all three) of an AP-1 complex subunit sigma-1A polypeptide (AP1S1), Gap junction beta-3 polypeptide (GJB3), and/or Gap junction beta-4 polypeptide (GJB4), e.g., one or more of a human AP1S1, GJB3, and/or GJB4 polypeptide. Signs and/or symptoms of erythrokeratodermia variabilis/MEDNIK syndrome may include, but are not limited to, hyperkeratosis and red patches of variable size, shape, and duration on the skin, sensorineural deafness, peripheral neuropathy, psychomotor retardation, elevations of very long chain fatty acids, upslanting palpebral fissures, hypotonia, ichthyosiform erythroderma, gastrointestinal problems, hepatic fibrosis, cirrhosis, cholestasis, cataracts, etc.

Other aspects of the present disclosure relate to a method of providing prophylactic, palliative, or therapeutic relief to one or more signs or symptoms of chondrodysplasia punctata 1 (CDPX1) in a subject in need thereof comprising administering to the subject an effective amount of any of the recombinant nucleic acids, viruses, medicaments, and/or pharmaceutical compositions or formulations described herein. In some embodiments, the subject is a human. In some embodiments, the subject's genome comprises a mutation (e.g., a loss-of-function mutation, a pathogenic variant) in the ARSE gene (one or both copies). In some embodiments, the recombinant nucleic acid (e.g., a recombinant herpes virus genome) comprises one or more polynucleotides encoding an Arylsulfatase E polypeptide (ARSE), e.g., a human ARSE polypeptide. Signs and/or symptoms of CDPX1 may include, but are not limited to, ichthyosis, an abnormal spine, anosmia, a depressed nasal bridge, epiphyseal stippling, hearing impairment, hypogonadism, microcephaly, shortened fingers, breathing abnormalities, etc.

Other aspects of the present disclosure relate to a method of providing prophylactic, palliative, or therapeutic relief to one or more signs or symptoms of chondrodysplasia punctata 2 (CDPX2) in a subject in need thereof comprising administering to the subject an effective amount of any of the recombinant nucleic acids, viruses, medicaments, and/or pharmaceutical compositions or formulations described herein. In some embodiments, the subject is a human. In some embodiments, the subject's genome comprises a mutation (e.g., a loss-of-function mutation, a pathogenic variant) in the EBP gene (one or both copies). In some embodiments, the recombinant nucleic acid (e.g., a recombinant herpes virus genome) comprises one or more polynucleotides encoding a 3-beta-hydroxysteroid-Delta(8),Delta(7)-isomerase polypeptide (EBP), e.g., a human EBP polypeptide. Signs and/or symptoms of CDPX2 may include, but are not limited to, congenital ichthyosiform erythroderma, erythema, hyperkeratotic scaling, follicular atrophoderma (particularly in the trunk, forearms, and dorsal aspect of the hands), cicatricial alopecia, asymmetric shortening of the limbs, facial dysmorphism (low nasal bridge, frontal bossing, hypertelorism, high arched palate), joint contractures, moderate to severe sclerosis of the vertebral column, cataracts, etc.

Other aspects of the present disclosure relate to a method of providing prophylactic, palliative, or therapeutic relief to one or more signs or symptoms of peeling skin syndrome (PSS) in a subject in need thereof comprising administering to the subject an effective amount of any of the recombinant nucleic acids, viruses, medicaments, and/or pharmaceutical compositions described herein. In some embodiments, the subject is a human. In some embodiments, the subject's genome comprises a mutation (e.g., a loss-of-function mutation, a pathogenic variant) in one or more of the CDSN, CHST8, CSTA, FLG2, and/or SERPINB8 genes (one or both copies). In some embodiments, the recombinant nucleic acid (e.g., a recombinant herpes virus genome) comprises one or more polynucleotides encoding one or more (e.g., one or more, two or more, three or more, four our more, or all five) of a Corneodesmosin polypeptide (CDSN), Carbohydrate sulfotransferase 8 polypeptide (CHST8), Cystatin-A polypeptide (CSTA), Filaggrin 2 polypeptide (FLG2), and/or SerpinB8 polypeptide (SERPINB8), e.g., one or more of a human CDSN, CHST8, CSTA, FLG2, and/or SERPINB8 polypeptide. Signs and/or symptoms of PSS may include, but are not limited to, spontaneous, painless shedding or peeling of the outermost layer of the skin, itching, short stature, blisters or erosions on the hands and feet, etc.

Other aspects of the present disclosure relate to a method of providing prophylactic, palliative, or therapeutic relief to one or more signs or symptoms of neonatal ichthyosis-sclerosing cholangitis (NISCH) syndrome in a subject in need thereof comprising administering to the subject an effective amount of any of the recombinant nucleic acids, viruses, medicaments, and/or pharmaceutical compositions or formulations described herein. In some embodiments, the subject is a human. In some embodiments, the subject's genome comprises a mutation (e.g., a loss-of-function mutation, a pathogenic variant) in the CLDN1 gene (one or both copies). In some embodiments, the recombinant nucleic acid (e.g., a recombinant herpes virus genome) comprises one or more polynucleotides encoding a Claudin-1 polypeptide (CLDN1), e.g., a human CLDN1 polypeptide. Signs and/or symptoms of NISCH may include, but are not limited to, ichthyosis with diffuse white scales, scalp hypotrichosis, cicatricial alopecia, sparse eyelashes/eyebrows, oligodontia, hypodontia, enamel dysplasia, neonatal sclerosing cholangitis with jaundice and pruritus, hepatomegaly, cholestasis, portal hypertension, patent extrahepatic bile duct obstruction, splenomegaly, leukocyte vacuolization, etc.

Other aspects of the present disclosure relate to a method of providing prophylactic, palliative, or therapeutic relief to one or more signs or symptoms of ichthyosis vulgaris in a subject in need thereof comprising administering to the subject an effective amount of any of the recombinant nucleic acids, viruses, medicaments, and/or pharmaceutical compositions or formulations described herein. In some embodiments, the subject is a human. In some embodiments, the subject's genome comprises a mutation (e.g., a loss-of-function mutation, a pathogenic variant) in the FLG gene (one or both copies). In some embodiments, the recombinant nucleic acid (e.g., a recombinant herpes virus genome) comprises one or more polynucleotides encoding a Filaggrin polypeptide (FLG), e.g., a human FLG polypeptide. Signs and/or symptoms of ichthyosis vulgaris may include, but are not limited to, flaky scalp, itchy skin, polygon-shaped white, brown, or gray scales on the skin, severely dry skin, thickened skin, etc.

Other aspects of the present disclosure relate to a method of providing prophylactic, palliative, or therapeutic relief to one or more signs or symptoms of keratitis-ichthyosis-deafness (KID) syndrome, Clouston syndrome, and/or palmoplantar keratoderma with sensorineural hearing loss (PPK/SNHL) in a subject in need thereof comprising administering to the subject an effective amount of any of the recombinant nucleic acids, viruses, medicaments, and/or pharmaceutical compositions or formulations described herein. In some embodiments, the subject is a human. In some embodiments, the subject's genome comprises a mutation (e.g., a loss-of-function mutation, a pathogenic variant) in the GJB2 and/or GJB6 gene (one or both copies). In some embodiments, the recombinant nucleic acid (e.g., a recombinant herpes virus genome) comprises one or more polynucleotides encoding one or both of a Gap junction beta-2 polypeptide (GJB2) and/or Gap junction beta-6 polypeptide (GJB6), e.g., a human GJB2 and/or GJB6 polypeptide. Signs and/or symptoms of keratitis-ichthyosis-deafness (KID) syndrome, Clouston syndrome, and/or palmoplantar keratoderma with sensorineural hearing loss (PPK/SNHL) may include, but are not limited to, palmoplantar keratoderma, erythrokeratoderma, ichthyosis, keratitis, sensitivity to light, extra blood vessel growth in the eye, scarring of the eye, visual loss or blindness, nail abnormalities, progressive hair loss, hyperpigmentation of the skin, clubbing of the fingers, etc.

Other aspects of the present disclosure relate to a method of providing prophylactic, palliative, or therapeutic relief to one or more signs or symptoms of progressive symmetric erythrokeratodermia in a subject in need thereof comprising administering to the subject an effective amount of any of the recombinant nucleic acids, viruses, medicaments, and/or pharmaceutical compositions or formulations described herein. In some embodiments, the subject is a human. In some embodiments, the subject's genome comprises a mutation (e.g., a loss-of-function mutation, a pathogenic variant) in the KDSR gene (one or both copies). In some embodiments, the recombinant nucleic acid (e.g., a recombinant herpes virus genome) comprises one or more polynucleotides encoding a 3-ketodihydrosphingosine reductase polypeptide (KDSR), e.g., a human KDSR polypeptide. Signs and/or symptoms of progressive symmetric erythrokeratodermia may include, but are not limited to, reddened plaques of thickened, rough, and/or scaly skin (especially on the face, buttocks, arms, and legs) with an almost perfectly symmetrical distribution, palmoplantar keratoderma, etc.

Other aspects of the present disclosure relate to a method of providing prophylactic, palliative, or therapeutic relief to one or more signs or symptoms of epidermolytic ichthyosis (EI), superficial epidermolytic ichthyosis (SEI), and/or epidermolytic palmoplantar keratoderma in a subject in need thereof comprising administering to the subject an effective amount of any of the recombinant nucleic acids, viruses, medicaments, and/or pharmaceutical compositions or formulations described herein. In some embodiments, the subject is a human. In some embodiments, the subject's genome comprises a mutation (e.g., a loss-of-function mutation, a pathogenic variant) in one or more of the KRT1, KRT2, KRT9, and/or KRT10 genes (one or both copies). In some embodiments, the recombinant nucleic acid (e.g., a recombinant herpes virus genome) comprises one or more polynucleotides encoding one or more (e.g., one or more, two or more, three or more, or all four) of a Keratin, type II cytoskeletal 1 polypeptide (KRT1), Keratin, type II cytoskeletal 2 epidermal polypeptide (KRT2), Keratin, type I cytoskeletal 9 polypeptide (KRT9), and/or Keratin, type I cytoskeletal 10 polypeptide (KRT10), e.g., one or more of a human KRT1, KRT2, KRT9, and/or KRT10 polypeptide. Signs and/or symptoms of epidermolytic ichthyosis (EI), superficial epidermolytic ichthyosis (SEI), and/or epidermolytic palmoplantar keratoderma may include, but are not limited to, thick, blistering, warty hardening of the skin (particularly in the skin creases over joints), scales forming in parallel rows of spines or ridges, skin fragility that may blister easily following injury, generalized erythroderma, recurrent skin infections (often Staphylococcus or Streptococcus), severe scalp involvement and hair loss, heat intolerance, even, widespread thickened skin (keratosis) over the palms and soles, a red band at the edges of the keratosis, keratotic lesions appearing on the tops of the hands, feet, knees, and elbows, excessive perspiration, nail thickening, etc.

Other aspects of the present disclosure relate to a method of providing prophylactic, palliative, or therapeutic relief to one or more signs or symptoms of loricrin keratoderma in a subject in need thereof comprising administering to the subject an effective amount of any of the recombinant nucleic acids, viruses, medicaments, and/or pharmaceutical compositions or formulations described herein. In some embodiments, the subject is a human. In some embodiments, the subject's genome comprises a mutation (e.g., a loss-of-function mutation, a pathogenic variant) in the LOR gene (one or both copies). In some embodiments, the recombinant nucleic acid (e.g., a recombinant herpes virus genome) comprises one or more polynucleotides encoding a Loricrin polypeptide (LOR), e.g., a human LOR polypeptide. Signs and/or symptoms of loricrin keratoderma may include, but are not limited to, diffuse palmoplantar keratoderma, honeycomb palmoplantar hyperkeratosis (associated with pseudoainhum of the fifth digit of the hand), deafness, ichthyosis, etc.

Other aspects of the present disclosure relate to a method of providing prophylactic, palliative, or therapeutic relief to one or more signs or symptoms of ichthyosis follicularis, alopecia, and photophobia (IFAP) syndrome in a subject in need thereof comprising administering to the subject an effective amount of any of the recombinant nucleic acids, viruses, medicaments, and/or pharmaceutical compositions or formulations described herein. In some embodiments, the subject is a human. In some embodiments, the subject's genome comprises a mutation (e.g., a loss-of-function mutation, a pathogenic variant) in the MBTPS2 gene (one or both copies). In some embodiments, the recombinant nucleic acid (e.g., a recombinant herpes virus genome) comprises one or more polynucleotides encoding a Membrane-bound transcription factor site-2 protease polypeptide (MBTPS2), e.g., a human MBTPS2 polypeptide. Signs and/or symptoms of IFAP syndrome may include, but are not limited to, dry, scaly skin, absence of hair, excessive sensitivity to light, short stature, mental retardation, seizures, a tendency for respiratory infections, etc.

Other aspects of the present disclosure relate to a method of providing prophylactic, palliative, or therapeutic relief to one or more signs or symptoms of congenital hemidysplasia with ichthyosiform nevus and limb defects (CHILD) syndrome in a subject in need thereof comprising administering to the subject an effective amount of any of the recombinant nucleic acids, viruses, medicaments, and/or pharmaceutical compositions or formulations described herein. In some embodiments, the subject is a human. In some embodiments, the subject's genome comprises a mutation (e.g., a loss-of-function mutation, a pathogenic variant) in the NSDHL gene (one or both copies). In some embodiments, the recombinant nucleic acid (e.g., a recombinant herpes virus genome) comprises one or more polynucleotides encoding a Sterol-4-alpha-carboxylate 3-dehydrogenase, decarboxylating polypeptide (NSDHL), e.g., a human NSDHL polypeptide. Signs and/or symptoms of CHILD syndrome may include, but are not limited to, dry, itchy, red and scaly skin on one side of the body, absence of hair on one side of the head, limb defects (e.g., underdevelopment of fingers and toes, complete absence of limbs) that often occur on the same side of the body as the major skin symptoms, skeletal defects (e.g., abnormal ribs, anomalies of the shoulder blades) webbing of the skin between joints, absence of muscles of the breast, defects in the walls between auricles and/or ventricles, abnormalities of the central nervous system, blood vessels, kidneys, thyroid, lungs, adrenal glands, reproductive system, and urinary system (often underdevelopment on the affected side of the body), etc.

Other aspects of the present disclosure relate to a method of providing prophylactic, palliative, or therapeutic relief to one or more signs or symptoms of Refsum disease in a subject in need thereof comprising administering to the subject an effective amount of any of the recombinant nucleic acids, viruses, medicaments, and/or pharmaceutical compositions or formulations described herein. In some embodiments, the subject is a human. In some embodiments, the subject's genome comprises a mutation (e.g., a loss-of-function mutation, a pathogenic variant) in one or both of the PEX7 and/or PHYH genes (one or both copies). In some embodiments, the recombinant nucleic acid (e.g., a recombinant herpes virus genome) comprises one or more polynucleotides encoding one or both of a Peroxisomal targeting signal 2 receptor polypeptide (PEX7) and/Phytanoyl-CoA dioxygenase, peroxisomal polypeptide (PHYH), e.g., one or both of a human PEX7 and/or PHYH polypeptide. Signs and/or symptoms of Refsum disease may include, but are not limited to, dry scaly skin, retinitis pigmentosa, anosmia, bone abnormalities of the hands and feet, progressive muscle weakness and wasting, ataxia, hearing loss, abnormal heart rhythm, etc.

Other aspects of the present disclosure relate to a method of providing prophylactic, palliative, or therapeutic relief to one or more signs or symptoms of Neu-Laxova syndrome in a subject in need thereof comprising administering to the subject an effective amount of any of the recombinant nucleic acids, viruses, medicaments, and/or pharmaceutical compositions or formulations described herein. In some embodiments, the subject is a human. In some embodiments, the subject's genome comprises a mutation (e.g., a loss-of-function mutation, a pathogenic variant) in one or both of the PHGDH and/or PSAT1 genes (one or both copies). In some embodiments, the recombinant nucleic acid (e.g., a recombinant herpes virus genome) comprises one or more polynucleotides encoding one or both of a D-3-phosphoglycerate dehydrogenase polypeptide (PHGDH) and/Phosphoserine aminotransferase polypeptide (PSAT1), e.g., one or both of a human PHGDH and/or PSAT1 polypeptide. Signs and/or symptoms of Neu-Laxova syndrome may include, but are not limited to, proptosis with eyelid malformations, nose malformations, round and gaping mouth, micrognathia, low set or malformed ears, cleft lip, cleft palate, syndactyly, edema and flexion deformities, ichthyosis and hyperkeratosis, microcephaly, lissencephaly, microgyria, hypoplasia of the cerebellum, agenesis of the corpus callosum, neural tube defects, etc.

Other aspects of the present disclosure relate to a method of providing prophylactic, palliative, or therapeutic relief to one or more signs or symptoms of keratosis linearis with ichthyosis congenita and sclerosing keratoderma (KLICK) syndrome in a subject in need thereof comprising administering to the subject an effective amount of any of the recombinant nucleic acids, viruses, medicaments, and/or pharmaceutical compositions or formulations described herein. In some embodiments, the subject is a human. In some embodiments, the subject's genome comprises a mutation (e.g., a loss-of-function mutation, a pathogenic variant) in the POMP gene (one or both copies). In some embodiments, the recombinant nucleic acid (e.g., a recombinant herpes virus genome) comprises one or more polynucleotides encoding a Proteasome maturation protein polypeptide (POMP), e.g., a human POMP polypeptide. Signs and/or symptoms of KLICK syndrome may include, but are not limited to, linear hyperkeratosis (without evidence of Koebner phenomenon), moderate, non-blistering ichthyosis, palmoplantar keratoderma, sclerosing flexion deformities of the fingers, noninflamed keratotic striae, etc.

Other aspects of the present disclosure relate to a method of providing prophylactic, palliative, or therapeutic relief to one or more signs or symptoms of ichthyosis prematurity syndrome (IPS) in a subject in need thereof comprising administering to the subject an effective amount of any of the recombinant nucleic acids, viruses, medicaments, and/or pharmaceutical compositions or formulations described herein. In some embodiments, the subject is a human. In some embodiments, the subject's genome comprises a mutation (e.g., a loss-of-function mutation, a pathogenic variant) in the SLC27A4 gene (one or both copies). In some embodiments, the recombinant nucleic acid (e.g., a recombinant herpes virus genome) comprises one or more polynucleotides encoding a Long-chain fatty acid transport protein 4 polypeptide (SLC27A4), e.g., a human SLC27A4 polypeptide. Signs and/or symptoms of IPS may include, but are not limited to, a thick caseous layer of skin, red endemic skin, spongy and desquamating skin, respiratory problems, eosinophilia, etc.

Other aspects of the present disclosure relate to a method of providing prophylactic, palliative, or therapeutic relief to one or more signs or symptoms of cerebral dysgenesis, neuropathy, ichthyosis, and palmoplantar keratoderma (CEDNIK) syndrome in a subject in need thereof comprising administering to the subject an effective amount of any of the recombinant nucleic acids, viruses, medicaments, and/or pharmaceutical compositions described herein. In some embodiments, the subject is a human. In some embodiments, the subject's genome comprises a mutation (e.g., a loss-of-function mutation, a pathogenic variant) in the SNAP29 gene (one or both copies). In some embodiments, the recombinant nucleic acid (e.g., a recombinant herpes virus genome) comprises one or more polynucleotides encoding a Synaptosomal-associated protein 29 polypeptide (SNAP29), e.g., a human SNAP29 polypeptide. Signs and/or symptoms of CEDNIK syndrome may include, but are not limited to, failure to thrive, roving eye movements, poor head and trunk control, progressive microcephaly and facial dysmorphism consisting of elongated facies, downward-slanting palpebral fissures, mild hypertelorism, flat, broad nasal root, palmoplantar keratosis, ichthyosis, psychomotor retardation, hypoplastic optic discs, sensorineural deafness, etc.

Other aspects of the present disclosure relate to a method of providing prophylactic, palliative, or therapeutic relief to one or more signs or symptoms of X-linked ichthyosis in a subject in need thereof comprising administering to the subject an effective amount of any of the recombinant nucleic acids, viruses, medicaments, and/or pharmaceutical compositions or formulations described herein. In some embodiments, the subject is a human. In some embodiments, the subject's genome comprises a mutation (e.g., a loss-of-function mutation, a pathogenic variant) in the STS gene (one or both copies). In some embodiments, the recombinant nucleic acid (e.g., a recombinant herpes virus genome) comprises one or more polynucleotides encoding a Steryl-sulfatase polypeptide (STS), e.g., a human STS polypeptide. Signs and/or symptoms of X-linked ichthyosis may include, but are not limited to, brownish scales that adhere to the skin (often affecting the back and legs), comma-shaped corneal opacities, etc.

Other aspects of the present disclosure relate to a method of providing prophylactic, palliative, or therapeutic relief to one or more signs or symptoms of arthrogryposis-renal dysfunction-cholestasis (ARC) syndrome in a subject in need thereof comprising administering to the subject an effective amount of any of the recombinant nucleic acids, viruses, medicaments, and/or pharmaceutical compositions or formulations described herein. In some embodiments, the subject is a human. In some embodiments, the subject's genome comprises a mutation (e.g., a loss-of-function mutation, a pathogenic variant) in the VPS33B gene (one or both copies). In some embodiments, the recombinant nucleic acid (e.g., a recombinant herpes virus genome) comprises one or more polynucleotides encoding a Vacuolar protein sorting-associated protein 33B polypeptide (VPS33B), e.g., a human VPS33B polypeptide. Signs and/or symptoms of ARC syndrome may include, but are not limited to, congenital joint contractures, renal tubular dysfunction, cholestasis, ichthyosis, central nervous system malformation, platelet anomalies, agenesis of the corpus callosum, deafness, recurrent sepsis, hypothyroidism, nephrogenic diabetes insipidus, etc.

Other aspects of the present disclosure relate to a method of providing prophylactic, palliative, or therapeutic relief to one or more signs or symptoms of restrictive dermopathy in a subject in need thereof comprising administering to the subject an effective amount of any of the recombinant nucleic acids, viruses, medicaments, and/or pharmaceutical compositions or formulations described herein. In some embodiments, the subject is a human. In some embodiments, the subject's genome comprises a mutation (e.g., a loss-of-function mutation, a pathogenic variant) in the ZMPSTE24 gene (one or both copies). In some embodiments, the recombinant nucleic acid (e.g., a recombinant herpes virus genome) comprises one or more polynucleotides encoding a CAAX prenyl protease 1 homolog polypeptide (ZMPSTE24), e.g., a human ZMPSTE24 polypeptide. Signs and/or symptoms of restrictive dermopathy may include, but are not limited to, very tight and thin skin with erosions, scaling, typical facial dysmorphism, arthrogryposis multiplex, fetal akinesia or hypokinesia deformation sequence (FADS), pulmonary hypoplasia, etc.

The recombinant nucleic acids, viruses, medicaments, and/or pharmaceutical compositions or formulations described herein may be administered by any suitable method or route known in the art, including, without limitation, by oral administration, sublingual administration, buccal administration, topical administration, rectal administration, via inhalation, transdermal administration, subcutaneous injection, intradermal injection, intravenous injection, intra-arterial injection, intramuscular injection, intracardiac injection, intraosseous injection, intraperitoneal injection, transmucosal administration, vaginal administration, intravitreal administration, intraorbital administration, subretinal administration, subconjunctival administration (e.g., the use of subconjunctival depots), suprachoroidal administration, intra-articular administration, peri-articular administration, local administration, epicutaneous administration, or any combinations thereof. In some embodiments, the recombinant nucleic acid, virus, medicament, and/or pharmaceutical composition or formulation is administered cutaneously, topically, transdermally, subcutaneously, or intradermally to the subject. In some embodiments, the recombinant nucleic acid, virus, medicament, and/or pharmaceutical composition or formulation is administered topically, transdermally, subcutaneously, or intradermally to the subject. In some embodiments, the recombinant nucleic acid, virus, medicament, and/or pharmaceutical composition or formulation is administered topically, transdermally, or intradermally to the subject. In some embodiments, the recombinant nucleic acid, virus, medicament, and/or pharmaceutical composition or formulation is administered topically to the subject. The present disclosure thus encompasses methods of delivering any of the recombinant nucleic acids, viruses, medicaments, and/or pharmaceutical compositions or formulations described herein to an individual (e.g., an individual having, or at risk of developing, one or more signs or symptoms of congenital ichthyosis).

In some embodiments, the recombinant nucleic acid, virus, medicament, and/or pharmaceutical composition or formulation is administered once to the subject. In some embodiments, the recombinant nucleic acid, virus, medicament, and/or pharmaceutical composition or formulation is administered at least twice (e.g., at least 2 times, at least 3 times, at least 4 times, at least 5 times, at least 10 times, etc.) to the subject. In some embodiments, at least about 1 hour (e.g., at least about 1 hour, at least about 6 hours, at least about 12 hours, at least about 18 hours, at least about 1 day, at least about 2 days, at least about 3 days, at least about 4 days, at least about 5 days, at least about 6 days, at least about 7 days, at least about 15 days, at least about 20 days, at least about 30 days, at least about 40 days, at least about 50 days, at least about 60 days, at least about 70 days, at least about 80 days, at least about 90 days, at least about 100 days, at least about 120 days, etc.) pass between administrations (e.g., between the first and second administrations, between the second and third administrations, etc.). In some embodiments, the recombinant nucleic acid, virus, medicament, and/or pharmaceutical composition or formulation is administered one, two, three, four, five or more times per day to the subject. In some embodiments, the recombinant nucleic acid, virus, medicament, and/or pharmaceutical composition or formulation is administered to one or more affected and/or unaffected areas of the subject.

In some embodiments, one or more portions of the skin of the subject is abraded or made more permeable prior to treatment with a recombinant nucleic acid, virus, medicament, and/or pharmaceutical composition or formulation described herein. Any suitable method of abrading the skin or increasing skin permeability known in the art may be used, including, for example, use of a dermal roller, repeated use of adhesive strips to remove layers of skin cells (tape stripping), scraping with a scalpel or blade, use of sandpaper, use of chemical permeation enhancers or electrical energy, use of sonic or ultrasonic energy, use of light (e.g., laser) energy, use of micron-sized needles or blades with a length suitable to pierce but not completely pass through the epidermis, etc.

### VII. Host Cells

Certain aspects of the present disclosure relate to one or more host cells comprising any of the recombinant nucleic acids described herein. Any suitable host cell (prokaryotic or eukaryotic) known in the art may be used, including, for example: prokaryotic cells including eubacteria, such as Gram-negative or Gram-positive organisms, for example Enterobacteriaceae such as Escherichia (e.g., E. coli), Enterobacter, Erminia, Klebsiella, Proteus, Salmonella (e.g., S. typhimurium), Serratia (e.g., S. marcescans), and Shigella, as well as Bacilli such as B. subtilis and B. licheniformis; fungal cells (e.g., S. cerevisiae); insect cells (e.g., S2 cells, etc.); and mammalian cells, including monkey kidney CV1 line transformed by SV40 (COS-7, ATCC CRL 1651), human embryonic kidney line (293 or 293 cells subcloned for growth in suspension culture), baby hamster kidney cells (BHK, ATCC CCL 10), mouse Sertoli cells (TM4), monkey kidney cells (CV1 ATCC CCL 70), African green monkey kidney cells (VERO-76, ATCC CRL-1587), human cervical carcinoma cells (HELA, ATCC CCL 2), canine kidney cells (MDCK, ATCC CCL 34), buffalo rat liver cells (BRL 3A, ATCC CRL 1442), human lung cells (W138, ATCC CCL 75), human liver cells (Hep G2, HB 8065), mouse mammary tumor (MMT 060562, ATCC CCL51), TRI cells, MRC 5 cells, FS4 cells, human hepatoma line (Hep G2), Chinese hamster ovary (CHO) cells, including DHFR″ CHO cells, and myeloma cell lines such as NS0 and Sp2/0. In some embodiments, the host cell is a human or non-human primate cell. In some embodiments, the host cells are cells from a cell line. Examples of suitable host cells or cell lines may include, but are not limited to, 293, HeLa, SH-Sy5y, Hep G2, CACO-2, A549, L929, 3T3, K562, CHO-K1, MDCK, HUVEC, Vero, N20, COS-7, PSN1, VCaP, CHO cells, and the like.

In some embodiments, the recombinant nucleic acid is a herpes simplex viral vector. In some embodiments, the recombinant nucleic acid is a herpes simplex virus amplicon. In some embodiments, the recombinant nucleic acid is an HSV-1 amplicon or HSV-1 hybrid amplicon. In some embodiments, a host cell comprising a helper virus is contacted with an HSV-1 amplicon or HSV-1 hybrid amplicon described herein, resulting in the production of a virus comprising one or more recombinant nucleic acids described herein. In some embodiments, the virus is collected from the supernatant of the contacted host cell. Methods of generating virus by contacting host cells comprising a helper virus with an HSV-1 amplicon or HSV-1 hybrid amplicon are known in the art.

In some embodiments, the host cell is a complementing host cell. In some embodiments, the complementing host cell expresses one or more genes that are inactivated in any of the viral vectors described herein. In some embodiments, the complementing host cell is contacted with a recombinant herpes virus genome (e.g., a recombinant herpes simplex virus genome) described herein. In some embodiments, contacting a complementing host cell with a recombinant herpes virus genome results in the production of a herpes virus comprising one or more recombinant nucleic acids described herein. In some embodiments, the virus is collected from the supernatant of the contacted host cell. Methods of generating virus by contacting complementing host cells with a recombinant herpes simplex virus are generally described in WO2015/009952 and/or WO2017/176336.

### VIII. Articles of Manufacture or Kits

Certain aspects of the present disclosure relate to an article of manufacture or a kit comprising any of the recombinant nucleic acids, viruses, medicaments, and/or pharmaceutical compositions or formulations described herein. In some embodiments, the article of manufacture or kit comprises a package insert comprising instructions for administering the recombinant nucleic acid, virus, medicament, and/or pharmaceutical composition or formulation to treat an ichthyosis-associated polypeptide deficiency (e.g., in a subject harboring an STS loss-of-function mutation) and/or to provide prophylactic, palliative, or therapeutic relief to one or more signs or symptoms of a congenital ichthyosis.

Suitable containers for the recombinant nucleic acids, viruses, medicaments, and/or pharmaceutical compositions or formulations may include, for example, bottles, vials, bags, tubes, and syringes. The container may be formed from a variety of materials such as glass, plastic (such as polyvinyl chloride or polyolefin), or metal alloy (such as stainless steel or hastelloy). In some embodiments, the container comprises a label on, or associated with the container, wherein the label indicates directions for use. The article of manufacture or kit may further include other materials desirable from a commercial and user standpoint, including other buffers, diluents, filters, needles, syringes, package inserts, and the like.

### EXAMPLES

The present disclosure will be more fully understood by reference to the following examples. It should not, however, be construed as limiting the scope of the present disclosure. It is understood that the examples and embodiments described herein are for illustrative purposes only and that various modifications or changes in light thereof will be suggested to persons skilled in the art, and are to be included within the spirit and purview of this application and scope of the appended claims.

### Example 1: Modified Herpes Simplex Virus Vectors Encoding One or More Ichthyosis-Associated Polypeptides

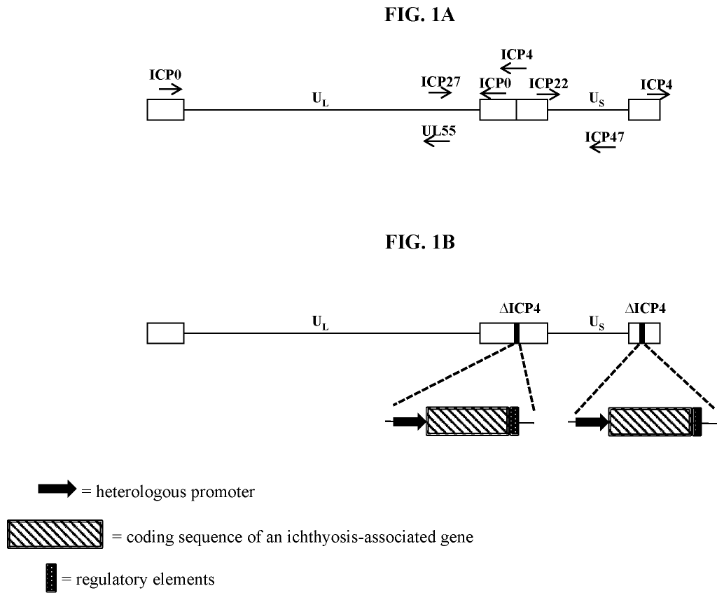

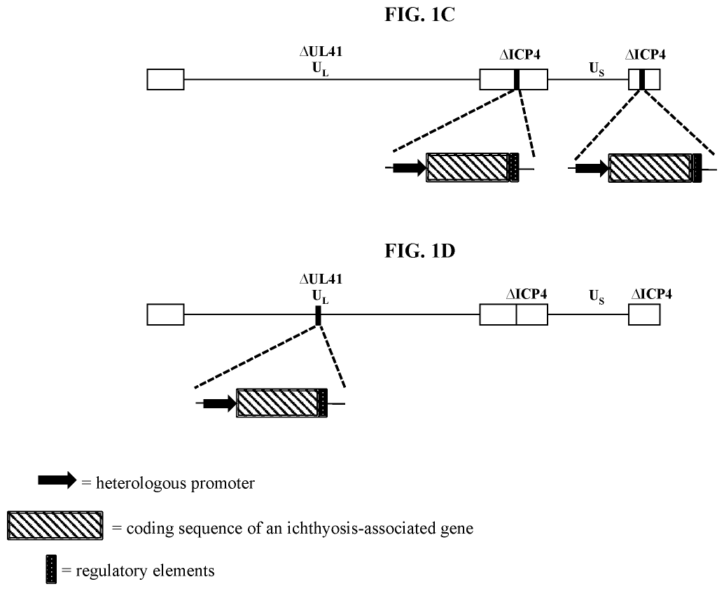

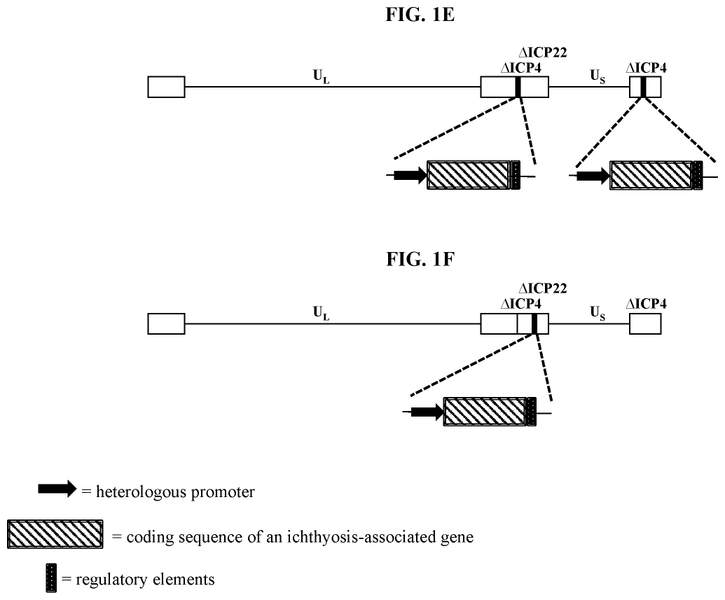

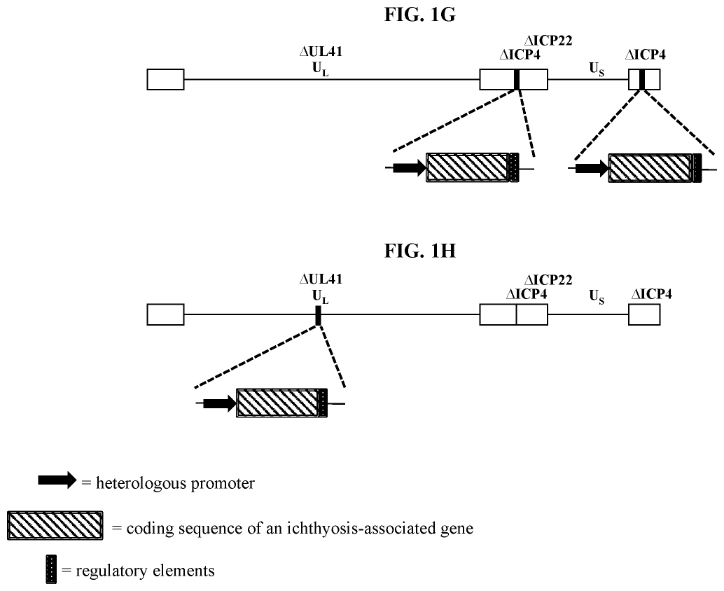

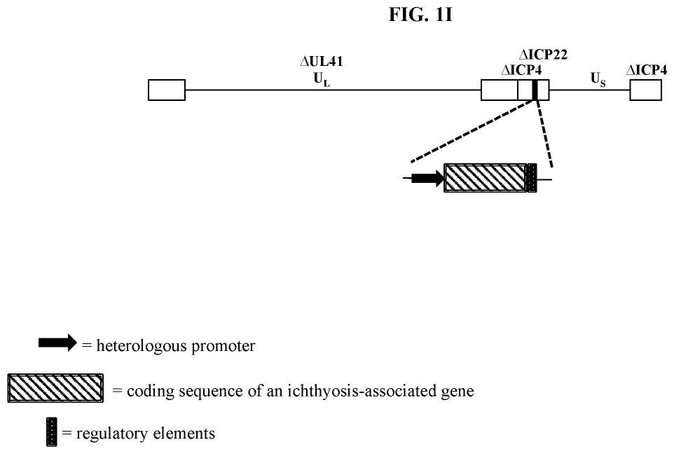

To make modified herpes simplex virus genome vectors capable of expressing functional polypeptides of ichthyosis-associated genes in a target mammalian cell (such as a cell of the skin), a herpes simplex virus genome (FIG. 1A) is first modified to inactivate one or more herpes simplex virus genes. Such modifications may decrease the toxicity of the genome in mammalian cells. Next, variants of these modified/attenuated recombinant viral constructs are generated such that they carry one or more polynucleotides encoding an ichthyosis-associated polypeptide. These variants include: 1) a recombinant ΔICP4-modified HSV-1 genome comprising expression cassettes containing the coding sequence of an ichthyosis-associated polypeptide under the control of a heterologous promoter integrated at each ICP4 locus (FIG. 1B); 2) a recombinant ΔICP4/ΔUL41-modified HSV-1 genome comprising expression cassettes containing the coding sequence of an ichthyosis-associated polypeptide under the control of a heterologous promoter integrated at each ICP4 locus (FIG. 1C); 3) a recombinant ΔICP4/ΔUL41-modified HSV-1 genome comprising an expression cassette containing the coding sequence of an ichthyosis-associated polypeptide under the control of a heterologous promoter integrated at the UL41 locus (FIG. 1D); 4) a recombinant ΔICP4/ΔICP22-modified HSV-1 genome comprising expression cassettes containing the coding sequence of an ichthyosis-associated polypeptide under the control of a heterologous promoter integrated at each ICP4 locus (FIG. 1E); 5) a recombinant ΔICP4/ΔICP22-modified HSV-1 genome comprising an expression cassette containing the coding sequence of an ichthyosis-associated polypeptide under the control of a heterologous promoter integrated at the ICP22 locus (FIG. 1F); 6) a recombinant ΔICP4/ΔUL41/ΔICP22-modified HSV-1 genome comprising expression cassettes containing the coding sequence of an ichthyosis-associated polypeptide under the control of a heterologous promoter integrated at each ICP4 locus (FIG. 1G); 7) a recombinant ΔICP4/ΔUL41/ΔICP22-modified HSV-1 genome comprising an expression cassette containing the coding sequence of an ichthyosis-associated polypeptide under the control of a heterologous promoter integrated at the UL41 locus (FIG. 1H); and 8) a recombinant ΔICP4/ΔUL41/ΔICP22-modified HSV-1 genome comprising an expression cassette containing the coding sequence of an ichthyosis-associated polypeptide under the control of a heterologous promoter integrated at the ICP22 locus (FIG. 1I)

These modified herpes simplex virus genome vectors are transfected or transduced into engineered cells that are modified to express one or more herpes virus genes. These engineered cells secrete into the supernatant of the cell culture a replication defective herpes simplex virus with the modified genomes packaged therein. The supernatant is then collected, concentrated, and sterile filtered through a 5 μm filter.

### Example 2: HSV-TGM1 Pharmacology In Vitro

Congenital ichthyoses are a diverse group of cornification diseases associated with often severe clinical complications and a decreased quality of life. Germline mutations in the TGM1 gene, which encodes the enzyme transglutaminase 1 (TGM1), are the predominant cause of autosomal recessive congenital ichthyosis (ARCI). These TGM1 mutations trigger the abnormal epidermal differentiation and impaired cutaneous barrier function observed in ARCI patients. Unfortunately, like many forms of congenital ichthyoses, current ARCI therapies focus solely on symptomatic relief. Here, the ability of HSV-TGM1, a gene therapy vector encoding full-length human TGM1, to deliver functional human TGM1 to keratinocytes was investigated.

Purified HSV-TGM1 was first evaluated for transduction efficiency and effector expression in two-dimensional cell-based assays. These assays employed immortalized human keratinocytes harvested from a TGM1-deficient ARCI patient homozygous for a c.877-2A>G splice-site mutation, the most commonly reported TGM1 mutation in humans (Herman et al, 2009). Cells were infected with HSV-TGM1 at multiplicities of infection (MOIs) ranging from 0.3 to 3.0 for 48 hours, and vector transduction and effector expression were analyzed by qPCR, qRT-PCR, western blot, and immunofluorescence. Negative controls included uninfected cells (mock) and cells infected with an mCherry-expressing vector (mCherry).

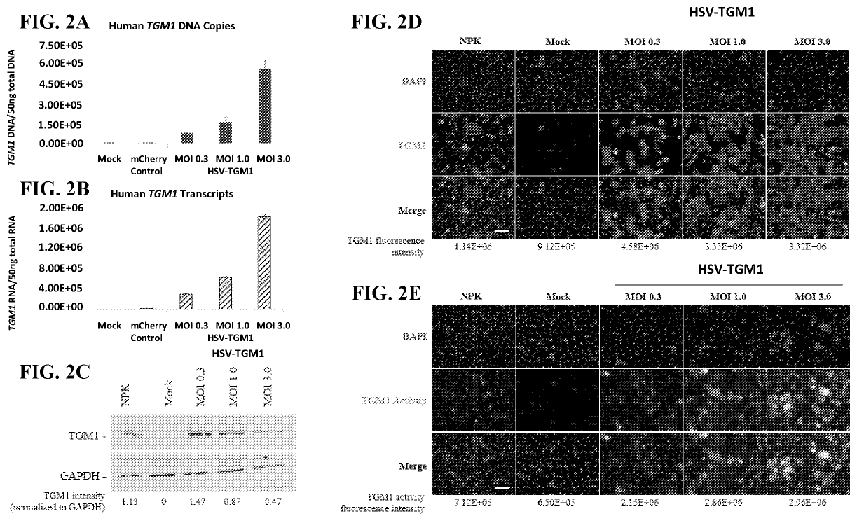

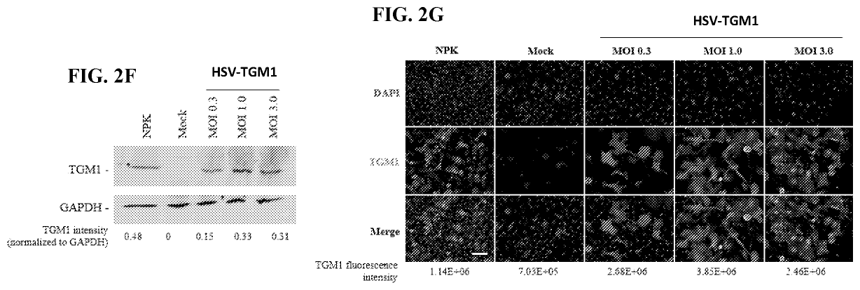

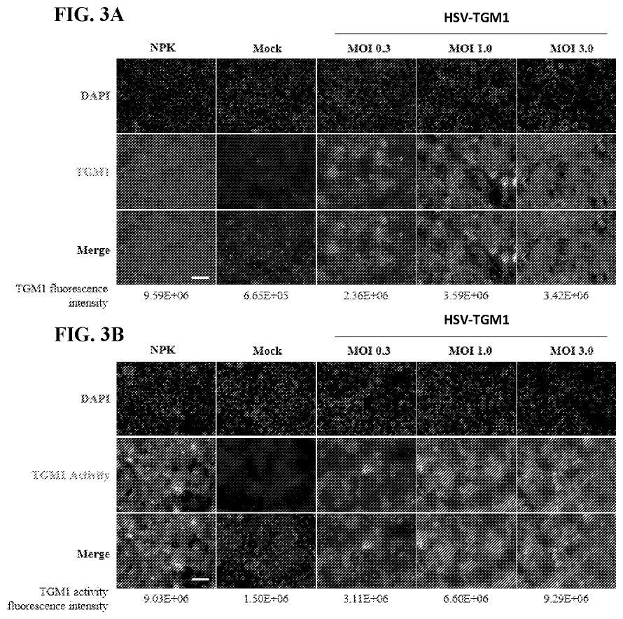

HSV-TGM1 vector genomes and TGM1 transcript expression were detected in TGM1-deficient ARCI patient-derived keratinocytes at an MOI as low as 0.3, and showed a dose-dependent increase in TGM1 DNA (FIG. 2A) and RNA (FIG. 2B) levels. Increased TGM1 protein expression was observed by western blot and immunofluorescent analysis relative to mock-infected controls (FIGS. 2C-2D). No detectable endogenous TGM1 was observed in the uninfected immortalized keratinocytes, confirming that these cells were isolated from a patient harboring a natural TGM1 deficiency.

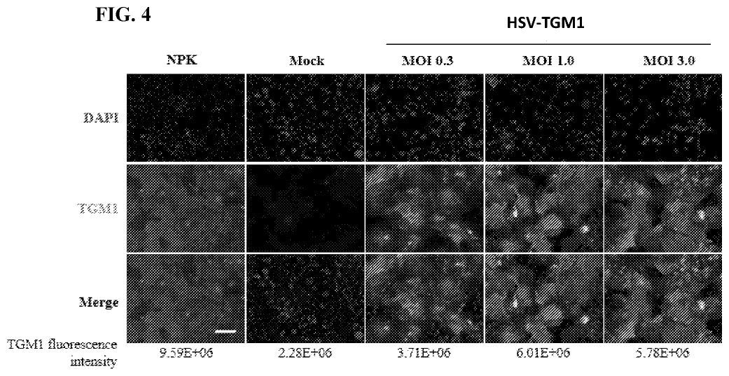

Functionality of the HSV-TGM1-expressed human TGM1 was next examined by determining whether the exogenous protein catalyzed covalent cross-linking between glutamine and lysine residues, a function essential for TGM1-mediated assembly of the cornified envelope. Protein functionality was assessed using an in situ TGM1-specific peptide cross-linking activity assay employing a biotinylated peptide that mimics a natural TGM1 substrate. TGM1-mediated conjugation of biotinylated peptides was visualized by incubating the treated cells with fluorescently labelled streptavidin. A dose-dependent increase in TGM1 enzymatic activity was observed in HSV-TGM1-infected cells by immunofluorescence, with TGM1-mediated peptide cross-linking in infected cells surpassing the levels of endogenous TGM1 activity in normal primary keratinocytes (NPKs) (FIG. 2E). Uninfected (mock) cells showed no detectable TGM1 activity. A similar trend in TGM1 protein expression and subsequent restoration of functional activity were observed in immortalized ARCI keratinocytes grown in high calcium medium to stimulate cell differentiation (FIGS. 3A-3B).

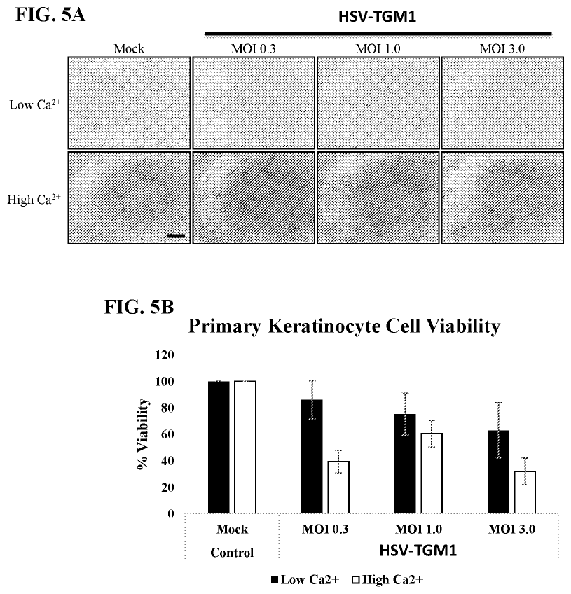

The ability of HSV-TGM1 to transduce a more clinically relevant cell type, i.e., primary TGM1-deficient patient keratinocytes, was next examined. Restoration of TGM1 protein expression was observed by western blot analysis in the HSV-TGM1-infected primary patient cells (FIG. 2F). As expected, no endogenous TGM1 was observed in the negative control primary ARCI keratinocytes. Supporting the western blot, immunofluorescence data revealed a dose-dependent increase in TGM1 protein between an MOI of 0.3 and 1.0 (FIG. 2G). Rescue of TGM1 protein expression in primary patient keratinocytes was also observed by IF after growth in high calcium cell culture medium (FIG. 4). No significant impact on cell morphology was observed upon HSV-TGM1 transduction in low or high calcium environments (FIG. 5A). Mild cytotoxic effects were observed at high dosages of the vector in primary cells, which may account for the decreased TGM1 protein levels observed at an MOI of 3 in the western blot and analyses (FIG. 5B).

In vitro, HSV-TGM1 efficiently infected TGM1-deficient human keratinocytes, produced TGM1 protein, and rescued transglutaminase enzyme function.

### Example 3: In Vivo Evaluation of HSV-TGM1

Because homozygous deletion of TGM1 is neonatal-lethal in mice (Matsuki et al., 1998), a mouse model harboring a genetic lesion in endogenous TGM1 was not practicable for preclinical evaluations. As such, in vivo pharmacology of HSV-TGM1 was conducted in immunocompetent BALB/c mice. This approach allowed determination of the vector's ability to deliver properly localized TGM1 after single or repeated topical administration while concurrently monitoring the vector's toxicity within a fully intact immune system.

First, an evaluation of HSV-TGM1 was conducted in mechanically or chemically disrupted dorsal skin of treated animals. Sequential tape stripping or wiping the skin surface with acetone are commonly used techniques for skin barrier disruption (Rissmann et al, 2009). A single low or high HSV-TGM1 dose formulated in a methylcellulose gel carrier was topically administered to two regions of the prepared skin on each mouse. Skin biopsies were harvested 48 hours after topical administration and processed for analysis.

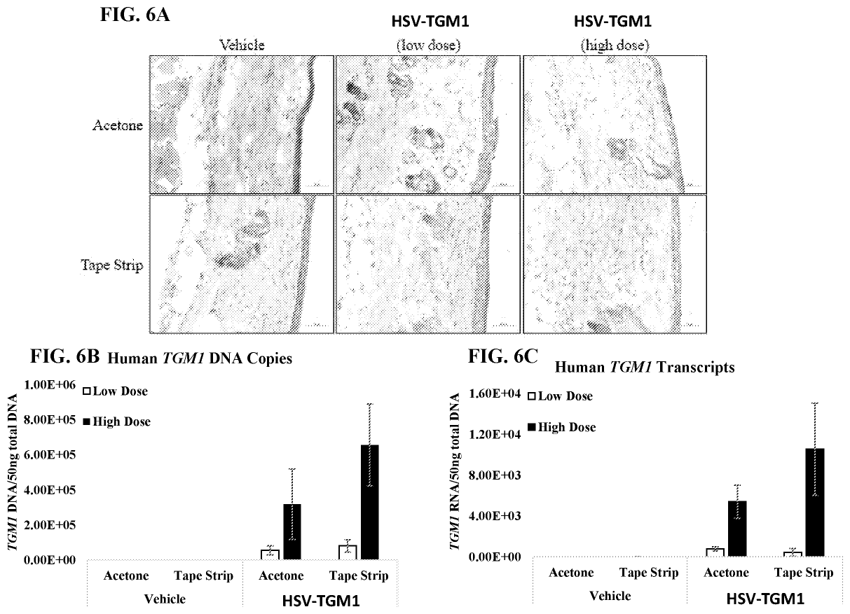

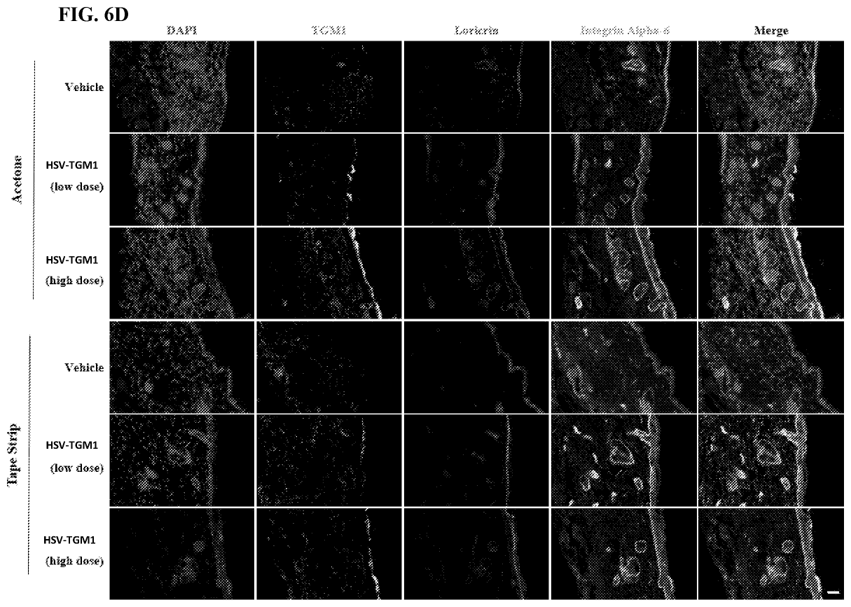

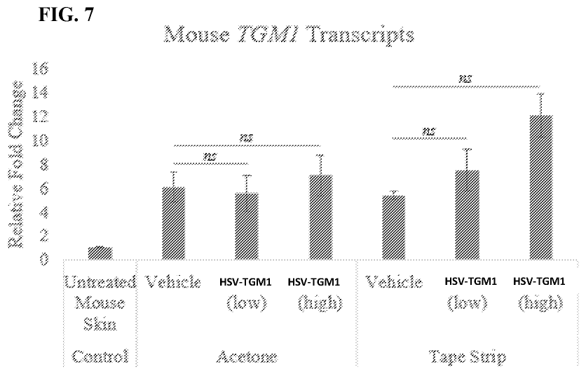

A histological examination of skin samples harvested from each treatment group was conducted to evaluate HSV-TGM1-induced physiological changes, which may indicate potential safety concerns in vivo. No obvious signs of fibrosis, necrosis, or acute inflammation were detected in any HSV-TGM1-treated samples as compared to vehicle control (FIG. 6A). Post-sacrifice quantitative PCR analysis of the topically treated skin indicated that HSV-TGM1 effectively transduced both the acetone treated- and tape strip-permeabilized skin (FIG. 6B), and high levels of human TGM1 transcripts were expressed after infection (FIG. 6C). While acetone treatment or tape stripping of the skin was found to induce endogenous mouse TGM1 transcription, no significant differences in endogenous TGM1 expression were observed between low or high dose HSV-TGM1 and vehicle control (FIG. 7).

Exogenous TGM1 protein expression and tissue localization were assessed by immunofluorescence. Human TGM1 was detected in mouse epidermis upon topical application of HSV-TGM1 (FIG. 6D). Paralleling the qPCR and qRT-PCR results, a qualitative increase in TGM1 protein was observed in the high vs. low dose samples. Samples were also co-stained for mouse loricrin (a natural substrate for TGM1) and mouse integrin alpha-6 (a marker of the basal layer of the epidermis) to determine whether the exogenously expressed TGM1, originating from HSV-TGM1, was correctly localized to the stratum granulosum, the tissue layer where endogenous TGM1 is expressed and functionally active. Mouse loricrin colocalized with human TGM1 in all HSV-TGM1-treated samples, while TGM1 was detected in a more superficial layer than mouse integrin alpha-6. Together, this data demonstrates that HSV-TGM1 successfully transduced the targeted epidermal layer.

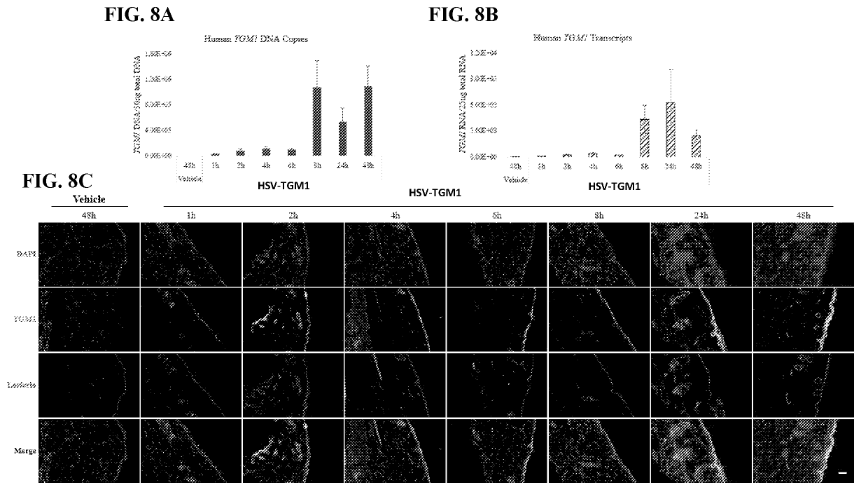

A short-term pharmacokinetics study was conducted in the tape stripped skin of BALB/c mice after topical administration of HSV-TGM1 (FIGS. 8A-8C). Vector genomes in the transduced cells remained relatively stable over the course of the 48-hour study, indicated by similar numbers of human TGM1 DNA copies between the 8- and 48-hour timepoints. Human TGM1 transcripts were detected as early as two hours after topical application, and steadily increased over time, peaking 24 hours after treatment before declining (while remaining detectable) at 48 hours. Similar transgene kinetics were observed at the protein level, as assessed by immunofluorescence.

The safety and feasibility of repeated in vivo vector applications using two different dosing intervals (Days 1 and 3 vs. Days 1 and 12) was next investigated. Briefly, mice were tape stripped and treated topically with HSV-TGM1 (or vehicle control) on Day 1. Skin tissues from a first cohort of animals were harvested on Day 3 to act as positive (HSV-TGM1) and negative (vehicle) controls. Additional animal cohorts were re-tape stripped and re-treated topically with HSV-TGM1 on Days 3 or 12, with tissues subsequently harvested on Days 5 and 14, respectively.

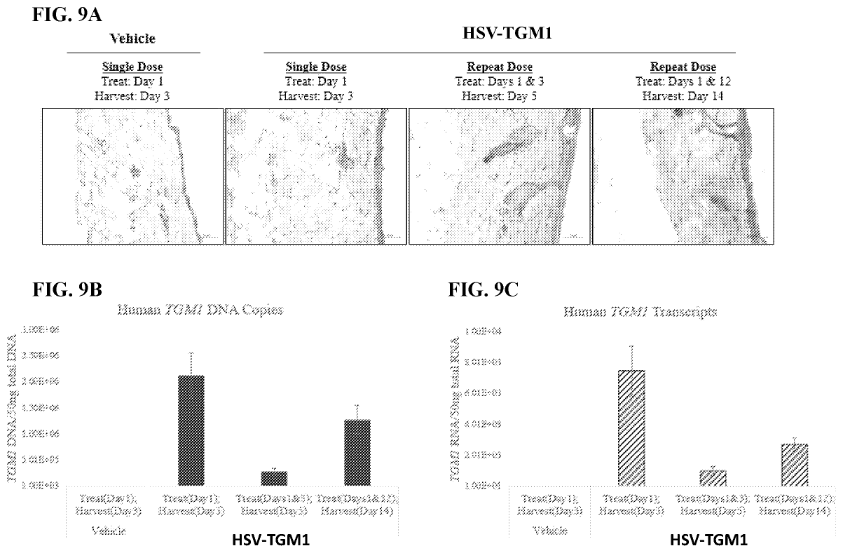

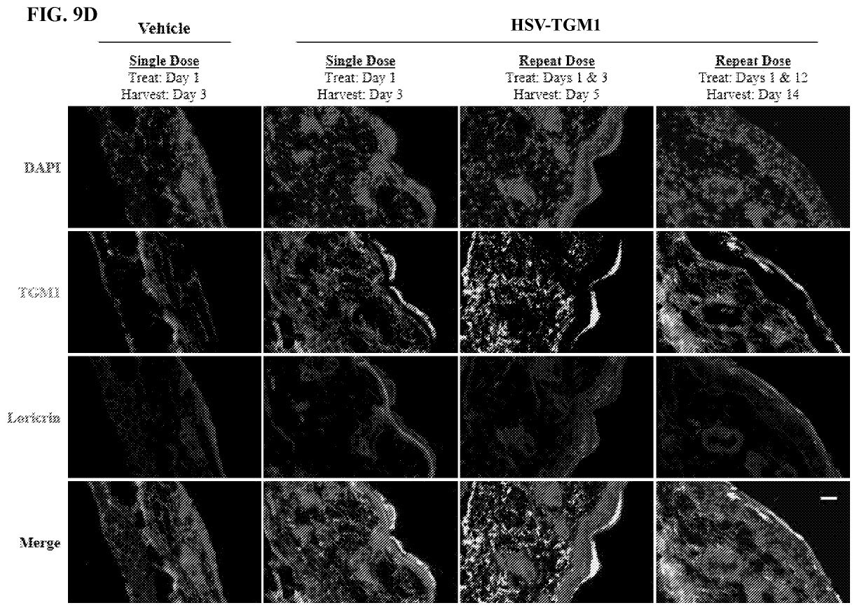

Histological examination of skin samples found no obvious signs of toxicity or tissue reorganization, including fibrosis, necrosis, or acute inflammation, in any of the single or repeat HSV-TGM1-treated samples (FIG. 9A). High vector genome copy numbers were detected 48 hours after a single administration of HSV-TGM1; comparable genome copy numbers were also detected 48 hours after a repeated HSV-TGM1 dose administered at either Day 3 or Day 12 (FIG. 9B). Similar human TGM1 transcript levels were detected after 48 hours in animals receiving a single HSV-TGM1 dose on Day 1 or a second dose on Days 3 or 12 (FIG. 9C).

Human TGM1 protein levels were qualitatively measured. Epidermal localization was assessed by colocalization with mouse loricrin. Significant levels of TGM1 protein, as well as proper epidermal localization, were detected in skin tissue biopsies harvested from mice treated either once or twice with HSV-TGM1 (FIG. 9D). While some variability was observed in TGM1 transcript numbers in these mouse cohorts, no gross differences in TGM1 protein expression were observed by immunofluorescence after single vs. repeat administration of HSV-TGM1.

Toxicity and biodistribution of HSV-TGM1 were evaluated in a Good Laboratory Practice (GLP) repeat-dose study in male and female BALB/c mice (Table 1). Animals were dosed once a week for five weeks with topical HSV-TGM1 (group 2) or vehicle control gel (group 1) after skin permeabilization via tape stripping. Six animals/sex/group were administered one dose on Day 1 and necropsied on Day 3. Six animals/sex/group were dosed on Days 1, 8, 15, 22, and 29 and necropsied on Day 30. All remaining surviving animals were dosed on Days 1, 8, 15, 22, and 29, and were then subjected to a 33-day recovery phase before necropsy.

TABLE 1
Design of the GLP repeat-dose biodistribution and toxicity study

| | No. of Animals^{a} | | Dose Level |
|---|---|---|---|
| Group No. | Male | Female | (PFU/application) |
| 1 (Vehicle Control)^{b} | 18 | 18 | 0 |
| 2 (HSV-TGM1) | 18 | 18 | 1.07 × 10⁹ |

PFU = plaque forming unit
^{a}Animals designated for interim sacrifice (six animals/sex/group) were euthanized on Day 3 of the dosing phase. Animals designated for terminal necropsy (six animals/sex/group) were euthanized on Day 30 of the dosing phase. All remaining surviving animals underwent a 33-day recovery phase following completion of dose administration and were euthanized on Day 34 of the recovery phase (Day 63 of the dosing phase).
^{b}Group 1 was administered vehicle control formulated in gel excipient only.

Assessment of toxicity was based on mortality, clinical observations, body weights, food consumption, dermal observations, and clinical and anatomic pathology. No HSV-TGM1-related mortality, clinical observations, body weight or food consumption changes, macroscopic findings, or effects on organ weight parameters were noted. All animals survived until their scheduled necropsy. Microscopic examination was limited to select tissues, including application/dose site, sternum with bone, bone marrow, brain, epididymis, heart, kidneys, liver, lungs, axillary lymph node, inguinal lymph node, ovaries, oviducts, prostate, spleen, testes, thymus, and uterus with cervix. Microscopic findings localized to the treated skin site, including hyperkeratosis, epithelial hyperplasia, inflammation, edema, and fibrosis, were evaluated at the interim and terminal sacrifices. These findings were consistent with repair following abrasion and were considered related to the dosing procedure. At the recovery sacrifice, most animals exhibited complete reversibility of the dosing procedure-related microscopic findings. As such, the no observed adverse effect level (NOAEL) for topical application of HSV-TGM1 was found to be 1.07×10⁹ PFU/day.

Blood and tissue samples were analyzed by qPCR for the determination of HSV-TGM1 biodistribution. Nearly all blood and tissue samples collected from the vehicle control animals (Group 1) over the three intervals were negative for HSV-TGM1, except for six dose site samples. A root cause analysis indicated that a contamination occurred during the preparation of vehicle specifically used for these 6 animals. Detection of high levels of HSV-TGM1 in Group 2 animals was generally limited to the dose site, with no pronounced accumulation of the vector in other analyzed tissues. Vector persistence was minimal, as indicated by low-to-negative detection of vector copies obtained in samples analyzed from recovery phase animals.

In vivo studies demonstrated that both single and repeated topical HSV-TGM1 administration induced TGM1 protein expression in the target epidermal layer without triggering fibrosis, necrosis, or acute inflammation. Toxicity and biodistribution assessments upon repeat-dosing indicated that HSV-TGM1 was well tolerated and restricted to the dose site. Without wishing to be bound by theory, these results provide a proof-of-concept for the use of a recombinant herpes virus as a gene therapy platform for safely and non-invasively treating ichthyosis.

1.-116. (canceled)

117. A pharmaceutical composition, comprising:
(a) a herpes simplex virus comprising recombinant herpes simplex virus genome, wherein the recombinant herpes simplex virus genome comprises one or more polynucleotides encoding an ichthyosis-associated polypeptide; and
(b) a pharmaceutically acceptable excipient,
wherein the ichthyosis-associated polypeptide is selected from the group consisting of a 1-acylglycerol-3-phosphate O-acyltransferase ABHD5 polypeptide (ABHD5), an Aldehyde dehydrogenase family 3 member A2 polypeptide (ALDH3A2), an Arachidonate 12-lipoxygenase 12R-type polypeptide (ALOX12B), a Hydroperoxide isomerase ALOXE3 polypeptide (ALOXE3), an AP-1 complex subunit sigma-1A polypeptide (AP1S1), an Arylsulfatase E polypeptide (ARSE), a Caspase-14 polypeptide (CASP14), a Corneodesmosin polypeptide (CDSN), a Ceramide synthase 3 polypeptide (CERS3), a Carbohydrate sulfotransferase 8 polypeptide (CHST8), a Claudin-1 polypeptide (CLDN1), a Cystatin-A polypeptide (CSTA), a Cytochrome P450 4F22 polypeptide (CYP4F22), a 3-beta-hydroxysteroid-Delta(8),Delta(7)-isomerase polypeptide (EBP), an Elongation of very long chain fatty acids protein 4 polypeptide (ELOVL4), a Filaggrin polypeptide (FLG), a Filaggrin 2 polypeptide (FLG2), a Gap junction beta-2 polypeptide (GJB2), a Gap junction beta-3 polypeptide (GJB3), a Gap junction beta-4 polypeptide (GJB4), a Gap junction beta-6 polypeptide (GJB6), a 3-ketodihydrosphingosine reductase polypeptide (KDSR), a Keratin, type II cytoskeletal 1 polypeptide (KRT1), a Keratin, type II cytoskeletal 2 epidermal polypeptide (KRT2), a Keratin, type I cytoskeletal 9 polypeptide (KRT9), a Keratin, type I cytoskeletal 10 polypeptide (KRT10), a Lipase member N polypeptide (LIPN), a Loricrin polypeptide (LOR), a Membrane-bound transcription factor site-2 protease polypeptide (MBTPS2), a Magnesium transporter NIPA4 polypeptide (NIPAL4), a Sterol-4-alpha-carboxylate 3-dehydrogenase, decarboxylating polypeptide (NSDHL), a Peroxisomal targeting signal 2 receptor polypeptide (PEX7), a D-3-phosphoglycerate dehydrogenase polypeptide (PHGDH), a Phytanoyl-CoA dioxygenase, peroxisomal polypeptide (PHYH), Patatin-like phospholipase domain-containing protein 1 polypeptide (PNPLA1), a Proteasome maturation protein polypeptide (POMP), a Phosphoserine aminotransferase polypeptide (PSAT1), a Short-chain dehydrogenase/reductase family 9C member 7 polypeptide (SDR9C7), a Serpin B8 polypeptide (SERPINB8), a Long-chain fatty acid transport protein 4 polypeptide (SLC27A4), a Synaptosomal-associated protein 29 polypeptide (SNAP29), a Suppressor of tumorigenicity 14 protein polypeptide (ST14), a Steryl-sulfatase polypeptide (STS), a Sulfotransferase 2B1 polypeptide (SULT2B1), a Vacuolar protein sorting-associated protein 33B polypeptide (VPS33B), and a CAAX prenyl protease 1 homolog polypeptide (ZMPSTE24).

118. The pharmaceutical composition of claim 117, wherein the herpes simplex virus comprising the recombinant herpes simplex virus genome is replication defective.

119. The pharmaceutical composition of claim 117, wherein the recombinant herpes simplex virus genome is a recombinant type 1 herpes simplex virus (HSV-1) genome, a recombinant type 2 herpes simplex virus (HSV-2) genome, or any derivatives thereof.

120. The pharmaceutical composition of claim 119, wherein the recombinant herpes simplex virus genome is a recombinant type 1 herpes simplex virus (HSV-1) genome.

121. The pharmaceutical composition of claim 117, wherein the recombinant herpes simplex virus genome has been engineered to reduce or eliminate expression of one or more toxic herpes simplex virus genes.

122. The pharmaceutical composition of claim 117, wherein the recombinant herpes simplex virus genome comprises an inactivating mutation in a herpes simplex virus gene selected from the group consisting of Infected Cell Protein (ICP) 0, ICP4, ICP22, ICP27, ICP47, thymidine kinase (tk), Long Unique Region (UL) 41, and UL55.

123. The pharmaceutical composition of claim 117, wherein the recombinant herpes simplex virus genome does not comprise a polynucleotide encoding a Collagen alpha-1 (VII) chain polypeptide, a Lysyl hydroxylase 3 polypeptide, and a Keratin type I cytoskeletal 17 polypeptide.

124. The pharmaceutical composition of claim 117, wherein the ichthyosis-associated polypeptide is not a transglutaminase (TGM) polypeptide.

125. The pharmaceutical composition of claim 117, wherein the recombinant herpes simplex virus genome comprises an inactivating mutation in one or both copies of the ICP4 gene.

126. The pharmaceutical composition of claim 117, wherein the recombinant herpes simplex virus genome comprises an inactivating mutation in the ICP22 gene.

127. The pharmaceutical composition of claim 117, wherein the recombinant herpes simplex virus genome comprises the one or more polynucleotides encoding the ichthyosis-associated polypeptide within one or both of the ICP4 viral gene loci.

128. The pharmaceutical composition of claim 117, wherein the recombinant herpes simplex virus genome comprises the one or more polynucleotides encoding the ichthyosis-associated polypeptide within the ICP22 viral gene locus.

129. The pharmaceutical composition of claim 117, wherein the recombinant herpes simplex virus genome comprises the one or more polynucleotides encoding the ichthyosis-associated polypeptide within the UL41 viral gene locus, within one or both of the ICP0 viral gene loci, within the ICP27 viral gene locus, within the ICP47 viral gene locus, and/or within the UL55 viral gene locus.

130. The pharmaceutical composition of claim 117, wherein the ichthyosis-associated polypeptide comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOS: 104-152 or 155.

131. A method of providing prophylactic, palliative, or therapeutic relief to one or more signs or symptoms of congenital ichthyosis in a subject in need thereof, the method comprising administering to the subject an effective amount of a pharmaceutical composition comprising:
(a) a herpes simplex virus comprising recombinant herpes simplex virus genome, wherein the recombinant herpes simplex virus genome comprises one or more polynucleotides encoding an ichthyosis-associated polypeptide; and
(b) a pharmaceutically acceptable excipient,
wherein the ichthyosis-associated polypeptide is selected from the group consisting of a 1-acylglycerol-3-phosphate O-acyltransferase ABHD5 polypeptide (ABHD5), an Aldehyde dehydrogenase family 3 member A2 polypeptide (ALDH3A2), an Arachidonate 12-lipoxygenase 12R-type polypeptide (ALOX12B), a Hydroperoxide isomerase ALOXE3 polypeptide (ALOXE3), an AP-1 complex subunit sigma-1A polypeptide (AP1S1), an Arylsulfatase E polypeptide (ARSE), a Caspase-14 polypeptide (CASP14), a Corneodesmosin polypeptide (CDSN), a Ceramide synthase 3 polypeptide (CERS3), a Carbohydrate sulfotransferase 8 polypeptide (CHST8), a Claudin-1 polypeptide (CLDN1), a Cystatin-A polypeptide (CSTA), a Cytochrome P450 4F22 polypeptide (CYP4F22), a 3-beta-hydroxysteroid-Delta(8),Delta(7)-isomerase polypeptide (EBP), an Elongation of very long chain fatty acids protein 4 polypeptide (ELOVL4), a Filaggrin polypeptide (FLG), a Filaggrin 2 polypeptide (FLG2), a Gap junction beta-2 polypeptide (GJB2), a Gap junction beta-3 polypeptide (GJB3), a Gap junction beta-4 polypeptide (GJB4), a Gap junction beta-6 polypeptide (GJB6), a 3-ketodihydrosphingosine reductase polypeptide (KDSR), a Keratin, type II cytoskeletal 1 polypeptide (KRT1), a Keratin, type II cytoskeletal 2 epidermal polypeptide (KRT2), a Keratin, type I cytoskeletal 9 polypeptide (KRT9), a Keratin, type I cytoskeletal 10 polypeptide (KRT10), a Lipase member N polypeptide (LIPN), a Loricrin polypeptide (LOR), a Membrane-bound transcription factor site-2 protease polypeptide (MBTPS2), a Magnesium transporter NIPA4 polypeptide (NIPAL4), a Sterol-4-alpha-carboxylate 3-dehydrogenase, decarboxylating polypeptide (NSDHL), a Peroxisomal targeting signal 2 receptor polypeptide (PEX7), a D-3-phosphoglycerate dehydrogenase polypeptide (PHGDH), a Phytanoyl-CoA dioxygenase, peroxisomal polypeptide (PHYH), Patatin-like phospholipase domain-containing protein 1 polypeptide (PNPLA1), a Proteasome maturation protein polypeptide (POMP), a Phosphoserine aminotransferase polypeptide (PSAT1), a Short-chain dehydrogenase/reductase family 9C member 7 polypeptide (SDR9C7), a Serpin B8 polypeptide (SERPINB8), a Long-chain fatty acid transport protein 4 polypeptide (SLC27A4), a Synaptosomal-associated protein 29 polypeptide (SNAP29), a Suppressor of tumorigenicity 14 protein polypeptide (ST14), a Steryl-sulfatase polypeptide (STS), a Sulfotransferase 2B1 polypeptide (SULT2B1), a Vacuolar protein sorting-associated protein 33B polypeptide (VPS33B), and a CAAX prenyl protease 1 homolog polypeptide (ZMPSTE24).

132. The method of claim 131, wherein the congenital ichthyosis is selected from the group consisting of autosomal recessive congenital ichthyosis (ARCI), lamellar ichthyosis (LI), congenital ichthyosiform erythroderma (CIE), Chanarin-Dorfman syndrome (CDS), Sjogren-Larsson syndrome (SLS), mental retardation, enteropathy, deafness, peripheral neuropathy, ichthyosis, and keratoderma (MEDNIK) syndrome, chondrodysplasia punctata 1 (CDPX1), chondrodysplasia punctata 2 (CDPX2), peeling skin syndrome (PSS), neonatal ichthyosis-sclerosing cholangitis (NISCH) syndrome, ichthyosis vulgaris, keratitis-ichthyosis-deafness syndrome (KID), palmoplantar keratoderma (PPK), palmoplantar keratoderma with sensorineural hearing loss (PPK/SNHL), epidermolytic palmoplantar keratoderma (EPPK), erythrokeratodermia variabilis (EKV), Clouston syndrome, progressive symmetric erythrokeratodermia, epidermolytic ichthyosis (EI), superficial epidermolytic ichthyosis (SEI), loricrin keratoderma, ichthyosis follicularis, alopecia, and photophobia (IFAP) syndrome, Olmsted syndrome, congenital hemidysplasia with ichthyosiform nevus and limb defects (CHILD) syndrome, Refsum disease, Neu-Laxova syndrome, keratosis linearis with ichthyosis congenita and sclerosing keratoderma (KLICK) syndrome, ichthyosis prematurity syndrome (IPS), cerebral dysgenesis, neuropathy, ichthyosis, and palmoplantar keratoderma (CEDNIK) syndrome, X-linked ichthyosis, arthrogryposis-renal dysfunction-cholestasis (ARC) syndrome, and restrictive dermopathy.

133. The method of claim 131, wherein the pharmaceutical composition is administered topically, transdermally, subcutaneously, intradermally, orally, intranasally, intratracheally, sublingually, buccally, rectally, vaginally, via inhalation, intravenously, intraarterially, intramuscularly, intracardially, intraosseously, intraperitoneally, transmucosally, intravitreally, subretinally, intraarticularly, peri-articularly, locally, or epicutaneously to the subject.

134. The method of claim 133, wherein the pharmaceutical composition is administered topically, transdermally, or intradermally.

135. The method of claim 131, wherein the herpes simplex virus comprising the recombinant herpes simplex virus genome is replication defective.

136. The method of claim 131, wherein the recombinant herpes simplex virus genome is a recombinant type 1 herpes simplex virus (HSV-1) genome, a recombinant type 2 herpes simplex virus (HSV-2) genome, or any derivatives thereof.

137. The method of claim 131, wherein the recombinant herpes simplex virus genome comprises an inactivating mutation in a herpes simplex virus gene selected from the group consisting of Infected Cell Protein (ICP) 0, ICP4, ICP22, ICP27, ICP47, thymidine kinase (tk), Long Unique Region (UL) 41, and UL55.

138. The method of claim 131, wherein the recombinant herpes simplex virus genome does not comprise a polynucleotide encoding a Collagen alpha-1 (VII) chain polypeptide, a Lysyl hydroxylase 3 polypeptide, and a Keratin type I cytoskeletal 17 polypeptide.

139. The method of claim 131, wherein the ichthyosis-associated polypeptide is not a transglutaminase (TGM) polypeptide.

140. The method of claim 131, wherein the ichthyosis-associated polypeptide comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOS: 104-152 or 155.
